(19) United States
(12) Patent Application Publication
(10) Pub. No.: US 2009/0312817 A1
(43) Pub. Date: Dec. 17, 2009

(54) SYSTEMS AND METHODS FOR ALTERING BRAIN AND BODY FUNCTIONS AND FOR TREATING CONDITIONS AND DISEASES OF THE SAME

(75) Inventors: Richard Hogle, Madison, WI (US); Scott Lederer, Verona, WI (US); Mitchell Eugene Tyler, Madison, WI (US)

Correspondence Address: Casimir Jones, S.C., 2275 DEMING WAY, SUITE 310, MIDDLETON, WI, 53562, US

(73) Assignee: Wicab, Inc., Middleton, WI (US)

(21) Appl. No.: 11/925,393

(22) Filed: Oct. 26, 2007

Related U.S. Application Data
(63) Continuation-in-part of application No. 11/234,635, filed on Sep. 23, 2005.
Application No. 11/234,635 is a continuation-in-part of application No. 10/998,222, filed on Nov. 26, 2004.
(60) Provisional application No. 60/854,676, filed on Oct. 26, 2006.
(60) Provisional application No. 60/525,359, filed on Nov. 26, 2003.
(60) Provisional application No. 60/605,988, filed on Aug. 31, 2004.
(60) Provisional application No. 60/615,305, filed on Oct. 1, 2004.

(51) Int. Cl. A61N 1/04

(52) U.S. Cl. 607/54

(57) ABSTRACT

The present invention relates to systems and methods for management of brain and body functions and sensory perception. For example, the present invention provides systems and methods of sensory substitution and sensory enhancement (augmentation) as well as motor control enhancement. The present invention also provides systems and methods of treating diseases and conditions, as well as providing enhanced physical and mental health and performance through sensory substitution, sensory enhancement, and related effects.

This application claims priority to U.S. Provisional Patent Application Ser. No. 60/854,676, filed Oct. 26, 2006, and is a continuation-in-part of U.S. patent application Ser. No. 11/234,635, filed Sep. 23, 2005, which is a continuation-in-part of U.S. patent application Ser. No. 10/998,222, filed Nov. 26, 2004, which claims priority to U.S. Provisional Patent Application Ser. Nos. 60/525,359 filed Nov. 26, 2003, 60/605,988, filed Aug. 31, 2004, and 60/615,305, filed Oct. 1, 2004. The entire contents of each of the aforementioned applications are hereby expressly incorporated herein by reference.

This invention was made with government support under IIS-0083347 awarded by the National Science Foundation; under R01-EY10019, R43/44-DC04738, and R43/44-EY13487 awarded by the National Institutes of Health; BD-8911 awarded by U.S. Defense Advanced Research Project Agency; and under R44 EY013487-03 and R44 EY013487-03 awarded by the National Eye Institute. The government has certain rights in the invention.

### FIELD OF THE INVENTION

The present invention relates to systems and methods for management of brain and body functions and sensory perception. For example, the present invention provides systems and methods of sensory substitution and sensory enhancement (augmentation) as well as motor control enhancement. The present invention also provides systems and methods for treating diseases and conditions, as well as providing enhanced physical and mental health and performance through sensory substitution, sensory enhancement, and related effects.

### BACKGROUND OF THE INVENTION

The mammalian brain, and the human brain in particular, is capable of processing tremendous amounts of information in complex manners. The brain continuously receives and translates sensory information from multiple sensory sources including, for example, visual, auditory, olfactory, and tactile sources. Through processing, movement, and awareness training, subjects have been able to recover and enhance sensory perception, discrimination, and memory, demonstrating a range of untapped capabilities. What are needed are systems and methods for better expanding, accessing, and controlling these capabilities.

### SUMMARY OF THE INVENTION

The present invention relates to systems and methods for management of brain and body functions as they relate to sensory perception, as well as other brain and body functions. For example, the present invention provides systems and methods of sensory substitution and sensory enhancement as well as motor control enhancement. The present invention also provides systems and methods of treating diseases and conditions, as well as providing enhanced physical and mental health and performance through sensory substitution, sensory enhancement, and related effects.

Experiments conducted during the development of the present invention have demonstrated that machine/brain interfaces may be used to, among other things, permit blind and vision impaired individuals to acquire advanced vision from a video camera or other video source, permit subjects with disabling balance-related conditions to approximate normal body function, permit subjects using surgical devices to feel the environment surrounding the ends of catheters or other medical devices, provide enhanced motor skills, and provide enhanced physical and mental health and sense of well-being. In some embodiments, the present invention provides methods for simulating meditative and stress relief benefits without the need for intense meditation training, concentration, and time commitment.

The present invention provides a wide range of systems and methods that allow sensory substitution, sensory enhancement, motor enhancement, and general physical and mental enhancement for a wide variety of application, including but not limited to, treating diseases, conditions, and states that involve the loss or impairment of sensory perception; researching sensory processes; diagnosing sensory diseases, conditions, and states; providing sensory enhanced entertainment (e.g., television, music, movies, video games); providing new senses (e.g., sensation that perceives chemicals, radiation, etc.); providing new communications methods; providing remote sensory control of devices; providing navigation tools; enhancing athletic, job, or general performance; and enhancing physical and mental well-being.

The benefits described herein are obtained, in some embodiments, through the transmission of information to a subject through a sensory route that is not normally associated with such information. For example, in the case of balance improvement, a physical sensor may be used to detect the physical position of the head or body of a subject with respect to the gravity vector. This information is sent to a processor that then encodes and transmits the information, for example, to a transducer array (e.g., stimulator array). The transducer array is contacted with the body of the subject in a manner that provides sensory stimulation (and thus, information)—for example, electrical stimulation on the tongue of the subject. The transducer array is configured such that different head or body perceptions trigger different stimulation to the subject. Through the use of training exercises that permit the subject to associate these patterns with head, body part, or body position, the subject learns to perceive, without conscious thought, the orientation of that body part relative to earth referenced gravity as it is relayed to their brain through their tongue. Experiments conducted during the development of the present invention demonstrated that subjects gained the ability to walk normally and carry out other balance functions (e.g., riding a bicycle) that were impossible without the addition of the new sense. Surprisingly, it was found that the brain became effectively reprogrammed for balance, as subjects were able to maintain the benefit after removal of the device. In a long-term study, true rehabilitation was observed, as benefits (e.g., improved balance) were maintained weeks after use of the device and training were discontinued. Thus, the systems of the present invention not only provide a means for sensory enhancement and substitution, but also provide a means to train the brain to function at a higher level, even in the absence of the device.

Experiment conducted during the development of the invention also demonstrated that the brain is able to integrate and extrapolate the new sensory information in complex ways, including integration with other senses, the ability to react on instinct to the new sensory information, and the ability to extrapolate the information beyond the complexity level actually received from the electrode array. For example, experiments conducted during the development of the invention demonstrated the ability of blind subjects to catch a rolling ball, a task that involves not only seeing the ball, but also coordinating arm movement with a visual cue in a natural manner.

Surprisingly, the system and methods of the present invention provide enhanced brain function that is not directly tied to the specific information provided by the methods. For example, Example 20 describes the treatment of a subject suffering from spasmodic dysphonia who was unable to speak normally prior to treatment, having his oral communication reduced to a whisper. The subject underwent treatment whereby information related to body position and orientation in space was transmitted to the subject's tongue via electrotactile stimulation while the subject maintained body position. The subject was asked to attempt to vocalize during training. Following training, the subject regained the ability produce vocalized speech. Thus, electrotactile information corresponding to body position with respect to the gravitational plane, in conjunction with activation of brain activity associated with speech, was used to increase brain function related to muscle control of the larynx (a motor control function). This example demonstrates that the systems and methods of the present invention find use in general brain function enhancement through the use of, for example, electrotactile stimulation associated with activation of specific brain activity. While an understanding of the mechanism is not necessary to practice the present invention and while the present invention is not limited to any particular mechanism of action, it is contemplated that the use of tactile stimulation (e.g., electrotactile stimulation of the tongue) conditions the brain for improving general function (e.g., motor control, vision, hearing, balance, tactile sensation) associated with a specific task and in general. While an understanding of the mechanism is not necessary to practice the present invention and while the present invention is not limited to any particular mechanism of action, it is contemplated that the systems and methods of the present invention provide or simulate long-term potentiation (long-lasting increase in synaptic efficacy which follows high-frequency stimulation) to provide enhanced brain function. The residual and rehabilitative effect of training seen in experiments conducted during the development of the present invention upon prolonged stimulation is consistent with long-term potentiation studies. Thus, the present invention provides systems and methods for physiological learning that extends for long periods of time (e.g., hours, days, weeks, etc.).

It is further contemplated that the tactile stimulation of the present invention (e.g., electrotactile stimulation of the tongue) provides benefits similar to those achieved by deep brain stimulation methods, and finds use in application where deep brain stimulation is used and is contemplated for use. Chronic deep brain stimulation in its present U.S. FDA-approved manifestation is a patient-controlled treatment for tremor that consists of a multi-electrode lead implanted into the ventrointermediate nucleus of the thalamus. The lead is connected to a pulse generator that is surgically implanted under the skin in the upper chest. An extension wire from the electrode lead is threaded from the scalp area under the skin to the chest where it is connected to the pulse generator. The wearer passes a hand-held magnet over the pulse generator to turn it on and off. The pulse generator produces a high-frequency, pulsed electric current that is sent along the electrode to the thalamus. The electrical stimulation in the thalamus blocks the tremor. The pulse generator must be replaced to change batteries. Risks of DBS surgery include intracranial bleeding, infection, and loss of function. The non-invasive systems and methods of the present invention provide alternatives to invasive deep-brain stimulation for the range of current and future deep-brain stimulation applications (e.g., treatment of tremors in Parkinson's patients, dystonia, essential tremor, chronic nerve-related pain, improved strength after stroke or other trauma, seizure disorders, multiple sclerosis, paralysis, obsessive-compulsive disorders, and depression). While an understanding of the mechanism is not necessary to practice the present invention and while the present invention is not limited to any particular mechanism of action, it is contemplated that the systems and methods of the present invention activate portions of the brain stem and mid-brain that are activated by deep-brain stimulation (e.g., by providing electrotactile stimulation to the tongue).

The present invention further provides systems and methods for enhancing the ability of the brain to utilize damaged tissue to accomplish tasks that it had lost the ability to accomplish or to acquire such abilities that were never previously accomplished. Experiments conducted during the development of the present invention demonstrated that damaged tissues, upon training using the systems and methods of the present invention had enhanced residual ability to re-acquire higher function. Thus, in some embodiments, the systems and methods of the present invention are used to regenerate function from damaged tissue by re-training the brain.

The systems and methods of the present invention may also be used in conjunction with other devices, aids, or methods of sensory enhancement to provide further enhancement or substitution. For example, subjects using cochlear implants, hearing aids, etc. may further employ the systems and methods of the present invention to produce improved function. The systems and methods of the present invention also find use with other devices, systems and methods used for neural monitoring (e.g., the NeuroPort™ System, disclosed in U.S. Pat. App. No. 20040249302, herein incorporated by reference in its entirety for all purposes). The systems and methods of the present invention also find use in combination with other forms of therapy, including, but not limited to rehabilitative therapy (e.g., physical therapy) following, among other thing, traumatic brain injury, stroke or onset of disease (e.g., Parkinson's disease, Alzheimer's disease, neurodegenerative disease, etc.).

Thus, the present invention provides a wide array of devices, software, systems, methods, and applications for treating diseases and conditions, as well as providing enhanced physical and mental health and performance.

In some embodiments, the present invention provides devices, software, systems, methods, and applications related to vestibular function. For example, the present invention provides a method for altering a subject's physical or mental performance related to a vestibular function, comprising: exposing the subject to tactile stimulation under conditions such that said physical or mental performance related to a vestibular function is altered (e.g., enhanced or reduced).

The present invention is not limited by the nature of the vestibular function. In some embodiments, the vestibular function comprises balance. Balance includes all types of balance, such as perception of body orientation with respect to the gravitational plane, to another body part, or to an environmental object (e.g., in low to no gravity environments, under water, etc.)

The present invention is also not limited by the nature of the subject. The subject may be healthy or may suffer from a disease or condition directly or indirectly related to vestibular function. For healthy subjects, the systems and methods of the present invention find use in enhancing vestibular function (e.g., balance) over normal. Athletes, soldiers, and others can benefit from such super-stability.

In some embodiments, the subject has a disease or condition. In some embodiments, the disease or condition is associated with a dysfunction of sensory-motor coordination. In some embodiments, the disease or condition is associated with vestibular function damage, including both peripheral nervous system dysfunction and central nervous system dysfunction. Subjects having a variety of diseases and conditions benefit from the systems and methods of the present invention, including subjects having, or predisposed to, unilateral or bilateral vestibular dysfunction, epilepsy, dyslexia, Meniere's disease, migraines, Mal de Debarquement syndrome, oscillopsia, autism, traumatic brain injury, Parkinson's disease, and tinnitus. The present invention finds use with subjects in a recovery period from a disease, condition, or medical intervention, including, but not limited to, subjects that have suffered traumatic brain injury (e.g., from a stroke) or drug treatment. The systems and methods of the present invention find use with any subject that has a loss of balance or is at risk for loss of balance (e.g., due to age, disease, environmental conditions, etc.).

In some preferred embodiments, the tactile stimulation (e.g., electrotactile stimulation via the tongue) communicates information to the subject, where the information pertains to orientation of the subject's body with respect to the gravitational plane.

The present invention is not limited to treatments that provide tactile information of body position. For example, in some embodiments, treatment and training involves maintaining stabilization of the body (e.g., head) with respect to a reference point (e.g., the gravitational plane) for a period of time (e.g., 10 minutes, 20 minutes, 30 minutes, etc). In some embodiments, the stabilization is facilitated by sensory information (e.g., a video screen) that conveys body position information. In some embodiments, the stabilization is coupled with electrotactile stimulation. In some embodiments, the electrotactile stimulation provides information about body position to the subject. In some embodiments, the position of the head is monitored and provided back to the head of the subject (e.g., via video, audio, tactile information (e.g., on the tongue)).

It is contemplated that, in some embodiments, the systems and methods of the present invention imitate functions of the vestibular system. The vestibular system is located within the head (in the vestibulum in the inner ear) and comprises monitoring components (e.g., semicircular canals that sense/monitor rotational movements and otoliths that sense/monitor linear translations) and information signaling components (e.g., nerves that send signals to the neural structures that control eye movement and to muscles involved in posture). Although an understanding of the mechanism is not necessary to practice the present invention and the present invention is not limited to any particular mechanism of action, in some embodiments, the systems and methods of the present invention provide vestibular-like monitoring components (e.g., balance sensing device) and information signaling components (e.g., arrayed electrotactile stimulation through the tongue) that provide a superior form of treatment because the systems and methods of the present invention use the head (e.g., for monitoring and providing information regarding orientation) to mimic the normal function of the vestibular system. Thus, in some embodiments, systems and methods of the present invention supplement, enhance and/or correct defects in the vestibular system of a subject (e.g., a subject using or being treated with the systems and methods of the present invention).

Experiments conducted during the development of the present invention demonstrated that improvements in vestibular function persisted for a period of time after exposure to tactile stimulation. Improvements were noted over an hour, six hours, twenty-four hours, a week, a month, and six months after exposure to tactile stimulation.

The present invention also provides systems for altering a subject's physical or mental performance related to a vestibular function. The systems find use in the methods described herein. In some preferred embodiments, the system comprises: a) a sensor that collects information related to body position or orientation with respect an environmental reference point; b) a stimulator configured to transmit information (e.g., tactile information) to a subject; and c) a processor configured to: i) receive information from the sensor; ii) convert the information into information to be sent to the subject; and iii) transmit the information to the stimulator in a form that communicates the body position or orientation to the subject. In some preferred embodiments, the sensor is a sensor of angular or linear motion (e.g., an accelerometer or a gyroscope). In some embodiments, the sensor (e.g., accelerometer) is located within the mouth of the subject.

The present invention is not limited by the nature of the stimulator used. In some preferred embodiments, the stimulator is provided on a mount configured to fit into a subject's mouth to permit tactile stimulation to the tongue. In some preferred embodiments, the communication between the processor and the stimulator is via wireless methods. In particular preferred embodiments, the processor is provided in a portable housing to permit a subject to easily transport the processor on or in their body.

The present invention further provides systems for training subjects to correlate tactile information with environmental or other information to be perceived to improve vestibular function. In some preferred embodiments, the system comprises: a) a stimulator configured to transmit tactile information to a subject, and b) a processor configured to i) run a training program that produces an perceivable event that correlates to the subject's body position or orientation, and ii) transmit tactile information to the stimulator in a form that correlates the body position or orientation to the perceivable event (e.g., visualized as a video image on a display screen).

The present invention further provides methods for diagnosing vestibular dysfunction. In some preferred embodiments, the method comprises measuring a skill of a subject associated with vestibular function in response to tactile stimulation. In some embodiments, the measured skill is compared to a predetermined normal skill value to determine increase or decrease in function. The predetermined normal skill value may be obtained from any source, including, but not limited to, population averages and prior measures from the subject. In some preferred embodiments, the skill comprises balance or sway stability. The method finds particular use in detecting vestibular damage during a treatment or procedure, such that, when detected, the treatment regimen may be altered to reduce or eliminate long-term damage. For example, bilateral vestibular dysfunction may be avoided in subjects undergoing treatment with medications (e.g., antibiotics such as gentamycin) that can cause bilateral vestibular dysfunction.

Experiments conducted during the development of the present invention demonstrated that the use of the systems and methods of the present invention provide subjects with the physical or emotional benefits associated with meditation and/or stress relief. Thus, the present invention provides methods comprising the step of contacting a subject with tactile stimulation (e.g., electrotactile stimulation via the tongue) under conditions that provide such benefits. In some embodiments, the subject is provided with 10 or more minutes (e.g., 15 minutes, 20 minutes, 30 minutes, 40 minutes, . . . ) of tactile stimulation. In some embodiments, the subject maintains a controlled body position while receiving tactile stimulation (e.g., upright, straight back; standing position). Exemplary physical and emotional benefits that can be achieved are described herein and include, but are not limited to, improved motor coordination, improved sleep, improved vision, improved cognitive skills, and improved emotional health (e.g., increased sense of well-being).

In some embodiments, the present invention provides a method of providing long-term (e.g., one hour, six hours, one day, one week, one month, six months, etc.) improvement in a brain function, comprising: providing electrotactile stimulation to a tongue of a subject for a period of 10 or more minutes (e.g., 15, 20, 30, 40, . . . ). The present invention is not limited by the nature of the brain function improved. Numerous examples are described herein (e.g., vestibular functions such as balance). In some embodiments, the improvement is achieved wherein the electrotactile stimulation conveys information (e.g., information about a subject's body position in one embodiment of balance improvement applications). In preferred embodiments, the long-term improvement comprises improved brain function after the electrotactile stimulation is discontinued.

In some embodiments, subjects having a disease or condition associated with loss of motor control are treated with the systems and methods of the present invention. For example, experiments conducted during the development of the present invention demonstrated improved ability to speak in a subject having spasmodic dysphonia.

The present invention also provides a sensory substitution device for providing visual information to a subject comprising: a sensor; a portable microcontroller; a device configured for electrical stimulation; and a mobile information gathering, processing, storing and distributing platform. In some embodiments, the device is configured to provide video information captured by the sensor to a subject. In some embodiments, the sensor comprises a video camera. In some embodiments, the portable microcontroller comprises means for controlling the sensor. In some embodiments, the means for controlling the sensor controls a sensor function. The present invention is not limited to any particular sensor function. Indeed, a variety of sensor functions are contemplated including, but not limited to, zoom, contrast, focus and inversion. In some embodiments, the sensor communicates with the mobile platform. In some embodiments, the device configured for electrical stimulation comprises an array of electrodes. In some embodiments, the array of electrodes are present on an intra-oral tongue display unit. In some embodiments, the array of electrodes is configured to provide visual information to a subject via the subject's tongue. In some embodiments, the device further comprises a power supply. In some embodiments, the power supply comprises a battery pack. In some embodiments, the sensor is utilized for infrared vision. In some embodiments, the sensor is utilized for ultraviolet vision. In some embodiments, the device creates a multidimensional electrical image on a subject's tongue.

The present invention also provides a method of providing visual information to a subject comprising: providing: a subject; and a sensory substitution device, wherein the sensory substitution device comprises: a sensor; a portable microcontroller; an device configured for electrical stimulation; and a mobile information gathering, processing, storing and distributing platform; and exposing the subject to the sensory substitution device under conditions such that the subject receives visual information from the sensory substitution device. In some embodiments, the visual information is real-time information regarding the subject's immediate surroundings. In some embodiments, the visual information is recorded information. In some embodiments, the subject is legally blind. In some embodiments, the subject is visually impaired. In some embodiments, the visual information is received from the device configured for electrical stimulation. In some embodiments, the device configured for electrical stimulation is an array of electrodes. In some embodiments, the array of electrodes provide visual information to the subject via the subject's tongue. In some embodiments, the visual information comprises information captured by the sensor that is processed by the mobile platform.

The present invention also provides a method of providing visual information to a visually healthy subject desiring visual stimulation, comprising: providing: a subject; and a sensory substitution device, wherein the sensory substitution device comprises: a device configured for electrical stimulation; and a mobile information gathering, processing, storing and distributing platform; and exposing the subject to the sensory substitution device under conditions such that the subject receives visual information from the sensory substitution device. In some embodiments, the subject desires visual stimulation associated with a video game. In some embodiments, the visual information is received from the device configured for electrical stimulation. In some embodiments, the device configured for electrical stimulation is an array of electrodes. In some embodiments, the array of electrodes provide visual information to the subject via the subject's tongue. In some embodiments, the visual information comprises recorded information present within video game software. In some embodiments, the subject perceives visual information that is not viewed by the subject's eyes.

The present invention also provides a method of providing visual information to a subject, wherein the subject is legally blind, comprising: providing: a subject; and a sensory substitution device, wherein the sensory substitution device comprises: a sensor; a portable microcontroller; an device configured for electrical stimulation; and a mobile information gathering, processing, storing and distributing platform; and exposing the subject to the sensory substitution device under conditions such that the subject receives visual information from the sensory substitution device. In some embodiments, the subject is able to visualize, using the system, images in space. In some embodiments, the subject is able to perform a coordination task selected from the group consisting of facial recognition, determine speed of a moving object, determine direction of movement of an object, and hitting a moving object. In some embodiments, the visual information is received from the device configured for electrical stimulation. In some embodiments, the device configured for electrical stimulation is an array of electrodes. In some embodiments, the array of electrodes provide visual information to the subject via the subject's tongue. In some embodiments, the visual information comprises information captured by the sensor that is processed by the mobile platform. In some embodiments, the sensor is a video camera.

Additional embodiments of the present invention are described below.

### DESCRIPTION OF DRAWINGS

FIG. 1 shows a schematic diagram of information flow to and from the brain.

FIG. 2 shows a schematic diagram of information flow to and from the brain from traditional means, and from employing systems and methods of the present invention.

FIG. 3 shows a schematic diagram of information flow from a video source to the brain using a tongue-based electrotactile system of the present invention.

FIG. 4 shows examples of different types of information that may be conveyed by the systems and methods of the present invention.

FIG. 5 shows a circuit configuration for an enhanced catheter system of the present invention.

FIG. 6 shows a waveform pattern used in some embodiments of the present invention.

FIG. 7 shows a sensor pattern in a surgical probe embodiment of the present invention.

FIG. 8 shows a testing system for testing a surgical probe system of the present invention.

FIG. 9 shows a sensor pattern in a surgical probe embodiment of the present invention.

FIG. 10 shows four trajectory error cues as displayed on the tongue display for use in a navigation embodiments of the present invention: (a) “On course; proceed.” (b) “Translate, step ‘Up’.” (c) “Translate ‘Right’.” (d) Rotate ‘Right’.” Forward motion along trajectory is indicated by flashing of displayed pattern. Black areas on diagrams represent active regions on 12×12 array. Gray arrows indicate direction of image on display.

FIG. 11 shows data from a tongue mapping experiment of the present invention.

FIG. 12 shows data from a tongue mapping experiment of the present invention.

FIG. 13 shows data from a tongue mapping experiment of the present invention.

FIG. 14 shows data from a tongue mapping experiment of the present invention.

FIG. 15 is a simplified perspective view of an exemplary input system wherein an array of transmitters 104 magnetically actuates motion of a corresponding array of stimulators 100 implanted below the skin 102.

FIG. 16 is a simplified cross-sectional side view of a stimulator 200 of a second exemplary input system, wherein the stimulator 200 delivers motion output to a user via a deformable diaphragm 212.

FIG. 17 is a simplified circuit diagram showing exemplary components suitable for use in the stimulator 200 of FIG. 16.

FIG. 18 shows an exemplary in-mouth electrotactile stimulation device of the present invention.

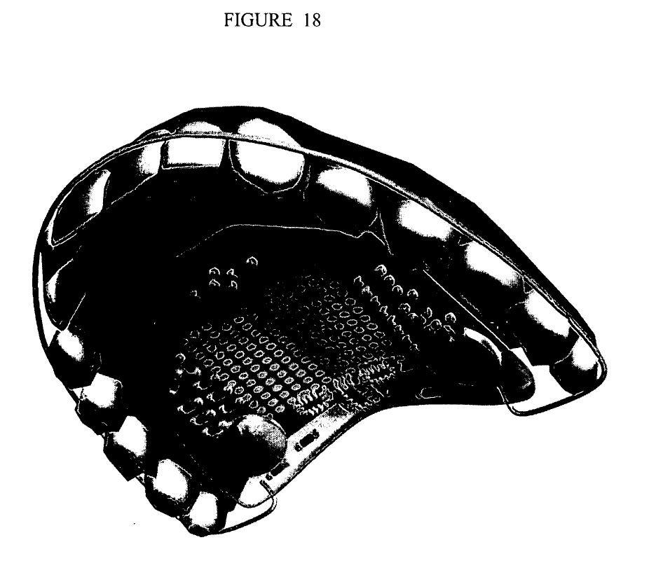

FIG. 19 shows an exemplary in-mouth signal output device of the present invention.

FIG. 20 shows a sample wave-form useful in some embodiments of the present invention.

FIG. 21 shows a power supply unit of some embodiments of the present invention.

FIG. 22 shows a stimulation circuit of some embodiments of the present invention.

FIG. 23 shows a cartoon that provides a general overview of how the brain receives sensory input from the spinal cord as well as from its own (e.g., cranial) nerves.

FIG. 24 shows a cartoon depicting various regions of the brain.

FIG. 25 shows a cartoon of the inner and its two membrane-covered outlets into the air-filled middle ear: the oval window and the round window.

FIG. 26 shows what the cochlea would look like were it to be unrolled.

FIG. 27 shows a picture of the membranous labyrinth.

FIG. 28 shows A) a cartoon of how the auditory nerve carries signal into the brainstem and synapses in the cochlear nucleus and B) how a second stream of information starts in the dorsal cochlear nucleus.

FIG. 29 shows that the auditory nucleus of the thalamus is the medial geniculate nucleus.

FIG. 30 shows a cartoon of A) the semicircular canal and B) how canals on either side of the head will generally be operating in a push-pull rhythm; when one is excited, the other is inhibited.

FIG. 31 shows A) a cartoon of the vestibulo-ocular reflex (VOR) and B) how the reflex functions during motion.

FIG. 32 shows an intraoral device and Controller device of one embodiment of the present invention. FIG. 32A shows a MEMS accelerometer mounted on the back of the tongue electrode array. FIG. 32B shows a 10×10 electrode array. FIG. 32C shows an entire device (e.g., comprising the intraoral device, tether, and controller) in one embodiment of the present invention. FIG. 32D shows a subject wearing one embodiment of a device of the present invention.

FIG. 33 shows a graph of the success rate (percent correct) of the performance of legally blind adults attempting various visual tasks while utilizing a system for providing visual information of the present invention.

FIG. 34 shows a schematic of software used to run a substitute sensory device of one embodiment of the present invention.

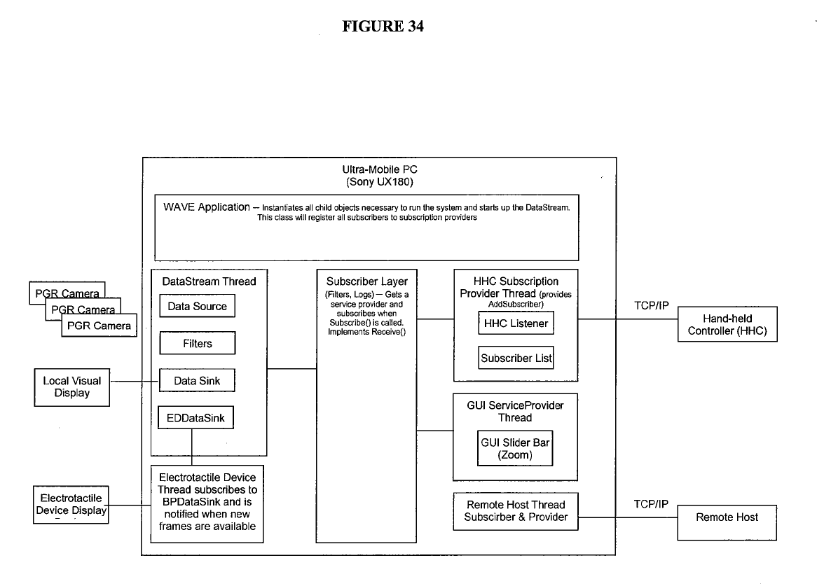

FIG. 35 shows a headband comprising a chain of sensors (e.g., cameras) in one configuration of a sensory substitution device of the present invention.

FIG. 36 shows one embodiment of a sensory substitution device of the present invention comprising (A) a chain of sensors (e.g., cameras); (B) a hand-held component; and (C) a mobile platform (e.g., ultra-compact personal computer).

FIG. 37 shows a sensory substitution device configuration in one embodiment of the present invention.

FIG. 38 shows the effect of vision impairment on quality of life.

FIG. 39 shows a schematic of vision distortion due to MD and ring scotoma caused by a magnifying vision device. Panel A. Normal vision—the entire FOV, especially the gaze point, is in focus. Panel B. Schematic of the same image, showing vision loss in the gaze point due to MD and blurred peripheral vision. Panel C. Image improvement with a magnifier. The magnified view is much larger than the FOV and blocks the view of other cars ahead.

FIG. 40 shows a schematic of how a vision assistance and/or augmentation device of the present invention can help individuals with macular degeneration. Panel A. A person with macular degeneration is unable to read a prescription label. Panel B. A person wearing a vision assistance and/or augmentation device can now read the label.

FIG. 41 shows a 611-pixel electrode array (2.5 cm×2.5 cm) that stimulates the tongue (the tongue display). Panel A. The electrodes that stimulate the tongue. Panel B. The underside of the electrode array faces the roof of the mouth.

FIG. 42 shows a Scotoma map. The darkly shaded areas indicate the regions of vision loss.

FIG. 43 shows a schematic of training/testing setup in one embodiment of the invention. Inset: Schematic of image presented to the tongue display.

### DEFINITIONS

To facilitate an understanding of the present invention, a number of terms and phrases are defined below:

As used herein, the term “subject” refers to a human or other vertebrate animal. It is intended that the term encompass patients.

As used herein, the term “amplifier” refers to a device that produces an electrical output that is a function of the corresponding electrical input parameter, and increases the magnitude of the input by means of energy drawn from an external source (i.e., it introduces gain). “Amplification” refers to the reproduction of an electrical signal by an electronic device, usually at an increased intensity. “Amplification means” refers to the use of an amplifier to amplify a signal. It is intended that the amplification means also includes means to process and/or filter the signal.

As used herein, the term “receiver” refers to the part of a system that converts transmitted waves into a desired form of output. The range of frequencies over which a receiver operates with a selected performance (i.e., a known level of sensitivity) is the “bandwidth” of the receiver.

As used herein, the term “transducer” refers to any device that converts a non-electrical parameter (e.g., sound, pressure or light), into electrical signals or vice versa.

As used herein, the terms “stimulator” and “actuator” are used herein to refer to components of a device that impart a stimulus (e.g., vibrotactile, electrotactile, thermal, etc.) to tissue of a subject. When referenced herein, the term stimulator provides an example of a transducer. Unless described to the contrary, embodiments described herein that utilize stimulators or actuators may also employ other forms of transducers.

The term “circuit” as used herein, refers to the complete path of an electric current.

As used herein, the term “resistor” refers to an electronic device that possesses resistance and is selected for this use. It is intended that the term encompass all types of resistors, including but not limited to, fixed-value or adjustable, carbon, wire-wound, and film resistors. The term “resistance” (R; ohm) refers to the tendency of a material to resist the passage of an electric current, and to convert electrical energy into heat energy.

The term “magnet” refers to a body (e.g., iron, steel or alloy) having the property of attracting iron and producing a magnetic field external to itself, and when freely suspended, of pointing to the magnetic poles of the Earth.

As used herein, the term “magnetic field” refers to the area surrounding a magnet in which magnetic forces may be detected.

As used herein, the term “electrode” refers to a conductor used to establish electrical contact with a nonmetallic part of a circuit, in particular, part of a biological system (e.g., human skin on tongue).

The term “housing” refers to the structure encasing or enclosing at least one component of the devices of the present invention. In preferred embodiments, the “housing” is produced from a “biocompatible” material. In some embodiments, the housing comprises at least one hermetic feedthrough through which leads extend from the component inside the housing to a position outside the housing.

As used herein, the term “biocompatible” refers to any substance or compound that has minimal (i.e., no significant difference is seen compared to a control) to no irritant or immunological effect on the surrounding tissue. It is also intended that the term be applied in reference to the substances or compounds utilized in order to minimize or to avoid an immunologic reaction to the housing or other aspects of the invention. Particularly preferred biocompatible materials include, but are not limited to titanium, gold, platinum, sapphire, stainless steel, plastic, and ceramics.

As used herein, the term “implantable” refers to any device that may be implanted in a patient. It is intended that the term encompass various types of implants. In preferred embodiments, the device may be implanted under the skin (i.e., subcutaneous), or placed at any other location suited for the use of the device (e.g., within temporal bone, middle ear or inner ear). An implanted device is one that has been implanted within a subject, while a device that is “external” to the subject is not implanted within the subject (i.e., the device is located externally to the subject's skin).

As used herein, the term “hermetically sealed” refers to a device or object that is sealed in a manner that liquids or gases located outside the device are prevented from entering the interior of the device, to at least some degree. “Completely hermetically sealed” refers to a device or object that is sealed in a manner such that no detectable liquid or gas located outside the device enters the interior of the device. It is intended that the sealing be accomplished by a variety of means, including but not limited to mechanical, glue or sealants, etc. In particularly preferred embodiments, the hermetically sealed device is made so that it is completely leak-proof (i.e., no liquid or gas is allowed to enter the interior of the device at all).

As used herein the term “processor” refers to a device that is able to read a program from a computer memory (e.g., ROM or other computer memory) and perform a set of steps according to the program. Processor may include non-algorithmic signal processing components (e.g., for analog signal processing).

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape, flash memory, and servers for streaming media over networks.

As used herein the terms “multimedia information” and “media information” are used interchangeably to refer to information (e.g., digitized and analog information) encoding or representing audio, video, and/or text. Multimedia information may further carry information not corresponding to audio or video. Multimedia information may be transmitted from one location or device to a second location or device by methods including, but not limited to, electrical, optical, and satellite transmission, and the like.

As used herein, the term “Internet” refers to any collection of networks using standard protocols. For example, the term includes a collection of interconnected (public and/or private) networks that are linked together by a set of standard protocols (such as TCP/IP, HTTP, and FTP) to form a global, distributed network. While this term is intended to refer to what is now commonly known as the Internet, it is also intended to encompass variations that may be made in the future, including changes and additions to existing standard protocols or integration with other media (e.g., television, radio, etc). The term is also intended to encompass non-public networks such as private (e.g., corporate) Intranets.

As used herein the term “security protocol” refers to an electronic security system (e.g., hardware and/or software) to limit access to processor, memory, etc. to specific users authorized to access the processor. For example, a security protocol may comprise a software program that locks out one or more functions of a processor until an appropriate password is entered.

As used herein the term “resource manager” refers to a system that optimizes the performance of a processor or another system. For example a resource manager may be configured to monitor the performance of a processor or software application and manage data and processor allocation, perform component failure recoveries, optimize the receipt and transmission of data, and the like. In some embodiments, the resource manager comprises a software program provided on a computer system of the present invention.

As used herein the term “in electronic communication” refers to electrical devices (e.g., computers, processors, communications equipment) that are configured to communicate with one another through direct or indirect signaling. For example, a conference bridge that is connected to a processor through a cable or wire, such that information can pass between the conference bridge and the processor, are in electronic communication with one another. Likewise, a computer configured to transmit (e.g., through cables, wires, infrared signals, telephone lines, etc) information to another computer or device, is in electronic communication with the other computer or device.

As used herein the term “transmitting” refers to the movement of information (e.g., data) from one location to another (e.g., from one device to another) using any suitable means.

As used herein, the term “electrotactile” refers to a means whereby sensory channels (e.g., nerves) responsible for sensory functions are stimulated by an electric current. In some embodiments, the term refers to a means by which sensory channels (e.g., nerves) responsible for human touch (and/or taste) perception are stimulated by an electric current (applied via surface (or implanted) electrodes). The term electrotactile may be used interchangeably with the terms “electrocutaneous” and “electrodermal.”

### DETAILED DESCRIPTION OF THE INVENTION

The present invention provides systems and methods for managing sensory information by providing new forms of sensory input to replace, supplement, or enhance sensory perception, motor control, performance of mental and physical tasks, and health and well being. The systems and methods of the present invention accomplish these results by providing sensory input from a device to a subject. The sensory input is provided in a manner such that, through the nature of the input, or through subject training, or a combination thereof, a subject receiving the input receives information and the intended benefit. Thus, the present invention provides a machine-brain interface for the transmission of sensory information (e.g., through the skin). Unlike methods that simply provide physical stimulation of a skin surface, preferred embodiments of the systems and methods of the present invention provide structure to the signal such that information is conveyed to the brain, affecting brain function.

Brain Computer Interface (BCI) technology is one of the most intensely developing areas of modern science and has created numerous significant crossroads between neuroscience and computer science. The goal of BCI technology is to provide a direct link between the human brain and a computerized environment. However, the vast majority of recent BCI approaches and applications have been designed to provide the information flow from the brain to the computerized periphery. The opposite or alternative direction of flow of information (computer to brain interface—CBI) remains almost undeveloped.

The systems of the present invention provide a Computer Brain Interface and other systems and methods for providing information to the brain that offers an alternative symmetrical technology designed to support a direct link from a computerized or machine environment (or from any other system that can provide information about the environment) to the brain and to do it, if desired, non-invasively.

In the majority of modern industrial and technological control processes, the human is still needed “in the loop”—perhaps even more urgently than ever before. This is because the complexity and scale of technologies requiring computer control is increasing in parallel to the exponential development of available computational power. Thus, rather than simplifying the human operator's environment, these advancing technologies make increasingly more complex demands on the operators (e.g., requiring increased interaction with stored memory capacity, increased speed of reaction while maintaining precision of decision making processes and attention to diverse tasks, rapid learning of new knowledge-based skills, etc.). These unavoidable and escalating demands can and do lead to critical psychological pressures on the human mind that can lead to weakening of the human link in the technological chain. The increasing information flow leads to the overloading of the human brain, increasing the risk of human malfunction, ranging, e.g., from decision-making errors to complete psychological break-down of the human operator.

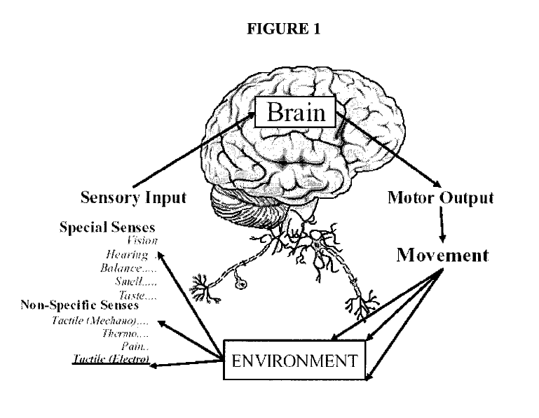

Why does this happen? FIG. 1 shows a simplified sketch of a human operator. In essence, this is an analog of the physical “black box” diagram, where the brain (as a central processing unit) receives inputs from the various sensory systems and generates outputs to various muscular systems (motor output), producing muscular movement. The product of the motor output is then sensed and compared with the original motor plan. Subsequent motor outputs may be generated depending upon how well the resultant movement fit the initial sensory-motor action plan. For the majority of mammals, environmental information input to the brain is typically organized by five special senses and a few non-specific ones. The five special senses are: vision, hearing, balance, smell and taste. They are “special” because the actual sensors (receptors) are localized and specialized (physically, chemically and anatomically) to acquire specific environmental data, but within a limited range of changes. For example, the sensitivity of photoreceptors is limited in terms of wavelength: humans cannot see in the infrared part of the spectrum (as do snakes) or the ultraviolet range (as do some insects). Similarly, humans cannot hear in the infra- or ultra-sonic ranges of sound frequency as do, respectively, elephants or bats.

Non-specific senses for mechanical signal, thermal changes, or pain, do not have a specific location or specialized apparatus for reception. Nevertheless, all non-specific senses are also limited in terms of the ranges of environmental information that can be sensed (frequency of vibration, temperature range, etc.).

During technological processes, humans encounter additional sensory limitations. In the execution of their duties, human operators mainly use vision, the most developed human sense, although other senses are occasionally used as principal inputs, typically as warning signals (e.g., auditory stimuli such as alarms, smell for detecting chemicals such as natural gas, and smell and taste as “quality control” during cooking or brewing processes), the vast majority of human/machine interfaces are designed to communicate information visually. In complex technical environments, competing visual inputs can tax the ability of the operator to handle the incoming information. For example, if one looks at the thousands of visual indicators and monitors that saturate the cockpit of a modern aircraft or a nuclear power station control room, it makes one wonder how it is possible to continuously look attentively at the entire console of instrumentation, much less to read, analyze, and understand all of the quantitative and qualitative information presented during the hours of a working shift or during an intercontinental flight. For this reason, modern computers are becoming indispensable for monitoring and controlling most complex routine processes and they are highly satisfactory when everything is operating smoothly. However, situations of unpredictable change can rapidly exceed the capabilities of computerized controllers. Unexpected fluctuations, equipment malfunctions, and environmental disturbances—any of these events necessitates immediate operator intervention employing the human brain's innate and massively parallel or simultaneous analytical capabilities for decision-making and creative problem solving—something that modern computational technology is still missing.

The output of the human operator is motor output, i.e., movement. In fact, the only output of the brain is a signal for control of movement. For example, just keeping the human body in an upright posture seems mundane, yet it is an astonishingly complicated pattern of continuous action involving nearly every skeletal muscle in the human body. Emotional reactions too, immediately change the tension in many muscles of the human face and/or internal body musculature. While voice commands might be perceived as a non-movement output, speech itself is the result of very sophisticated combination of movement patterns in different muscles in the tongue, laryngeal area, lungs and diaphragm.

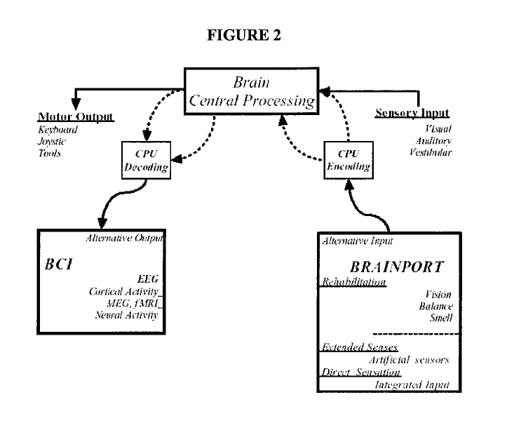

The most complex and sophisticated output apparatus available to the human operator, including both natural parts of the body and external devices, is the human hand—specifically the fingers. Pressing a button, turning a switch, keyboard typing, using a joystick control—all are complicated movement patterns, involving synchronous action of thousands of muscular fibers. The result can be as coarse as turning a valve handle, or as subtle as sensing the friction of a computer mouse. Yet humans typically have only two hands—consequently the human operator can perform only a limited number of tasks at one time. These various motor outputs are shown in the upper left-hand portion of FIG. 2. Clearly, the natural biological limitations of the human are key factors in creating input/output information saturation and operator overload. The results can be likened to a traffic jam in the technological information loop.

It is doubtful that following the present path of increasing technological development will lead to a reduction in information flow to the operator in the near future. Thus, there are two basic ways to address the present situation: 1) Improve the information processing capacity through education and training, to improve the operator's capacity and efficiency in solving process problems and thereby improve their analytical brain power; and 2) Improve the operator's input and output information processing capacity by optimizing the ways in which the data is presented to the operator. One aspect of the present invention is to alleviate or correct information bottlenecks, e.g., at overused input channels such as the visual input channel, distributing a portion of the information flow to the operator's brain over one or more alternative sensory channels.

A contemporary technological solution to the latter challenge is to implement a Brain Computer Interface (BCI)— that is, to utilize an interface technology designed to transfer information from the brain to the computer or vice versa, by employing alternate but underutilized natural biological pathways. The present invention provides systems and methods that address this approach. This novel approach is diagrammed in the FIG. 2. As described in the Examples, below, these systems and methods have achieved tremendous results in a wide range of human enhancements for healthy and disabled subjects.

The majority of modern BCI technologies are designed to provide alternative outputs from the brain to a computer. An early application of BCIs was to aid completely paralyzed patients, who have lost ability to move, speak, or otherwise communicate. Various levels of neuronal activity can be considered as potential sources for output, from single fibers and neurons up to the sum total of signals from large cortical and subcortical areas, such as EEG or fMRI signals, the integrated output of which can range as high as thousands and even millions of neurons.

In the vast majority of these BCI scenarios, the main goal is to use “internal” brain signals derived from the outputs of various areas of the brain to control computer-based peripherals, e.g., to control cursor movement on a computer monitor, to select icons or letters, to operate neuroprosthesises. There are many successful examples of such an approach. Microchips implanted in a human hand or animal brain can be used to transfer electronic copies of neural spike flows from goal-directed movements to an artificial limb to produce an exact replica of the original movement. Another example involves using certain components of acquired EEG signals that can be extracted, digitized, and applied as supplemental flight controls for drones or other unmanned aircraft.

However, few BCI's address alternate information inputs to the brain, or to be more precise —CBI's (Computer Brain Interface). This technology is realized in the systems and methods of the present invention. The present invention provides unique ways of presenting meaningful information to the brain by, for example, electrotactile stimulation of the tongue. The present invention is not limited to electrotactile stimulation of the tongue, however. A wide variety of sensory input methods may be used in the various methods of the present invention. In some embodiments, the sensory input provided by the present invention is tactile input. In some embodiments, the tactile input is vibrotactile input. In particularly preferred embodiments, the tactile input is electrotactile input. In some embodiments, the sensory input is audio input, visual input, heat, or other sensory input. The present invention is not limited by the location of the sensory input. For audio inputs, the input may be from an external audio source to a subject's ears. In alternative embodiments, the input may be from an implanted audio source. In yet other audio inputs, the audio source may provide input by non-implanted contact with a bony portion of the head, such as the teeth. For tactile inputs, any external or internal surface of a body may be used, including, but not limited to, fingers, hands, arms, feet, legs, back, abdomen, genitals, chest, neck, and face (e.g., forehead). In particularly preferred embodiments, the surface is located in the mouth (e.g., tongue, gums, palette, lips, etc.). In some embodiments, the input source is implanted, e.g., in the skin or bone. In other embodiments, the input source is not implanted.

The present invention is not limited by the nature of the device used to provide the sensory input. A device that finds use for electrotactile input to the tongue is described in U.S. Pat. No. 6,430,450, herein incorporated by reference in its entirety. Many of the embodiments of the present invention are illustrated below via a discussion of electrotactile input to the tongue. While this mode of input is a preferred embodiment for many applications, it should be understood that the present invention is not limited to input to the tongue, electrotactile input, or tactile input.

For example, the present invention is not limited to a particular method of delivering stimulation (e.g., signals (e.g., for sensory input)) to the tongue. Indeed, a variety of methods of delivering stimulation (e.g., signals (e.g., for sensory input)) to the tongue can be used including, but not limited to, tactile (e.g., electrotactile) stimulation, temperature (e.g., heat or cooling) stimulation, chemical stimulation, mechanical force stimulation and pressure stimulation. Furthermore, any one method of delivering stimulation (e.g., signals (e.g., for sensory input)) to the tongue may be combined with one or more other methods for such delivery.

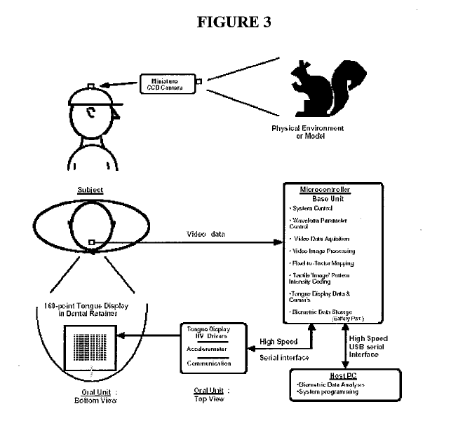

A specific preferred embodiment of the present invention is shown in FIG. 3 and discussed herein to highlight various features of the present invention. FIG. 3 shows a tongue-based electrotactile input of the present invention configured to provide video information. Such a system finds use in transferring video information to blind or vision-impaired subjects or to enhance or supplement the perception of sighted subjects. The configuration of the device shown comprises two main components: an intra-oral tongue display unit, and a microcontroller base-unit. These two elements are connected by a thin 12-strand tether that carries power, communication, and stimulation control data between the base and oral units, as shown in the schematic diagram (FIG. 3).

In the embodiment shown, the oral unit contains circuitry to convert the controller signals from the base unit into individualized zero to +60 volt monophasic pulsed stimuli on a 160-point distributed ground tongue display. The gold plated electrodes are on the inferior surface of a PTFE circuit board using standard photolithographic techniques and electroplating processes. This board serves as both a false palate for the tongue and the foundation to the surface-mounted devices on the superior side that drives the electrotactile (ET) stimulation. This unit also has a MEMS-based 1, 2, 3, 6-axis accelerometer for tracking head motion during visual image scanning and for vestibular feedback applications. This configuration utilizes the vaulted space above the false palate to place all necessary circuitry to create a highly compact and wearable sub-system that can be fit into individually molded oral retainers for each subject. With this configuration, only a slender 5 mm diameter cable protrudes from the corner of the subject's mouth and connects to the belt-mounted base unit. Alternatively, wireless communication systems may be used.

The base unit in the embodiment shown in FIG. 3 is built around a Motorola 5249 controller running compiled code to manage all control, communications, and data processing for pixel-to-tactor image conversion. It is user configurable for personalized stimulation iso-intensity mapping, camera zooming and panning, and other features. The unit has a removable 512 MB compact flash memory cards on board that can be used to store biometric data or other desired information. Programming and experimental control is achieved by a high-speed USB between the controller and a host PC. An internal battery pack supplies the 12 volt power necessary to drive the 150 mW system (base+oral units) for up to 8 hours in continuous use.

In preferred embodiments, the system is designed with electrical safety protection measures for both the power supply and electrical stimulation components of the system. Other modes of electrical protection required by consensus standards may also be included (e.g., physical and environmental protection) and are well known by those of skill in the art.

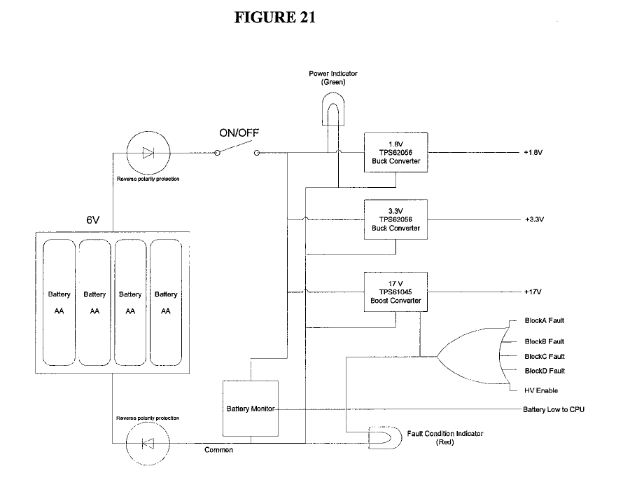

An exemplary power supply unit is depicted in FIG. 21. The power supply unit can be configured to accept multiple safety triggers thereby ensuring a proper controlled power-down sequence (e.g., in the event of a failure or occurrence of a risk event) including the ability to individually power down the analog and digital portions of the circuit.

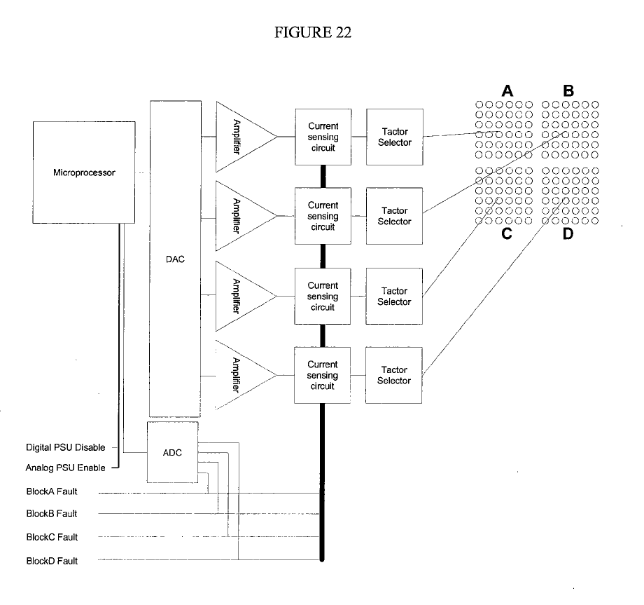

A stimulation circuit of some embodiments of the present invention is depicted in FIG. 22. In some preferred embodiments, the stimulation circuit comprises a microprocessor, a digital to analog converter, an amplifier, a current sensing circuit, addressing logic and electrodes. In some embodiments, the stimulation circuit comprises 144 electrodes with 4 amplifiers that drive tongue stimulation (e.g., wherein only four electrodes can be active at any one time). The present invention is not limited to this particular configuration. Indeed, in other embodiments, the stimulation circuit may comprise more (e.g., 150-200 or more) or less (e.g., 1-140) electrodes, or more (e.g., 5-20 or more) or less (e.g., 1-3) amplifiers.

The stimulation circuit may be configured such that an independent current sensing circuit exists for each of the amplifiers (e.g., for each of the 4 amplifiers). The current sensing circuit may consist of an instrumentation amplifier, voltage reference, resistor, and comparator. The comparator can be calibrated to shut down the analog portion of the power supply if a predetermined threshold is reached (e.g., 8.5 mA). Under these circumstances, the digital portion of the circuit could still be powered (e.g., allowing the processor time to log the conditions under which the over current condition occurred and to shut down in a controlled manner).

The current sensed can also be captured by an analog to digital converter (e.g., to allow the processor to monitor current in real time). In some embodiments, an additional layer of protection can be provided by a fault detection subroutine (e.g., that monitors the values sent to the analog to digital converter).

Multiple configurations of the intra-oral tongue display assembly are contemplated to be useful in the systems of the present invention. In some embodiments, a potting technique may be used for encapsulation of the intra-oral display assembly. For example, a medical grade silicone (e.g., SILASTIC) can be used to fill the volume between the back side of the electrode array and a rigid plastic cap. Configuring in this manner protects electronic components from saliva. It may be desirable, in some embodiments, after this assembly is complete to apply a second coating (e.g., with a medical grade silicone or similar material) thereby encapsulating the rigid cap. In some preferred embodiments, this layer of coating is thin (e.g., ˜0.05 inches) and dried to a smooth (e.g., glossy) surface thereby improving the aesthetics of the device. In other embodiments, a plastic injection molding technique can be used to encapsulate the intra-oral display assembly (e.g., to generate an overmolded intra-oral display).

In some embodiments, a removable cap or cover is generated for components of the intra-oral display assembly (e.g., for the electrode array, rigid plastic cap, or both). Caps/covers can be configured in multiple ways that do not interfere with the systems and methods of the present invention. For example, caps/covers can be generated that are disposable, or may comprise a coating that permits sterilization (e.g., by submersion in alcohol or autoclaving). Furthermore, caps/covers may be optimized for individual patients (e.g., for a child) or for unique characteristics of a specific patient's tongue (e.g., a cap/cover my comprise means—e.g., a ridge, bump, or other tactile marker—that permits a user to place the intra-oral tongue display on his or her tongue in the same location each time the display is used).

In some embodiments, the device is configured to permit any portion that comes in contact with the subject (e.g., an intra-oral component) to be detachable from the rest of the system. This may have several advantages. For example, it permits each subject using a device (e.g., at a physician's office) to have a personal (e.g., sterile, optimized, etc.) device. Each user need only attach their personal component to the system when using the system and detach when completed. The same process may be accomplished with detachable caps or covers (e.g., disposable, sterilizable, etc.) that shield the user from the intra-oral component. In some embodiments, the cap or cover entirely encompasses the portion of the system that contacts the subject. In some such embodiments, the cap or cover is made of conductive plastics to permit electrotactile stimulation through the material. In some embodiments, the system is configured such that multiple different detachable (or wireless) components may be used simultaneously with the same base unit. For example, multiple users may “plug in” to a single base unit to receive training, therapy, etc. With wireless systems in particular, a single base system may serve many users in parallel without, for example, being in the same room or area.

Electrodes of the intra-oral tongue display can be plated with any medically compatible metal (e.g., gold or platinum) to protect a patient from material (e.g., copper) used to make the circuit. Finite element analysis has revealed hotspots (e.g., spots of increased electrical current density) at the edges of electrodes (e.g., active and ground path return electrodes). These points of increased current density may be responsible for pain or discomfort perceived by a user when high amounts of energy are used. Thus, reduction of current density (e.g., at the edges of the electrodes while supplying the same voltage stimulus) may be used to increase the dynamic range.

One way this can be achieved is by changing the resistivity of the electrode as a function of the radius of the electrode. For example, to reduce the hot spots, the resistivity of the electrode can be increased as a function of radius such that the outer edge of the electrode are more resistive than the center of the electrode. This reduces current density by spreading current across the full area of the electrode so that it can enter or exit the tongue over a larger surface area. Several coating techniques or other fabrication processes can be used to accomplish a desired change in electrical resistivity as a function of radius including, but not limited to, generating a gradient electrical resistant electrode (GERE) (e.g., that is similar to a gradient index of refraction optical lenses (GRIN)).

Another way to avoid or decrease the occurrence of hotspots is through tactor shape. Certain shapes (e.g., circles) are known to distribute current density better than other shapes (e.g., squares). Thus, in some embodiments, tactor shape is used to decrease hot spots on the electrode terminal, wherein the tactor shape is circular. Furthermore, tactor shape can be combined with wave-form schemes (see below) to optimize the delivery of information to a user. Thus, decreasing the occurrence of hot spots expands the dynamic range, thereby permitting an increase in energy delivered (e.g., range of usable current), that in turn permits an increase in information conveyable to a patient. In some preferred embodiments, electrodes are 1.7 mm diameter, flat, spaced 2.3 mm apart, and arranged in a square grid. However, the present invention is not limited to this configuration. Other configurations are also useful, including, but not limited to, smaller electrodes (e.g., between 1.7 mm and 0.3 mm in diameter) arranged in a hexagonal grid (e.g., allowing an increase in number of tactors). Thus, in some embodiments, there are 300-500 tactors per square centimeter. Additionally, different tactor material may be used in order to decrease hotspost (e.g., conductive plastics and/or conductive epoxy mixed in with insulating plastic and/or epoxy). Furthermore, instead of tactors having a flat terminus, tactors may be curved at the end (e.g., generating a small bump).

Multiple wave-form schemes can be delivered to a user and find use with the systems of the present invention. In some embodiments, square-pulse is used for tactile stimulation. However, the present invention is not limited to square-pulse schemes. Specifically, any signal monotonicly rising from zero that has some portion of stable duration before monotonicly falling to zero again is useful with the present invention. For example, in some embodiments, a damped-sinusoid pulse can be used. Use of a sinusoid pulse is contemplated to permit an improved dynamic range as the sinusoid pulse more resembles a natural signal (e.g., a pulse shape similar to natural nerve signaling). Furthermore, a wavelet may be provided to a patient (e.g., that resembles natural nerve firing of biological system thereby permitting a broader dynamic range). In some embodiments, use of wavelets avoid sharply defined edges of time and amplitude (See, e.g., Chui, An Introduction to Wavelets (Wavelet Analysis and Its Applications, Volume 1), Academic Press (1992); Debnath, Wavelet Transforms and Time-Frequency Signal Analysis, Birkhä user Boston Inc. (2001); Fernandes et al., IEEE Trans Image Process. January; 14(1):110-24 (2005)).

The damped sine is

Amplitude=c×e^{-at}×sin(2π·f·t).

In some preferred embodiments, sine f=20 kHz and damping parameter a=2.218*f=4.436×10⁴, providing an amplitude of 12 volts peak with 0.05 volts after 2.5 cycles (or 125 microseconds). Thus, in some embodiments the present invention provides duplication or simulation of natural nerve firing. For example, the systems and methods of the present invention can duplicate natural nerve pulse form that has a smooth starting, rapid rise to peak and then slower fall. In some embodiments, the time course is about 1 millisecond start to finish, with pulse amplitude of 0.1 volts measured on the surface of the nerve. Although an understanding of the mechanism is not necessary to practice the present invention and the present invention is not limited to any particular mechanism of action, duplicating natural nerve firing improves the dynamic range of the systems and methods of the present invention because a patient's pain threshold is higher with replicated natural firings.

In some embodiments, systems and methods of the present invention present the same wave form on every tactor with variable amplitude (e.g., eliminating the need to raster scan the image). For example, one module will create the wave form, and other modules will act as multipliers.

Also useful in the present invention is the damped lorentzian:

$$Amplitude = c \frac{\frac{Γ}{2} \times sin ( 2 π \cdot f \cdot t )}{{t}^{2} + {( \frac{Γ}{2} )}^{2}}$$

In these cases, it is the rising portion of the sine function that determines how the wave rises, and its peak amplitude is modified by the damping portion. The parameters c, a, f and Γ determine peak amplitude and time before zero crossing.

A simple wave form that finds use with the present invention is a square pulse with a fixed width. In some embodiments, square pulse with a fixed width can be used wherein the time and amplitude are varied, or a fixed amplitude with variable width (e.g., pulse width modulation).

In some embodiments, the amount of wave-form energy provided to any particular patient is variable. Thus, a range of wave-form energy (e.g., sub-detectable up to painful) is useful in the systems of the present invention. For example, because each patient is unique, different amounts of energy may be provided to each user (e.g., taking into account electrode shape, position, energy form, and sensitivity of the patient). In some preferred embodiments, the systems and methods of the present invention provide between 100 microwatts (0.1 milliwatts) in 1 microsecond (i.e., 100 picojoules) and 1 Joule. Furthermore, the present invention provides the ability to map the dynamic range of each user. Once determined, such a map allows an optimized amount of wave-form energy to be delivered to each patient (e.g., maximizing the amount of information conveyable to each patient), should this be desired.

Thus, this system is a computer-based environment designed to represent qualitative and quantitative information on the superior surface of the tongue, by electrical stimulation through an array of surface electrodes. The electrodes form what can be considered an “electrotactile screen,” upon which necessary information is represented in real time as a pattern or image with various levels of complexity. The surface of the tongue (usually the anterior third, since it has been shown experimentally to be the most sensitive area), is a universally distributed and topographically organized sensory surface, where a natural array of mechanoreceptors and free nerve endings (e.g. taste buds, thermo sensitive receptors, etc.) can detect and transmit the spatially/temporally encoded information on the tongue display or ‘screen’, encode this information and then transfer it to the brain as a “tactile image.” With only minimal training the brain is capable of decoding this information (in terms of spatial, temporal, intensive, and qualitative characteristics) and utilizing it to solve an immediate need. This requires solving numerous problems of signal detection and recognition.

To detect the signal (as with the ability to detect any changes in an environment), it is useful to have systems of the highest absolute or differential sensitivity, e.g. luminance change, indicator arrow displacement, or the smell of burning food. Additionally, the detection of the sensory signals, especially from survival cues (about food, water, prey or predator), usually must be fast if reaction times are to be small in life threatening situations. It is important to note that the sensitivity of biological and artificial sensors is usually directly proportional to the size of the sensor and inversely proportional to the resolution of the sensorial grid.

Information utilized during this type of detection task is usually qualitative information, the kind necessary to make quick alternative decisions (Yes/No), or simple categorical choices (Small/Medium/Large; Green/Yellow/Red).

The recognition process is typically based on the comparison of given stimuli (usually a complex one such as a pattern or an image, e.g. a human face) with another one (e.g. a stand alone image or a set of original alphabet images). To solve the recognition problem it is useful to have sensors with maximal precision (or maximal resolution of the sensorial grid) to gather as much information as possible about small details.

Often this is related to the measurement of signal parameters, gathering quantitative information (relative differences in light intensity, color wavelength, surface curvature, speed and direction of motion, etc.), where and when precision is more important than speed.

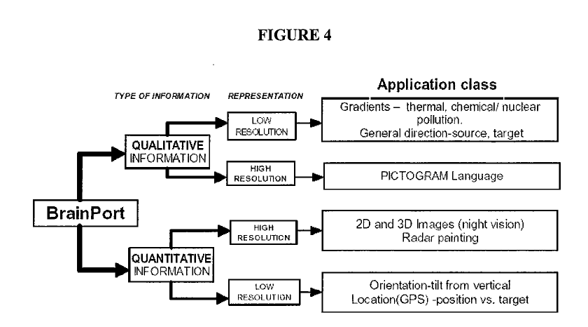

The systems of the present invention are capable of transferring both qualitative and quantitative information to the brain with different levels of a “resolution grid,” providing basic information for detection and recognition tasks. The simple combination of two kinds of information (qualitative and quantitative) and two kinds of a stimulation grid (low and high resolution) results in four different application classes. Each class can be considered as a root (platform) for multiple applications in research, clinical science and industry, and are shown in FIG. 4.

The first class (qualitative information, low resolution) can be illustrated by the combination of external artificial sensors (e.g., radiation, chemical) with the systems of the present invention for detection of environmental changes (chemical or nuclear pollution) or explosives detection. The presence of selected chemical compounds (or sets of compounds) in the air or water can be detected using the systems of the present invention simply as “Yes/No” paradigms. By using a distributed array of stimulators and a corresponding presentation of signal gradients on the system array it is also possible to use the system for source orientation relative to the operator. With minimal training, the existence of the otherwise undetectable analyte in the environment is perceived by the subject as though it were detectable by the normal senses.

The second class (qualitative information, high resolution) can be illustrated by an application for underwater navigation and communication. A simple alphabet of images or tactile icons (sets of moving bars in four directions, a flashing bar in the center and flashing triangles on left and right sides of system array) constitute a system of seven navigation cues that are used to correct deviation and direction of movement along a designated path. In experiments conducted during the development of the present invention, after less than five to ten minutes of preliminary training, blindfolded subjects were capable of navigating through a computer generated 3-D maze using a joystick as a controlling device and a tongue-based electrotactile device for navigation signal feedback.

The third class (quantitative information, low resolution) can be illustrated by another existing application for the improvement of balance and the facilitation of posture control in persons with bilateral damage of their vestibular sensory systems (BVD-causing postural instability or “wobbling”, and characterized by an inability to walk or even stand without visual or tactile cues). A quantitative signal acquired from a MEMS accelerometer (positioned on the head of subject) is transferred through the oral electrotactile array as a small, focal stimulus on the tongue array. Tilt and sway of the head (or the body) are perceived by the subject as deviations of the stimulus from the center of the array, providing artificial dynamic feedback in place of the missing natural signals critical for posture control.

The fourth class (quantitative information, high resolution) can be illustrated by another existing system that implements a great scientific challenge—that of ‘vision’ through the tongue. Signals from a miniature CCD video camera (worn on the forehead) are processed and encoded on a PC and transferred through the array as a real-time electrotactile image. Using this electrotactile display, subjects are capable of solving many visual detection and recognition tasks, including navigation and catching a ball. The system may also be used for night (infrared) or ultraviolet vision, among other applications.

On the basis of the four strategic classes of applications it is possible to develop multiple practical industrial applications that can include a human operator in the loop. The present invention provides for the development of alternative information interfaces so that the brain capacity of the human operator in the loop can be more fully and efficiently utilized in the technological process.

As described above, the modern tendency is toward designing instrumentation with increased density and complexity of visual representations. For example, the numerous light and arrow indicators of past displays are being replaced by computer monitors that condense the information into lumped static and dynamic 2D and 3D images or video streams. There are various rationales behind the development of these kinds of cumulative information presentations. One is to decrease the physical area of the visual information field, thereby limiting the space the operator must scan to monitor the instrument. Some size reduction is accomplished by condensing multiple parameters into a single image. However, to control modern technological processes, an operator must be able to efficiently observe and make decisions about hundreds of changing parameters. If each parameter is represented by a simple indicator, like a light, arrow, or dial, the control panel will consist of hundreds of the same kinds of indicators. By miniaturizing and grouping all of these indicators, the resultant ergonomically designed displays become extremely intensive information panels, like the ones presently found in modern aircraft (Electronic Flight Instrument Systems, EFIS) or nuclear power stations.

The main problem with these approaches is the distribution of attention required by observer. In the presence of multiple visual stimuli, the operator is forced to limit his/her attention capacity to one or a few of the elements being displayed. The operator must shift attention from one element to another in order to perceive all of the information contained in the complex display. Such complex information display requires that the operator be systematic in monitoring the panel, to minimize the chances of overlooking any particular element. Anything that distracts the operator can cause a failure in the system. In addition, the ability of an operator to monitor a complex display tends to diminish during extended periods of observation (e.g., over the course of a work shift). One possible solution is to decrease the number of indicators and replace them with more condensed, more complicated visual images that combine multiple parameters into a single image. For example, a single 3D scatter plot can represent up to 12 simultaneously changing parameters, using multiple features of single elements as coding variables (e.g. size, dimension, shape, color, orientation, opacity, pattern of single elements, etc.) Although useful, this approach still relies on distributing the information using exclusively visually representable features.

An alternative approach is to use the systems and methods of the present invention as a supplemental input for processing information.

As previously mentioned, the systems are capable of working in various modes of complexity: As a simple indicator, such for (first application class) signal detection; as a target location device (third application class) for position control of signals on a 2D array, much like a “long range” target location radar plot; in almost all computer action games; as a simple GPS monitor. The systems can also work in more complex modes such as for more complete vision substitution device, an infrared or ultraviolet imaging system creating complex electrotactile images using in addition to two dimensions of its electrode array, the amplitude and frequency of the main signal, the spatial and temporal frequency of the signal modulation, and a few internal parameters of the signal waveform. In other words the systems and methods of the present invention are capable of creating a complex multidimensional electrotactile image—similar to that of visual imagery.

Thus, the present invention provides systems that afford processing of artificial sensory signals (from any source) by natural brain circuitry and organizational behavioral, thereby providing direct sensation or direct perception by the operator.

People usually do not think about such natural behavioral acts like breathing or digestion as fully “automatic”, internally “built-in” processes. Even if we think about them, we cannot stop or permanently change them. Walking, swimming, riding a bike or driving a car are other examples of very complex biomechanical processes that also use multiple sensory and motor coordination, but we learn them early in our lives; performing them also almost naturally (without thinking about each component), quickly and with great precision and efficiency. The present invention provides means for efficiently training the brain to carry out new tasks and perceive and utilize new information “automatically.” Experiments conducted using the technology of the present invention demonstrated after training with the systems, fMRI screening of the brain activity in blind subjects during the electrotactile presentation of visual images revealed strong activation in areas of the primary visual cortex. This means that after training with systems, the blind person's brain begins to use the most sophisticated analytical part of the cortex for analysis of electrotactile information displayed on the tongue during visual tasks. Before training, it is contemplated that these areas were not active. The activation of normal analytical resources (e.g. the ‘visual’ part of the brain) in response to artificial sensory stimulation was “automatic” in that it did not rely on the use of the eyes for directing the information to the primary visual cortex.

With the systems of the present invention, a blind person can navigate, a BVD patient can walk, a video game player or fighter pilot can perceive objects outside of their field of view, a doctor can conduct remote surgery, a diver can sense direction underwater, a bomb squad member can sense the presence of explosive chemicals, all as naturally as an experienced person would ride a bike, play an instrument reading sheet music, or drive a car.

In some embodiments, the systems and methods of the present invention find use in numerous applications for sensory substitution. In such embodiments, sensory perception is provided to a subject to compensate for a missing or deficient sense or to provide a novel sense.

In some such embodiments, the sensory substitution provides the subject with improved balance or treats a balance-associated condition. In such embodiments, subjects are trained to associate tactile or other sensory inputs with body position or orientation. The brain learns to use this added sensory input to compensate for a deficiency. For example, the systems and methods may be used to treat bilateral vestibular dysfunction (BVD) (e.g., caused by ototoxicity, trauma, cancer, etc.). Example 1, below, describes successful treatment of a number of BVD patients using the systems and methods of the present invention. Examples 2-8 describe additional benefits imparted on one or more of the subjects during or following their clinical rehabilitation. Based on these results, the present invention finds use in the treatment of other diseases and conditions related to the vestibular system, including but not limited to, Meniere's disease (see Example 25), migraine (see Example 26), motion sickness, MDD syndrome, dyslexia, and oscillopsia. The systems and methods also provide the tangential benefits of improved sleep recovery, fine movement recovery, psychological recovery, quality of life improvement, and improved emotional well-being.

The balance-related sensory substitution methods may be applied to a wide range of subjects and uses. For example, the methods find use in ameliorating or eliminating aging related balance problems for both fall prevention and general enhancement. The methods also find use in balance recovery after injury.

The present invention also provides systems and methods for the treatment of a variety diseases and conditions including, but not limited to, sicknesses or conditions in which a subject suffers from a defect in vestibular function (e.g., balance), proprioception, motor control, vision, posture, cognitive functions, tinnitus, emotional conditions and/or sleep. Subjects known to experience these defects include those diagnosed with, experiencing symptoms of and/or displaying symptoms of multiple diseases, sicknesses or conditions, including, but not limited to, vestibular disease, autism, traumatic brain injury, stroke, attention deficit disorder, hyperactivity, addiction, narcolepsy, coma, schizophrenia, shaken baby syndrome, Alzheimer's, Parkinson's, Gerstmann's Syndrome, dementia, delusion, Fetal alcohol syndrome, Cushing's disease, Creutzfeldt-Jakob Disease, Huntington's Disease, Keams-Sayre Syndrome, Metachromatic Leukodystrophy, Mucopolysaccharidosis, Niemann-Pick disease, Pelizaeus-Merzbacher Disease, phobias, Persistent Vegetative State, Postpartum depression, depression of any kind, Reye's Syndrome, Rett's syndrome, Sandhoff Disease, developmental disorders, Meniere's disease, balance disorders, Septo-Optic Dysplasia, Soto's Syndrome, Spastic disorders, migraine, Sturge-Weber Syndrome, Subacute Sclerosing Panencephalitis, Toxic Shock Syndrome, Transient Ischemic Attack, Williams Syndrome, Wilson's Disease, Down Syndrom, Limbic encephalitis, Vascular dementia, Heavy metal exposure, Lewy body disease, Normal pressure hydrocephalus, Post-traumatic dementia, Pick's disease, Multiple sclerosis, Jakob-Idiopathic basal ganglia calcification, Neurosyphilis and Acquired immune deficiency syndrome (AIDS).

For example, in some embodiments, the present invention provides systems and methods for improving or correcting vestibular function (e.g., balance), proprioception, motor control, vision, posture, cognitive functions, tinnitus, emotional conditions and/or sleep in a subject with traumatic brain injury (See, e.g., Example 21).

In some embodiments, the present invention provides systems and methods for correcting or improving verbal and non-verbal communication, social interactions, sensory integration (e.g., tactile, vestibular, proprioceptive, visual and auditory), and leisure or play activities in a subject with a Pervasive Developmental Disorder (PDD), including, but not limited to an Autistic Disorder, Asperger's Disorder, Childhood Disintegrative Disorder (CDD), Rett's Disorder, and PDD-Not Otherwise Specified (PDD-NOS) (See, e.g., Example 22).

In some embodiments, the present invention provides systems and methods for correcting or improving symptoms associated with Parkinson's disease (e.g., defects in motor control, including, but not limited to, walking, talking, or completing simple tasks that depend on coordinated muscle movements) (See, e.g., Example 23).

In some embodiments, the present invention provides systems and treatments for correcting or improving weakness of the face, arm or leg, (e.g., on one side of the body), correcting or improving numbness of the face, arm, or leg, especially on one side of the body; correcting or improving confusion, trouble speaking or understanding speech; correcting or improving vision disturbances, trouble seeing in one or both eyes; correcting or improving trouble walking, dizziness, loss of balance or coordination; correcting or improving severe headache; correcting or improving slurred speech, inability to speak or the ability to understand speech; correcting or improving difficulty reading or writing; correcting or improving swallowing difficulties or drooling; correcting or improving loss of memory; correcting or improving vertigo (spinning sensation); correcting or improving personality changes; correcting or improving mood changes (depression, apathy); correcting or improving drowsiness, lethargy, or loss of consciousness; and correcting or improving uncontrollable eye movements or eyelid drooping in a stroke subject or subject displaying stroke-like symptoms (See, e.g., Example 24).

While an understanding of the mechanism is not necessary to practice the present invention and while the present invention is not limited to any particular mechanism of action, in some embodiments, it is contemplated that the use of tactile stimulation (e.g., electrotactile stimulation of the tongue) conditions the brain for correcting or improving a general function (e.g., motor control, vision, hearing, balance, tactile sensation). The preferred route is electrotactile stimulation of the tongue.

For example, in some embodiments, it is contemplated that systems and methods of the present invention correct, improve and/or activate residual tissue (e.g., neurological cells and tissue) not otherwise active or, to the contrary, overloaded with information. In some embodiments, the present invention provides a clarifying effect, reducing the signal to noise ratio and thereby providing beneficial effects to a subject. In some embodiments, the systems and methods of the present invention act to repair or reprogram the machinery (e.g., through patterned electrical currents embedded with information) required for motor control, vision, hearing, balance, tactile sensation, etc. In some embodiments, the present invention provides the brain access to signals (e.g., weak signals), that, over time and with treatment (e.g., training on the systems herein) permits the brain to respond to the signals (e.g., sensory signals, balance, motor coordination information, etc.). In some embodiments, access to these signals and/or treatment (e.g., training on the systems herein) provides a subject a new or improved function (e.g., motor control, balance, etc.).

While an understanding of the mechanism is not necessary to practice the present invention and while the present invention is not limited to any particular mechanism of action, it is contemplated that, in some embodiments, the systems and methods of the present invention provide or simulate long-term potentiation (long-lasting increase in synaptic efficacy which follows high-frequency stimulation) to provide enhanced brain function. The residual and rehabilitative effect of training seen in experiments conducted during the development of the present invention upon prolonged tactile stimulation is consistent with long-term potentiation studies. For example, in some embodiments, the systems and methods of the present invention utilize electrical currents similar to those used in long-term potentiation studies (e.g., 50-200 Hz).

In some embodiments, the tongue is relevant for improving or correcting residual balance. In some embodiments, one or more nerves present in the tongue function to conduct information from the systems and methods of the invention to the brain. In some embodiments, the signals (e.g., electrical) sent through the tongue provide the brain access to signals it otherwise has difficulty (e.g., does not or cannot) perceive. Although an understanding of the mechanism is not necessary to practice the present invention and the present invention is not limited to any particular mechanism of action, in some embodiments, signals presented to the tongue (e.g., via an electrotactile screen) are “seen” by the brain via channeling of the signals through nerves present within and/or sending signals to or from the tongue (e.g., the facial nerve, the hypoglossal nerve, the glossopharyngeal nerve, etc). The present invention is not limited by the form of stimulation of the nerves within the tongue. Indeed, a variety of stimulation (e.g., signals capable of communicating with the tongue) are contemplated to be useful in the systems and methods of the present invention including, but not limited to, signals distal to the nerves of the tongue and signals in direct contact with the nerves of the tongue. In some embodiments, the benefit a subject receives through the systems and methods of the present invention are correlated with the length of exposure the subject receives treatment (e.g., electrical stimulation through the tongue using the system). In some embodiments, benefits occur immediately. In some embodiments, the benefit is additive as training continues. In some embodiments, systems and methods of the present invention are used in combination with other treatments or procedures. In some embodiments, a synergistic beneficial effect is seen when a combinatorial approach is taken (e.g., when the systems and methods of the present invention are used in combination with other known therapies or treatments).

In some embodiments, systems and methods of the present invention benefit a subject through molecular events (e.g., activation or repression of genes present in brain tissue or cells). In some embodiments, cfos is activated. It is contemplated that gene expression patterns are altered through repetitive training using the systems and methods of the present invention. The expression of such genes may also be used diagnostically to monitor treatment or identify subjects suitable for treatment.

Thus, the present invention provides systems and methods for physiological learning that extends for long periods of time (e.g., hours, days, weeks, etc.). While the present invention is not limited to any mechanism of action and an understanding of the mechanism of action is not necessary to practice the present invention, it is contemplated that in some embodiments the systems and methods of the present invention function via sensitizing/energizing the component machinery required for motor control, vision, hearing, balance, tactile sensation, etc. In other embodiments, the systems and methods of the present invention sensitize/energize the brain in general, thereby producing brain physiology that is able to function properly or in an enhanced fashion. In some embodiments, the systems and methods of the present invention work via physical stimulation (e.g., chemically or electrically). In other embodiments, the invention works through means similar to the benefits received through meditation or other forms of focus or stress relief (e.g., yoga). In still other embodiments, the systems and methods of the present invention provide improved brain (e.g., cerebellum) function (e.g., activation of brain regions) (See, e.g., Ptito et al., Brain, 128(Pt 3):606-14 (2005), herein incorporated by reference in its entirety).

For example, the central nervous system comprises the brain and the spinal cord. All other nerves in the body comprise the peripheral nervous system. Efferent nerves carry messages from the central nervous system to all parts of the body (the periphery) whereas afferent nerves carry information such as pain intensity from the periphery to the central nervous system. There are two types of efferent nerves: somatic, which go to skeletal muscles, and autonomic, which go to smooth muscles, glands and the heart. Messages in the form of electrical activity are conducted along the nerve fibers or axons. Between the terminus of the axon and the muscle or gland that the nerve controls (innervates), there is a gap called the synapse or synaptic cleft. When the conducted electrical impulse (action potential) reaches the nerve terminus, it provokes the release of chemicals called neurotransmitters. These chemicals diffuse across the synaptic cleft and react with a specialized structure (receptor) on the postjunctional membrane. The receptor is then said to be activated or excited, and its activation triggers a series of chemical events resulting ultimately in a biological response such as muscle contraction. The processes involving neurotransmitter release, diffusion and receptor activation are referred to collectively as transmission. There are many types of transmission, and they are named for the specific neurotransmitter involved. Thus, cholinergic transmission involves the release of the neurotransmitter, acetylcholine, and its activation of the postsynaptic receptor. Things that bind to and activate receptors are called agonists. Thus, acetylcholine is the endogenous agonist for all cholinergic receptors.

After leaving the central nervous system, somatic nerves to skeletal muscles have only one synapse, namely, that between the nerve terminus and the muscle it innervates. The neurotransmitter at that synapse is acetylcholine. Thus, this myo-(for muscle)-neural junction is one site of cholinergic transmission. The postjunctional receptor is called the motor end plate. Autonomic nerves, in contrast to somatic nerves, have an additional synapse between the central nervous system and the innervated structure (end organ). These synapses are in structures called ganglia, and these are nerve-to-nerve junctions instead of nerve-to-end organ junctions. Like somatic nerves, however, autonomic nerves also have a final nerve-to-end organ synapse. The neurotransmitter in autonomic ganglia is also acetylcholine; hence, this represents another site of cholinergic transmission. The motor end plate and the ganglionic receptors can also be activated by exogenously added nicotine. Thus, nicotine is an agonist for this particular subfamily of cholinergic receptors which are called nicotinic, cholinergic receptors.

There are two anatomically and functionally distinct divisions of the autonomic nervous system: the sympathetic division and the parasympathetic division. The preganglionic fibers of the two divisions are functionally identical, and they innervate nicotinic, cholinergic receptors in ganglia to initiate action potentials in the postganglionic fibers. Only the postganglionic fibers of the parasympathetic division, however, are cholinergic. The postganglionic fibers of the sympathetic division generally, but not always, secrete norepinephrine. The cholinergic receptors innervated by the postganglionic fibers of the parasympathetic division of the autonomic nervous system can also be activated by exogenously added muscarine, an agonist found in small amounts in the poisonous mushroom, Amanita muscaria. These constitute a second subset of cholinergic receptors which are called muscarinic, cholinergic receptors.

Although the receptors in ganglia and the motor end plate both respond to nicotine, they actually constitute two distinct subgroups of nicotinic receptors. Each of the three families of cholinergic receptors can be blocked by specific receptor antagonists to prevent their activation by endogenous acetylcholine or added agonists. Thus, specific blockers are known for cholinergic, muscarinic receptors innervated by postganglionic fibers of the parasympathetic division of the autonomic nervous system, for cholinergic, nicotinic receptors in both sympathetic and parasympathetic ganglia, and for cholinergic nicotinic receptors at the myoneural junction (motor end plates) of the somatic nervous system. When these receptors are blocked, the on-going biological activity associated with their normal and continuous activation is lost. For example, blockade of the motor end plate leads to generalized, flaccid paralysis.

There are some anomalous fibers in the sympathetic division of the autonomic nervous system. For example, the sympathetic postganglionic nerves that go to sweat glands are cholinergic instead of adrenergic, like most other sympathetic fibers, and they innervate mucarinic receptors. The sympathetic nerve to the adrenal gland innervates a receptor that is nicotinic like all autonomic ganglia, but there is no postganglionic fiber. The gland itself is analogous to a postganglionic sympathetic fiber, but, instead of secreting a neurotransmitter, it secretes epinephrine and norepinephrine into the blood stream, where they function as hormones. These hormones activate adrenergic receptors throughout the body.

Cholinergic drugs are medications that produce the same effects as the parasympathetic nervous system. Cholinergic drugs produce the same effects as acetylcholine. Acetylcholine is the most common neurohormone of the parasympathetic nervous system, the part of the peripheral nervous system responsible for the every day work of the body. While the sympathetic nervous system acts during times of excitation, the parasympathetic system deals with everyday activities such as salivation, digestion, and muscle relaxation.

Cholinergic drugs usually act in one of two ways. Some directly mimic the effect of acetylcholine, while others block the effects of acetylcholinesterase. Acetylcholinesterase is an enzyme that destroys naturally occurring acetylcholine. By blocking the enzyme, the naturally occurring acetylcholine has a longer action.

The spinal cord conducts sensory information from the peripheral nervous system (e.g., both somatic and autonomic) to the brain, and it also conducts motor information from the brain to various effectors (e.g., skeletal muscles, cardiac muscle, smooth muscle, or glands). The spinal cord also serves as a minor reflex center.

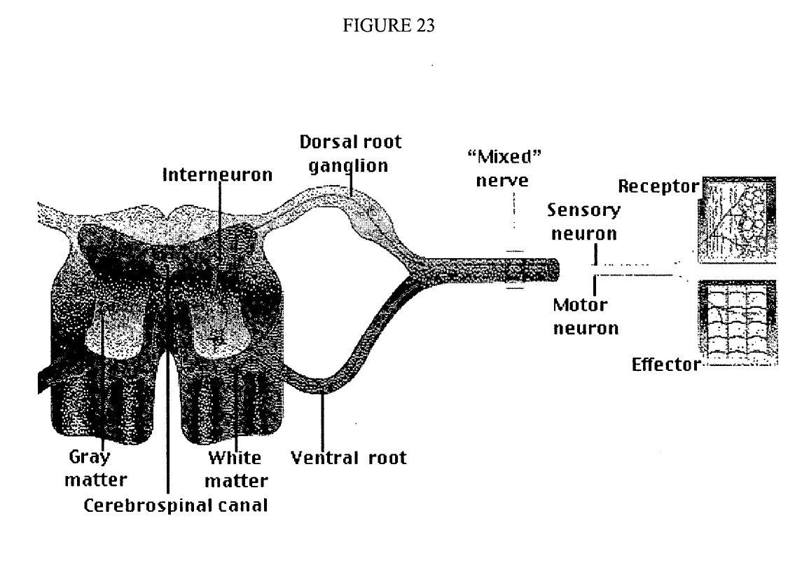

The brain receives sensory input from the spinal cord as well as from its own (e.g., cranial) nerves (e.g., trigeminal, vestibulocochlear nerve, olfactory and optic nerves) and devotes most of its volume and computational power to processing its various sensory inputs and initiating appropriate and coordinated motor outputs. Both the spinal cord and the brain comprise white matter (e.g., bundles of axons each coated with a sheath of myelin) and gray matter (e.g., masses of cell bodies and dendrites each covered with synapses). In the spinal cord, the white matter is at the surface, the gray matter inside (See FIG. 23). In the brain of mammals, this pattern is reversed. However, the brains of “lower” vertebrates like fish and amphibians have their white matter on the outside of their brain as well as their spinal cord.

Both the spinal cord and brain are covered in three continuous sheets of connective tissue known as the meninges. From outside in, these are the dura mater pressed against the bony surface of the interior of the vertebrae and the cranium; the arachnoid; and the pia mater. The region between the arachnoid and pia mater is filled with cerebrospinal fluid (CSF).

This CSF of the central nervous system is unique. Cells of the central nervous system are bathed in CSF that differs from fluid serving as the ECF of the cells in the rest of the body. The fluid that leaves the capillaries in the brain contains far less protein than “normal” because of the blood-brain barrier, a system of tight junctions between the endothelial cells of the capillaries. This barrier creates problems in medicine as it prevents many therapeutic drugs from reaching the brain. The cerebrospinal fluid (CSF) is a secretion of the choroid plexus. CSF flows uninterrupted throughout the central nervous system through the central cerebrospinal canal of the spinal cord and through an interconnected system of four ventricles in the brain. CSF returns to the blood through veins draining the brain.

The Spinal Cord comprises 31 pairs of spinal nerves that align the spinal cord. These are “mixed” nerves as each contain both sensory and motor axons. However, within the spinal column, sensory axons pass into the dorsal root ganglion where their cell bodies are located and then on into the spinal cord itself, whereas motor axons pass into the ventral roots before uniting with the sensory axons to form the mixed nerves.

The spinal cord carries out two main functions. It connects a large part of the peripheral nervous system to the brain. Information (e.g., nerve impulses) reaching the spinal cord through sensory neurons are transmitted up into the brain. Signals arising in the motor areas of the brain travel back down the cord and leave in the motor neurons. The spinal cord also acts as a minor coordinating center responsible for some simple reflexes like the withdrawal reflex.

Signals cross over the spinal tracts. For example, impulses reaching the spinal cord from the left side of the body eventually pass over to tracts running up to the right side of the brain and vice versa. In some cases this crossing over occurs as soon as the impulses enter the cord. In other cases, it does not take place until the tracts enter the brain itself.

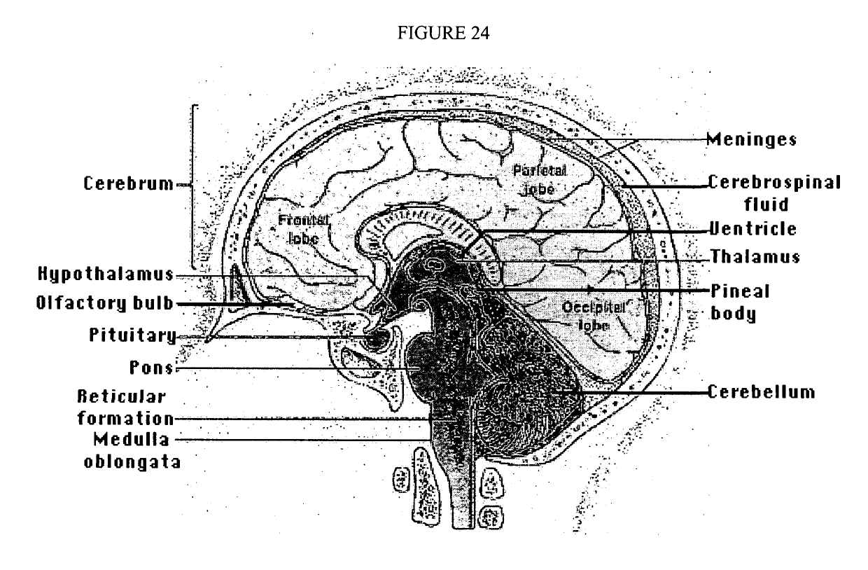

The brain of all vertebrates (e.g., humans) develops from three swellings at the anterior end of the neural canal of the embryo. From front to back these develop into the forebrain (also known as the prosencephalon), the midbrain (also known as the mesencephalon), and the hindbrain (also known as the rhombencephalon) (See FIG. 24). The brain receives nerve impulses from the spinal cord and 12 pairs of cranial nerves. Some of the cranial nerves are “mixed”, containing both sensory and motor axons (See, e.g., a description of each cranial nerve, below). Some of the cranial nerves (e.g., the optic and olfactory nerves) contain sensory axons only whereas some of the cranial nerves (e.g., the oculomotor nerve (e.g., that controls eyeball muscles)), contain motor axons only.

The cranial nerves emanate from the nervous tissue of the brain. In order to reach their targets they ultimately exit/enter the cranium through openings in the skull. Hence, their name is derived from their association with the cranium. The function of the cranial nerves is similar to the spinal nerves, the nerves that are associated with the spinal cord. The motor components of the cranial nerves are derived from cells that are located in the brain. These cells send their axons (e.g., bundles of axons outside the brain, the bundles themselves comprising the nerve) out of the cranium where they ultimately control muscle (e.g., eye movements, diaphragm muscles, muscles used for posture, etc.), glandular tissue (e.g., salivary glands), or specialized muscle (e.g., heart or stomach). The sensory components of cranial nerves originate from collections of cells that are located outside the brain. These collections of nerve cell bodies are called sensory ganglia. They are similar functionally and anatomically to the dorsal root ganglia which are associated with the spinal cord. In general, sensory ganglia of the cranial nerves send out a branch that divides into two branches: a branch that enters the brain and one that is connected to a sensory organ. Examples of sensory organs are pressure or pain sensors in the skin and more specialized ones such as taste receptors of the tongue. Electrical impulses are transmitted from the sensory organ through the ganglia and into the brain via the sensory branch that enter the brain. In summary, the motor components of cranial nerves transmit nerve impulses from the brain to target tissue outside of the brain. Sensory components transmit nerve impulses from sensory organs to the brain. Each cranial nerve (CN) is described below.

CN I. Olfactory Nerve. The olfactory nerve is a collection of sensory nerve rootlets that extend down from the olfactory bulb and pass through the many openings of the cribriform plate in the ethmoid bone. These specialized sensory receptive parts of the olfactory nerve are located in the olfactory mucosa of the upper parts of the nasal cavity. During breathing air molecules attach to the olfactory mucosa and stimulate the olfactory receptors of cranial nerve I and electrical activity is transduced into the olfactory bulb. Olfactory bulb cells transmit electrical activity to other parts of the central nervous system via the olfactory tract.

CN II. Optic Nerve. The optic nerve originates from the bipolar cells of the retina that are connected to the specialized receptors in the retina (rod and cone cells). Light strikes the rod and cone cells and electrical impulses are transduced and transmitted to the bipolar cells. The bipolar cells in turn transmit electrical activity to the central nervous system through the optic nerve. The optic nerve exits the back of the eye in the orbit and enters the optic canal and exits into the cranium. It enters the central nervous system at the optic chiasm (crossing) where the nerve fibers become the optic tract just prior to entering the brain.

CN III. Oculomotor Nerve. The oculomotor nerve originates from motor neurons in the oculomotor (somatomotor) and Edinger-Westphal (visceral motor) nuclei in the brainstem. Nerve cell bodies in this region give rise to axons that exit the ventral surface of the brainstem as the oculomotor nerve. The nerve passes through the two layers of the dura mater including the lateral wall of the cavernous sinus and then enters the superior orbital fissure to access the orbit. The somatomotor component of the nerve divides into a superior and inferior division. The superior division supplies the levator palpebrae superioris and superior rectus muscles. The inferior division supplies the medial rectus, inferior rectus and inferior oblique muscles. The visceromotor or parasympathetic component of the oculomotor nerve travels with inferior division. In the orbit, the inferior division sends branches that enter the ciliary ganglion where they form functional contacts (e.g., synapses) with the ganglion cells. The ganglion cells send nerve fibers into the back of the eye where they travel to ultimately innervate the ciliary muscle and the constrictor pupillae muscle.

CN IV. Trochlear Nerve. The trochlear nerve is purely a motor nerve and is the only cranial nerve to exit the brain dorsally. The trochlear nerve supplies one muscle: the superior oblique. The cell bodies that originate the fourth cranial nerve are located in the ventral part of the brainstem in the trochlear nucleus. The trochlear nucleus gives rise to nerves that cross to the other side of the brainstem just prior to exiting the brainstem. Thus, each superior oblique muscle is supplied by nerve fibers from the trochlear nucleus of the opposite side. The trochlear nerve fibers curve forward and enter the dura mater at the angle between the free and attached border of the tentorium cerebelli. The nerve travels in the lateral wall of the cavernous sinus and then enters the orbit via the superior orbital fissure. The nerve travels medially and diagonally across the levator palpebrae superioris and superior rectus muscle to innervate the superior oblique muscle.

CN V. Trigeminal Nerve. The trigeminal nerve as the name indicates is composed of three large branches. They are the ophthalmic (V₁, sensory), maxillary (V₂, sensory), and mandibular (V₃, motor and sensory) branches. The large sensory root and smaller motor root leave the brainstem at the midlateral surface of the pons. The sensory root terminates in the largest of the cranial nerve nuclei which extends from the pons all the way down into the second cervical level of the spinal cord. The sensory root joins the trigeminal or semilunar ganglion between the layers of the dura mater in a depression on the floor of the middle crania fossa. This depression is the location of the so called Meckle's cave. The motor root originates from cells located in the masticator motor nucleus of trigeminal nerve located in the midpons of the brainstem. The motor root passes through the trigeminal ganglion and combines with the corresponding sensory root to become the mandibular nerve. It is distributed to the muscles of mastication, the mylohyoid muscle and the anterior belly of the digastric. The mandibular nerve also innervates the tensor veli palatini and tensor tympani muscles. The three sensory branches of the trigeminal nerve emanate from the ganglia to form the three branches of the trigeminal nerve. The ophthalmic and maxillary branches travel in the wall of the cavernous sinus just prior to leaving the cranium. The ophthalmic branch travels through the superior orbital fissure and passes through the orbit to reach the skin of the forehead and top of the head. The maxillary nerve enters the cranium through the foramen rotundum via the pterygopalatine fossa. Its sensory branches reach the pterygopalatine fossa via the inferior orbital fissure (face, cheek and upper teeth) and pterygopalatine canal (soft and hard palate, nasal cavity and pharynx). There are also meningeal sensory branches that enter the trigeminal ganglion within the cranium. The sensory part of the mandibular nerve is composed of branches that carry signals (e.g., electrical currents (e.g., encoding general sensory information)) from the mucous membranes of the mouth and cheek, anterior two-thirds of the tongue, lower teeth, skin of the lower jaw, side of the head and scalp and meninges of the anterior and middle cranial fossae.

CN VI. Abducens Nerve. The abducens nerve originates from neuronal cell bodies located in the ventral pons. These cells give rise to axons that follow a ventral course and exit the brain at the junction of the pons and the pyramid of the medulla. The nerve of each side then travels anteriorly where it pierces the dura lateral to the dorsum sellae. The nerve continues forward and bends over the ridge of the petrous part of the temporal bone and enters the cavernous sinus. The nerve passes lateral to the carotid artery prior to entering superior orbital fissure. The abducens nerve passes through the common tendonous ring of the four rectus muscles and then enters the deep surface of the lateral rectus muscle. The function of the abducens nerve is to contract the lateral rectus which results in abduction of the eye. The abducens nerve in humans is solely a somatomotor nerve.

CN VII. Facial Nerve. The facial nerve is a mixed nerve containing both sensory and motor components. The nerve emanates from the brain stem at the ventral part of the pontomedullary junction. The nerve enters the internal auditory meatus where the sensory part of the nerve forms the geniculate ganglion. The greater petrosal nerve branches from the facial nerve in the internal auditory meatus. The facial nerve continues in the facial canal where the chorda tympani branches from it. The facial nerve leaves the skull via the styolomastoid foramen. The chorda tympani passes through the petrotympanic fissure before entering the infratemporal fossae. The main body of the facial nerve is somatomotor and supplies the muscles of facial expression. The somatomotor component originates from neurons in the facial motor nucleus located in the ventral pons. The visceral motor or autonomic (parasympathetic) part of the facial nerve is carried by the greater petrosal nerve. The greater petrosal nerve leaves the internal auditory meatus via the hiatus of the greater petrosal nerve which is found on the anterior surface of the petrous part of the temporal bone in the middle cranial fossa. The greater petrosal nerve passes forward across the foramen lacerum where it is joined by the deep petrosal nerve (sympathetic from superior cervical ganglion). Together these two nerves enter the pterygoid canal as the nerve of the pterygoid canal. The greater petrosal nerve exits the canal with the deep petrosal nerve and synapses in the pterygopalatine ganglion in the pterygopalatine fossa. The ganglion then provides nerve branches that supply the lacrimal gland and the mucous secreting glands of the nasal and oral cavities. The other parasympathetic part of the facial nerve travel with the chorda tympani which joins the lingual nerve in the infratemporal fossa. They travel with lingual nerve prior to synapsing in the submandibular ganglion which is located in the lateral floor of the oral cavity. The submandibular ganglion originates nerve fibers that innervate the submandibular and sublingual glands. The visceral motor components of the facial nerve originate in the lacrimal or superior salivatory nucleus. The nerve fibers exit the brainstem via the nervus intermedius. The nervus intermedius is so called because of its intermediate location between the eighth cranial nerve and the somatomotor part of the facial nerve just prior to entering the brain. There are two sensory (special and general) components of facial nerve both of which originate from cell bodies in the geniculate ganglion. The special sensory component carries information from the tongue (e.g., taste buds in the tongue) and travel in the chorda tympani. The general sensory component conducts signals (e.g., electrical signals (e.g., encoding sensation from skin) in the external auditory meatus, a small area behind the ear, and external surface of the tympanic membrane. These signals (e.g., sensory components) are connected with cells in the geniculate ganglion. Both the general and visceral signals (e.g., sensory components) travel into the brain with nervus intermedius part of the facial nerve. The signals (e.g., general sensory component) enter the brainstem and eventually synapses in the spinal part of trigeminal nucleus. Other signals (e.g., special sensory or taste signals) enter fibers in the brainstem and terminate in the gustatory nucleus, a rostral part of the nucleus of the solitary tract.

CN VIII. Vestibulocochlear Nerve. The vestibulocochlear nerve is a sensory nerve that conducts two senses: hearing (audition) and balance (vestibular). The receptor cells for these senses are located in the membranous labyrinth that is embedded in the petrous part of the temporal bone. There are two specialized organs in the bony labyrinth, the cochlea and the vestibular apparatus. The cochlear duct is the organ that is connected to the three bony ossicles that transduce sound waves into fluid movement in the cochlea. This ultimately causes movement of hair cells that activate (e.g., provide signals (e.g., electrical signals) to) the auditory part of the vestibulocochlear nerve. As described herein, the vestibular apparatus is the organ that senses head position changes relative to gravity. Movement causes fluid vibration resulting in hair cell displacement that activates the vestibular part of the vestibulocochlear nerve. The peripheral parts of the vestibulocochlear nerve travel a short distance to nerve cell bodies at the base of the corresponding sense organs. From these peripheral sensory nerve cells the central part of the nerve then travels through the internal auditory meatus with the facial nerve. The eighth nerve enters the brain stem at the junction of the pons and medulla lateral to the facial nerve. The auditory component of the vestibulocochlear nerve terminates in a sensory nucleus called the cochlear nucleus that is located at the junction of the pons and medulla. The vestibular part of the eighth nerve ends in the vestibular nuclear complex located in the floor of the fourth ventricle.

CN IX. Glossopharyngeal Nerve. The glossopharyngeal nerve is related to the tongue and the pharynx. The glossopharyngeal cranial nerve exits the brain stem as the most rostral of a series of nerve rootlets that protrude between the olive and inferior cerebellar peduncle. These nerve rootlets come together to form the glossopharyngeal cranial nerve and leave the skull through the jugular foramen. The tympanic nerve is a branch that occurs prior the glossopharyngeal nerve exiting the skull. The visceromotor or parasympathetic part of the glossopharyngeal nerve originate in the inferior salivatory nucleus. Nerve fibers from this nucleus join the other components of the ninth nerve during their exit from the brain stem. They branch in the cranium as the tympanic nerve. The tympanic nerve exits the jugular foramen and passes by the inferior glossopharyngeal ganglion. It re-enters the skull through the inferior tympanic canaliculus and reaches the tympanic cavity where it forms a plexus in the middle ear cavity. The nerve travels from this plexus through a canal and out into the middle cranial fossa adjacent to the exit of the greater petrosal nerve. It is here the nerve becomes the lesser petrosal nerve. The lesser petrosal nerve exits the cranium via the foramen ovali and synapses in the otic ganglion. The otic ganglion provides nerve fibers that innervate and control the parotid gland, an important salivary gland. The branchial motor component supplies the stylopharyngeas muscle that elevates the pharynx during swallowing and talking. In the jugular foramen are two sensory ganglion connected to the glossopharyngeal nerve: the superior and inferior glossopharyngeal ganglia. General sensory components from the skin of the external ear, inner surface of the tympanic membrane, posterior one-third of the tongue and the upper pharynx join either the superior or inferior glossopharyngeal ganglia. The ganglia send central processes into the brain stem that terminate in the caudal part of the spinal trigeminal nucleus. Visceral sensory nerve fibers originate from the carotid body (e.g., oxygen tension measurement) and carotid sinus (e.g., blood pressure changes). The visceral sensory nerve components connect to the inferior glossopharngeal ganglion. The central process extend from the ganglion and enter the brain stem to terminate in the nucleus solitarius. Signals (e.g., encoding taste sensations) from the posterior one-third of the tongue travels via nerve fibers that enter the inferior glossopharnygeal ganglion. The central process that carry this special sense travel through the jugular foramen and enter the brain stem. They terminate in the rostral part of the nucleus solitarius (gustatory nucleus).

CN X. Vagus Nerve. The vagus nerve is the longest of the cranial nerve. The vagus nerve travels from the brain stem through organs in the neck, thorax and abdomen. The nerve exits the brain stem through rootlets in the medulla that are caudal to the rootlets for the glossopharyngeal nerve. The rootlets form the vagus nerve and exit the cranium via the jugular foramen. Similar to the ninth cranial nerve there are two sensory ganglia associated with the vagus nerve. They are the superior and inferior vagal ganglia. The branchial motor component of the vagus nerve originates in the medulla in the nucleus ambiguus. The nucleus ambiguus contributes to the vagus nerve as three major branches that leave the nerve distal to the jugular foramen. The pharyngeal branch travels between the internal and external carotid arteries and enters the pharynx at the upper border of the middle constrictor muscle. It supplies all of the muscles of the pharynx and soft palate except the stylopharyngeas and tensor palati. These include the three constrictor muscles, levator veli palatini, salpingopharyngeus, palatopharyngeus and palatoglossal muscles. The superior laryngeal nerve branches distal to the pharyngeal branch and descends lateral to the pharynx. It divides into an internal and external branch. The internal branch is purely sensory. The external branch travels to the cricothyroid muscle that it supplies. The third branch is the recurrent branch of the vagus nerve and it travels a different path on the left and right sides of the body. On the right side the recurrent branch leaves the vagus anterior to the subclavian artery and wraps back around the artery to ascend posterior to it. The right recurrent branch ascends to a groove between the trachea and esophagus. The left recurrent branch leaves the vagus nerve on the aortic arch and loops posterior to the arch to ascend through the superior mediastinum. The left recurrent branch ascends along a groove between the esophagus and trachea. Both recurrent branches enter the larynx below the inferior constrictor and supply intrinsic muscles of larynx excluding the cricothyroid. The visceromotor or parasympathetic component of the vagus nerve originates from the dorsal motor nucleus of the vagus in the dorsal medulla. These cells give rise to axons that travel in the vagus nerve. The visceromotor part of the vagus innervates ganglionic neurons located in or adjacent to each target organ. The target organs in the head and neck include glands of the pharynx and larynx (via the pharyngeal and internal branches). In the thorax, branches travels into the lungs for bronchoconstriction, the esophagus for peristalsis and the heart for slowing of heart rate. In the abdomen branches enter the stomach, pancreas, small intestine, large intestine and colon for secretion and constriction of smooth muscle. The viscerosensory component of the vagus are derived from nerves that have receptors in the abdominal viscera, esophagus, heart and aortic arch, lungs, bronchia and trachea. Nerves in the abdomen and thorax join the left and right vagus nerves to ascend beside the left and right common carotid arteries. Sensation from the mucous membranes of the epiglottis, base of the tongue, aryepiglottic folds and the upper larynx travel via the internal laryngeal nerve. Sensation below the vocal folds of the larynx is carried by the recurrent laryngeal nerves. The cell bodies that give rise to the peripheral processes of the visceral sensory nerves of the vagus are located in the inferior vagal ganglion. The central process exits the ganglion and enters the brain stem to terminate in the nucleus solitarius. The general sensory components of the vagus nerve conduct sensation from the larynx, pharynx, skin the external ear and external auditory canal, external surface of the tympanic membrane, and the meninges of the posterior cranial fossa. Sensation from the larynx travels via the recurrent laryngeal and internal branches of the vagus to reach the inferior vagal ganglion. Sensory nerve fibers from the skin and tympanic membrane travel with auricular branch of the vagus to reach the superior vagal ganglion. The central processes from both ganglia enter the medulla and terminate in the nucleus of the spinal trigeminal tract.

CN XI. Spinal Accessory Nerve. The spinal accessory nerve originates from neuronal cell bodies located in the cervical spinal cord and caudal medulla. Most are located in the spinal cord and ascend through the foramen magnum and exit the cranium through the jugular foramen. They are branchiomotor in function and innervate the sternocleidomastoid and trapezius muscles in the neck and back. The cranial root of the accessory nerve originates from cells located in the caudal medulla. They are found in the nucleus ambiguus and leave the brainstem with the fibers of the vagus nerve. They join the spinal root to exit the jugular foramen. They rejoin the vagus nerve and distribute to the same targets as the vagus.

CN XII. Hypoglossal Nerve. The hypoglossal nerve as the name indicates can be found below the tongue. It is a somatomotor nerve that innervates all the intrinsic and all but one of the extrinsic muscles of the tongue. The neuronal cell bodies that originate the hypoglossal nerve are found in the dorsal medulla of the brain stem in the hypoglossal nucleus. This nucleus gives rise to axons that exit as rootlets that emerge in the ventrolateral sulcus of the medulla between the olive and pyramid. The rootlets come together to form the hypoglossal nerve and exit the cranium via the hypoglossal canal. The nerve passes laterally and inferiorly between the internal carotid artery and internal jugular vein. The hypoglossal nerve travels lateral to the bifurcation of the common carotid and loops anteriorly above the greater horn of the hyoid bone to run on the lateral surface of the hyoglossus muscle. It then travels above the edge of the mylohyoid muscle. The hypoglossal nerve then separates into branches that supply the intrinsic muscles and three of the four extrinsic muscles of the tongue.

The main structures of the hindbrain are the medulla oblongata, pons and cerebellum. The medulla oblongata (or simply medulla) looks like a swollen tip to the spinal cord. The medulla is continuous with the upper part of the spinal cord and contains portions of both motor and sensory tracts. Decussation of pyramids occurs in the medulla, wherein ascending and descending tracts cross. The medulla contains nuclei that are reflex centers (e.g., for regulation of heart rate (e.g., that rhythmically stimulate the intercostal muscles and diaphragm), respiration rate, vasoconstriction, swallowing, coughing, sneezing, vomiting, and hiccupping). The medulla also contains nuclei of origin for cranial nerves VIII-XII. Nuclei are a collection of somas (e.g., nerve cell bodies) with the nerve tract within the central nervous system (e.g., that relay body sensory information (e.g., balance) to parts of the brain (e.g., thalamus)). The medulla also contains olivary (e.g., that insure precise, voluntary movements and maintain equilibrium) and vestibular (e.g., those that maintain equilibrium) nuclei.

For example, the rate of cellular respiration (e.g., oxygen consumption and carbon dioxide production) varies with the level of activity. Vigorous exercise can increase by 20-25 times the demand of tissues for oxygen. This is met by increasing the rate and depth of breathing. However, it is a rising concentration of carbon dioxide, and not a declining concentration of oxygen, that plays the major role in regulating the ventilation of the lungs. The concentration of CO₂ is monitored by cells in the medulla oblongata. If the level rises, the medulla responds by increasing the activity of the motor nerves that control the intercostal muscles and diaphragm. The neurons controlling breathing have mu (g) receptors (e.g. the receptors to which opiates (e.g., heroin, morphine, codeine) bind). This accounts for the suppressive effect of opiates on breathing. Destruction of the medulla causes instant death.

The pons is superior to the medulla and connects the spinal cord with the brain. The pons also acts as a relay station carrying signals from various parts of the cerebral cortex to the cerebellum. Nerve impulses coming from the eyes (e.g., from the oculomotor nerve), ears (e.g., from the vestibulocochlear nerve), and touch receptors (e.g., trigeminal and facial nerves) are sent to the cerebellum via the pons. The pons also relays nerve impulses related to voluntary skeletal movements from the cerebral cortex to the cerebellum. The pons contains the nuclei for cranial nerves V through VII. The pons also contains pneumotaxic and apneustic areas that help control respiration along with the respiratory center of the medulla.

The reticular formation is a region running through the middle of the hindbrain and on into the midbrain. It receives sensory input (e.g., sound) from higher in the brain and passes these back up to the thalamus. The reticular formation is involved in sleep, consciousness, muscle tone, arousal, and vomiting. A large portion of the brain stem (e.g., comprising the medulla, pons, and midbrain) consists of small areas of gray matter interspersed among fibers of white matter, the reticular formation. The reticular formation has both sensory and motor functions. The reticular formation helps to regulate muscle tone, alerts the cortex to incoming sensory signals (e.g., from the reticular activating system, or RAS), and is responsible for maintaining consciousness and awakening from sleep.

The brain stem is a compact stalk through which most information flowing to and from the brain travels. The brainstem is also the site of many important nuclei involved with cranial nerve function (e.g., cranial nerves (e.g., nuclei of cranial nerves) II-XII are associated with the brainstem). Thus, the brainstem is important for maintaining consciousness, cerebellar circuitry, muscle tone and posture, and for homeostatic control of respiration and cardiac function.

The cerebellum consists of two deeply-convoluted hemispheres. Although it represents only 10% of the weight of the brain, it contains as many neurons as all the rest of the brain combined. The cerebellum functions to coordinate body movements. For example, people with damage to their cerebellum have reported being unable to perceive the world as before (e.g., without damage), have difficulty contracting their muscles, and display jerky and uncoordinated motions. Furthermore, the cerebellum is a center for attaining implicit memory (e.g., motor skills) and laboratory studies have demonstrated the role of the cerebellum in both long-term potentiation (LTP) and long-term depression (LTD).

The limbic system receives input from various association areas in the cerebral cortex and passes signals on to the nucleus accumbens. The limbic system comprises the hippocampus. The hippocampus is also important for the formation of long-term memories (e.g., long term potentiation).

Long term potentiation (LTP) of neurotransmission at glutamatergic synapses comprises multiple steps. Glutamate (glutamic acid) is an excitatory neurotransmitter released from primary afferent sensory nerves in the spinal cord. In the brain it is the neurotransmitter in cortical pyramidal (output) neurons whose axons form association and commisural pathways (e.g., linking, respectively, different areas of the same cortex and corresponding areas of different cortices), corticothalamic and thalamocortical pathways (e.g., forming reciprocal connections between thalamus and cortex), and corticostriatal pathways linking the cortex with the basal ganglia. Glutamate is a synaptic organiser as well as a synaptic transmitter.

Thus, short term potentiation (STP) and LTP refer to the enhanced transmission that occurs at glutamatergic synapses following initial stimulation within certain frequency ranges. STP and LTP can occur after adjacent glutamatergic and nonglutamatergic synapses are activated concurrently. The enhanced activity involves both NMDA and AMPA type glutamate receptors. LTP has been implicated in wind-up of nociception in the spinal cord, kindling of epileptic seizures and in memory.

The midbrain occupies a small region in humans (e.g., it is relatively much larger in “lower” vertebrates). The midbrain comprises the reticular formation (e.g., that collects input from higher brain centers and passes it on to motor neurons), the substantia nigra (e.g., that helps “smooth” out body movements (e.g., damage to the substantia nigra can cause Parkinson's disease)), and the ventral tegmental area (VTA) that is packed with dopamine-releasing neurons activated by nicotinic acetylcholine receptors and whose projections synapse deep within the forebrain. The VTA appears to be involved in pleasure (e.g., nicotine, amphetamines and cocaine bind to and activate VTA dopamine-releasing neurons and account, at least in part, for their addictive qualities).

The human forebrain is made up of a pair of large cerebral hemispheres, called the telencephalon. Because of crossing over of the spinal tracts, the left hemisphere of the forebrain deals with the right side of the body and vice versa. The forebrain also comprises a group of unpaired structures located deep within the cerebrum, called the diencephalon.

The diencephalons comprises the thalamus, lateral geniculate nucleus, hypothalamus and the posterior lobe of the pituitary. The thalamus, located superior to the midbrain, contains nuclei that serve as relay stations for all sensory impulses, except smell, to the somatic-sensory regions of the cerebral cortex. The thalamus also registers conscious recognition of pain and temperature and some awareness of light touch and pressure. Also, signals from the cerebellum pass through the thalamus on the way to the motor areas of the cerebral cortex.

All signals entering the brain from the optic nerves enter the lateral geniculate nucleus (LGN) and undergo some processing before moving onto the various visual areas of the cerebral cortex.

The hypothalamus is inferior to the thalamus, has four major regions (mammilary, tuberal, supraoptic, and preoptic), controls many body activities, and is one of the major regulators of homeostasis (e.g., of the autonomic nervous system). Damage to the hypothalamus is quickly fatal as the normal homeostasis of body temperature, blood chemistry, etc. spirals out of control. The hypothalamus is the source of various hormones, two of which pass into the posterior lobe of the pituitary gland (e.g., antidiuretic hormone (ADH) and oxytocin) from the hypothalamus before they are released into the blood.

The vestibular and auditory systems innervate multiple portions of the central nervous system. Furthermore, the auditory and vestibular systems themselves are intimately connected. Receptors for both are located in the temporal bone in a convoluted chamber called the bony labyrinth. A delicate continuous membrane is suspended within the bony labyrinth, creating a second chamber within the first. This chamber is called the membranous labyrinth. The entire fluid-filled structure is called the inner ear.

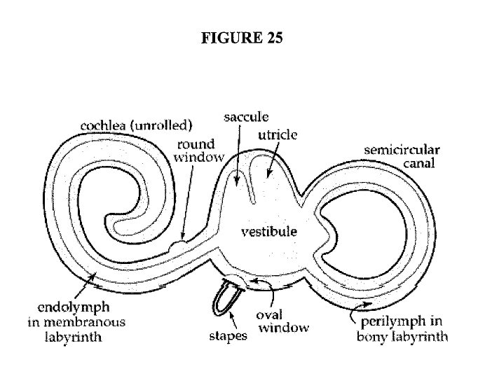

The inner ear has two membrane-covered outlets into the air-filled middle ear: the oval window and the round window (See FIG. 25). The oval window is filled by the plate of the stapes, the third middle ear bone. The stapes vibrates in response to vibrations of the eardrum, setting the fluid of the inner ear in motion back and forth. The round window serves as a pressure valve, bulging outward as pressure rises in the inner ear.

The oval window opens into a large central area within the inner ear called the vestibule. All of the inner ear organs branch off from this central chamber. On one side is the cochlea, on the other the semicircular canals. Additional vestibular organs (e.g., the utricle and saccule) are adjacent to the vestibule.

The membranous labyrinth is filled with a special fluid called endolymph. Endolymph is very similar to intracellular fluid: it is high in potassium and low in sodium. The ionic composition is important for vestibular and auditory hair cells to function optimally. The space between the membranous and bony labyrinths is filled with perilymph, which is very much like normal cerebral spinal fluid.

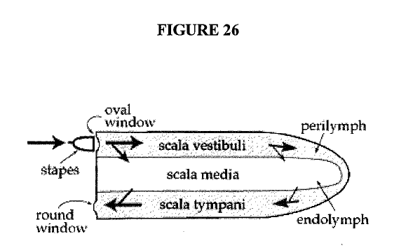

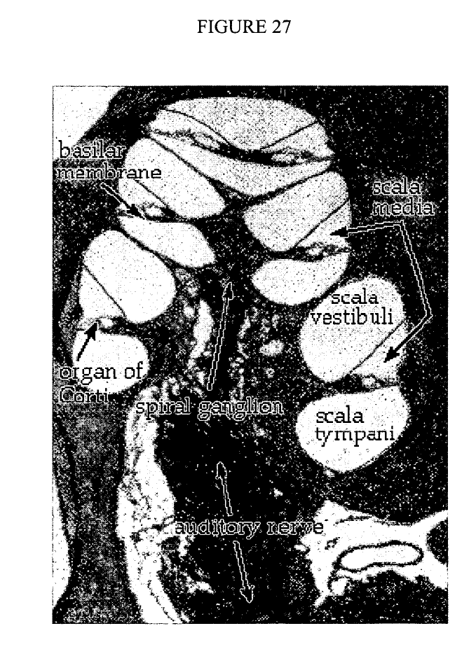

The transduction of sound into a neural signal occurs in the cochlea. If the snail-shaped cochlea were unrolled, it would look FIG. 26. As the stapes vibrates the oval window, the perilymph moves (e.g., sloshes) back and forth, vibrating the round window in a complementary rhythm. The membranous labyrinth is caught between the two, and bounces up and down with the motion (e.g., sloshing). A closer look at the membranous labyrinth is shown in FIG. 27 in which a cross section of the cochlea is shown.

The membranous labyrinth of the cochlea encloses the endolymph-filled scala media. The two compartments of the bony labyrinth that house the perilymph are called the scalae vestibuli and tympani. Within the scala media is the receptor organ, the organ of Corti. It rests on part of the membranous labyrinth, the basilar membrane. The auditory hair cells sit within the organ of Corti. There are inner hair cells, that are the auditory receptors, and outer hair cells, that help to “tune” the cochlea, as well as supporting cells. The sensitive stereocilia of the inner hair cells are embedded in a membrane called the tectorial membrane. As the basilar membrane bounces up and down, the fine stereocilia are sheared back and forth under the tectorial membrane. When the stereocilia are pulled in the right direction, the hair cell depolarizes. This signal (e.g., electrical signal) is transmitted to a nerve process lying under the organ of Corti. This neuron transmits the signal back along the auditory nerve to the brainstem. As with almost all sensory neurons (the exception is in the retina), the auditory cell body lies outside the CNS in a ganglion. In this case, the ganglion is stretched out along the spiralling center axis of the cochlea, and is named the spiral ganglion.

The basilar membrane is actually thinner and narrower at the base of the cochlea than at the tip (apex). The properties of the basilar membrane change as its shape changes. This means that the basilar membrane vibrates to high frequencies at the base of the cochlea and to low frequencies at the apex. A hair cell at the base of the cochlea will respond best to high frequencies, since at those frequencies the basilar membrane underneath it will vibrate the most. Thus, although the hair cells are arranged in order along the basilar membrane, from high-frequency to low-frequency, it is the properties of the basilar membrane that set up this gradient, not the properties of the hair cells.

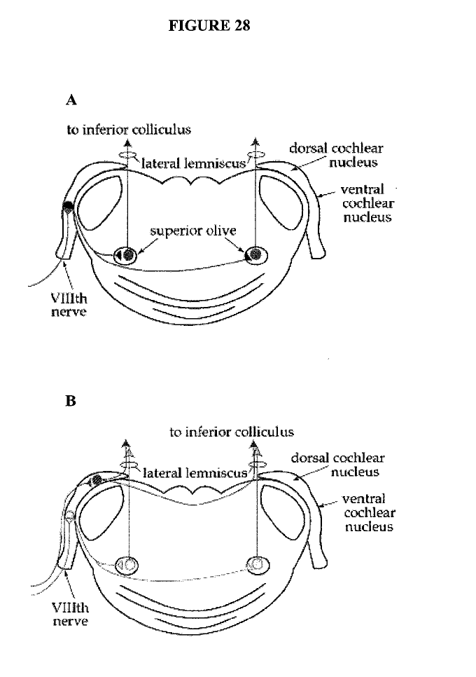

The auditory nerve carries the signal into the brainstem and synapses in the cochlear nuclei (See FIG. 28A). From the cochlear nuclei, auditory information is split into at least two streams, much like the visual pathways are split into motion and form processing. Auditory nerve fibers going to the ventral cochlear nucleus synapse on their target cells with giant, hand-like terminals. The ventral cochlear nucleus cells then project to a collection of nuclei in the medulla called the superior olive. In the superior olive, the minute differences in the timing and loudness of the sound in each ear are compared, and from this the direction the sound came from can be determined. The superior olive then projects up to the inferior colliculus via a fiber tract called the lateral lemniscus.

The second stream of information starts in the dorsal cochlear nucleus (See FIG. 28B). This stream analyzes the quality of sound. The dorsal cochlear nucleus picks apart tiny frequency differences (e.g., that distinguish “hat” from “bat” and “cat”). This pathway projects directly to the inferior colliculus, also via the lateral lemniscus.

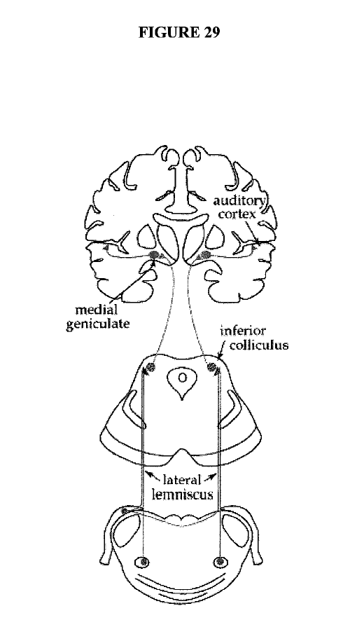

From the inferior colliculus, both streams of information proceed to sensory thalamus. The auditory nucleus of the thalamus is the medial geniculate nucleus (See FIG. 29). The medial geniculate projects to primary auditory cortex, located on the banks of the temporal lobes.

As stated above, the auditory and vestibular systems are intimately connected. One function of the vestibular system is to provide orientation to a subject on the position and motion of his or her head in space. One must be able to detect rotation, such as what happens when the head is shaken or nodded. This type of movement is termed angular acceleration. One must also be able to detect motion along a line (e.g., when the body begins to lean to one side). This is called linear acceleration. The vestibular systems comprises two separate receptor organs to accomplish these tasks, semicircular canals (e.g., that detect angular acceleration) and the utricle and saccule (e.g., that detect linear acceleration).

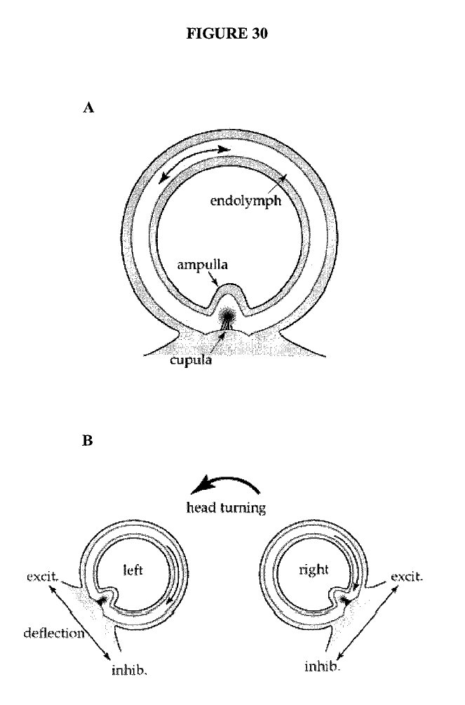

The semicircular canals can detect angular acceleration. There are three canals, corresponding to the three dimensions in which the body moves, so that each canal can detect motion in a single plane. Each canal is set up as shown in FIG. 30A, as a continuous endolymph-filled hoop. The actual hair cells sit in a small swelling at the base called the ampula.

The hair cells are arranged as a single tuft that projects up into a gelatinous mass, the cupula. When the head is turned in the plane of the canal, the inertia of the endolymph causes it to move (e.g., slosh) against the cupula, deflecting the hair cells. If one were to continue turning in circles, eventually the fluid would catch up with the canal, and there would be no more pressure on the cupula. When one stops after spinning, the moving fluid would move against a suddenly still cupula (e.g., and one would perceive that he or she were turning in the other direction). This same arrangement is mirrored on both sides of the head. Each tuft of hair cells is polarized (e.g., if the tufts are pushed one way, they become excited, but if pushed in the other direction, they become inhibited). This means that the canals on either side of the head will generally be operating in a push-pull rhythm; when one is excited, the other is inhibited (See FIG. 30B). To maintain a sense of homeostasis (e.g., balance, security, and/or orientation), it is important that both sides agree as to what the head is doing. If there is disagreement (e.g., if both sides push at once, or if the brain perceives that both sides are pushing at once (e.g., in the absence of both sides doing so)) dizziness (e.g., debilitating vertigo) and nausea may result. For example, this is the reason that infections of the endolymph or damage to the inner ear can cause dizziness (e.g., vertigo). Thus, each side acts in concert with the other side to constantly sense head position and orientation.

A large role of the semicircular canal system is to keep the eyes still in space while the head moves around them. The semicircular canals exert direct control over the eyes, so they can directly compensate for head movements. The eye is controlled by three pairs of muscles; the medial and lateral rectus, the superior and inferior rectus, and the inferior and superior oblique. Each of these muscles direction of motion is at a diagonal. These diagonals are matched closely by the three planes of the semicircular canals so that, in general, a single canal interacts with a single muscle pair. The entire compensatory reflex is called the vestibulo-ocular reflex (VOR).

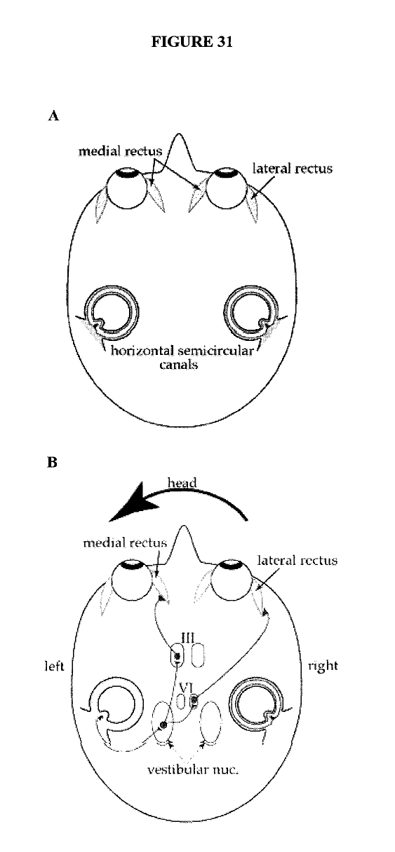

The VOR works on all three muscle pairs. For example, the medial-lateral rectus pair, coupled to the horizontal canal, is shown in FIG. 31A looking down at a person's head. The lateral rectus muscle pull the eye laterally, and the medial rectus pull the eye medially, both in the horizontal plane. The horizontal canal detects rotation in the horizontal plane.

Thus, if one moves their head to the left, they will excite the left horizontal canal, inhibiting the right. In order to keep the eyes fixed on a stationary point, one needs to fire the right lateral rectus and the left medial rectus (e.g., thereby moving the eyes to the right) (See FIG. 31B).

For example, the pathway may be as follows: the vestibular nerve enters the brainstem and synapses in the vestibular nucleus. Cells that received information from the left horizontal canal project to the abducens nucleus on the right side, to stimulate the lateral rectus. They also project to the oculomotor nucleus on the left side, to stimulate the medial rectus. These same vestibular cells also inhibit the opposing muscles (e.g., in the example provided above, the right medial rectus, and the left lateral rectus). Thus, the right horizontal canal is wired to the complementary set of muscles. Since it is inhibited, it will not excite its target muscles (the right medial rectus and the left lateral rectus), nor will it inhibit the muscles used (the right lateral rectus and the left medial rectus).

A great deal of the VOR axon traffic travels via a fiber highway called the MLF (medial longitudinal fasciculus). The integrity of this tract is crucial for the VOR to work properly. When the VOR is damaged (e.g., by medial brainstem strokes, or injury), dizziness (e.g., incapacitating vertigo) and nausea may occur.

The utricle and saccule detect linear acceleration. Each organ has a sheet of hair cells, the macula, whose cilia are embedded in a gelatinous mass (e.g., similar to the semicircular canals). Unlike the canals, however, this gel has a clump of small crystals embedded in it, called an otolith. The otoliths provide the inertia, so that when one moves to one side, the otolith-gel mass drags on the hair cells. Once moving at a constant speed (e.g., such as in a car), the otoliths come to equilibrium and a subject no longer perceives the motion.

The hair cells in the utricle and saccule are polarized, but they are arrayed in different directions so that a single sheet of hair cells can detect motion forward and back, side to side. Each macula can therefore cover two dimensions of movement. The utricle lays horizontally in the ear, and can detect any motion in the horizontal plane. The saccule is oriented vertically, so it can detect motion in the sagittal plane (up and down, forward and back). Thus, a major role of the saccule and utricle is to provide vertical orientation to a subject with respect to gravity. If the head and body start to tilt, the vestibular nuclei will automatically compensate with the correct postural adjustments.

The vestibular afferent pathways display a great deal of convergence (See, e.g., Fitzpatrick and Day, J Appl Physiol 96, 2301-2316 (2004)). Each primary afferent innervates many hair cells (See, e.g., Fernandez et al., J Neurophysiol 60: 167-181, (1988); J Neurophysiol 73: 1253-1269, (1995). J Neurophysiol 60: 167-181, 1988.

The secondary vestibular neurons of the vestibular nuclei project to many areas of the central nervous system. For example, the nuclei project to the oculomotor nuclei, the spinal cord, and the flocculus of the cerebellum (See, e.g., Highstein et al., J Neurophysiol 58: 719-738, 1987), as well as to the thalamus and cortex areas (e.g., the thalamocortical pathway). Even by the level of the secondary neuron, there is convergence of afferents from the semicircular canals and otolith organs (See, e.g., Dickman and Angelaki, J Neurophysiol 88: 3518-3533, (2002); Kasper et al., J Neurophysiol 60: 1753-1764, (1988)) and from otolith afferents from both sides of the striola and both sides of the head (See, e.g., Uchino et al., Ann NY Acad Sci 871: 162-172, (1999); Uchino et al., Exp Brain Res 136: 421-430, (2001)). Thus spinal projecting neurons of the lateral vestibular nucleus respond optimally to movement in directions such as pure roll that are not encoded by any single canal (Kasper et al., J Neurophysiol 60: 1753-1764, (1988)), and a higher level of spatial tuning increases the direction specificity of secondary otolith neurons to linear acceleration (Angelaki and Dickman, J Neurophysiol 84: 2113-2132, (2000)). Also at this level, there is a large convergence of afferents from the neck (Kasper et al., J Neurophysiol 60: 1765-1778, (1988); Wilson et al., J Neurophysiol 64: 1695-1703, (1990)) so that a complex descending output of these neurons can come from a mix of signals denoting head on body and head in space.

There also exists temporal filtering of the vestibular signal at the secondary neuron level. The transduction mechanics of the semicircular canals act as a low-pass filter so that the afferent canal signal largely resembles an angular velocity response. The process, known as velocity storage (See, e.g., Raphan et al., Exp Brain Res 35: 229-248, 1979), is a further neuronal filtering or integration, so that, even at very low frequencies, the vestibular secondary neuron's response is related to angular velocity. A similar filtering exists for otolith signals. Whereas primary afferents respond in proportion to linear acceleration, most central otolith neurons respond in proportion to linear velocity (Angelaki and Dickman, J Neurophysiol 84: 2113-2132, (2000)). This is particularly so at low frequencies (<0.5 Hz), which are most significant for balance control.

Areas within the somatosensory cortex as well as areas within the parietal cortex also receive vestibular projections (See, e.g., Odkvist et al., Exp Brain Res 21, 97-105 (1974); Fredrickson et al., Exp Brain Res 2, 318-327 (1966)). The ventral-posterior and lateral-posterior nuclei of the posterolateral thalamus are the thalamic areas concerned with this vestibular sensory function and cortical projection (See, e.g., Karnath et al., Proc Natl Acad Sci 97, 13931-13936 (2000)). It is contemplated that these areas are able to modulate vestibular reflexes acting on the neck and limbs (See, e.g., Wilson et al., Exp Brain Res 125, 1-13 (1999)).

Accordingly, in some embodiments, systems and methods of the present invention are used to stimulate the central nervous system (e.g., the brain and or spinal cord). In some embodiments, the stimulation is direct. In some embodiments, the stimulation is indirect (e.g., indirect stimulation of the spinal cord via stimulation of the brain, or, indirect stimulation of the vestibular nerve via stimulation (e.g., tactile (e.g., elctrotactile)) of the tongue). In some embodiments, the systems and methods of the present invention stimulate afferent and/or efferent nerves (e.g., the VIII cranial nerve, or other nerves described herein). In some embodiments, systems and methods of the present invention correct abnormal neurotransmitter release in a subject (e.g., a subject with a vestibular disorder). Although an understanding of the mechanism is not necessary to practice the present invention and the present invention is not limited to any particular mechanism of action, in some embodiments, systems and methods of the present invention provide signals (e.g., stimulation of the central nervous system) important for neurotransmitter (e.g., acetylcholine) release (e.g., at a site of a postsynaptic receptor (e.g., at a muscle or an organ (e.g., organs of the vestibular system (e.g., cochlea, semicircular canals, utricle or saccule)))). In some embodiments, neurotransmitter release generated by signals provided by the systems and methods of the present invention are involved with long term memory (e.g., of beneficial effects provided to a subject training with systems and methods of the present invention).

In some embodiments, systems and methods of the present invention stimulate (e.g., provide signals to) the brain. In some embodiments, signals to the brain induce cholinergic transmission (e.g., acetylcholine release (e.g., at the site of skeletal muscle)). In some embodiments, signals (e.g., provided by systems and methods of the present invention (e.g., via electrotactile stimulation of the tongue, or auditory nerve stimulation with sound (e.g., music))) provided to the brain induce muscarinic and/or cholinergic receptor activity. In some embodiments, the cholinergic receptor so activated is a cholinergic muscarinic receptor innervated by postganglionic fibers of the parasympathetic division of the autonomic nervous system, a cholinergic nicotinic receptor (e.g., in sympathetic or parasympathetic ganglia), and/or a cholinergic nicotinic receptor at the myoneural junction (e.g., motor end plates) of the somatic nervous system. In some embodiments, signals (e.g., provided by systems and methods of the present invention (e.g., via electrotactile stimulation of the tongue, or auditory nerve stimulation with sound (e.g., music))) provided to the brain induce adrenergic receptor activity.

In some embodiments, systems and methods of the present invention stimulate (e.g., provide signals to (e.g., an electrical signal, a nerve impulse, an electrical signal that appears (e.g., is perceived by the brain) as a nerve impulse, an electrical impulse (e.g., that provokes a nerve impulse), and/or both an electrical signal and nerve impulse)) the brain (e.g., via sensory ganglia of a cranial nerve (e.g., any one or more of cranial nerves I-XII)). In some embodiments, the brain detects and processes the signal and transmits a nerve impulse (e.g., via a cranial nerve) to a target (e.g., muscle (e.g., controlling eye movements, diaphragm muscles, muscles used for posture), glandular tissue, or specialized tissue (e.g., heart or stomach tissue)).

In some embodiments, systems and methods of the present invention stimulate (e.g., provide signals to (e.g., an electrical signal, a nerve impulse, an electrical signal that appears (e.g., is perceived by the brain) as a nerve impulse, an electrical impulse (e.g., that provokes a nerve impulse), and/or both an electrical signal and nerve impulse)) the medulla (e.g., via sensory ganglia of any one or more of cranial nerves VIII, IX, X, XI and XII). In some embodiments, stimulation of the medulla comprises stimulating nuclei involved in regulating heart rate (e.g., that stimulate the intercostals muscles and diaphragm), respiration rate, vasoconstriction, swallowing, and/or vomiting. In some embodiments, stimulation of nuclei (e.g., nuclei involved in regulating heart rate, respiration rate, vasoconstriction, swallowing, and/or vomiting) permits a subject to enjoy precise, voluntary movement and/or to maintain equilibrium (e.g., homeostasis). In some embodiments, stimulation of nuclei (e.g., nuclei involved in regulating heart rate, respiration rate, vasoconstriction, swallowing, and/or vomiting) permits a subject to experience better respiratory (e.g. breathing) function.

In some embodiments, systems and methods of the present invention stimulate (e.g., provide signals to (e.g., an electrical signal, a nerve impulse, an electrical signal that appears (e.g., is perceived by the brain) as a nerve impulse, an electrical impulse (e.g., that provokes a nerve impulse), and/or both an electrical signal and nerve impulse)) the pons (e.g., via sensory ganglia of any one or more of cranial nerves V through VIII). Although an understanding of the mechanism is not necessary to practice the present invention and the present invention is not limited to any particular mechanism of action, stimulation of the pons provides a subject with information related to voluntary skeletal (e.g., muscle) movements, thereby making such movements easier, less jerky and more controlled. In some embodiments, stimulation of the pons assists a subject to process information from the cerebral cortex to the cerebellum. In some embodiments, stimulation of the pons comprises stimulating nuclei of cranial nerves V, VI, VII and/or VIII. In some embodiments, stimulation of the pons permits a subject to experience better respiratory function.

In some embodiments, systems and methods of the present invention stimulate (e.g., provide signals to (e.g., an electrical signal, a nerve impulse, an electrical signal that appears (e.g., is perceived by the brain) as a nerve impulse, an electrical impulse (e.g., that provokes a nerve impulse), and/or both an electrical signal and nerve impulse)) the reticular formation. In some embodiments, stimulation of the reticular formation provides a subject with improved muscle tone. In some embodiments, stimulation of the reticular formation provides a subject with improved sleep. Although an understanding of the mechanism is not necessary to practice the present invention and the present invention is not limited to any particular mechanism of action, the stimulation of the reticular system provided by the systems and methods of the present invention mimic normal signals (e.g., electrical signals or nerve impulses) received by the reticular formation.

In some embodiments, systems and methods of the present invention stimulate (e.g., provide signals to (e.g., an electrical signal, a nerve impulse, an electrical signal that appears (e.g., is perceived by the brain) as a nerve impulse, an electrical impulse (e.g., that provokes a nerve impulse), and/or both an electrical signal and nerve impulse)) the brain stem (e.g., via sensory ganglia of any one or more of cranial nerves II through XII). Thus, in some embodiments, stimulation of the brain stem comprises stimulation of the vestibular nuclei complex (e.g., located between the trigeminal nuclei and the solitary nuclear complex). In some embodiments, stimulation of the brainstem provides a subject enhanced consciousness (e.g., corrects a defect in consciousness), increased cerebellar activity (e.g., corrects a defect in cerebellar circuitry (e.g., caused by disease, aging or injury), improved muscle tone, posture, and/or respiration. In some embodiments, stimulation of the brainstem comprises stimulating nuclei of cranial nerves II, III, IV, V, VI, VII, VIII, IX, X, XI, and/or XII. In some embodiments, stimulation of a cranial nerve (e.g., cranial nerve V (trigeminal/lingual nerve) or cranial nerve VII (taste nerve or chorda tympani)) stimulates the vestibular nuclei complex (e.g., located between the trigeminal nuclei and the solitary nuclear complex).

In some embodiments, systems and methods of the present invention stimulate (e.g., provide signals to (e.g., an electrical signal, a nerve impulse, an electrical signal that appears (e.g., is perceived by the brain) as a nerve impulse, an electrical impulse (e.g., that provokes a nerve impulse), and/or both an electrical signal and nerve impulse)) the cerebellum (e.g., indirectly via signals from the pons). In some embodiments, stimulation of the cerebellum provides a subject (e.g., a subject receiving stimulation of the cerebellum with the systems and methods of the present invention) an enhanced ability to control muscle movement (e.g., permitting a subject with jerky and/or uncoordinated muscle movements (e.g., resulting from disease, aging or injury) to experience less jerky, controlled and coordinated movements) and an increased capability for long term potentiation (e.g., permitting a subject to experience long term benefits from using and training with the systems and methods of the present invention).

In some embodiments, systems and methods of the present invention stimulate (e.g., provide signals to (e.g., an electrical signal, a nerve impulse, an electrical signal that appears (e.g., is perceived by the brain) as a nerve impulse, an electrical impulse (e.g., that provokes a nerve impulse), and/or both an electrical signal and nerve impulse)) the midbrain (e.g., via sensory ganglia of a cranial nerve III and/or IV). In some embodiments, stimulation of the midbrain comprises stimulating the reticular formation. In some embodiments, stimulation of the midbrain comprises stimulating the substantia nigra. In some embodiments, stimulation of the substantia nigra provides a subject (e.g., a subject with Parkinson's disease or other disease, an aged subject, an athlete, or an injured subject) with an enhanced ability to control body movements (e.g., systems and methods of the present invention provide a subject with Parkinson's the ability to “smooth” out body movements, or provide an athlete superior control of body movements to those achievable without the systems and methods of the present invention).

In some embodiments, systems and methods of the present invention stimulate (e.g., provide signals to (e.g., an electrical signal, a nerve impulse, an electrical signal that appears (e.g., is perceived by the brain) as a nerve impulse, an electrical impulse (e.g., that provokes a nerve impulse), and/or both an electrical signal and nerve impulse)) the vestibular and/or auditory nerves of a subject. In some embodiments, the signal targets (e.g., activates) the vestibular nuclei complex (e.g., located between the trigeminal nuclei and the solitary nuclear complex). Although an understanding of the mechanism is not necessary to practice the present invention and the present invention is not limited to any particular mechanism of action, in some embodiments, systems and methods of the present invention stimulate (e.g., provide signals to (e.g., an electrical signal, a nerve impulse, an electrical signal that appears (e.g., is perceived by the brain) as a nerve impulse, an electrical impulse (e.g., that provokes a nerve impulse), and/or both an electrical signal and nerve impulse)) the brain through the trigeminal (lingual nerve) and facial (taste or chorda tympani) nerves, thereby activating one or more regions of the brain (e.g., the brainstem (e.g., the trigeminal nuclei or nucleus of solitary tract)). In some embodiments, stimulation of the vestibular and/or auditory nerves (e.g., via stimulation of the trigeminal and facial nerves) and/or stimulation (e.g., activation) of the vestibular nuclei complex provides a subject an enhanced ability to maintain a sense of homeostasis (e.g., balance, security and/or orientation).

Because the auditory and vestibular systems are intimately connected, it is contemplated that a subject being treated with systems and methods of the present invention (e.g., that are being used to treat vestibular disorders) may also benefit from sound therapy (e.g., listening to music that strengthens, focuses, and or calms the brain). Thus, in some embodiments, systems and methods of the present invention are used in combination with sound therapy (e.g., music or other auditory element) to treat a subject. In some embodiments, treating a subject with a combination of systems and methods of the present invention and sound therapy stimulate (e.g., provide signals to (e.g., an electrical signal, a nerve impulse, an electrical signal that appears (e.g., is perceived by the brain) as a nerve impulse, an electrical impulse (e.g., that provokes a nerve impulse), and/or both an electrical signal and nerve impulse)) the medulla and/or thalamus of the subject. In some embodiments, using a combination of systems and methods of the present invention and sound therapy provide additive stimulation to the medulla and/or thalamus of a subject. In some embodiments, using a combination of systems and methods of the present invention and sound therapy provide synergistic (e.g., more than additive) stimulation to the medulla and/or thalamus of a subject. In some embodiments, stimulating the medulla comprises stimulating the superior olive. In some embodiments, stimulating the thalamus comprises stimulating the medial geniculate nucleus. Although an understanding of the mechanism is not necessary to practice the present invention and the present invention is not limited to any particular mechanism of action, stimulation of the medulla and/or thalamus is contemplated to provide a subject with the information (e.g., an electrical signal, a nerve impulse, an electrical signal that appears (e.g., is perceived by the brain) as a nerve impulse, an electrical impulse (e.g., that provokes a nerve impulse), and/or both an electrical signal and nerve impulse) needed for the subject to overcome the vestibular disorder (e.g., vestibular symptoms associated with disease, injury or aging).

In some embodiments, treating a subject with a combination of systems and methods of the present invention and sound therapy stimulates (e.g., provide signals to (e.g., an electrical signal, a nerve impulse, an electrical signal that appears (e.g., is perceived by the brain) as a nerve impulse, an electrical impulse (e.g., that provokes a nerve impulse), and/or both an electrical signal and nerve impulse)) the vestibular nerve of the subject. In some embodiments, the stimulation generates a synapse in the vestibular nuclei. In some embodiments, stimulation with a combination of systems and methods of the present invention and sound therapy provides a subject a superior ability to maintain a sense of homeostasis (e.g., balance, security and/or orientation) than when either therapy (e.g., systems and methods of the present invention or sound therapy) is used alone. Although an understanding of the mechanism is not necessary to practice the present invention and the present invention is not limited to any particular mechanism of action, in some embodiments, stimulation of the vestibular nerve of a subject with the systems and methods of the present invention provide synapses within the vestibular nuclei that are absent or impaired due to disease, injury or aging.

Systems and methods of the present invention (e.g., used alone or in combination with other treatments (e.g., sound therapy, pharmaceuticals, etc.)) find use in vestibular therapy (e.g., vestibular rehabilitation therapy associated with chronic (e.g., aging or disease) or acute (e.g., injury induced) impairment of the vestibular system). In some preferred embodiments, such therapy is most effective when customized to an individual patient (e.g., systems and methods are customized (e.g., provide individualized amounts (e.g., total amounts of electrical energy) and type of stimulus (e.g., electrotactile stimulation of the tongue, auditory nerve stimulation with sound (e.g., with music or other form of sound therapy, etc.)) to the individual needs of a subject). In some embodiments, therapy is supervised by an appropriately trained professional (e.g., a trained therapist (e.g., physical or occupational) or physician). In some embodiments, therapy with the systems and methods of the present invention are used in combination with other types of therapy for vestibular dysfunction (See, e.g., therapies described in Shepard et al., Otolaryngol Head Neck Surg 112, 173-182 (1995); Shepard et al., Ann Otol Thinol Laryngol 102, 198-205 (1993), and Shumway-Cook and Horak, Neurol Clin 8, 441-457 (1990), each of which is herein incorporated by reference). Systems and methods of the present invention provide treatment (e.g., therapeutic, prophylactic, and/or sensory enhancing treatment) for a subject experiencing or susceptible to experiencing vestibular dysfunction (e.g., a subject with disease, injury and/or that is aging), or a subject wishing to enhance vestibular function (e.g., an athlete or member of the armed forces), for a number of reasons.

For example, a unique feature of the central nervous system (e.g., comprising the brain and spinal cord) is its capacity for adaptation to asymmetries (e.g., in peripheral vestibular afferent activity). This process is referred to as vestibular compensation and results from active neuronal and neurochemical processes in the cerebellum and the brain stem in response to sensory signals (e.g., that are harmonized in a “healthy” or “normal” subject) that may be conflicted due to vestibular impairment (e.g., pathology caused by disease, age and/or injury) (See, e.g., Telian and Shepard, Otolaryngol Clin North Am 29, 359-371 (1996)). Thus, in general (e.g., in a healthy or normal subject), vestibular compensation is able to relieve vestibular symptoms (e.g., dizziness, disorientation, nausea, respiratory and speech problem, instability, ability to focus eyes and/or attention, etc.). However, vestibular symptoms may persist in certain individuals suffering from disease (e.g., including, but not limited to, Meniere's disease), injured (e.g., traumatic brain injured) subjects, subjects who have had a stroke, a subject with vestibular neuritis, a subject with viral endolymphatic labyrinthitis, a subject with benign paroxysmal positional vertigo, a subject with delayed onset vertigo syndrome, a subject with labyrinthine complications of otitis media, a subject with a perilymph fistula, a subject with an acoustic neuroma, a subject with migraine, a subject with epilepsy, a subject with demyelinating disease (e.g., multiple sclerosis), a subject with unilateral or bilateral vestibular dysfunction, a subject with epilepsy, a subject with dyslexia, a subject with migraines, a subject with Mal de Debarquement syndrome, a subject with oscillopsia, a subject with autism, a subject with Parkinson's disease, or a subject with tinnitus. Systems and methods of the present invention can be used to treat these types of subjects. Thus, in preferred embodiments, the systems and methods of the present invention find particularly beneficial use (e.g., by an injured person, a person with a disease (e.g., including, but not limited to those described above and elsewhere herein) or an aging person) for accelerating, correcting and/or enhancing (e.g., pushing to better than normal (e.g., for healthy people)) vestibular compensation.

The present invention also finds use with subjects in a recovery period from a disease, condition, or medical intervention, including, but not limited to, subjects that have suffered traumatic brain injury (e.g., from a stroke) or drug treatment. The systems and methods of the present invention find use with any subject that has a loss of balance or is at risk for loss of balance (e.g., due to age, disease, environmental conditions, etc.). Systems and methods of the present invention are able to treat (e.g., correct and/or relieve vestibular symptoms, or, enhance the normal function of) the vestibular system of a subject.

Systems and methods of the present invention find use in treating subjects in need of acute (e.g., a subject with a vestibular lesion (e.g., due to traumatic brain injury)) and chronic (e.g., a subject with vertigo (e.g., caused by any of the diseases or conditions described herein)) vestibular compensation. Vertigo of acute onset usually results from pathology (e.g., caused by disease and/or injury) associated with the vestibular nerve or the labyrinth. The vertigo may be accompanied by nystagmus and a variety of undesirable vegetative symptoms (e.g., nausea and/or vomiting). As acute compensation for the peripheral vestibular insult proceeds, vestibular symptoms may be reduced with nystagmus observed after visual fixation is eliminated (See, e.g., Igarashi, Acta Otolaryngol (Stockn) 406, 78-82 (1984); Smith and Curthoys, Brain Res Brain Res Rev 14, 155-180, (1989)). Generally, acute compensation occurs initially by the influence of the cerebellum as well as neurochemical changes at the level of the vestibular nuclei (See, e.g., Smith and Darlington, Brain Res Brain Res Rev 17, 117-133 (1991)). These changes are thought to be produced in order to minimize side to side discrepancies between the tonic firing rates in the second-order neurons originating in the nuclei. The compensation process may provide relief from symptoms (e.g., the most intense symptoms) within 24-72 hours. However, many subjects continue to have considerable disequilibrium (e.g., because the inhibited system is unable to respond appropriately to the labyrinthine input produced by head movements involved in normal daily activities). Even after intense vertigo has been controlled, it is not uncommon for subjects to have continued motion-provoked vertigo (e.g., until chronic (e.g., dynamic) vestibular compensation is achieved).

Accordingly, the present invention provides systems and methods for a subject to achieve vestibular compensation (e.g., chronic (e.g., dynamic) vestibular compensation). In some embodiments, systems and methods of the present invention provide a subject with the ability to respond appropriately to labyrinthine input (e.g. produced by head movements (e.g., movements involved with normal daily activities)). In some embodiments, the present invention provides systems and methods that accelerate acute vestibular compensation. Although an understanding of the mechanism is not necessary to practice the present invention and the present invention is not limited to any particular mechanism of action, in some embodiments, systems and methods of the present invention stimulate the cerebellum and other parts of the central nervous system (e.g., the brain stem (e.g., the midbrain, pons or medulla) thereby enabling the subject to achieve vestibular compensation. In some embodiments, systems and methods of the present invention induce neurochemical changes (e.g., neurotransmitter release) at the level of the vestibular nuclei (e.g., thereby equilibrating the tonic firing rate of second-order neurons originating in the nuclei).

Systems and methods of the present invention can also be utilized to treat a subject in need of chronic (e.g., a subject with vertigo (e.g., caused by any of the diseases or conditions described herein)) vestibular compensation. Research has shown that in order to eliminate disequilibrium and residual motion-provoked vertigo, the vestibular system needs to reestablish symmetric tonic firing rates in the vestibular nuclei and accurate responses to head movements (See, e.g., Smith and Curthoys, Brain Res Brain Res Rev 14, 155-180, (1989)). If the vestibular system fails extensively (e.g., due to disease, injury, or aging), the ipsilateral vestibular nucleus can become responsive to changes in the contra-lateral eighth nerve firing rate by activation of commissural pathways (See, e.g., Telian and Shepard, Otolaryngol Clin North Am 29, 359-371 (1996)). This feature of the compensation process is important to regaining vestibular function (e.g., following ablative vestibular surgery (e.g., labyrinthectomy or vestibular nerve section)). If the vestibular systems fails somewhat (e.g., less than extensively (e.g., an incomplete peripheral lesion or abnormality caused by disease, injury or aging)), the injured labyrinth can produce a disordered response to movements requiring adjustments in the central nervous system to properly reinterpret the input from the damaged side. If the lesion is an unstable lesion (e.g., as observed with Meniere's disease or a progressive labyrinthitis), vestibular compensation has heretofore been difficult to achieve.

The vestibular compensation process requires consistency in the inputs to properly utilize them for habituation. It appears that the central compensation process is enhanced by head movement but delayed by inactivity (See, e.g., Mathog and Peppard, Am J Otolaryngol 3, 397-407 (1982)). For example, medications that are typically provided to a subject for acute symptoms of vertigo, such as meclizine, scopolamine, and benzodiazepine all cause sedation and central nervous system depression (See, e.g., Bienhold et al., Lesion-Induced Neuronal Plasticity in Sensorimotor Systems, Flohr and Precht (eds), 265-273 (1981); Zee, Arch Otolaryngol 111, 609-612 (1985)). Thus, although these medications may provide satisfactory short term relief (e.g., during the initial stages of an acute labyrinthine crisis), they are counterproductive with respect to vestibular compensation, especially when used for extended periods (See, e.g., Peppard, Laryngoscope 96 878-898 (1986)).

Accordingly, the present invention provides systems and methods for a subject suffering from chronic vestibular symptoms to achieve vestibular compensation (e.g., chronic (e.g., dynamic) vestibular compensation). Although an understanding of the mechanism is not necessary to practice the present invention and the present invention is not limited to any particular mechanism of action, in some embodiments, systems and methods of the present invention provide a subject with the ability to respond (e.g., versus not responding, or, when capable of responding, enhancement of the response) to firing of the eighth cranial nerve. In some embodiments, the present invention provides signals that compensate for or augment normal firing of the eighth cranial nerve. In some embodiments, systems and methods of the present invention correct disordered labyrinth responses to movements. In some embodiments, systems and methods of the present invention permit a subject to properly interpret the input from a damaged or otherwise non-functional vestibular system. In some embodiments, the present invention provides systems and methods that provide compensation (e.g., adjustment) to the central nervous system (e.g. in order to properly interpret input from an injured labyrinth). In some embodiments, the systems and methods of the present invention overcome existing limitations of other types of therapy (e.g., heretofore existing therapies used to treat vestibular abnormalities) in that systems and methods of the present invention are able to compensate for unstable lesions (e.g., as observed in Meniere's disease or a progressive labyrinthitis). Although an understanding of the mechanism is not necessary to practice the present invention and the present invention is not limited to any particular mechanism of action, systems and methods of the present invention provide stimulation to regions of the central nervous system (e.g., to the cerebellum and/or the brain stem (e.g., the midbrain, pons and medualla)) thereby providing signals to the subject important for vestibular compensation (See, e.g., Example 28).

For example, the vestibular system is not silent until stimulated. Rather, the vestibular system is constantly accepting, processing and sending signals representing the status of a subject. Specifically, the vestibular system constantly accepts (e.g., from ganglia of the vestibulochlear nerve) signals (e.g., stimulation/depolarization of hair cells) and discharges a pattern of signals to the brain. Acceleration or a change in acceleration deviates the cupula and produces a change in this pattern of signals and it is this change that is distributed to the brain for interpretation. It is important to note that the vestibular system comprises left and right sided signals that are in a constant, dynamic balance, one checking against the other, informing a subject of movements and head positions and adjusting the body to new conditions. The brain learns (e.g., during development) what signals (e.g., patterns of signals) to expect from the vestibular system (e.g. the vestibular organs).

Thus, when something happens that alters (e.g., inhibits) normal functioning of the vestibular system (e.g., disease, injury, or deterioration with age), the system may no longer be capable of discharging at rest at equal right and left intensities (e.g., a loss of equilibrium (e.g., homeostasis) occurs). This unequal intensity of discharge has specific meaning to the brain. Thus, the sequelae of this imbalance may be manifestations of a relative hyperfunction of an intact side with uncontrolled and prolonged vestibular reflexes resulting. The disparate messages arrive at the brain (e.g., at the midbrain (e.g., the pons)) and are processed (e.g., by the cerebral cortex) in the way that the brain knows how to (e.g., through past experience). Thus, the brain interprets these signals as a condition of constant motion (e.g., generating dizziness (e.g., vertigo)). This same imbalance in discharge of signal also arrives at the eye muscle nuclei and the reticular formation. The imbalance (e.g., interpreted in the light of past experience and training) directs the eye muscle nuclei to deviate the eyes in the direction of last gaze to retain orientation (e.g., generating nystagmus). The imbalance information also transmits from the vestibular nuclei down the spinal cord to anterior horn cells, instructing the postural and locomotor muscles to meet a new situation that never arrives (e.g., generating staggering and ataxia).

Accordingly, the present invention provides systems and methods that are useful for restoration of normal functioning of the vestibular system. Although an understanding of the mechanism is not necessary to practice the present invention and the present invention is not limited to any particular mechanism of action, the systems and methods of the present invention generate new electrical activity in the improperly discharging (e.g., under-discharging or over-discharging) system thereby balancing the system (e.g., balancing the normal but relatively hyperactive (e.g., perceived as hyperactive) side). In some embodiments, systems and methods of the present invention stimulate (e.g., provide signals to (e.g., an electrical signal, a nerve impulse, an electrical signal that appears (e.g., is perceived by the brain) as a nerve impulse, an electrical impulse (e.g., that provokes a nerve impulse), and/or both an electrical signal and nerve impulse)) the vestibular and/or auditory nerves of a subject in order to balance the vestibular system. In some embodiments, stimulation of vestibular and/or auditory nerves in a subject with the systems and methods of the present invention provides the subject with new, resting electrical activity (e.g., in nuclei associated with motion and hearing (e.g., in a denervated vestibular nuclei, or a vestibular or auditory nuclei that is damaged or diseased)). Although an understanding of the mechanism is not necessary to practice the present invention and the present invention is not limited to any particular mechanism of action, the systems and methods of the present invention regenerate (e.g., re-set) the resting activity in the vestibular and/or auditory nuclei. The regeneration of the resting activity in turn cause vestibular symptoms to disappear.

The systems and methods of the present invention uniquely supply the signals necessary to overcome vestibular symptoms. Although an understanding of the mechanism is not necessary to practice the present invention and the present invention is not limited to any particular mechanism of action, the vestibular input (e.g., using systems and methods of the present invention) provides long term benefits (e.g., disappearance of vestibular symptoms and the appearance of other effects (e.g., improved posture, improved gait (e.g., through improved muscle coordination), improved breathing, an enhanced ability to perceive and concentrate, and other benefits described herein) to a subject by supplying signals to the brain (e.g., the vestibular system). The route, type and duration of stimulation provided to a subject by the systems and methods of the present invention are important for providing these benefits.

Systems and methods of the present invention are able to supplement, enhance and/or correct defects in the vestibular system of a subject when used by the subject for certain, specific amounts of time. For example, subjects that used (e.g., trained with) the systems and methods of the present invention for certain amounts of time (e.g., 20 minutes) reported long term benefits lasting from over an hour, six hours, twenty-four hours, a week, a month, and six months after use (e.g., after exposure to electrotactile stimulation) (See Example 21). Thus, in some embodiments, stimulation of the brain (e.g., the brainstem (e.g. the midbrain, medulla, and pons)) for a period of, for example, 20 minutes using systems and methods of the present invention is sufficient for bestowing treatment benefits to a subject. In some embodiments, stimulation of the brain (e.g., the brainstem (e.g. the midbrain, medulla, and pons)) for a period of, for example, 20 minutes using systems and methods of the present invention is sufficient to regenerate (e.g., re-set) the resting activity in the vestibular and/or auditory nuclei. However, it is contemplated that additional exposure (e.g., training with the systems and methods of the present invention (e.g., using the systems and methods of the present invention to stimulate the brain 20 or more minutes daily for a week, two weeks or more; and/or 5, 10 or 20 minutes two or more times a day (e.g., for a total of 20, 40, 60, or more minutes at day)) provides additional stimulation to the brain and increases the beneficial effects enjoyed by subjects (e.g., increases long term potentiation (e.g., of a return to homeostasis)).

In some embodiments, the systems and methods of the present invention are used to treat various symptoms or improve normal body function. The present invention is not limited by the type of symptom treated. Indeed a variety of symptoms can be treated using the systems and methods of the present invention including, but not limited to, dizziness, headache, inability to walk on uneven surfaces, loss of memory, inability to walk in a crowd, inability to walk up or down stairs, inability to look up or down, impaired vision, impaired speech, rigid or otherwise disturbed gait, shaking, nervousness, twitching, anxiety, depression, sleeplessness, tremor, motion sickness, confusion, insomnia, numbness, pain, achiness, paralysis, blurry vision, difficulty breathing (e.g., dyspnea), dementia, difficulty concentrating, swallowing problems (e.g., dysphagia), discomfort, lack of confidence, drowsiness, forgetfulness, hallucination, hypersensitivity, hyposensitivity, impaired balance, impaired memory, inattentiveness, neurosis, jerkiness, lack of feeling or sensation, manic, moodiness, tingling, difficulty with speech, paranoid, peripheral vision problems, respiration problems, tingling, unsteadiness, lack of ability to multitask, vision problems, delusion, detachment, disorientation, problems with posture, lack of strength, lack of tone, seizure, tunnel vision, weakness, lack of alertness, inability to concentrate, difficulty comprehending or understanding speech and/or spoken words, vertigo, apathy, lethargy, unconsciousness, and uncontrolled eye movements.

In some embodiments, it is contemplated that the systems and methods of the present invention provide direct effects beneficial to a subject. These include, but are not limited to, immediate correction or improvement of vestibular function (e.g., balance), proprioception, motor control, vision, posture, cognitive functions, tinnitus, emotional conditions, and correction or improvement (e.g., lowering the level or elimination) of the symptoms listed above. In some embodiments, the correction or improvement occurs over time after training with the systems and methods mentioned herein. In addition to direct effects, it is also contemplated that the systems and method of the present invention provide indirect effects that benefit a subject. These indirect effects include, but are not limited to, regaining or acquiring a physical, cognitive, emotional, and/or neurologic function, and/or overall sense of well-being. Thus, in some embodiments, a direct effect targeted at a specific function is provided (e.g., improved balance in response to body position information provided to a subject by the systems of the present invention), an indirect effect that relates to the specific function is provided (e.g., improved motor control that is at least partially independent of the nature of the information provided), and indirect effects not directly related to the specific function is provided (e.g., improved sense of well-being, sleep, etc.). In some embodiments, the direct effect and associated benefits sensitize the subject to allow receipt of the indirect effects. In other embodiments, the indirect effects sensitize the subject to obtain direct effect. Thus, in some embodiments, all effects, over time, enhance the benefits achieved by the others. For example, in some embodiments, improvement to vestibular function are provided by the systems of the present invention as described in Example 1. While not being limited to any particular mechanism of action, it is contemplated that this improvement permits additional physical and mental improvements, as many other brain functions are associated directly or indirectly with the vestibular system. Likewise, the indirect effects provide a more general enhancement of brain function, permitting, for example, better reception for training and improvement of the direct effect.

In some embodiments, systems and methods of the present invention are used to treat (e.g., independently, or, in combination with other treatments) a subject undergoing therapy for nerve damage (e.g., nerve damage caused by traumatic injury (e.g., spinal cord injury), or nerve damage caused by diabetes, stroke, disease or other causes). Although an understanding of the mechanism is not necessary to practice the present invention and the present invention is not limited to any particular mechanism of action, it is contemplated that the systems and methods of the present invention will assist a nerve damaged subject to respond (e.g., more accurately and/or rapidly) to neural signals (e.g., ascending signals via a somatosensory neuron or descending signals via a motor neuron (e.g., signals that are generated or regenerated using existing treatments for nerve damage (e.g., that regulate nerve (e.g., neuron) growth at a site of injury) in combination with the systems and methods of the present invention.

The systems and methods may also be used in research application to study balance and balance-associated conditions, including, but not limited to, the study of the central mechanisms associated with balance and balance-associated conditions, sensory integration, and sensory motor integration. Example 15 provides methods of studying brain function by MRI in response to the systems of the present invention.

Healthy individuals may also use such systems and methods to enhance or alter balance. Such applications include use by athletes, soldiers, pilots, video game players, and the like.

The vestibular uses of the present invention may be used alone or in conjunction with other sensory substitution and enhancement applications. For example, blind subjects may use systems and methods that improve vestibular function as well as vision. Likewise, video game players may desire a wide variety of sensory information including, for example, balance, vision, audio, and tactile information.

In some embodiments, the sensory substitution provides the subject with improved vision or treats a vision-associated condition. In such embodiments, subjects are trained to associate tactile or other sensory inputs with video or other visual information, for example, provided by a camera or other source of video information. In some embodiments, blind subjects are trained to visualize objects, shapes, motion, light, and the like. Such applications have particular benefit for subjects with partial vision loss and provides methods for both enhancement of vision and rehabilitation. Training of blind subjects can occur at any time. However, in preferred embodiments, training is conducted with babies or young children to maximize the ability of the brain to process complex video information and to coordinate and integrate the information higher cognitive functions that develop with aging. Example 12 describes the use of the methods of the invention to allow a blind subject to catch a baseball, perceive doors, and the like. The present invention also finds use in vision enhancement for subjects that are losing vision (e.g., subjects with macular degeneration).

In some embodiments, the sensory substitution provides the subject with improved audio perception or clarity or treats an audio-associated condition. In such embodiments, subjects are trained to associate tactile or other sensory inputs, directly or indirectly, with audio information, to reduce unwanted sounds or noises, or to improve sound discrimination. Example 11 describes the use of the methods of the present invention to enhance the ability of deaf subjects to lip read. More advanced hearing substitution systems may also be applied. Example 8 describes the successful use of the invention to reduce tinnitus in a subject. In some embodiments, arm bands (electrotactile or vibrotactile) or tongue-based devices are used to communicate various qualities of music or other audio (e.g., rhythm, pitch, tone quality, volume, etc.) to subjects either through location of or intensity of signal.

In some embodiments, the sensory substitution provides the subject with improved tactile perception or treats a condition associated with loss or reduction of tactile sensation. In such embodiments, subjects are trained to associate tactile or other sensory inputs at one location, directly or indirectly, with tactile sensation at another location. Example 9, below, describes the use of tactile substitution for use in generating sexual sensation, for, for example, persons with paralysis. Other applications include providing enhanced sensation for subjects suffering from diabetic neuropathy (to compensate for insensitive legs and feet), spinal stenosis, or other conditions that cause disabling or undesired tactile insensitivity (e.g., insensitive hands). The systems and methods of the present invention also find use in sex application for healthy individuals. Example 9 further describes sex applications, including Internet-based sex applications that permit remote subjects to have a wide variety of remote “contact” with one another or with programmed or virtual partners.

In some embodiments, the sensory substitution provides the subject with improved ability to perceive taste or smell. Sensors that collect taste or olfactory information (e.g., chemical sensors) are used to provide information that is transmitted to a subject to enhance the ability to perceive or identify tastes or smells. In some such embodiments, the system is used to mask or otherwise alter undesirable tastes or smells to assist subjects in eating or in working in unpleasant environments.

In addition to applications that provide sensory substitution, the present invention provides systems and methods for sensory enhancement. In sensory enhancement applications, the systems and methods supply improvement to existing senses or add new sensory information that permits a subject to perform tasks in an enhanced manner or in a manner that would not be possible without the sensory enhancement.

In some embodiments, the sensory enhancement is used for entertainment or multimedia applications. Example 10, below, describes the enhancement of videogame and television or movie applications by transmitting novel non-traditional sensory information to the user in addition to the normal audio and video information. For example, video game players can be given 360 degree “vision,” visual images received from tactile stimulation can be provided with music or can be provided along with normal video. Users can be made to feel unbalanced or otherwise altered in response to events occurring in a movie or theme park ride. Deaf subject can be provided with information corresponding to music playing in a dance venue to permit them to perceive simple or advanced aspects of the music being played or performed. For example, in some embodiments, a tactile patch is provided on the arm (or other desired body location) that transmits music information. In some embodiments, the patch further provides aesthetic appeal.

In some embodiments, the sensory enhancement provides a new sense by training the user to associate a tactile or other sensory input with a signal from an external device (e.g. a piece of equipment or machine) that perceives an object or event. For example, subjects can be provided with the ability to “see” infrared light (night vision) by associating tactile input with signals received from an infrared camera. Ultraviolet light, ultrasonic noise (e.g., as detected by sonar), radiation or other particles or waves acquired by artificial sensors (e.g., radar or instruments capable of monitoring sound wave time of flight, for example, ultrasonic sensors) can likewise be detected and sensed. Any material or event that can be identified by a sensory device can be combined with the systems of the present invention to provide new senses. For example, chemical sensors (e.g., for volatile organic compounds, explosives, carbon monoxide, oxygen, etc.) are adapted to provide, for example, an electrotactile signal to a subject (e.g., via the tongue). Similarly, sensors for detection of biological agents (e.g., environmental pathogens or pathogens used in biological weapons) are adapted to provide such a signal to a subject (e.g., from molecular detection or other types of biological equipment). In addition to the presence of a detected compound or agent, the amount, nature of, and/or location may also be perceived by the subject. Such sensors may also be used to monitor biological systems. For example, diabetic subjects can use the system associated with a glucose sensor (e.g., implanted blood or saliva-based glucose sensor) to “see” or “feel” their blood glucose levels. Athletes can monitor ketone body formation. Organ transplant patients can monitor and feel the presence of cytokines associated with chronic rejection in time to seek the appropriate medical care or intervention. Likewise, an individual can monitor and feel the presence of a pathogen (e.g., a virus such as HIV or a bacterium such as N. gonorrhoeae and/or C. trachomatis) in their own self or in others (e.g., through intimate contact). The present invention can similarly be adapted to blood alcohol level (e.g., providing a user with accurate indication of when blood alcohol level exceeds legal limits for driving or machine operation). Numerous other physical and physiochemical measurements (e.g., standard panels conducted during routine medical testing that are indicative of health-related conditions are equally as adaptable for “sensing” using the present invention).

In preferred embodiments, a new sense is provided to a user through training the user to use the systems and methods of the present invention to associate a tactile or other sensory input with a signal from an external device. In some preferred embodiments, the sensory or tactile input is provided to the user through the tongue. It is contemplated that systems of the present invention are capable of monitoring and/or receiving information from an external, artificial sensor, and translating the information into tactile or other sensory input to the user via the tongue. For example, in some embodiments, the external, artificial sensor is an ultrasonic sensor (e.g., sonar) capable of sending and receiving signals (e.g., sound wave signals). In some embodiments, the ultrasonic sensor further comprises means (e.g., software and a computer processor) for calculating sound wave time of flight. In some embodiments, the sensor may emit a burst (e.g., a short or long burst) of ultrasonic sound (e.g., 40 kHz) from a transducer (e.g., a piezoelectric transducer). In preferred embodiments, the sensor further comprises a detector (e.g., another piezoelectric transducer). In some embodiments, the sound (e.g., generated by the transducer) is reflected by objects in front of the device, returned to the sensor unit and detected (e.g., by a detector). In some embodiments, the sound burst emitted by the transducer is detected by a detector present on a second separate sensor (e.g., on a second user such as a hiking companion or fellow soldier in an active zone). In some embodiments, the ultrasonic sensor further comprises a receiver amplifier that sends the signals (e.g., either a reflected signal/echo, or, a direct signal from a separate sensor) to a micro-controller (e.g., a microprocessor) that calculates (e.g., times the sound waves) how far away an object is (e.g., using the speed of sound in air). In preferred embodiments, the calculated range is converted into a constant current signal (e.g. that can be further translated into a discrete bundle of information) that is then provided to a user as a sensory or tactile input through the tongue.

In some embodiments, the sound waves sent from a transducer are at a constant interval such that if two or more persons are all using systems of the present invention that are capable of sending and receiving signals, the users are able to determine (e.g., through ultrasonic sensors and the sensory or tactile input translated therefrom provided to the users) the real-time location of each person using only the “sense” provided to the user from the systems and methods of the present invention.

In some embodiments, the sensory enhancement provides a new means of communication by training the user to associate a tactile or other sensory input with some form of wireless, visual, audio, or tactile communication. Such systems find particular use with soldiers, emergency response personnel, hikers, mountain climbers and the like. In some embodiments, coded information is provided via wireless communication to a user through, for example, an electrotactile tongue system. With prior training, the user perceives the signal as language and understands the message. In some embodiments, two-way communication is provided. Examples 14 and 17, below, describe such embodiments in more detail. In some such embodiments, the user encodes a return message through the device located in the mouth through, for example, movement of the tongue or the touching of teeth. In addition to standard languages and coded languages, the system may be used to send alarm messages in a wide array of complexities. Additional information may also be provided, including, for example, the relative physical location of co-workers (e.g., firemen, soldiers, stranded persons, enemies). In some embodiments, the language transmitted by the system is a pictographic language. In some embodiments, information sent to the device (e.g., for covert communication) can come from any source (e.g., wireless Internet or telecommunications). It is contemplated that the device have two-way communication means (e.g., that allows the user to activate buttons or their equivalent with the tongue). Thus, in some embodiments, a subject can monitor and communicate with the Internet (e.g., perceive sports scores, stock prices, weather, etc.) or another user through the use of an in-mouth or under skin device.

In some embodiments, the sensory enhancement provides remote tactile sensations to a user. For example, surgeons may use the device to gain increased “touch” sensitivity during surgery or for remote surgery. An example of the former embodiments is described in Example 13. An example of the latter embodiments is also described in Example 13. In some such embodiments, the tactile interface with the user is a glove that provides tactile information to the fingers and/or hand. The glove receives signals from a remove location and permits the user to “feel” the remote environment. In other embodiments, the tactile interface is an alternative input, e.g., an electrotactile tongue array, that provides the user with sensitivity to a non-touch related aspect of the remote environment (e.g., electroconductivity of local tissue, or the presence or absence of chemical or biological indicators of tissue condition or type). In addition to medical uses, such application find use in distant robot control, remote sensing, space applications (grip control, surface texture/structure monitoring), and work in aggressive or hostile environments (e.g., work with pathogens, chemical spills, low-oxygen environment, battle zones, etc.). Thus, in some embodiments, the present invention provides brain-controlled robots. The robots can have a wide variety of sensors (e.g., providing position, balance, limb position, etc. information) including specific chemical, temperature, and/or tactile sensors. With the interface and with sufficient training, the human user will sense the robots environment on multiple levels as though the users brain occupied the robot's body.

In some embodiments, the sensory enhancement provides navigation information to a user. By associated the systems of the present invention with global positioning technology or other devices that provide geographic position or orientation information, users gain enhanced navigation abilities (See e.g., Example 14). Information about geographic features of the surrounding environment may also be provided to enhance navigation. For example, pilots or divers can sense hills, valleys, current (water or air), and the like. Firefighters can sense temperature and oxygen levels in addition to information about position and information about the structure or structural integrity of the surrounding environment.

In some embodiments the sensory enhancement provides improved control of industrial processes. For example, an operator in an industrial setting (e.g., manufacturing plant, nuclear power plant, warehouse, hospital, construction site, etc.) is provided with information pertaining to the status, location, position, function, emergency state, etc. of components in the industrial setting such that the operator has an ability to perceive the environment beyond sensory input provided by their vision, hearing, smell, etc. This finds particular use in settings where a controller is expected to manage complex instrumentation or systems to ensure safe or efficient operation. By sensing status or problems (e.g., unsafe temperatures or pressure, the presence of gas, radiation, chemical leakage, hardware or software failures, etc.) through, for example, information flow from monitoring device to the an electrotactile array on the operators body, the operator can respond to problems in real time with additional sensory bandwidth.

In addition to sensory substitution and sensory enhancement applications, the present invention also provides motor enhancement applications.

Experiments conducted during the development of the present invention identified improved motor skills subjects undergoing training with the systems and methods of the present invention (see e.g., Example 2). Subjects reported more fluid body movement, more fluid, confident, light, relaxed and quick reflexes, improved fine motor skills, stamina and energy, as well as improved emotional health. In particularly preferred embodiments, subjects undergo training (see e.g., Example 1) in a seated or standing position. Training includes maintaining body position while concentrating on a body position training procedure. An understanding of the mechanism is not necessary to practice the present invention and the present invention is not limited to any particular mechanism of action. However, it is contemplated that such training provides the benefits achieved by meditation and stress management exercises. Unlike meditation however, which takes substantial training and time commitment to achieve the benefits, the methods of the present invention achieve the same benefits with minimal training and time commitment. With little training and short exposure, subject obtain a wide range of improvements to physical and mental well-being. Thus, such methods find use by athletes, pilots, martial artists, sharp shooters, surgeons, and the general public to improve motor skills and posture control. The methods find particular use in embodiments where subjects seek to regain normal physical capabilities, such as after flight rehabilitation or in flight enhancement for astronauts. Such uses may be coupled with sensory enhancement and/or substitution. For example, a sharp shooter may use the system to gain enhanced motor control and focus, but also to use the system to transmit aiming information and/or to allow the shooter to sense their heart rate (to pull the trigger between heart beats) or environmental conditions to enhance accuracy.

In some embodiments, the present invention provides systems and methods for treating (e.g., independently or in combination with other programs or therapeutic treatments) individuals recovering from addiction to a substance (e.g., drugs, alcohol, and the like.). For example, in some embodiments, systems and methods of the present invention are used in rehabilitation settings (e.g., drug and alcohol rehabilitation programs). In some embodiments, systems and methods of the present invention reduce and/or correct symptoms (e.g., headache, nausea, dizziness, disorientation, and the like) associated with recovery (e.g., withdrawal) from an addictive substance (e.g., drug or alcohol).

The methods also find use in general enhancement of physical and emotional well-being. Examples 2-8 describe a wide range of benefits achieved by subjects. These benefits include, but are not limited to, relaxation, pain relief, improved sleep and the like. Thus, the methods find use in any area where meditation has shown benefit (e.g., post menopause recovery).

In some embodiments, the systems and methods of the present invention are used in combination with other therapies to provide an enhanced benefit. Such uses may, for example, allow for the lowering of drug dose of the complementary therapy to reduce side effects and toxicity.

In some embodiments, the systems are used diagnostically, to predict or monitor the onset or regression of systems or to otherwise monitor performance (e.g., by athletes). For example, the systems may be used to test proficiency in training exercise and to compare results to a database of “normal” and “non-normal” results to predict onset of an undesired physical state. For example, subjects taking gentamycin are monitored for loss of vestibular function to permit physicians to discontinue or alter treatment so as to prevent or reduce unwanted side effects of the drug. In such embodiments, head displacement as a function of body position may be monitored and compared to a normal baseline or to look for variation in a particular subject over time. Because posture and balance deteriorate with age, the system may also be used to as a biomarker of biological age of a subject. Diagnostic methods may be used as an initial screening method for subject or may be used to monitor status during or after some treatment course of action.

The systems and methods of the present invention also find use in providing a feeling of alternative reality through, for example, a combination of sensory substitution and sensory enhancement. Through balance training exercises, subjects can be made to experience a loss of balance or orientation. Images can also be projected to the subject to enhance the state of alternate reality. When combined with other sensory stimulation, the effect can provide entertainment or provide a healthy alternative for illegal drugs.

### Vision Applications

Various types of functional vision loss exist. In general, vision loss or visual loss refers to the absence of vision where it existed before. Such loss can happen either acutely (e.g., abruptly) or chronically (e.g., over a long period of time). The effects of visual loss can be devastating to a subject. Various scales have been developed to describe the extent of vision and vision loss based on visual acuity (See, e.g., International Council of Opthalmology. “International Standards: Visual Standards: Aspects and Ranges of Vision Loss with Emphasis on Population Surveys.” April 2002). Examples of vision loss include, but are not limited to, macular degeneration (e.g., adult macular degeneration), central vision loss, peripheral vision loss, media opacity, ring scotomas, incomplete scotomas, absolute scotomas, retinitis pigmentosa, glaucoma, homonymous hemianopsia, retinal disease, optic nerve disease, hypoxia, visual pathway disorder and other types of vision loss (e.g., caused by disease and/or disorder).

Macular degeneration (MD), a progressive disease that can gradually destroy vision (e.g., central vision) affects more than 1.75 million people in the U.S. The deteriorating retina creates a blind spot (e.g., a scotoma) that may eventually obscure a person's vision (e.g., in spots, centrally, peripherally, etc.). While age-related MD (AMD) is the leading cause of vision loss in people older than 60, hereditary diseases (e.g., Stargardt's Disease) and toxic side effects of some medications (e.g., mellaril, chloroquine) can cause MD in much younger people.

Macular degeneration (MD) is a progressive disease characterized by high acuity central visual field loss. The macula, the central portion of the retina, encompasses the fovea and has a high density of cone cells, which are important for seeing color and fine detail (See, e.g., Fine et al., N. Engl. J. Med. 342, 483-492 (2000)). Age-related MD (AMD) is the leading cause of vision loss in people older than 60. In the United States, 1.75 million people currently suffer from AMD and that number is expected to grow to nearly 3 million by 2020 as the population ages (See, e.g., Friedman et al., Arch. Opthalmol. 122, 564-572 (2004)). While MD is commonly associated with older adults, Stargardt's Disease (sometimes referred to as juvenile macular degeneration) causes MD in a much younger population. Stargardt's Disease is the most common hereditary form of MD. People with Stargardt's Disease often notice vision problems when in their 20s or 30s. Macular degeneration is also found as a toxic side effect of certain drugs (e.g., mellaril, chloroquine). Typical MD can be ‘dry’ or ‘wet’ (See, e.g., Fine et al., N. Engl. J. Med. 342, 483-492 (2000)). Dry MD affects about 90% of people with AMD and results when multiple drusen deposits (lipid-containing accretions in the form of nodules and lamina) appear throughout the posterior pole of the retina, including the macula. The healthy eye naturally has a very small ‘blind spot’ also known as a scotoma (e.g., an area of decreased or lost vision), where the optic nerve leaves the eye. In MD, however, degeneration in the central regions of the retina can cause an enlarged scotoma in each eye, thereby affecting a person's ability to visually perceive the field of view (FOV) directly in front of the eye. Each eye can have a different scotoma and scotoma map (e.g., also referred to as scotomata). Although the peripheral vision remains unaffected (See, e.g., Mitchell et al., Health & Qual. Life Outcomes 4, (2006)), its acuity cannot fully compensate for the loss of central vision, even with low-vision aids, resulting in legal blindness (20/200 vision) (See, e.g., Quillen, Am. Fam. Physician 60, 99-108 (1999); Bressler et al., Invest. Opthalmol. Vis. Sci. 41, 624-628 (2000); and Fletcher et al., Optom. Vis. Sci. 83, 178-189 (2006)). AMD progresses to the wet form when new blood vessels, formed to provide nourishment and oxygen to the drusen deposits, leak and degenerate photoreceptors and the retinal pigment epithelium. Wet AMD accounts for more than 90% of those who suffer significant visual impairment from MD (See, e.g., Quillen, Am. Fam. Physician 60, 99-108 (1999)). Untreated, vision can decline to that of the remaining peripheral vision (See, e.g., Quillen, Am. Fam. Physician 60, 99-108 (1999)).

Despite recent advances in the treatment to halt its progression, no proven therapies exist for dry MD, although some instances of wet MD can be treated by laser or pharmacological injections (Smith et al., Curr. Opin. Ophthal. 18, 240-244 (2007)). However, these treatments do not cure the disease, but only prevents further vision loss. In the early stages of MD, people do not always recognize their vision loss, because the brain compensates for some loss of perception (e.g., perceptual fill-in) (See, e.g., Cohen et al., Graefes Arch. Clin. Exp. Opthalmol. 241, 785-791 (2003); and Weil et al., Proc. Natl. Acad. Sci. U.S. A 104, 5211-5216 (2007)), just as it does for the eye's natural blind spot.

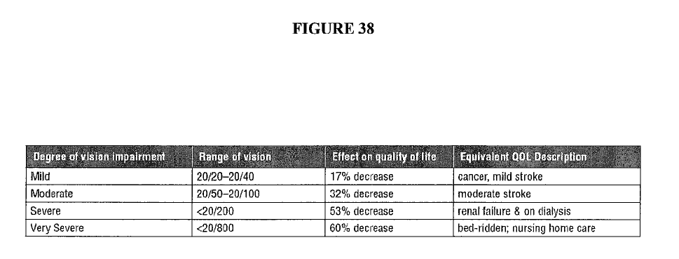

It has been shown (See, e.g., Ramachandran et al., Nature 350, 699-702 (1991); Ramachandran, Curr. Dir. Psychol. Sci. 1, 199-205 (1992); and Ramachandran, Curr. Dir. Psychol. Sci. 2, 56-65 (1993)) that the filling-in process is powerful and can extrapolate brightness and color information as well as various forms and textures of the surrounding image. However, with a very large blind spot, the brain might perform improper perceptual fill-in, causing hallucinations (e.g., Charles Bonnet syndrome) and photopsias (flickering or flashing lights) (See, e.g., Ffytche et al., Brain 122, 1247-1260 (1999); Jacob et al., Br. Med. J. 328, 1552-1554 (2004); and Abbott et al., Invest. Opthalmol. Vis. Sci. 48, 1416-1423 (2007)). More commonly, as the size of a scotoma increases (e.g., in advanced MD) or as vision loss increases (e.g., due to central vision loss, peripheral vision loss, media opacity, ring scotomas, incomplete scotomas, absolute scotomas, retinitis pigmentosa, glaucoma, homonymous hemianopsia, retinal disease, optic nerve disease, hypoxia, visual pathway disorder and other types of disorders), the visual system can no longer accurately extrapolate missing information. As disease progresses, activities of daily living (ADL) (e.g., reading, writing, walking, etc.) become more and more difficult. Indeed, for most people, the quality of life plummets as vision deteriorates (See, e.g., FIG. 38).

Annual eye exams can detect disease in its early stages. Whether naturally or through rehabilitation training, some individuals (e.g., those with MD) rely upon the least impaired portion of their FOV (e.g. the preferred retinal location), to compensate for central vision loss. This low-technology solution works well for some, but many opt for vision-enhancing assistive devices. However, these systems suffer from multiple limitations.

There are several technologies currently utilized for enhancing vision. Some assistive devices on the market today (e.g., magnifiers, CCTVs, binoculars, etc.) magnify an image so that less-acute peripheral vision can detect and recognize the image. For example, telescopic lenses can be added to eyeglasses to increase the working distance, hand-held magnifiers can be used for near vision tasks (e.g., adjusting a thermostat), and stand magnifiers may aid reading. In addition, closed-circuit television (CCTV) products include desktop magnifiers such as the MERLIN LCD, portable magnifiers such as the AMIGO, and computer magnifiers such as the JORDY. Computer programs to help those with poor vision are also commercially available. Examples include screen readers such as WINDOW-EYES or JAWS and computer magnifiers such as ZOOMTEXT (See, e.g., Virgili and Acosta, Cochrane Database of Systematic Reviews 2006 1-28 (2006)).

These systems suffer from multiple limitations. First, they are often large, unwieldy, difficult to use, and/or context-specific (e.g., one device for reading, another for watching TV, etc.). Second, as vision loss progresses, the need for magnification increases. Consequently, these technologies magnify one small detail at the expense of seeing the whole context; the magnified FOV can create a new blind spot or ‘ring scotoma’ that actually obscures the residual vision by reducing the overall FOV. Thus, the very device that enhances vision in some areas can block vision of part of the surrounding environment.

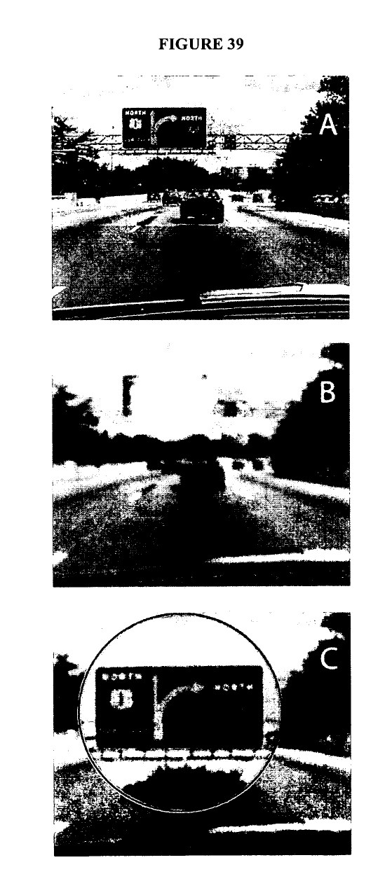

For example, Panel A of FIG. 39 illustrates normal vision from the perspective of a person being driven in a car; Panel B schematically shows what a person with MD might see; the damaged central vision cannot clearly discern the highway sign and the periphery is blurry; Panel C demonstrates how an eye-based magnifying technology can capture the missing central vision, but in so doing, creates a ring scotoma that blocks other potentially important objects ordinarily in the FOV (e.g., other cars). Although the magnified image is in focus in the figure, it would not be for a person with MD (See, e.g., Peli, Optom. Vis. Sci. 79, 569-580 (2002)).

Third, many users report getting ‘lost’ because the magnifying devices offer such restricted FOV information. For some, the very small FOV can cause dizziness or nausea because of the mismatch between movement of the magnified image and the innate vestibular knowledge of balance.

Several invasive devices also exist. Cortical implants have offered some promise. Examples include the artificial visual prosthesis developed at The Dobelle Institute, which improved the vision of a completely blind man to 20/400 (See, e.g., Dobelle, ASAIO J. 46, 3-9 (2000)) and the Artificial Vision system under development at the University of Utah. This cortically based visual neuroprothesis system will use five main components: a micro-video camera to record light detected in the visual field; signal processing electronics; a small power source; an implanted multichannel stimulator delivering power and data to the implant system; and a microelectrode array (See, e.g., Hossain et al., Br. Med. J. 330, 30-33 (2005)).

Retinal chips (SECONDSIGHT, Sylmar, Calif.) have been designed to provide an artificial replacement of the damaged retina in the exact location affected by the disorder. These chips have been tested in individuals suffering from retinitis pigmentosa, and may eventually include individuals with MD. Currently, chip resolution is very low (a 16-electrode array producing 16 phosphemes (light flashes) (See, e.g., Javaheri et al., Ann. Acad. Med. Singapore 35, 137-144 (2006))).

Implantable miniature telescopes (VISIONCARE Technologies, Inc., Saratoga, Calif.) exist as another invasive technology. The implantable device is approximately pea-sized and provides 2.2- to 3-fold magnification. Recipients of miniature telescope implants have experienced a 3-line increase in their best-corrected distance visual acuity (BCDVA), an 50% improvement in their best-corrected near vision acuity (BCNVA), and a 7-point change in their quality of life as reflected by NEI VFQ-25 scores. However, recipients also suffered a 20% endothelial cell loss in the first 3 months after implantation, which contributed to termination of an on-going clinical trial (See, e.g., Hudson et al., Opthalmology 113, 1987-2001 (2006); Lane and Kuppermann, Curr. Opin. Ophthal. 17, 94-98 (2006)).

A significant limitation of these technologies is that each requires surgical implantation. The surgical implantation itself, as well as the potential for subsequent infection and biological incompatibility, are significant risks. Importantly, many of these implantable devices suffer from low resolution information and future device enhancements or modifications would subject the user to repeated surgical procedures.

Accordingly, in some embodiments, the present invention provides a vision assistance and/or augmentation device (e.g., a MD assistive/augmentation device) that can be used to supplement a subject's vision (e.g., supplement and/or augment vision in a subject with vision loss). In some embodiments, a device of the present invention augments vision loss associated with disease (e.g., augments a user's existing (e.g., peripheral) vision (e.g., without obscuring it) and/or provides a high-resolution image of a user's environment (e.g., that permits a user to conduct activities of daily living))). The present invention is not limited to any particular disease or type of vision loss that can be supplemented, corrected and/or enhanced using a device of the present invention. Many different types of vision loss can be supplemented, corrected and/or enhanced including, but not limited to, macular degeneration (e.g., adult macular degeneration), central vision loss, peripheral vision loss, media opacity, ring scotomas, incomplete scotomas, absolute scotomas, retinitis pigmentosa, glaucoma, homonymous hemianopsia, retinal disease, optic nerve disease, hypoxia, visual pathway disorders and other types of disorders.

In some embodiments, the present invention provides a vision assistance and/or augmentation device (e.g., for MD or other type of vision loss (e.g. that is lightweight, portable/wearable, and/or that is unobtrusive)). In some embodiments, a vision assistance and/or augmentation device is configured to track with a user's gaze point (e.g., as described in Example 32). For example, in some embodiments, a vision assistance and/or augmentation device of the present invention is a VIEW POINT PC-60 EYEFRAME SCENE CAMERA package (ARRINGTON Research, Scottsdale, Ariz.), or other type of eye tracking device (e.g., a device described in U.S. Pat. No. 6,943,754, a device described in U.S. Pat. No. 6,421,185, a device described in Sandor and Leger, Aviat Space Environ Med. 1991 November; 62(11):1026-31; or a device described by Sparto et al., Engineering in Medicine and Biology Society, 2004. IEMBS apos; 04. 26th Annual International Conference of the IEEE Volume 2, Issue, 1-5 Sep. 2004 Page(s): 4836-4839), or similar device.

In some embodiments, a vision assistance and/or augmentation device of the present invention captures information about a user's environment from an area of vision loss (e.g., a region of a user's field of view in which vision is lost and/or impaired) and displays the information regarding the user's environment (e.g., from an area of vision loss (e.g., due to MD or other type of aging or disease associated with vision loss described herein) to a region of the user's body (e.g., on the tongue of the user). In some embodiments, a user is able to perceive the information displayed on the region of the user's body (e.g., on the tongue) as that portion of the region of the user's field of view that is lost and/or impaired. For example, in some embodiments, information provided to a user (e.g., to the tongue of a user) fills in one or more areas of vision loss (e.g., in the user's field of view (e.g., scotoma caused by MD)).

A vision assistance and/or augmentation device and/or methods of the present invention are not limited to MD. Indeed, a variety of different types of subjects may benefit from a device, system and/or method of the present invention, including, but not limited to, those who are blind or have low vision (e.g., due to conditions including glaucoma, diabetic retinopathy, MD, AMD, or Retinitis Pigmentosa). In some embodiments, the present invention provides a user of a device and/or method described herein the ability to recognize letters, words, objects, people and/or other things (e.g., that a user has difficulty reading or seeing and/or is not able to read and/or see without a device of the present invention (e.g., that permits a user to conduct activities of daily living)). In some embodiments, the present invention is compatible with a user's own corrective eyewear or other vision-assisting devices (e.g., one or more vision-assisting devices described herein). In some embodiments, a device of the present invention can be easily customizable and/or upgradeable. In some embodiments, the present invention provides a vision assistance and/or augmentation device (e.g., that is lightweight, fully portable/wearable, and/or that is unobtrusive (e.g., that tracks with a user's gaze point, captures information about the environment from an area of vision loss, and/or displays information from an area of vision to a subject (e.g., displays information on the tongue)).

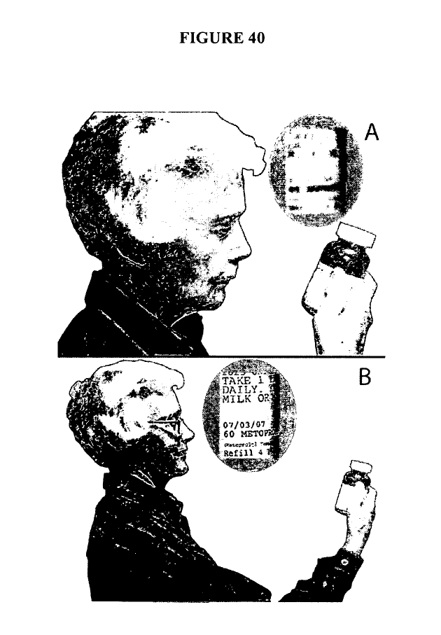

For example, in some embodiments, a vision assistance and/or augmentation device of the present invention (e.g., a device of Example 32) does not obstruct a user's existing peripheral vision but rather fills in an area of vision loss. For example, FIG. 40B shows a schematic of difficulties a person with MD undergoes in straining to read the label on a prescription bottle. In some embodiments, using a device of the present invention, the individual can clearly read the label (See, e.g., FIG. 40B).

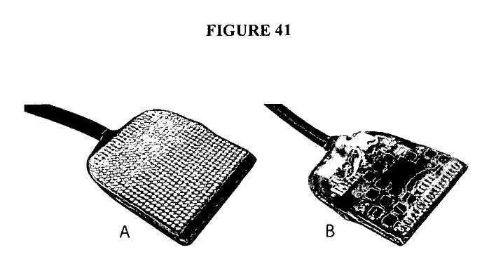

In some embodiments, the present invention provides a device comprising an electrode array on the tongue (e.g., tongue display) (See FIG. 41) and the merging of eye tracking (e.g., central gaze tracking) with an individual's scotoma map (See, e.g., Example 32).

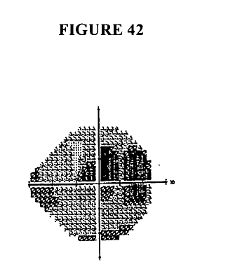

In some embodiments, a scotoma map (See, e.g., FIG. 42) is used to map and/or mark the areas of preserved and/or compromised vision (e.g., central and/or peripheral vision (e.g., for each eye)). The present invention is not limited by the method or means of creating a user's scotoma map. Indeed, a variety of different methods and devices can be utilized for generating a user's scotoma map (e.g., of each eye) including, but not limited to, the SITA-24 visual field test, a computerized perimetry instrument (e.g., INTERZEAG Octopus 500EZ (INTERZEAG, Schlieren, Switzerland), Humphrey Field Analyzer (Carl Zeiss Meditec, Dublin, Ireleand), MP-1-Micro Perimeter (Nidek Technologies, Inc.), or others known in the art (e.g., described in U.S. Pat. No. 5,035,500).

A vision assistance and/or augmentation device of the present invention (e.g., described in Example 32) can be used, for example, to supplement, augment, correct and/or enhance a user's vision (e.g., via filling in one or more areas of lost vision in a user's field of view (e.g., due to disease (e.g., macular degeneration))). The present invention is not limited to any particular mechanism of filing in one or more areas of lost vision in a user's field of view. In some embodiments, a vision assistance and/or augmentation device of the present invention generates and/or leads to neural computation and/or activation that occurs in a user's brain (e.g., in response to signals (e.g., electrical signals) provided to a subject (e.g., via an array of electrodes) by the device). In some embodiments, neural activation and/or computation occurs in early visual cortical areas (e.g., cortical areas involved in “filling-in” visual features not perceived without use of a system of the present invention) that are not activated and/or in which computations do not occur without use of a vision assistance and/or augmentation device of the present invention. In some embodiments, a vision assistance and/or augmentation device of the present invention provides (e.g., “fills in”) a perceptual phenomenon and/or a perceptual event to a user (e.g., including, but not limited to, one or more visual features and/or characteristics (e.g., shape, color, brightness, texture, motion, rigidness, contrast, focus, etc.)). In some embodiments, a device of the present invention provides a manifestation of a visual function of surface interpolation to a user.

In some embodiments, a device of the present invention (e.g., a vision assistance and/or augmentation device) fills in missing information within a user's field of view (e.g., in a blind spot or at a scotoma (e.g., due to disease or condition (e.g., macular degeneration))). In some embodiments, a device of the present invention (e.g., a vision assistance and/or augmentation device) corrects, supplements and/or enables steady fixation and stabilized retinal images (e.g., in situations where there is no deficit of visual inputs (e.g., stabilization of the border of a surface on the retina)). In some embodiments, a device of the present invention (e.g., a vision assistance and/or augmentation device) reduces, corrects and/or eliminates neon color spreading and/or other illusions (e.g., caused by damage and or disease). In some embodiments, a device of the present invention (e.g., a vision assistance and/or augmentation device) provides luminance contrast information (e.g., detected and/or perceived at visual borders) and/or enables a user to detect, perceive and/or interpolate the brightness of a surface between borders (e.g., allowing brightness filling in to occur). In some embodiments, a device of the present invention (e.g., a vision assistance and/or augmentation device) is involved with filling in a subject's perceptual and/or cognitive processes (e.g., visual cortical areas in a human subject (e.g., See, e.g., Komatsu, Nature Reviews Neuro, 7 220-231 (2006))). For example, in some embodiments, a device of the present invention (e.g., a vision assistance and/or augmentation device) provides a neural mechanism involved in processing brightness of a surface and/or the illusory brightness filled in from a contrast border (e.g., to share the same neural mechanisms at the level of the V2 thin stripe). In some embodiments, a device of the present invention (e.g., a vision assistance and/or augmentation device) stimulates and/or provides information to one or more visual cortical areas (e.g., primary visual cortex (also known as striate cortex or V1) and/or extrastriate visual cortical areas such as V2, V3, V4, and V5 (described, e.g., in Martinez et al., Nature Neuroscience 2, 364-369 (1999), hereby incorporated by reference).

The primary visual cortex, V1, is the koniocortex (sensory type) located in and around the calcarine fissure in the occipital lobe. It receives information directly from the lateral geniculate nucleus. V1 transmits information to two primary pathways, called the dorsal stream and the ventral stream. The dorsal stream begins with V1, goes through Visual area V2, then to the dorsomedial area and Visual area MT (also known as V5) and to the inferior parietal lobule. The dorsal stream, sometimes called the “Where Pathway”, is associated with motion, representation of object locations, and control of the eyes and arms, especially when visual information is used to guide saccades or reaching (See, e.g., Goodale & Milner (1992), Trends in Neuroscience 15: 20-25).

The ventral stream begins with V1, goes through Visual area V2, then through Visual area V4, and to the inferior temporal lobe. The ventral stream, sometimes called the “What Pathway”, is associated with form recognition and object representation. It is also associated with storage of long-term memory.

Neurons in the visual cortex fire action potentials when visual stimuli appear within their receptive field. By definition, the receptive field is the region within the entire visual field which elicits an action potential. But for any given neuron, it may respond to a subset of stimuli within its receptive field. This property is called tuning. In the earlier visual areas, neurons have simpler tuning. For example, a neuron in V1 may fire to any vertical stimulus in its receptive field. In the higher visual areas, neurons have complex tuning. For example, in the inferior temporal cortex (IT), a neuron may only fire when a certain face appears in its receptive field. The visual cortex receives its blood supply primarily from the calcarine branch of the posterior cerebral artery.

The primary visual cortex is the best studied visual area in the brain. In all mammals studied, it is located in the posterior pole of the occipital cortex (the occipital cortex is responsible for processing visual stimuli). It is the simplest, earliest cortical visual area. It is highly specialized for processing information about static and moving objects and is excellent in pattern recognition. The functionally defined primary visual cortex is approximately equivalent to the anatomically defined striate cortex. The name “striate cortex” is derived from the stria of Gennari, a distinctive stripe visible to the naked eye that represents myelinated axons from the lateral geniculate body terminating in layer 4 of the gray matter. The primary visual cortex is divided into six functionally distinct layers, labelled 1 through 6. Layer 4, which receives most visual input from the lateral geniculate nucleus (LGN), is further divided into 4 layers, labelled 4A, 4B, 4Cα, and 4Cβ, Sublamina 4Cα receives most magnocellular input from the LGN, while layer 4Cβ receives input from parvocellular pathways.

V1 has a very well-defined map of the spatial information in vision. For example, in humans the upper bank of the calcarine sulcus responds strongly to the lower half of visual field (below the center), and the lower bank of the calcarine to the upper half of visual field. Conceptually, this retinotopy mapping is a transformation of the visual image from retina to V1. The correspondence between a given location in V1 and in the subjective visual field is very precise: even blind spots can be mapped into V1. Evolutionarily, this correspondence is very basic and found in most animals that possess a V1. In human and animals with a fovea in the retina, a large portion of V1 is mapped to the small, central portion of visual field, a phenomenon known as cortical magnification. Perhaps for the purpose of accurate spatial encoding, neurons in V1 have the smallest receptive field size of any visual cortex regions.

The tuning properties of V1 neurons (e.g., what the neurons respond to) differ greatly over time. Early in time (40 ms and further) individual V1 neurons have strong tuning to a small set of stimuli. That is, the neuronal responses can discriminate small changes in visual orientations, spatial frequencies and colors. Furthermore, individual V1 neurons in human and animals with binocular vision have ocular dominance, namely tuning to one of the two eyes. In V1, and primary sensory cortex in general, neurons with similar tuning properties tend to cluster together as cortical columns. It is currently accepted that early responses of V1 neurons consists of tiled sets of selective spatiotemporal filters. In the spatial domain, the functioning of V1 can be thought of as similar to many spatially local, complex Fourier transforms. Theoretically, these filters together can carry out neuronal processing of spatial frequency, orientation, motion, direction, speed (e.g., temporal frequency), and many other spatiotemporal features.

Later in time (after 100 ms) neurons in V1 are also sensitive to the more global organization of the scene. These response properties can stem from recurrent processing (e.g., the influence of higher-tier cortical areas on lower-tier cortical areas) and lateral connections from pyramidal neurons. The visual information relayed to V1 is not coded in terms of spatial (or optical) imagery, but rather as the local contrast. As an example, for an image comprising half side black and half side white, the divide line between black and white has strongest local contrast and is encoded, while few neurons code the brightness information (e.g., black or white). As information is further relayed to subsequent visual areas, it is coded as increasingly non-local frequency/phase signals. At these early stages of cortical visual processing, spatial location of visual information is well preserved.

Lesions to primary visual cortex can lead to a scotoma or blindspot/hole in the visual field. Subjects with scotomas are often able to make use of visual information presented to their scotomas, despite being unable to consciously perceive it. This phenomenon has been termed blindsight. Accordingly, in some embodiments, a device, system and/or methods of the present invention are used to provide information (e.g., visual information) to a scotoma, blindspot and/or hole in a subject's visual field (e.g., that can be perceived (e.g., consciously perceived) by the subject)).

Visual area V2 is the second major area in the visual cortex, and first region within the visual association area. It receives strong feedforward connections from V1 and sends strong connections to V3, V4, and V5. It also sends strong feedback connections to V1. Anatomically, V2 is split into four quadrants, a dorsal and ventral representation in the left and the right hemispheres. Together these four regions provide a complete map of the visual world. Functionally, V2 has many properties in common with V1. Cells are tuned to simple properties such as orientation, spatial frequency, and color. The responses of many V2 neurons are also modulated by more complex properties, such as the orientation of illusory contours and whether the stimulus is part of the figure or the ground (See, e.g., Qiu and von der Heydt, Neuron. 2005 Jul. 7; 47(1):155-66).

Visual area V3 is a term used to refer to the region of cortex located immediately in front of V2. Some researchers propose that V3 is in fact a complex of two or three functional subdivisions. For example, some researchers have proposed the existence of a “dorsal V3” in the upper part of the cerebral hemisphere, is distinct from the “ventral V3” (or ventral posterior area, VP) located in the lower part of the brain. Dorsal and ventral V3 have distinct connections with other parts of the brain, appear different in sections stained with a variety of methods, and contain neurons that respond to different combinations of visual stimulus (for example, color-selective neurons are more common in the ventral V3). Dorsal V3 is normally considered to be part of the dorsal stream, receiving inputs from V2 and from the primary visual area and projecting to the posterior parietal cortex. It may be anatomically located in Brodmann area 19. Adjacent areas 3A and 3B may also exist. Recent work with functional magnetic resonance imaging (fMRI) has suggested that area V3NV3A may play a role in the processing of global motion (See, e.g., Braddick and O'Brian, et al (2001). Perception 30: 61-7). Other studies prefer to consider dorsal V3 as part of a larger area, named the dorsomedial area (DM), which contains a representation of the entire visual field. Neurons in area DM respond to coherent motion of large patterns covering extensive portions of the visual field (See, e.g., Lui et al., Eur J. Neurosci. 2007 March; 25(6):1780-92). Ventral V3 (VP), has much weaker connections from the primary visual area, and stronger connections with the inferior temporal cortex. While earlier studies proposed that VP only contained a representation of the upper part of the visual field (above the point of fixation), more recent work indicates that this area is more extensive than previously appreciated, and like other visual areas it may contain a complete visual representation. The revised, more extensive VP is referred to as the ventrolateral posterior area (VLP) (See, e.g., Rosa and Tweedale R (2000) J Comp Neurol 422:621-51).

Visual area V4 is one of the visual areas in the extrastriate visual cortex (e.g., of the macaque monkey). It is located anterior to V2 and posterior to visual area PIT. It comprises at least four regions (left and right V4d, left and right V4v), and contains rostral and caudal subdivisions as well. V4 is the third cortical area in the ventral stream, receiving strong feedforward input from V2 and sending strong connections to the posterior inferotemporal cortex (PIT). It also receives direct inputs from V1, especially for central space. In addition, it has weaker connections to V5 and visual area DP (the dorsal prelunate gyrus). V4 is the first area in the ventral stream to show strong attentional modulation. Most studies indicate that selective attention can change firing rates in V4 by about 20% (See, e.g., Moran and Desimone. Science 229(4715), 1985). Like V1, V4 is tuned for orientation, spatial frequency, and color. Unlike V1, it is tuned for object features of intermediate complexity, like simple geometric shapes. Visual area V4 is not tuned for complex objects such as faces, as areas in the inferotemporal cortex are. V4 exhibits long-term plasticity, encodes stimulus salience, is gated by signals coming from the frontal eye fields, and shows changes in the spatial profile of its receptive fields with attention.

Visual area V5, also known as visual area MT (middle temporal), is a region of extrastriate visual cortex that is thought to play a major role in the perception of motion, the integration of local motion signals into global percepts and the guidance of some eye movements (See, e.g., Born and Bradley, Annu Rev Neurosci 28: 157-89). MT is connected to a wide array of cortical and subcortical brain areas. Its inputs include the visual cortical areas V1, V2, and dorsal V3 (dorsomedial area) (See, e.g., Ungerleider and Desimone, (1986). J Comp Neurol 248 (2): 190-222), the koniocellular regions of the LGN (See, e.g., Sincich et al., (2004). Nat Neurosci 7 (10): 1123-8), and the inferior pulvinar. The pattern of projections to MT changes somewhat between the representations of the foveal and peripheral visual fields, with the latter receiving inputs from areas located in the midline cortex and retrosplenial region (See, e.g., Palmer and Rosa (2006). Eur J Neurosci 24(8): 2389-405). V1 provides the important input to MT. Neurons in MT are also capable of responding to visual information, often in a direction-selective manner, even after V1 has been destroyed or inactivated (See, e.g., Rodman et al., (1989) J Neurosci 9(6):2033-50). Certain types of visual information may reach MT before it even reaches V1.

MT sends its major outputs to areas located in the cortex immediately surrounding it, including areas FST, MST and V4t (middle temporal crescent). Other projections of MT target the eye movement-related areas of the frontal and parietal lobes (e.g., frontal eye field and lateral intraparietal area). The first studies of the electrophysiological properties of neurons in MT showed that a large portion of the cells were tuned to the speed and direction of moving visual stimuli (See, e.g., Dubner and Zeki, (1971). Brain Res 35 (2): 528-32; Maunsell and Van Essen (1983). J Neurophysiol 49 (5): 1127-47). These results suggest that MT plays a significant role in the processing of visual motion. Lesion studies have also supported the role of MT in motion perception and eye movements and neuropsychological studies of a patient who could not see motion, seeing the world in a series of static “frames” instead, suggests that MT in the primate is homologous to V5 in the human (See, e.g., Hess et al., (1989). Journal of Neuroscience 9 (5): 1628-1640; Baker et al., (1991). Journal of Neuroscience 11 (2): 454-461).

MT also appears to integrate local visual motion signals into the global motion of complex objects (See, e.g., Movshon et al., (1985)). The analysis of moving visual patterns. In: C. Chagas, R. Gattass, & C. Gross (Eds.), Pattern recognition mechanisms (pp. 117-151), Rome: Vatican Press).

In some embodiments, the present invention provides systems, methods and/or devices for use in research (e.g., vision research (e.g., on the primary visual cortex and/or cortical areas (e.g., involving recording action potentials from electrodes within the brain or through recording intrinsic optical signals or fMRI signals (e.g., from V1)))).

In some embodiments, a system of the present invention is utilized for neural imaging (e.g., of visual cortical areas of a subject's brain). In some embodiments, early visual areas are activated (e.g., representing the boundary of the surface and the interior of the surface)). In some embodiments, neural activation correlates with a user's perception.

The present invention is not limited to any particular mechanism of filling-in (e.g., using a device (e.g., vision assistance and/or augmentation device) of the present invention to fill in (e.g., provide information (e.g., visual information) to one or more scotomas in a subject's visual field (e.g., via any one of the cortical areas described herein). Although an understanding of the mechanism is not necessary to practice the present invention and the present invention is not limited to any particular mechanism of action, in some embodiments, a vision assistance and/or augmentation device of the present invention induces activity in one or more cortical regions that topographically corresponds to (e.g., that are topographically mapped to) the visual field where filling-in occurs. In some embodiments, filling in occurs for a monocular scotoma. In some embodiments, filling in occurs for a binocular scotoma. In some embodiments, a device of the present invention provides information (e.g., visual information) to and/or activates neurons located in the cortical region that corresponds to the scotoma (e.g., inducing and/or creating receptive fields around the scotoma). In some embodiments, a device of the present invention induces and/or generates reorganization of the retinotopic map of the visual cortex (e.g., in proximity to the region around the scotoma).

Although an understanding of the mechanism is not necessary to practice the present invention and the present invention is not limited to any particular mechanism of action, in some embodiments, a device of the present invention provides information (e.g., visual information) to and/or activates neurons in higher cortical areas (e.g., that receive signals from V1 (e.g., a reorganized V1 or non-reorganized V1) regions and/or interpret it (e.g., according to the original retinotopic map or a reorganized retinotopic map) and treat it as if the signal originated from visual input within the scotoma). Although an understanding of the mechanism is not necessary to practice the present invention and the present invention is not limited to any particular mechanism of action, in some embodiments, a subject can perceive information in a scotoma using a device of the present invention (e.g., visual features (e.g., that would be present in a subject's normal field of view) present within and at the surround of the scotoma exist within the scotoma). In human patients with macular degeneration, a large degree of reorganization of the retinotopic map in the visual cortex has been observed using fMRI measurements (See, e.g., Baker et al., J. Neurosci. 25, 614-618 (2005).51. In some embodiments, a device of the present invention is utilized to reduce and/or eliminate distortion of the visual space accompanying reorganization of the cortical retinotopic map (e.g., that is related, at least in part, to the filling-in at the scotoma).

Although an understanding of the mechanism is not necessary to practice the present invention and the present invention is not limited to any particular mechanism of action, in some embodiments, a device of the present invention (e.g., a visual assistance and/or augmentation device (e.g., a device described in Example 32) provides information (e.g., visual information) to and/or activates neurons that potentiate neural filling in within a subject. The present invention is not limited to any particular neural mechanism of filling in. Several different types of neural filling in are contemplated including, but not limited to, the ability of early visual areas to extract contrast information at the surface border, with color and shape of a surface reconstructed in higher areas on the basis of this information (e.g., symbolic or cognitive theory of filling in); spread of activation that occurs across the retinotopic map of the visual cortex from the border to the interior of the surface, and a two-dimensional array of neurons with a pointwise representation of visual features, such as color or brightness, activated in early visual areas (e.g., isomorphic theory of filling in); and wherein different sets of neurons in deep layers are selectively activated depending on the stimulus to be filled in (e.g., scale sensitive mechanism of filling in deep layers).

For example, neurophysiological and neuroimaging studies have shown that in most situations in which filling-in occurs, early visual areas are activated. Thus, in some embodiments, a device (e.g., vision assistance and/or augmentation device (e.g., a device of Example 32) activates neurons in a region of the retinotopic map of early areas representing not only the boundary of the surface but also the interior of the surface. In some embodiments, these neural activations are correlated with perception.

In some embodiments, a system, device and/or methods of the present invention provide ‘symbolic’ or ‘cognitive’ filling in (See, e.g., Pessoa et al., Behav. Brain Sci. 21, 723-748; discussion 748-802 (1998). For example, in some embodiments, early visual areas extract contrast information at the surface border, and the color and shape of the surface are reconstructed in higher areas on the basis of this information. A blind spot or scotoma does not generate border signals by itself, but the surface covering these regions generates contrast information at its border. Higher areas use this information to represent the entire surface, filling in the blind spot or scotoma. In the Troxler effect and stabilized retinal image, border signals of a surface that is stationary on the retina diminish and the signal from the un-stabilized outer border is used to construct the entire surface, resulting in the perceptual filling-in of visual features from the un-stabilized border. According to this theory, there is no need for the activity change to occur in the surface region where filling in of visual features is perceived. However, in some embodiments, filling in at a scotoma (e.g., blind spot) accompanies neural activation as early as V1 or V2.

In some embodiments, a system, device and/or methods of the present invention provide ‘isomorphic’ filling in. For example, in some embodiments, when perceptual filling-in occurs, spread of activation occurs across the retinotopic map of the visual cortex from the border to the interior of the surface, and a two-dimensional array of neurons with a pointwise representation of visual features (e.g., color or brightness) is activated in early visual areas (See, e.g., Gerritas et al., Exp. Brain Res. 11, 411-430 (1970); Pessoa et al., Behav. Brain Sci. 21, 723-748; discussion 748-802 (1998); Cohen et al., Percept. Psychophys. 36, 428-456 (1984); Arrington, Vision Res. 34, 3371-3387 (1994); and Friedman et al., J. Physiol. (Lond.) 548, 593-613 (2003). In some embodiments, a similar two-dimensional array of feature-sensitive neurons is activated when the real surface is perceived in the normal visual field (See, e.g., Rossi et al., Science 273, 1104-1107 (1996); Kinoshita et al., Science 273, 1104-1107 (1996); and Friedman et al., J. Physiol. (Lond.) 548, 593-613 (2003)). In some embodiments, when a uniform surface presented in the normal visual field is viewed or when a surface covering the blind spot is viewed through the fellow eye, neurons in the superficial layer and those with small receptive fields are also activated. In some embodiments, the perception of real surface and that of filled-in surface share the same neural processes at some stage beyond V1. In some embodiments, activity of V1 correlates with perception at a region corresponding to the filled-in surface (See, e.g., Sasaki and Watanabe, Proc. Natl. Acad. Sci. USA 101, 18251-18256 (2004); Meng et al., Nature Neurosci. 8, 1248-1254 (2005); Tong and Engel, Nature 411, 195-199 (2001)). In some embodiments, filling in relates to characteristics of neural responses observed in V1 during filling-in at the blind spot (See, e.g., Komatsu, Nature Reviews, Neuroscience, 7, 220-266 (2006). For example, in some embodiments, neurons from the BS region in V1 respond when a uniform surface is presented to the blind spot (See, e.g., Komatsu et al. J. Neurosci. 20, 9310-9319 (2000). In some embodiments, when perceptual filling in occurs at a scotoma/blind spot, different sets of neurons in deep layers are selectively activated depending on the stimulus to be filled in (See, e.g., Matsumoto and Komatsu, J. Neurophysiol. 93, 2374-2387 (2005). In some embodiments, surface perception filling-in is related to the function of surface interpolation based on border contrast information (See, e.g., Kellman et al. J. Exp. Psychol. Hum. Percept. Perform. 24, 859-869 (1998)). In some embodiments, the occurrence of filling-in is closely related to three-dimensional interpretation of a scene (e.g., in a user's (e.g., of a vision assistance and/or augmentation device of the present invention) field of view).

In some embodiments, surface perception includes constructing the surface based on available contour information and the interpolation of incomplete data. Although an understanding of the mechanism is not necessary to practice the present invention and the present invention is not limited to any particular mechanism of action, in some embodiments, both early visual areas and higher areas are involved. Processes related to contour formation, such as contextual modulation of contours and border ownership assignment, have been shown to be formed in early visual areas, and neural responses related to figure—ground segregation have been observed in both early and late areas. In some embodiments, a process such as this occurs for any surface, regardless of whether it is modal or amodal. For example, many representations of surfaces may emerge in each direction in visual space, and these might be maintained through a recurrent feedforward-feedback loop between early and late visual areas (See, e.g., Mumford, in Large-Scale Neuronal Theories of the Brain (eds Koch, C. & Davis, J.) 125-152 (MIT Press, Cambridge, Mass., 1994); Rao et al. Nature Neurosci. 2, 79-87 (1999); Mendola, J. in Filling-in (eds Pessoa, L. & De Weerd, P.) 38-58 (Oxford Univ. Press, New York, 2003).

In some embodiments, surface perception gives visible features, such as color, brightness and texture, to a surface that was assigned the top priority position in a depth order (e.g., nearest surface) as a result of surface perception that constructs the surface based on available contour information and the interpolation of incomplete data. Thus, in some embodiments, one mechanism of filling-in relates to generating modal perception of the surface. For example, in a process of filling in, visual signals transmitted through horizontal connections in V1 or through feedback projection from higher areas selectively activate specific neurons in deep layers of V1, and modal surface perception is experienced.

In some embodiments, the present invention provides an easily customizable and upgradeable, fully portable, easy-to-use, and effective visual assistance and/or augmentation device (e.g., a device described in Example 32). In some embodiments, a device will includes a wireless oral unit (e.g., with a plurality of electrodes (e.g., from 100-500 electrodes, from 500-1000 electrodes, from 1000-2000 electrodes, from 2000-5000 electrodes, from 5000-7500 electrodes, from 7500-10000 electrodes, or more). In some embodiments, each electrode is referred to as a ‘pixel’ (e.g., analogous to digital videography) on the tongue display. In some embodiments, cameras are integrated (e.g., invisibly) in a pair of eyeglasses, and a miniature iPOD-size controller is unit is part of the device. Thus, in some embodiments, a visual assistance and/or augmentation device is configured to increase safety and ease of use (e.g., for a specific population of users (e.g., users that are at least 55, users that are at least 60, or users that are at least 65 years of age and/or users who suffer from a form of vision loss described herein (e.g., macular degeneration).

In some embodiments, a vision assistance and/or augmentation device (e.g., described in Example 32) can be used to improve a user's results on a standardized questionnaire that assesses quality of life (QOL) issues surrounding a person's ability to deal with vision loss (e.g., a NEI visual functioning questionnaire-25 (NEI VFQ-25). In some embodiments, a vision assistance and/or augmentation device (e.g., described in Example 32) can be used to improve an eye refraction vision test (e.g., that determines a person's best visual acuity with corrective lenses). In some embodiments, a vision assistance and/or augmentation device (e.g., described in Example 32) can be used to improve a ETDRS visual acuity test (e.g., that measures a person's ability to discern letters of decreasing size on a standard letter chart from a standard distance under standard lighting conditions). In some embodiments, a vision assistance and/or augmentation device (e.g., described in Example 32) can be used to improve results on a Pelli-Robson contrast sensitivity test (e.g., that measure a person's ability to discern letters of decreasing gray-scale contrast). In some embodiments, a vision assistance and/or augmentation device (e.g., described in Example 32) can be used to improve results on a EVA letter acuity test (e.g., that presents one letter at a time on a display, with or without distracting flanker bars around the letter to simulate surrounding letters (e.g., that generates an overall acuity score)).

### Sensory Input Devices

A wide range of sensory input devices find use with the present invention. In some preferred embodiments, the device provides one or more tactile stimulators that communicate (e.g., physically, electronically) with the surface of a subject (e.g., skin surface, tongue, internal surface). The number, size, density, and position (e.g., location and geometry) of stimulators are selected so as to be able to transmit the desired information to the subject for any particular application. For example, where the device is used as a simple alarm, a single stimulator may be sufficient. In embodiments where visual information is provided, more stimulators may be desired. In embodiments where only direction needs to be perceived, a limited ring of stimulators indicating 180-degree, 360-degree direction may be used (or 4 stimulators for N, W, E, S direction, used in combination to indicate intersections). In some embodiments, stimulators are positioned and signals are timed to produce a tactile phi phenomenon (i.e., an optical illusion in which the rapid appearance and disappearance of two stationary objects is perceived as the movement back and forth of a single object). With correct placement and timing, a “phantom” or apparent movement can be achieved in one or more directions. Using such a method increases the amount of information that can be conveyed with a limited number of stimulators. Increase in complexity of information with a limited set of stimulators may also be achieved by varying gradients of signal (intensity, pitch, spatial attribute, depth) to create a palette of tactile “colors” or sensations (e.g., paraplegics perceive one level of gradient as a “bladder full” alarm and another level of gradient with the same stimulator or stimulators as a “object in contact with skin” perception).

The nature of the sensors and devices may be dictated by the application. Examples include use of a microgravity sensor to provide vestibular information to an astronaut or a high performance pilot, and robotic and minimally invasive surgery devices that include MEMS technology sensors to provide touch, pressure, shear force, and temperature information to the surgeon, so that a cannula being manipulated into the heart could be “felt” as if it were the surgeon's own finger.

Particularly preferred embodiments of the present invention employ electrotactile input devices configured to transmit information to the tongue (See, e.g., U.S. Pat. No. 6,430,450, incorporated herein by reference in its entirety, which provides devices for electrotactile stimulation of the tongue). The present invention makes use of, but is not limited to, such devices. In some embodiments, a mouthpiece providing a simulator or an array of stimulators in used. In other embodiments, stimulators are implanted in the skin or in the mouth (see, e.g., WO 05/040989, incorporated by reference herein in its entirety). Additional devices are described in the Examples section, below.

Preferred devices of the present invention receive information via wireless communication to maximize ease of use.

The following embodiments are provided by way of example and are not intended to limit the invention to these particular configurations. Numerous other applications and configurations will be appreciated by those skilled in the art.

In preferred embodiments, the tongue display unit (TDU) has output coupling capacitors in series with each electrode to guarantee zero dc current to minimize potential skin irritation. The output resistance is approximately 1 kΩ. The design also employs switching circuitry to allow all electrodes that are not active or “on image” to serve as the electrical ground for the array, affording a return path for the stimulation current.

In preferred embodiments, electrotactile stimuli are delivered to the dorsum of the tongue via flexible electrode arrays placed in the mouth, with connection to the stimulator apparatus via a flat cable passing out of the mouth or through wireless communication technology. The electrotactile stimulus involves 40-μs pulses delivered sequentially to each of the active electrodes in the pattern. Bursts of three pulses each are delivered at a rate of 50 Hz with a 200 Hz pulse rate within a burst. This structure yields strong, comfortable electrotactile percepts. Positive pulses are used because they yield lower thresholds and a superior stimulus quality on the fingertips and on the tongue.

In some embodiments, electrodes comprise flat disc surfaces that contact the skin. Other embodiments employ different geometries such as concave or convex surfaces or pointed surfaces.

Experiments conducted during the development of the present invention have determined that the threshold of sensation and useful range of sensitivity, as a function of location on the tongue, is significantly inhomogeneous. Specifically, the front and medial portions of the tongue have a relatively low threshold of sensation, whereas the rear and lateral regions of the stimulation area are as much as 32% higher. Example 16 describes methods to optimize signaling for any particular application. The differences are likely due to the differences in tactile stimulator density and distribution. Concomitantly, the useful range of sensitivity to electrotactile stimulation varies as a function of location, and in a pattern similar to that for threshold.

To compensate for sensory inhomogeneity, the system utilizes a dynamic algorithm that allows the user to individually adjust both the mean stimulus level and the range of available intensity (as a function of tactor location) on the tongue. The algorithms are based on a linear regression model of the experimental data obtained. The results from the tests show that this significantly improved pattern perception performance.

The sensory input component of the system is either part of or in communication with a processor that is configured to: 1) receive information from a program or detector (e.g., accelerometer, video camera, audio source, tactile sensor, video game console, GPS device, robot, computer, etc.); 2) translate received information into a pattern to be transmitted to the sensory input component; 3) transmit information to the sensory input component; and/or 4) store and run training exercise programs; and/or 5) receive information from the sensory input component or other monitor of the subject; and/or 6) store and record information sent and received; and/or 7) send information to an external device (e.g., robotic arm).

Electrode arrays of the present invention may be provided on any type of device and in any shape or form desired. In some embodiments, the electrode arrays are included as part of objects a subject may otherwise possess (e.g., clothing, wristwatch, dental retainer, arm band, phone, PDA, etc.). For babies (e.g., to train blind infants), electrode arrays may be included in the nipples of food bottles or on pacifiers. In some embodiments, electrode arrays are implanted under the skin (an array tattoo) (See e.g., Example 18). In preferred embodiments, the device containing the array is in wireless communication with the processor that provides external information. In some preferred embodiments, the array is provided on a small patch or membrane that may be positioned on any external (including mucosal surfaces) or internal portion of the subject.

The devices may also be used to output signals, for example, by using the tongue as a controller of external systems or devices or to transmit communications. Example 17 provides a description of some such applications. In some embodiments, the tongue, via position, pressure, touching of buttons or sensor (e.g., located on the inside of the teeth) provides output signal to, for example, operate a wheelchair, prosthetic limb, robot device, medical device, vehicle, external sensor, or any other desired object or system. The output signal may be sent through cables to a processor or may be wireless.

### Training Systems and Methods

Many of the applications described herein utilize a training program to permit the user to learn to associate particular patterns of sensory input information with external events or objects. The Examples section describes numerous different training routines that find use in different applications of the invention. The present invention provides software and hardware that facilitate such training. In some embodiments, the software not only initiates a training sequence (e.g., on a computer monitor), but also monitors and controls the amount of and location of signal sent to the tactile sensory device component. In some embodiments, the software also manages signals received from the tactile sensory device. In some embodiments, the training programs are tailored for children by providing a game environment to increase the interest of the children in completing the training exercises.

### EXAMPLES

The following Examples are provided in order to demonstrate and further illustrate certain preferred embodiments and aspects of the present invention and are not to be construed as limiting the scope thereof.

### Example 1

### Vestibular Substitution for Posture Control

The vestibular system detects head movement by sensing head acceleration with specialized peripheral receptors in the inner ear that comprise semicircular canals and otolith organs. The vestibular system is important in virtually every aspect of daily life, because head acceleration information is essential for adequate behavior in three-dimensional space not only through vestibular reflexes that act constantly on somatic muscles and autonomic organs (see Wilson and Jones, Mammalian Vestibular Physiology, 2002, New York, Plenum), but also through various cognitive functions such as perception of self-movement (Buttner and Henn, Circularvection: psychophysics and single-unit recordings in the monkey, 374:274 (1981); Guedry et al., Aviat. Space Environ. Med., 50:205 (1979); Guedry et al., Aviat. Space Environ. Med., 52:304 (1981); Guedry et al., Brian Res. Bull., 47:475 (1998); Jell et al., Aviat. Space Environ. Med., 53:541 (1982); and Mergner et al., Patterns of vestibular and neck responses and their interaction: a comparison between cat cortical neurons and human psychophysics, 374:361 (1981)), spatial perception and memory (Berthoz et al., Spatial memory of body linear displacement: what is being stored? 269:95 (1995); Berthoz, The role of inhibition in the hierarchical gating of executed and imagined movements, 3:101 (1996); Bloomberg et al., Vestibular-contingent voluntary saccades based on cognitive estimates of remembered vestibular information, 41:71 (1988); and Nakamura and Bronstein, The perception of head and neck angular displacement in normal and labyrinthine-defective subjects. A quantitative study using a ‘remembered saccade’ technique, 188:1157 (1995)), visual spatial constancy (Anderson, Exp. Psychol. Hum. Percept. Perform., 15:363 (1989) and Bishop, Stereopsis and fusion, 26:17 (1974)), visual object motion perception (Mergner, Role of vestibular and neck inputs for the perception of object motion in space, 89:655 (1992) and Mesland, Object motion perception during ego-motion: patients with a complete loss of vestibular function vs. normals, 40:459 (1996)), and even locomotor navigation (Wiener, Spatial and behavioral correlates of striatal neurons in rats performing a self-initiated navigation task, 13:3802 (1993)). Vestibular input functions also include: egocentric sense of orientation, coordinate system, internal reference center, muscular tonus control, and body segment alignment (Honrubia and Greenfield, A novel psychophysical illusion resulting from interaction between horizonal vestibular and vertical pursuit stimulation, 19:513 (1998)).

Persons with bilateral vestibular damage, such as from an adverse reaction to antibiotic medications, experience functional difficulties that include postural “wobbling” (both sitting and standing), unstable gait, and oscillopsia that make it difficult or impossible, for example, to walk in the dark without risk of falling. Bilateral vestibular loss can be caused by drug toxicity, meningitis, physical damage or a number of other specific causes, but is most commonly due to unknown causes. It produces multiple problems with posture control, movement in space, including unsteady gait and various balance-related difficulties, like oscillopsia (Baloh, Changes in the human vestibulo-occular reflex after loss of peripheral sensitivity, 16:222 (1991)). Unsteady gait is especially evident at night (or in persons with low visual acuity). The loss is particularly incapacitating for elderly persons.

Oscillopsia, due to the loss of vestibulo-ocular reflexes is a distressing illusory oscillation of the visual scene (Brant, Man in motion. Historical and clinical aspects of vestibular function. A review. 114:2159 (1991)). Oscillopsia is a permanent symptom. When walking, patients are unable to fixate on objects because the surroundings are bounding up and down. In order to see the faces of passerbies, they learn to stop and hold their heads still. When reading, such patients learn to place their hand on their chin to prevent slight movements associated with pulsation of blood flow.

In the absence of a functional vestibular system, the roles of the remaining inputs to the multisensory integration process of normal upright posture are amplified. Under these circumstances, subjects extensively use the fingertips to provide additional spatial orientation cues.

The systems and methods of the present invention provide alternative, and substantially better cues. The use of vestibular sensory substitution produces a strong stabilization effect on head and body coordination in subjects with BVD. Under experimental conditions, three characteristic and unique motion features (mean-position drift, sway, and periodic large-amplitude perturbations) were identified that consistently appear in the head-postural behavior of BVD subject. With vestibular substitution, however, the magnitude of these features are greatly reduced or eliminated. During the experiments, the BVD subjects reported feeling normal, stable, or having reduced perceptual “noise” while using the system and for periods after removing the stimulation.

For experiments conducted during the development of the present invention, subjects with bilateral vestibular loss, the most severe damage possible to the balance sensory system, were selected. All of the subjects were identified as disabled or handicapped.

Device: A miniature 2-axis accelerometer (Analog Devices ADXL202) was mounted on a low-mass plastic hard hat. Anterior-posterior and medial-lateral angular displacement data (derived by double integration of the acceleration data) were fed to a tongue display unit (TDU) that generates a patterned stimulus on a 144-point electrotactile array (12×12 matrix of 1.5 mm diameter gold-plated electrodes on 2.3 mm centers) held against the superior, anterior surface of the tongue (Tyler et al., J. Integr. Neurosci., 2:159 (2003)).

Head-Motion Sensing

The accelerometer is nominally oriented in the horizontal plane. In this position, it normally senses both rotation and translation. However, given the nature of the task-quiet upright sitting, at least to a first approximation, all non-zero acceleration data recorded in both the x- and y-axis (the M/L and A/P direction, respectively), can be ascribed to angular displacement or tilt of the head and not translation. After instructing the subject to assume the test position, the initial value of the sensor is recorded at the start of each trail and subsequently used as the zero-reference. Using a small angle approximation, and given that the sensor output is proportional to the angular displacement from the zero position, the instantaneous angle is calculated as:

Θₓ=sin⁻¹aₓ/g (Eq. 1)

Θ_{y}=sin⁻¹a_{y}/g (Eq. 2)

where g is the gravity vector and both aₓ and a_{y} are the vector components in the respective axis.

### “Target” Motion Control

The tilt data from the accelerometer is used to drive the position of both the visual and tactile stimulus pattern or ‘target’ presented on the respective displays. The data is sampled at 30 Hz and the instantaneous x and y vales for the target position is calculated as the difference between the values of the position vector at tₙ and tₒ, by:

xₙ=c sin(Θ_{x|n}−Θ_{x|0}) (Eq 3)

yₙ=c sin(Θ_{y|n}−Θ_{y|0}) (Eq. 4)

where the values for Θ_{x|n}, Θ_{x|0}, Θ_{y|n}, and Θ_{y|0} are the instantaneous and initial tile angles in x and y, respectively. A linear scaling factor, ‘c’, is used to adjust the range of target movement to match that of the subject's anticipated or observed head-tilt. To prevent disorientation due to stimulus transits off the display in the event the subject momentarily exceeds the maximum range initially calculated, the maximum displacement of the target is band limited to the physical area of the display. This gain can be easily adjusted to the match maximum expected range of motion. The actual stimulation pattern on the tongue display is a factor (2×2) square array whose area centroid is located at xₙ, yₙ at any instant in time. After calibration at the initial upright condition, the subject then moves the head to keep the target centered in the middle of the display to maintain proper posture. For initial training a visual analog of the outside edge of the square tactile array is presented on an LCD monitor. The resultant position vector used to drive the visual target motion is low pass filtered at 10 Hz, and smoothed using a 20-sample moving-window average to make the image more stable.

Subjects readily perceived both position and motion of a small ‘target’ stimulus on the tongue display, and interpreted this information to make corrective postural adjustments, causing the target stimulus to become centered.

Signals from the accelerometer, located in the hat on top of the head, deliver position information to the brain via an array of gold plated electrodes in contact with the tongue. Continuous recording from the accelerometer produced the head base stabilogram (HBS). The HBS is the major component of the data recording and analysis system.

Subjects: Ten individuals with bilateral vestibular dysfunction (BVD) tested and trained using the Electro-tactile Vestibular Substitution System (EVSS). Five participants were female and five were male. The average age of the female group was 51.4 years with the average age of the male group being 64.4 years.

Of both groups, the dysfunction of seven of the participants was a result of ototoxicity from the use of the aminogylcoside antibiotic gentamycin. One subject had a Mal de Debarquement syndrome, one patient had vestibular dysfunction as a result of bilateral surgery to correct perilymphatic fistulas, and one subject's loss of vestibular functions bilaterally was a result of an unknown phenomenon.

Testing and training procedure: To determine abilities prior to testing, each subject completed a health questionnaire as well as a task ability questionnaire, along with the required informed consents forms. Prior to testing, each individual was put through a series of baseline tests to observe their abilities in regards to balance and visual control (oscillopsia). These baseline tests were videotaped.

Prior to undergoing any 20-minute trials, each individual underwent a series of data captures with the EVSS designed to obtain preliminary balance ability baselines as well as to train them in the feel and use of the system. These data captures included 100, 200 and 300-second trials both sitting and standing, eyes open and eyes closed.

Upon completion of the balance ability baselines and confirmation from the subjects that they fully understood the EVSS and how it operates, each individual proceeded into the 20 minute trials and/or were trained to stand on soft materials or in tandem Romberg posture. For all patients, both conditions were “unimaginable” to perform. Indeed, none of the subjects could complete more than 5-10 seconds stance in any conditions.

Typical testing/training included 9 sessions 1.5-2 hours long (depending on patient stamina and test difficulty). The shortest series a patient completed was five sessions, while the longest for 65 sessions.

Results: As a result of training procedures with the EVSS, all ten patients demonstrated significant improvement in balance control. However, speed and depth of balance recovery varied from subject to subject. Moreover, it was found that training with the EVSS demonstrated not one, but rather several different effects or levels of balance recovery.

Balance recovery effects of EVSS training can be separated into at least two groups: direct balance effects and residual balance effect. In addition to balance recovery effects, it was found that multiple effects directly or indirectly related to the vesitibular system were observed (see Examples 2-8).

Immediate effect: The immediate effect was observed in the sitting and standing BVD subjects almost immediately (after 5-10 minutes of familiarization with EVSS) and included the ability to control stable vertical posture and body alignment (sitting or standing with closed eyes) during extended periods (up to 40 minutes after 1-2 experimental sessions).

Training effect: Some of the BVD patients, especially after long periods of compensation and extensive physical training during many years, had developed the ability to stand straight, even with closed eyes, on hard surface. However, even for well-compensated BVD subjects standing on soft or uneven surfaces or stance with limited bases such as during a tandem Romberg stance, standing was challenging, and unthinkable with closed eyes.

Using the EVSS, BVD patients not only acquired the ability to control balance and body alignment standing on hard surfaces, but also the ability to extend the limits of their physical conditioning and balance control. As an example, standing in the tandem Romberg stance with closed eyes became possible. After one training session of 18 training trials each 100 seconds long (total EVSS exposure time 30 minutes), a BVD patient was capable of standing in the tandem Romberg stand with closed eyes for 100 seconds.

Residual balance effects: Residual balance effects also were observed in all tested BVD patients; however strength and extent of effects significantly varied from subject to subject depending on the severity of vestibular damage, the time of subject recovery, and the length and intensity of EVSS training.

At least three groups of residual balance effects were noted: short term residual effects (sustained for a few minutes), long term residual effects (sustained for 1 to 12 hours) and a rehabilitation effect that was observed during several months of training in a subject. All residual effects were observed after complete removal of EVSS from the subject's mouth.

Short term after effects: This effect usually was observed during the initial stages of EVSS training. Subjects were able to keep balance for some period of time, without immediately developing an abnormal sway; as it usually occurred after any other kind of external tactile stabilization, like touching a wall or table. Moreover, the length of short term aftereffects was almost linearly dependent on the time of EVSS exposure. After 100 seconds of EVSS exposure, stabilization continued during 30-35 seconds, after 200 seconds EVSS exposure 65-70 seconds and after 300 seconds EVSS trial the subject was able to maintain balance for more than 100 seconds. Short term after-effect continued during approximately 30-70% of the EVSS exposure time.

Long Term after Effects:

This group of effects developed after longer (e.g., up to 20-40 minutes) sessions of EVSS training in sitting or standing subjects and continued for a few hours. The duration of the balance improvement after-effect was much longer than after the observed short-term after effect: instead of the expected seven minutes of stability (if one were to extrapolate the 30% rule on 20 minute trials), from one to six hours of improved stability was observed. During these hours BVD subjects were able to not only stand still and straight on a hard or soft surface, but were also able to accomplish completely different kinds of balance-challenging activities, like walking on a beam, standing on one leg, riding a bicycle, and dancing. However, after a few hours all symptoms returned.

The strength of long term after effects was also dependent on the time of EVSS exposure: 10 minute trials were much less efficient than 20 minute trials, but 40 minutes trails had about the same efficiency as 20 minutes. Usually, 20-25 minutes was the longest comfortable and sufficient interval for standing trials with closed eyes. Sitting trials were less effective than standing trials.

The shortest effects were observed during initial training sessions, usually 1-2 hours. The longest effect after a single EVSS exposure was 11-12 hours. The average duration of long term after effects after single 20 minute EVSS exposure was 4-6 hours.

Rehabilitation effect: It was possible to repeat two or three 20-minute EVSS exposures to a single subject during one day. After the second exposure, the effect was continued in average about 6 hours. In total, after two 20-minute EVSS stabilization trials, BVD subjects were capable of feeling and behaving what they described as “normal” for up to 10-14 hours a day.

One BVD subject was trained continuously during 20 weeks, using one or two 20-minute EVSS trials a day. The data collected on this subject demonstrated a systematic improvement and gradual increase of the long-term aftereffect during consistent training. Moreover, it was found that repetitive EVSS training produced both accumulated improvement in balance control, and global recovery of the central mechanisms of the vestibular system.

For the same BVD subject, after two months of intensive training, EVSS exposure was completely stopped. Regular checking of the subject's balance and posture control were continued. During the 14 weeks after the last EVSS training, the subject was able to stay perfectly still with closed eyes, while standing for 20 minutes on hard or soft surfaces. This demonstrated rehabilitation capability of the method. Effects have been seen for over six months.

Summary of effects: Subjects experienced the return of their sense of balance, increased body control, steadiness, and a sense of being centered. The constant sense of moving disappeared. The subjects were able to walk unassisted, reported increased ability to walk in dark environments, to walk briskly, to walk in crowds, and to walk on patterned surfaces. Subjects gained the ability to stand with their eyes closed with or without a soft base, to walk a straight line, to walk while looking side-to-side and up and down. Subjects gained the ability to carry items, walk on uneven surfaces, walk up and down embankments, and to ride a bike. Subjects became willing to attempt new challenges and, in general, became much more physically active.

Although discussed above in the context of persons with bilateral vestibular loss, the invention finds use with many types of vestibular dysfunction and persons with Meniere's disease, Parkinson's disease, persons with diabetic peripheral neuropathy, and general disability due to aging. The invention also has applicability to the field of aviation to avoid spatial disorientation in aircraft pilots or astronauts.

Additional data. A subject with BVL due to gentamicin ototoxicity was treated for one week with the systems and methods of the present invention. The subject's response to treatment is documented in Table 1 below.

TABLE 1

| Test | Pre-treatments Score | Post-treatment Score |
|---|---|---|
| Neurocom SOT composite | 31 | 47 |
| Total # of falls on SOT | 7 | 6 |
| # of falls on SOT 5 and 6 | 6 | 6 |
| Dynamic Gait Index | 21/24 | 24/24 (24 best) |
| Activities-Specific Balance | 64/100 | 85/100 (100 best) |
| Confidence Scale | | |
| Dizziness Handicap | 74/100 | 0/100 (0 best) |
| Inventory | | |

As described in Table 1 above, the subject demonstrated improvements with the quality of life indicators (ABC, DHI), and on the SOT. Walking in crowds became significantly easier for the subject.

### Example 2

### Improved Posture, Proprioception and Motor Control

Experiments conducted during the development of the present invention identified unexpected benefits in improved posture, proprioception, and motor control of subjects. Training was conducted with an EVSS as described in Example 1. Observation of and questioning of subjects demonstrated that body movements became more fluid, confident, light, relaxed and quick. Stiffness disappeared, with limbs, head and body feeling lighter and less constricted. Fine motor skills returned, and gait returned to normal. Posture and body segment alignment returned to normal. Stamina and energy increased. There was an increased ability to drive both for daytime and night driving.

### Example 3

### Improved Vision

Experiments conducted during the development of the present invention identified unexpected benefits in vision of subjects. Training was conducted with an EVSS as described in Example 1. Observation of and questioning of subjects demonstrated that vision became more stable, clearer, and brighter. Colors were also brighter and sharper, and peripheral vision widened. Reading became smoother and easier, and it was possible to read in a moving vehicle. There were strong improvements in adaptation during transition from light to dark conditions. There was a reduction of oscillopsia and an improved depth perception.

### Example 4

### Improved Cognitive Functions

Experiments conducted during the development of the present invention identified unexpected benefits in cognitive function of subjects. Training was conducted with an EVSS as described in Example 1. Observation of and questioning of subjects demonstrated increases in mental awareness, creativity, clarity of thinking, confidence, multitasking skills, memory retention, concentration ability, and ability to track conversations and stay on task. Subjects felt more alert and energized, and ceased the constant awareness of balance. There was less “noise” in the head, much improvement in intensity of thinking, problem solving and decision-making.

### Example 5

### Improved Emotional Well being

Experiments conducted during the development of the present invention identified unexpected benefits in emotional conditions of subjects. Training was conducted with an EVSS as described in Example 1. Observation of and questioning of subjects demonstrated that subjects felt calmer, aware, confident, happy, quiet, refreshed, relaxed, a strong sense of well being, and elimination of fear.

### Example 6

### Improved Sleep

Experiments conducted during the development of the present invention identified unexpected benefits in sleep of subjects. Training was conducted with an EVSS as described in Example 1. Observation of and questioning of subjects demonstrated that a majority of patients noticed sleep improvement. Sleep became fuller, longer, and more restful, often with no awakenings during the night.

### Example 7

### Improved Sense of Physical Well Being

Experiments conducted during the development of the present invention identified unexpected benefits in sense of physical well being of subjects. Training was conducted with an EVSS as described in Example 1. Observation of and questioning of subjects demonstrated a feeling of youth and vibrancy, with brighter eyes and a reduction of stress, lifting and relaxation of face muscles resulting in a “younger look.” Some subject reported fewer visits to a chiropractor and increased activity.

### Example 8

### Treatment Tinnitus

Experiments conducted during the development of the present invention identified unexpected benefits in relieving tinnitus. Training was conducted with an EVSS as described in Example 1. A subject with tinnitus reported a reduction in symptoms.

### Example 9

### Sex Sensation Substitution

In some embodiments, the present invention provides systems and methods for sex sensation tactile substitution for, for example, persons with spinal chord injury that have lost sensation below the level of the injury. With training, such subjects recover, at least to some extent, sexual sensation.

Experiments conducted during the development of the present invention have demonstrated that tactile human-machine interfaces (HMI) allow artificial sensors to deliver information to the brain to mobilize the capacity of the brain to permit functional sensory and motor reorganization in persons who are bind, deaf, have loss of vestibular system, or skin sensation loss from Leprosy. Experiments also demonstrated that a substitute system can re-establish natural function is a small amount of surviving tissue is present after a lesion. Thus, in addition to providing sensory substitution, the systems of the present invention achieve a therapeutic effect. While this example describes application to sex sensation substitution, it is understood that the same techniques may be used for other sensory losses and for recovery of motor functions in spinal chord injury (SCI).

Decrease in sexual function after spinal cord injury is a major cause of decreased quality of life for both men and women. Treatment of sexual dysfunction in the SCI population has focused on the restoration of erectile function. However, sensation is impaired in the vast majority of the SCI population, which is much more difficult to treat. Loss of orgasm appears to be the major SCI sexual problem, the loss mainly being due to loss of sensation. Women with complete loss of vaginal sensation can reach orgasm by caressing of other parts of the body that have intact sensibility for touch (e.g., ear-lobes, nipples) and some men can be taught to achieve orgasm (not to be confused with ejaculation) from comparable caressing. However, there is no known technique available to re-establish or substitute penile sensibility in these patients. Such sensibility is, for most men, a prerequisite to reaching orgasm.

With sensory substitution systems of the present invention, information reaches the perceptual levels for analysis and interpretation via somatosensory pathways and structures. In some embodiments, a genital sensor with pressure and/or temperature transducers is utilized to relay the pressure and/or temperature patterns experienced by the genitals via tactile stimulation to an area of the body that has sensation (e.g., tongue, forehead, etc.). With training, subjects are able to distinguish rough versus smooth surfaces, soft and hard objects, and structure and pressure. The subject perceives the information as coming from the genitals. Thus, even though that actual man-machine interface is not on the genitals, the subject perceives the sensation on the genitals, as his/her perception over the placement of the substitute tactile array directs the localization in space to the surface where the stimulation.

In some embodiments, the present invention provides a penile sheath with embedded sensors and radiofrequency (e.g., BlueTooth) transmission to an electrotactile array built into a dental orthodontic retainer that is contacted by the tongue of the user. This system, with minimum training, provides sexual sensation for spinal cord injured men and women (for whom the penile sheath will be worn by her partner).

In one embodiment, the electrotactile array has 16 stimulators. The sheath likewise has 16 sensors. The sheath is made of an elastic and cloth matrix, such as that used in stump socks for amputees. The sheath is molded over an artificial penis, with the sensors arranged in four rings of four, each sensor at in π/2 increments (radially) about the principal axis of the cylinder. Each sensor is approximately 5 mm in diameter and the ring is placed at 10 mm intervals, beginning at the distal end of the cylindrical portion of the sheath. The sensors are attached with a silicon adhesive with the lead wires traveling to the base of the sheath from where a BlueTooth device transmits the sensory information to the tongue interface. Over this entire sheath structure is applied an off-the-shelf condom. The system is thus designed to prevent the subjects from coming into direct contact with the sensing array electronics, to provide as natural as possible sensation, and to avoid contaminating the sheath in the event that the subject ejaculates.

In some embodiments, a more advance system is used with shear sensitive semiconductor-based tactile sensors and miniaturized integrated electronics. The advanced system has a greater number of sensors and refinement of an application of the Phi effect (perception moving in between stimulating electrodes) and the ability to control the type of input signal. Because shear is a vector, it is contemplated that the components of the sensory output create a more sophisticated stimulation signal, allowing for the addition of a greater variety of possible sensations or ‘color’ qualities to the electrotactile stimulus. In some embodiments, the system includes multiplexed input from several sensory substitution systems simultaneously, such as for foot and lower limb position information to aid in ambulation, and for bladder, bowel and skin input.

The tongue electrode array is built into an esthetically designed clamshell that is held in the mouth and contains 16 stimulus electrodes. The pulses are created by a 16-channel electrotactile waveform generator and accompanying scripting software that specifies and controls stimulus waveforms and trial events. A custom voltage-to-current converter circuit provides the driving capability (5-15 V) for the tongue electrode, having an output resistance of this circuit of approximately 500 kΩ. Active or ‘on’ electrodes (according to the particular pattern of stimulation) deliver bursts of positive, functionally-monophasic (zero net dc) current pulses to the exploring area on the tongue, each electrode having the same waveform. The nominal stimulation current (0.4-4.0 mA) is identical for all active or ‘on pattern’ electrodes on the array, while inactive or ‘off pattern’ electrodes are effectively open circuits. Preliminary experiments identified this waveform as having the best sensation quality for the particular electrode size, array configuration, and timing requirements for stimulating all electrodes. The quality and intensity of the sensation on the tongue display is controlled by manipulating the parameters of the waveform and may be done by input from external devices (both analog and digital) as well as computers or related devices (e.g., signals sent over an Internet).

In some embodiments, subjects are trained to use the equipment. As a first exercise, subjects are instructed how to place the tongue array in the mouth and to set/optimize the comfort level of the stimulus. With an artificial penis as a model, the subjects then are shown how to place the sensory sheath over an erect penis. Sexual encounters are then used with the system to optimize settings for manual stimulation, vaginal stimulation, and the like, intensity, etc.

### Example 10

### Tactile Multimedia

The present invention provides system and methods for enhanced multimedia experiences. In some embodiments, existing multimedia information is transmitted via the systems of the present invention to provide enhanced, replacement, or extra-sensory perception of the multimedia event. In other embodiments, multimedia applications are provided with a layer of additional information intended to create enhanced, replacement, or extra-sensory perception.

Experiments conducted during the development of the present invention have demonstrated that visual information not perceived by the eyes can be imparted by the systems of the present invention. In particular, subjects lacking vision or with closed eyes were able to navigate a graphic maze through the transmission of the maze information from a computer program to the subject through a tongue-based electrotactile system.

One application of the systems of the present invention is to provide enhanced perception for video game play. For example, a game player can gain “eyes in the back of their head” through the transmission of information pertaining to the location of a video object not in the field of view to a stimulator array configured to relay the information to the tongue of the user. With minimal training, the user will “see” and respond to both the presence and location of video objects outside of their normal field of vision. The sensory information may be imparted through tactile stimulation to the hands via a traditional joystick or game controller, or may be through the tongue or other desired location. The ability to operate extra-sensorialy may be integrated into game play. For example, games or portion of games may be conducted “blind” (e.g., closing of eyes, blackout of audio and/or video, etc.). Such games find use for entertainment, but also for training (e.g., flight simulation training, military training to operate in night vision mode, under water, etc.). Balance, emotional comfort level, physical comfort level, etc. may all be altered to enhance game play.

Thus, in some embodiments, the present invention provides game modules (e.g., PlayStation, XBox, Nintendo, PC, etc.) that comprise, or are configured to receive, a hardware component that contains a stimulator array for transmitting information to a subject through, for example, electrotactile stimulation (e.g., via a tongue array, a glove, etc.). In some embodiments, software is provided that is compatible with such game modules or configured to translate signal provided by such game modules, wherein the software encodes information suitable for use with the systems and methods of the present invention. In some embodiments, the software encodes a training program that provides a training exercise that permits the user to learn to associate the transmitted information with the intended sensory perception. The subject proceeds to actual gameplay after completing the training the exercise or exercises.

In some embodiments, media content is layered with sensate information. Certain non-limiting embodiments include:

Sensate movies that carry any kind of sensory messages: the sensation of a kiss; the heat of a fire; or the scratch of a cat.

Sensate Internet that allows the user at home to feel the texture of a dress or suit; allows a surgeon to perform a telerobotic operation; and provides sexual feedback to one or more body parts from a long distance partner.

Sensate telephones, video games, etc.

In some embodiments, the present invention provides a body suit (e.g., full-body suit) that contains stimulators on multiple body parts (e.g., all over the body). Subsets of the stimulators are triggered in response to information obtained from a program, movie, interactive Internet site, etc. For example, in Internet sex applications a subject receives information from a program or from an individual located elsewhere that activates stimulator groups to simulate touching, body to body contact, other types of contact, kissing, and intercourse. Visual information may also be conveyed either through sensory substitution or directly through a visor (providing video, snapshot images, virtual reality images, etc.). Sound (e.g., voice) may be provided by sensory substitution or traditional channels (e.g., telephone line, realtime via streaming media, etc.). In some embodiments, the body suit has higher stimulator density in regions typical engaged in sexual contact. The suit may cover the entire body or particular desired portions. In some embodiments, the user sets a series of parameters in the control software to designate levels of stimulation desired or undesired, activities desired or undesired, and the like. In some embodiments, the system provides privacy features and security features, to, for example, only permit certain partners to participate. In some embodiments, a registry service is provided to ensure that participates are honest and legal with respect to age, gender, or other criteria.

### Example 11

### Lipreading Applications

Many people with hearing impairment recognize the spoken word by the process of lipreading, i.e., recognizing the words being spoken by the movement of the lips and face of the speaker. Lipreaders, however, cannot resolve all spoken words and have difficulty with meaning that is carried in intonation. In addition, lipreaders do not have access to the full syllabic structure of speech.

Word spotting, as it is called in the speech-processing field, is a difficult computational task. For example, some different sounds do not to look very different on the lips. Lipreading is plagued by homophenes, i.e., speech sounds, words, phrases, etc., that are identical or nearly identical on the lips. For example, the bilabial consonants “p”, “b”, and “m” sound different, but they are identical on the lips. For the words “park”, “bark”, and “mark”, the difference between /b/ and /p/ is that in the former the vocal folds start vibrating upon lip opening, whereas they remain open for around 30 ms longer with /p/. This cannot be seen, so these words appear identical. The nasal /m/ is produced by lowering the velum and allowing the air stream to escape via the nasal cavity. Again, this action cannot be seen, so /p, b, m/ form one homophenous group.

There are 24 consonants in English. Each one is a distinct unit to the normal hearing listener, but the information available via lipreading is much less. For example, when the consonants are presented to a lipreader, e.g., sound grouping such as [apa], [aba], [ama], etc., even the best lipreaders have difficulties. Lipreaders will confuse those consonants that share the same place of articulation where the sound is produced, for example, the lips, the alveolar, etc. This means that the set of 24 is reduced to a much smaller number. Sets of sounds that appear the same to a lipreader include the following:

| | | |
|---|---|---|
| 1. | Bilabials | p, b, m |
| 2. | Labio-dentals | f, v |
| 3. | Interdentals | th, th |
| 4. | Rounded labials | w, r |
| 5. | Alveolars | t, d, n, l, s, z |
| 6. | Post-alveolars | sh, zh, ch, j |
| 7 | Palatals and velars | y, k, g, ng |
| 8 | Glottal | h |

Vowels are also a great problem because many appear to be almost identical on the lips. The lipreader has very little access to suprasegmental information intonation, pitch changes, rate, etc. and this again makes the task of understanding potentially ambiguous sentences so much harder. The lack of access to many cues obviously results in a reduced amount of sensory information. As a result, lipreaders have to work harder to derive understanding from speech.

Part of the problem though is that syllable boundaries are blurred by the presence of voicing continuant consonants. Information that would enable the lipreader to reliably identify whether a consonant is voiced or voiceless is found in the low frequencies of speech (100 500 Hz). Information on high frequency speech energy (the region above 5 kHz) can allow the lipreader to reliably identify the sibilant consonants /s, z, sh, zh/ and their affricate cousins.

There have been numerous tactual devices developed to aid lip-readers, two examples being the Tactaid (Audiological Engineering, Somerville, Mass.) and the Minivib (KTH, Stockholm, Sweden). Both of these are vibrotactile (i.e., vibrating) devices for use on the hand or wrist. These devices present one or two channels of limited information, they do not remove a sufficient amount of ambiguity in lipreading mentioned earlier and they are not convenient to use.

Other approaches to lipreading technology include systems to permit lipreading while using a telephone by presenting the remote caller as a speaking avatar whose lips can be read on the computer screen (The SpeechView (Tikva, Israel), and speech-to-text processors. The KTH at the Royal Swedish Academy in Stockholm speech processing group is working on a quasi speech-to-text project, Syn-Face, under license with Microsoft. Microsoft purchased the Entropics Software company that developed products called wave surfer and waves+for word spotting using pitch and formant algorithms. Commercially available speech-to-text word processing software IBM Via Voice and Dragon Naturally Speaking are useful products but they require specific-speaker training for use, and thus are not applicable to the problem of reading the lips of speakers in general. The lipreading system of the present invention provides more useful information in a higher quality and more flexible display format than is currently available.

Cues from tactile aids for lipreading can provide access to the syllabic structure of speech and, when used together with lip-reading cues, can improve the speed and accuracy of lip reading. For example, a tactile aid cue may be triggered when the intensity or another measurable feature of a speech unit falls within predetermined range or level, e.g., every time a particular vowel or a vowel-like consonant such (e.g., w, r, l, y) is produced. A cue of this kind to the listener from the tactile aid provides additional information on the syllabic structure, and thus the meaning, of the speech.

In preferred embodiments, the present invention makes use of electrotactile input devices using the tongue as a stimulation site. In some embodiments, a mouthpiece providing a simulator or an array of stimulators in used. In other embodiments, stimulators are implanted in the skin or in the mouth.

The detected speech signal is processed for transmission to the sensory input device. Processing may be done, e.g., with the software-based virtual instrument environment Labview, National Instruments (Austin, Tex.). Labview transfers the processed information to the tongue display stimulator e.g., via a dll-driven USB interface (DLP Design, San Diego, Calif.). The stimulator processes the information into four channels of spatial and amplitude display for the tongue.

### Supplemental Information Supplied Via the Tongue

In some embodiments, the following information is provided via the tongue, with the intention of reducing the inherent ambiguity in lipreading.
1) Partial access to the word structure of speech.
   High-pass filtering of raw speech above 500 Hz to give cues about word spotting. Together with item #4 below this gives access the syllabic structure of speech
2) Determine whether a consonant is voiced or voiceless
   Band pass filtering 100 Hz to 500 Hz—this cues whether a consonant was oral or nasal. Activity in this range indicates a nasal consonant.
3) High frequency information to identify the sibilant consonants /s, z, sh, zh/ and the related sounds of /ch, j/.
   High pass filter above 5 kHz.
4) Recognition of vowels and vowel-like consonants /w, r, l, y/—gives good cues to the syllabic structure of speech.
   Amplitude threshold sensor such that a signal is given each time the threshold is crossed.

The information is presented to the tongue in two major forms:
1. A signal similar to an oscilloscope tracing. A moving time tracing 6 electrodes wide (approximately 12 mm) with 3 electrodes above and 2 electrodes below the baseline for amplitude deviations.
2. An indicator of activity, such a blinking dot, to indicate the presence of sound energy in a particular frequency band like above 5 kHz to distinguish fricatives or that an amplitude threshold has be crossed to indicate the presence of a vowel.

In the case of amplitude thresholds relative amplitude threshold compared to a moving average can be used to compensate for mean changes in speech volume and ambient noise.

In addition to the all the visual information available to lip readers, the subjects perceive speech with their tongues and integrate the additional information into their linguistic interpretation. The supplemental information feels like unobtrusive buzzing on the tongue with varying spatial and intensity information. Experience with the tongue display has shown that subjects learn to ignore the tongue sensations while attending to the information presented.

In some embodiments, a fifth channel of higher complexity level sound and word identification via more information-rich codes memorized by the subjects may be used to further reduce ambiguity in lip reading.

### Training

In some embodiments, the present invention comprises specific training. In some embodiments, the trainin comprises:

1:1 training: A training program comprising practice in the use of the tactile device as a supplement to lipreading. In each session the subject receives training in the following areas:

Consonants—practice recognition of consonants in the /aCa/ environment only—1 list (5 random presentations of each consonant) via lipreading alone, and lipreading plus the tactile device.

Words—practice recognition of the 500 most common words in English via lipreading alone and lipreading plus the tactile device. The words are presented in blocks of 10 words with the subject having to attain a criterion level of 90% correct for 10 random presentations of each word before proceeding to the next block. At the completion of five blocks, each of the words is presented for identification twice in a random order.

Phrases and Sentences—provide practice in the recognition of phrases and sentences consisting of the 500 most frequently used words of English. The sentences are presented in blocks of 10, and the subject is expected to score 95% correct before proceeding to the next block.

Speech Tracking—the subject is administered multiple tracking sessions, e.g., 4×5 minutes, via lipreading alone and lipreading plus the tactile device using the KTH modification of the Speech Tracking procedure. This is a computer-assisted procedure that allows live-voice presentation, but computer scoring of all errors and responses. Speech Tracking (De Filippo and Scott, 1978) requires the talker to present a story phrase by phrase for identification. The receiver's task is to repeat the phrase/sentence verbatim, no errors are allowed. If the receiver is unable to identify a word correctly it will be repeated twice. If s/he is still unable to identify the word, it will be shown to her/him via a computer monitor. At the completion of each five-minute block, the following measures are made automatically:
1. Tracking Rate in words-per-minute
2. Ceiling Rate in words-per-minute
3. The Proportion of Words in the passage that have to be repeated
4. The number of words displayed via the monitor
5. The identity of ALL words repeated once, twice, and three times.

### Example 12

### Vision Sensory Substitution

Mediated by the receptors, energy transduced from any of a variety of artificial sensors (e.g., camera, pressure sensor, displacement, etc.) is encoded as neural pulse trains. In this manner, the brain is able to recreate “visual” images that originate in, for example, a TV camera. Indeed, after sufficient training subjects, who were blind, reported experiencing images in space, instead of on the skin. They learned to make perceptual judgments using visual means of analysis, such as perspective, parallax, looming and zooming, and depth judgments. Although the systems used with these subjects have only had between 100 and 1032-point arrays, the low resolution has been sufficient to perform complex perception and “eye”-hand coordination tasks. These have included facial recognition, accurate judgment of speed and direction of a rolling ball with over 95% accuracy in batting the ball as it rolls.

We see with the brain, not the eyes; images that pass through our pupils go no further than the retina. From there image information travels to the rest of the brain by means of coded pulse trains, and the brain, being highly plastic, can learn to interpret them in visual terms. Perceptual levels of the brain interpret the spatially encoded neural activity, modified and augmented by nonsynaptic and other brain plasticity mechanisms. However, the cognitive value of that information is not merely a process of image analysis. Perception of the image relies on memory, learning, contextual interpretation (e.g. we perceive intent of the driver in the slight lateral movements of a car in front of us on the highway), cultural, and other social factors that are probably exclusively human characteristics that provide “qualia.”

The systems of the present invention may be characterized as a humanistic intelligence system. They represent a symbiosis between instrumentation, e.g., an artificial sensor array (TV camera) and computational equipment, and the human user. This is made possible by “instrumental sensory plasticity”, the capacity of the brain to reorganize when there is: (a) functional demand, (b) the sensor technology to fill that demand, and (c) the training and psychosocial factors that support the functional demand. To constitute such a systems then, it is only necessary to present environmental information from an artificial sensor in a form of energy that can be mediated by the receptors at the human-machine interface, and for the brain, through a motor system (e.g., a head-mounted camera under the motor control of the neck muscles), to determine the origin of the information.

A simple example of sensory substitution system is a blind person navigating with a long cane, who perceives a step, a curb, a foot and a puddle of water, but during those perceptual tasks is unaware of any sensation in the hand (in which the biological sensors are located), or of moving the arm and hand holding the cane. Rather, he perceives elements in his environment as mental images derived from tactile information originating from the tip of the cane. This can now be extended into other domains with systems of the present invention associated with artificial sensory receptors such as a miniature TV camera for blind persons, a MEMS technology accellerometer for providing substitute vestibular information for persons with bilateral vestibular loss, touch and shear-force sensors to provide information for spinal cord injured persons, from an instrumented condom for replacing lost sex sensation, or for a sensate robotic hand.

Although the systems used in experiments conducted during the development of the present invention have only had between 100 and 1032 point arrays, the low resolution has been sufficient to perform complex perception and “eye”-hand coordination tasks. These have included facial recognition, accurate judgment of speed and direction of a rolling ball with over 95% accuracy in batting a ball as it rolls over a table edge, and complex inspection-assembly tasks.

In the studies cited above, the stimulus arrays presented only black-white information, without gray scale. However, the tongue electrotactile system does present gray-scaled pattern information, and multimodal and multidimensional stimulation is may be used. Variations of different parameters provide “colors,” for example, by varying the current level, the pulse width, the interval between pulses, the number of pulses in a burst, the burst interval, and the frame rate. All six parameters in the waveforms can be varied independently within certain ranges, and may elicit distinct responses.

A tongue interface presents a preferred method of providing visual information. Experiments with skin systems have shown practical problems. The tongue interface overcomes many of these. The tongue is very sensitive and highly mobile. Since it is in the protected environment of the mouth, the sensory receptors are close to the surface. The presence of an electrolytic solution, saliva, assures good electrical contact. The results obtained with a small electrotactile array developed for a study of form perception with a finger tip demonstrated that perception with electrical stimulation of the tongue is somewhat better than with finger-tip electrotactile stimulation, and the tongue requires only about 3% of the voltage (5-15 V), and much less current (0.4-2.0 mA), than the finger-tip.

For blind persons, a miniature TV camera, the microelectronic package for signal treatment, the optical and zoom systems, the battery power system, and an FM-type radio signal system to transmit the modified image wirelessly are included, for example, in a glasses frame. For the mouth, an electrotactile display, a microelectronics package, a battery compartment and the FM receiver is built into a dental retainer. The stimulator array is a sheet of electrotactile stimulators of approximately 27×27 mm. All of the components including the array are a standard package that attaches to the molded retainer with the components fitting into the molded spaces of standard dimensions. Although the present system uses 144 tactile stimulus electrodes, other systems have four times that many without substantial changes in the system's conceptual design For blind persons the system would preferably employ a camera sensitive to the visible spectrum. For pilots and race car drivers whose primary goal is to avoid the retinal delay (much greater than the signal transduction delay through the tactile system) in the reception of information requiring very fast responses, the source is built into devices attached to the automobile or airplane; and robotics and underwater exploration systems use other instrumentation configurations, each with wireless transmission to the tongue display.

For mediated reality systems using visible or infrared light sensing, the image acquisition and processing can now be performed with advanced CMOS based photoreceptor arrays that mimic some of the functions of the human eye. They offer the attractive ability to convert light into electrical charge and to collect and further process the charge on the same chip. These “Vision Chips” permit the building of very compact and low power image acquisition hardware that is particularly well suited to portable vision mediation systems. A prototype camera chip with a matrix of 64 by 64 pixels within a 2×2 mm square has been developed (Loose, Meier, & Schemmel, Proc. SPIE 2950:121 (1996)) using the conventional 1.2 μm double-metal double-poly CMOS process. The chip features adaptive photoreceptors with logarithmic compression of the incident light intensity. The logarithmic compression is achieved with a FET operating in the sub-threshold region and the adaptation by a double feedback loop with different gains and time constants. The double feedback system generates two different logarithmic response curves for static and dynamic illumination respectively following the model of the human retina.

The user can use the system in a number of ways. At one level, the system can provide actual “pattern vision” enabling the user to recognize objects displayed. In such a case the quality of the vision depends on the resolution (acuity) of such system and on the dynamic range of the system (number of discriminable gray levels). If the field of view of the camera is more than 30 degrees in diameter and there are about 30 elements square in the system, the resolution is low but comparable to peripheral visual resolution.

The native resolution of such system is extended by the user by using zoom (magnification) to explore in more details objects of interest (effectively reducing the field of view and increasing field resolution temporarily). The “static” resolution and dynamic range of the system is further increased by scanning the system and integrating the results over time.

Scanning is possible in two ways: either by scanning the display with the tongue or by scanning the camera using head movements. It is expected that head movement scanning will provide more benefit than tongue scanning but will require more training. Last the system may be used as a radar system exploring the environment with a fairly narrow aperture and enabling the user to detect and avoid obstacles.

### High Performance Blind Subjects

Experiments were conducted with a blind subject that is an extreme athlete who lost vision in his teenage years and presently has 2 artificial eyes. He is a mountain climber, a hang glider and skier. In his initial session with the tongue system he very quickly learned to perform recognition and hand “eye” coordination tasks. He was able to discern a ball rolling across a table to him and to reach out and grasp the ball, he was able to reach for a soft drink on a table, and he was able to play the old game of rock, paper, scissors. He walked down a hallway, saw the door openings, examined a door and its frame, noting that there was a sign on the door. He identified door frames that were painted the same color as the walls, merely due to the very slight shadow cast by the overhead light. The subject equated the learning process to that which he encountered with Braille. At first, the dots under his fingertips were just that, dots. Eventually the dots, through a laborious thinking process, became actual letters and words. And eventually, the physical aspect of the dots was bypassed and the dots were transmitted effortlessly to the brain as words and sentences. The brain had re-circuited itself. It is contemplated that the sensory substitution provided by the present invention has the same result.

### Camera System Design and Development

In some embodiments, image data comes from one of two sources; either an standard CCD miniature video camera (e.g. modified Philips “ToUCam-2”, 240×180 pixel resolution, 30 Hz full-frame rate, 14-bit), or a long-infrared sensing microbolometer set to image in the 7.5-13.5 μm wavelength (Indigo Systems “Omega”, 160×128 pixels resolution, 30 Hz, 14-bit). Either input to the base unit is via high-speed USB for continuous streaming. Using interleaving and odd-line scanning techniques allows frame rates of up to 60 Hz. (or greater) without significant image data degradation due to the high pixel-to-tactor mapping ratio (300 [symbol] 150:1). Both are capable of low power operation, a pixel by pixel address mode, and accommodate lenses with a 40 to 50 angle of view. The focus preferably is adjustable either mechanically or electronically. Depth of field is important, but not as significant as the other criteria.

The camera is mounted to a stable frame of reference, such as an eyeglass frame that is individually fitted to the wearer. The mounting system for the camera uses a mount that is adjustable, maintains a stable position when worn, and is comfortable for the wearer. An adjustable camera alignment system is useful so that the field of view of the camera can be adjusted.

### External Camera Control and TDU Interface

The oral unit contains sub-circuitry to convert the controller signals from the base unit into individualized zero to +60 volt monophasic pulsed stimuli on the 160-point distributed ground tongue display. Gold-plated electrodes are created and formed on the inferior surface of the PTFE circuit board using standard photolithographic techniques and electroplating processes. This board serves as both a false palate for the tongue array and the foundation to the surface-mounted devices on the superior side that drives the ET stimulation. The advantage of this configuration is that one can utilize the vaulted space above the false palate to place all necessary circuitry and using standard PC board layout and fabrication techniques, to create a highly compact and wearable sub-system that can be fit into individually-molded oral retainers for each subject. With this configuration, only a slender 5 mm diameter cable protrudes from the corner of the subject's mouth and connect to the chest- or belt-mounted base unit.

The unit has a single removable 512 MB compact flash memory cards on board that can be used to store biometric data. Subsequent downloading and analysis of this data is achieved by removing the card and placing it in a compact flash card reader. Programming and experimental control is achieved by a high-speed USB between the Rabbit and host PC. An internal battery pack already used on the present TDU supplies the 12-volt power necessary to drive the 150 mW system (base+oral units) for up to 8 hours in continuous use.

### Waveform Control System

The electrotactile stimulus comprises 40-μs pulses delivered sequentially to each of the active electrodes in the pattern. Bursts of three pulses each are delivered at a rate of 50 Hz with a 200 Hz pulse rate within a burst. This structure was shown previously to yield strong, comfortable electrotactile percepts. Positive pulses are used because they yield lower thresholds and a superior stimulus quality on the fingertips and on the tongue.

### Orthodontic Appliance

The present electrode array is positioned in the mouth by holding it lightly between the lips. This is fatiguing and makes it difficult for the subject to speak during use. Thus, a preferred configuration is a orthodontic retainer, individually molded for each subject that stabilizes the downward-facing electrode array on the hard palate. Integrated circuits to drive the electrode elements are incorporated into the mouthpiece so as to minimize the number of wires used to connect the interface to the TDU. One embodiment employs the Supertex HV547 (can drive 80 electrodes). Four such devices can be implanted in the orthodontic mouthpiece. This also provides more repeatable placement of the electrode array in the mouth. Devices with 160 electrodes and 320 electrodes are used in some embodiments.

In particularly preferred embodiments, the orthodontic dental retainer has a large standard cut-out into which a standard instrumentation and stimulator package is inserted. To make the device wireless and cosmetically acceptable, an electronics microchip, battery and a RF receiver are built into a dental orthodontic retainer.

### Training

During adjustment tests, participants are first given an opportunity to adjust an intensity control knob from zero intensity up to the point where they could detect a weak electrotactile stimulation. Once this level is attained, they are instructed to increase and decrease the intensity slightly, to observe how the percept changes with changes in stimulation intensity.

Minimum intensity adjustment test (MIAT). Purpose: a fast estimate of perceptual threshold for electrotactile stimulation. Once participants are familiar with how the stimulation felt and changed with increases in intensity, they practice obtaining their sensation threshold, defined as the weakest level of intensity that can barely be perceived. They are instructed to tweak the knob up and down to obtain the most precise measurement possible in a reasonable period of time (up to 60 sec. in the practice trials, reduced to 30 sec. for the experimental trials). For all measurements of sensation threshold using knob adjustment, a random offset (30%) is applied to the knob so that participant are not able to use knob position as a cue. The average reading of 5 repetitions is considered as a minimum intensity level for future considerations.

Maximum intensity test (MXAT). Purpose: A fast estimate of maximum comfortable level for electrotactile stimulation. After several practice trials, participants are instructed to set a higher level of intensity, but one not so high as to be uncomfortable. The average reading of 5 maximum intensity levels without discomfort is considered as a maximum intensity range for future considerations. Difference between maximum and minimum intensities is considered as dynamic range data.

Two alternative force choice (2AFC) task training. Purpose: to train participants for more precise procedures of threshold measurements, important for waveform optimization. For the 2AFC task, each trial consists of two temporal intervals, separated by tones. Each interval lasts approximately 3 sec. In a randomly determined one of the intervals, an electrotactile stimulus is presented. At the end of the two interval sequence, the participant is instructed to respond with which interval they believed contained the stimulus and is informed that every trial contains a stimulus in a random one of the two intervals. For practice, the higher level is used as a starting value to make the task relatively easy and straightforward for the participant. In the actual experimental trials, a method of threshold adjustment is used as the starting value as a reasonable approximation of threshold. The computer employs an algorithm to maintain an overall 75% correct level of performance across a run of 2AFC trials. The algorithm is such that the intensity increases by 3% following an incorrect response and decreased by 3% following 3 correct responses (not necessarily consecutive). This procedure is referred to as forced-choice tracking.

Array Mapping test. Purpose: To measure non-linearity of tongue sensation thresholds across the TDU array. After training with full array stimulation MIAT and MXAT tests are repeated for each fragment of TDU array. Therefore, the initial TDU array (144 electrodes) is fragmented at 16 parts (group 3×3 electrodes). Dynamic range measurements are repeated for each fragment. For the tip of the tongue, the test is repeated with smaller fragment size. Results of the tests are used in developing perceived pattern intensity compensation procedures. The individual (experiment to experiment) and population (across participants) variability are considered.

Training. A program is used to provide a number of aspects of visual perception with the stimulator. The program includes basic testing aimed at determining the level of pattern vision provided by the system in ways similar to testing of basic visual function in sighted observers starting with static stimuli generated by the computer, as well as full function assessments enabling the user to combined all of the flexibility and active exploration provided by head mounted camera in a simulated environment.

Basic functions to be assessed include:
1) Two line separation (1-D function)
2) Two point separation in a 2-D plane (unknown orientation)
3) CSF—grating detection
4) Orientation discrimination
5) Suprathreshold contrast magnitude estimation for the determination of the dynamic range
6) Direction of motion in 1-D

Complex pattern vision and acuity will be tested
1) Letter acuity
2) Tumbling E
3) Pediatric shapes acuity
   All these functions are tested in a few modes:
1) Direct feed from the computer into the tongue display providing fixed stimuli that can only be explored with tongue motion over the display.
2) Direct feed from the computer including jitter or oscillatory motion of the stimuli providing a scanning of the stimuli on the display as would be with head motion but the movement is passive not active
3) Feed of the stimuli through camera movements. Head mounted camera aimed at a visual display of the stimuli.
   Virtual environment testing includes two types of tests:
1) Perception of visual direction by pointing
2) Obstacle avoidance while walking in a virtual environment (virtual Shopping Mall while walking on a treadmill)

For complex pattern vision testing, one may use a clinical vision testing device: the BVAT (Waltuck et al 1991). This system, providing a standard NTSC output, provides a complete set of targets for acuity testing. These include a random letter presentation testing at various sizes. A tumbling E test and pediatric test patterns with shapes such as Cake, Jeep, Telephone. The ability of the subject to recognize these various shapes can be easily assessed with this system and the level of “visual” acuity for such performance can also be determined over a wide range.

A recently developed system for testing visual direction is available and may be tailored for the tongue study. A large screen rear projection system provide stimuli and a mouse on very large graphic tablet placed under a wooden cover that locks the view of the hand from the eyes (or here the camera) is used to measure pointing in the direction of perceived objects. A virtual walking system developed includes a treadmill and a virtual shopping mall projected on a large screen. The user may walk through the full range of the mall, change direction with a hand held mouse and respond to obstacles (static or dynamic) that appear in his/her path. Head tracking is available as well to correct for the mall perspective in accordance with user's head position.

For the purpose of navigation the user needs to perceive correctly direction in space as displayed on the tongue and corrected for the subject's own head movements. To train this ability the subject sits in front of a large rear projected screen on which visual targets are superimposed on a video picture. The picture and the target are acquired by the TVS video camera and are provided to the subject via the tongue display. The subject arm is placed on a mouse on the surface of a large graphic tablet under a wooden cover that blocks view of the arm from the camera avoiding visual feedback. Following camera adjustment and calibration that are verified with visual feedback the subject is asked to point to the direction target which appeared following audio tone and click the mouse button. After clicking the subject takes his arm all the way to the right to reduce the possibility of mechanical propriecptive feedback. This movement triggers the initiation of the next target presentation. In separate trials the subject is directed to aim his head in three different directions straight ahead and to the right and left. Feedback is provided on the accuracy of the pointing.

### Learning and Adaptation for Reaching in 3-D Space

Subjects are asked to reach for a 1″ cube in their immediate reaching space. The cube is placed in one of 5 locations for each of 100 trials. Cube placement is randomized. Subjects wear sound attenuating devices and the TVSS camera is occluded between trials. Then the direction of the camera is shifted 15° laterally and subjects and the procedures repeated to determine rate and means of adaptation.

### Learning to Catch Moving Stimuli

Subjects are asked to capture a 2″ ball moving across their immediate work space. The ball is controlled by a variable torque motor capable of generating 5 different speeds. A ready cue is given prior to the ball coming into view. Subjects wear sound attenuating devices and the TVSS camera is occluded between trials. The speed and delay of ball presentation is randomly varied.

### Orientation and Mobility

The TVSS is used continuously during testing sessions. It may worn with the camera covered for testing skills without TVSS information. Testing is done with and without the benefit of each subject's other assistive devices (guide dog, white cane . . . ).

Task 1. The Ability to Locate a Metal Pole and Walk to it without Veering

In a laboratory setting utilizing only the TDU, the subject is tested on recognition, localization, and approach of a variety of metal poles of varying diameter. Distance traveled is held at 40-50 feet to simulate the distance of crossing a street. Outdoor training and testing is conducted and tested as possible.

Task 2. the Ability to Shoreline a Vertical Wall

In an indoor environment the subject is asked to follow a wall in a corridor of approximately 60 feet in length, without contacting it with their cane, while wearing the TDU, and locate an open doorway. Testing involves being able to locate open versus closed doorways in an unfamiliar part of the building.

Task 3. the Ability to Follow a Curved Grass Line

In an outdoor environment utilizing a cane, the subject learns to differentiate between the concrete and the grass using the TDU and locate intersecting sidewalks over an area of 120 feet.

Results with Blind Children

Experiments were conducted with congenitally blind children between the ages of 8 and 18 on a tongue based system. Past studies and training programs have indicated that 15-20 hours of training is generally useful to develop perceptual competency. Subject characteristics and progress are indicated in Table 2. The number of hours trained and lesson number accomplished are also shown. The subjects have been listed in order of the number of hours of training they received. The number of lessons accomplished relate closely to the number of hours available for training with the exception of Subject 5.

TABLE 2

| Subject | | | | | | Most advanced |
|---|---|---|---|---|---|---|
| No. | Age | Gender | Vision status training | Time | | learning |
| 1 | 16 | F | Distinguishes direction of bright light. | 30 | Hrs. | Exceeded Curriculum |
| | | | Small L Nasal area of retina capable | | | |
| | | | of edge detection with adequate | | | |
| | | | contrast. | | | |
| | | | Onset 19 months | | | |
| 2 | 18 | F | Blind from Birth | 17 | hrs | Pursuit Tracking |
| | | | No light detection | | | Shape Recognition |
| | | | | | | Overlapping Shapes |
| 3 | 11 | F | Blind from 6.5 months | 16 | hrs | Shape Recognition |
| | | | secondary to tumor | | | Beginning Letters |
| | | | Juvenile Pilocytic Astrocytoma | | | Linear Perspective |
| | | | No light Detection | | | Interposition |
| 4 | 18 | F | Blind From Birth secondary to | 12.8 | hrs | Intersecting Lines |
| | | | Prematurity | | | |
| | | | No light Detection | | | |
| 5 | 11 | M | Blind from Birth | 10 | hrs | Pursuit tracking |
| | | | No light detection | | | Moving object |
| | | | | | | recognition |
| | | | | | | Shape recognition. |
| 6 | 9 | M | Blind from Birth | 7 | hrs | Size discrimination of |
| | | | No light detection | | | curved lines |

### Subject 1:

Subject 1 demonstrated that the tongue interface system meets and exceeds the capabilities of earlier vibrotactile versions of the TVSS. She finished and surpassed the curriculum. She developed signature skills and was beginning to develop tracing skills at 25 hours of training. She progressed from being unable to do any of the pre-tests to passing all tests of spatial ability, dynamic perception and use of information given to her. She generated uses for the system, asking to use the system to observe cars moving on her street in the winter and to follow the movements of her choir director conducting with flashlights in his hands. She plans to major in music and wants to use the system for conducting classes.

Subject 1 met and exceeded all expectations and goals of the project. There were a number of contributing factors to her success. First, she was frequently able to train 2-3 times a week, was consistently available for training and could work for over and hour at the task. Thus, she had 30 hours of training. Second, she is very bright and verbal. She would consistently tell the trainer what she was feeling on her tongue and how she was approaching the tasks. Finally, she is the only subject with light perception and who knew the alphabet. She has a small area on her left retina located in on the nasal aspect with which she can detect edges if they are of high enough contrast. She had learned the alphabet by having letters (about 18″) projected onto a screen. She would then capture an edge and follow it to derive the full form through her movement along the edge. She talked to the trainer as she viewed displays by biting down on the strip to hold it in her mouth as she talked with a kind of gritted teeth sound. This was very helpful. For example, in pre-testing, when asked to trace a line that went down diagonally to the right she produced a line generally going down and to the left. As she drew she described the line “jumping” to the left each time she tracked to the right. She would go back to “capture” it and direct her pencil in the direction it seemed to move. Thus, one could tell that she initially did not know moving one direction would result in the image moving across the visual field in the opposite direction.

Subjects 2 through 6:

The remaining five subjects could not be trained sufficiently long for most of the formal testing. Learning rates suggest a linear trend with the exception of Subject 5. This bright 11 year-old boy who was an accomplished drummer and pianist (self-taught) enjoyed using the system but had difficulty attending to tasks either becoming tired or anxious after a short time. The curriculum was circumvented a bit and moved right into the 3-D reaching, moving and pursuit tracking to keep his interest. Investigators could then backtrack using shapes to develop differentiation skills in these tasks. His rate of accomplishment was much higher using the perceptually richer 3-D context. The progress of Subject 3 was consistent with this approach also, as she developed spatial understanding prior to adequate shape recognition for formal testing. All of the children needed instructions to move their heads either up and down or side to side for initial scanning. Subjects 2 and 3 had the most difficulty with this and experienced the greatest difficulty interpreting the sensations on their tongue. Subject 2 had the additional problem of making ballistic head movements and overshooting target positions most of the time. In spite of her age and keen intelligence she still could not move through her own home with ease either. Her highest skill was pursuit tracking which she found quite easy, perhaps due to the fact that it give feedback for controlling head movements. Subjects 4 and 6 had good head control and both made nice progress relative to the amount of time they were available for training. Subject 4 attended a residential school two hours away and came in on the weekends. Subject 6 was the youngest child with a low attention span, distracting training environment and frequent congestion. He was a mouth breather even when free of congestion and this made use of the system more difficult for longer periods of time.

Task: reduce or eliminate developmental delays in spatial cognition

### Subject 1 Accomplishments: Pre-Test 0%, Post-Test 100%:

She was 100% accurate in a Piagetian perspective taking tests at 0 degrees, 180 degrees, 90 degrees and 270 degrees when tested with 22 hours of training. She was not testable on the task prior to training. Understanding of linear perspective was demonstrated as she by consistently using size and height cues for placement of objects on the table in front of her. For example, when three candles were placed diagonally in front of her she asked “why did you place them diagonally?” When asked how she knew she replied, “the bottoms of the one on the center and left candles are higher up and besides the one on the left is smaller looking.” She used the same type of cue to judge items interposed like a square placed in front and overlapping a triangle.

### Subject 3:

This 11 year-old girl was informally tested on interposition and perspective taking.

She demonstrated understanding of 3-D space that exceeded her learning in 2-D. She was consistently able to use cues of relative height and size in performing the interposition test to place shapes in their relative overlapping positions. Her ability to differentiate individual forms, however, was deficient so that she would place the wrong shape but in the right orientation. For example, when given a display of a square in front of a circle she would select a triangle but place it in the correct position that would have replicated the target display. Thus, she developed an understanding of 3-D concepts without having the differentiation and conceptual understanding of forms that may or not hold relevant information for guiding action. She could tell if shapes were “curved” or “pointed” but as she reported she could not distinguish within these two broad categories.

Task: use dynamic spatial information from the TVSS for trajectory prediction and intercept for capture.

### Subject 1 Accomplishments: Pre-Test 0%, Post-Test 90%.

She was tested in a task with a ball rolling down a ramp aimed to roll off of the table in front of her in one of five different positions. The ball always began at midline with each path being about 15 degrees from the neighboring paths. The time from ball release to falling off the table was 2 seconds. Trials were randomized. She wore headphones with white noise and her camera was covered between trails to control for auditory cues or observation of the tester. Pre-testing score was 0% on five trials. Posttesting (@26 hours of training) score was 90% correct on 20 trials. She became skilled at rolling a ball back and forth with the trainer. She demonstrated preparatory placement and hand opening for capture of the ball. She was tested informally by moving the angle of the camera she was wearing and observing that she made initial errors consistent with the previous camera position for 8-10 captures and then self-corrected or recalibrated.

Subjects 2-6: all accomplished at pursuit tracking of stimuli across the frontal plane.
Subjects 3 and 5: were both learning ball capture with the rolling task and showed some calibration of space but did not reach the level of making aimed anticipatory reaches to moving stimuli.
Task: accuracy and processing time for recognition of 2-dimensional figures.
Subject 1 Accomplishments: Pre-Test Unable. Post-Test Mean Time to Recognition 3.4 Seconds, 100% Correct.

She became very good and fast at letter recognition. On ten randomized trials she identified letters with an average time of 3.4 seconds in a range from 1.2-6.7 seconds. Her strategy was to center the image and then with one quick up and down movement determine the letter. Through observation and her excellent reporting one could determine that she frequently recognized the letter immediately but adopted the strategy of movement to disambiguate the image. Because of the relatively poor resolution of 144 pixels diagonal lines would look curved to her as a stair-step pattern appeared and reappeared. Moving helped her to tell if the stair patterns were part of the image or an artifact of the system.

Subject 3: was the only other child, beside Subject 1, to have any exposure to alphanumeric characters prior to training on the TVSS. Subject 3 had decided she wanted to learn letters and was using her hands to explore signs and other displays with raised letters. Using the TVSS system helped but she had difficulty differentiating letters in part, because she tended to tilt her head making rectilinear forms fall on the diagonal. Diagonal lines tend to flicker or appear more rounded because of the low resolution of the TDU.
Subjects 2, 3 & 5: all became proficient at recognizing and differentiating the shapes of circle, oval, square, rectangle, and triangle as both solid shapes and outlined shapes. Recognition times were not formally tested.

### General Summary

While group data analyses were not possible, the data from Subject 1 and the rates of progress of the other five subjects demonstrate that the tongue based TVSS is an effective technology for delivering pictorial and video images for functional interpretation and use. Perceptual acuity of the tongue was sufficient for all of the subjects to use the 144-pixel array for differentiation and perception of forms. Indeed, the low resolution of the system was frequently a problem with subjects describing a “sparkle” effect with diagonal and curved forms that would make particular pixels turn off and on with a stair-step pattern. The subjects compensated by moving or jiggling the image to determine what was artifact from the system. All of the subjects enjoyed the training and were excited about being able to perceive things that they had not been able to without the TVSS.

### Gray Scale Perception

At around 20 hours of training Subject 1 began to ask questions that suggested she perceived gray scale with the system. The TVSS generates small electrical currents relative to the luminance of each pixel. Optimal conditions are of high contrast and have always been used in training with white forms against black backgrounds. When she was viewing a set of nesting dolls for size discrimination and placement she asked “what is that in the middle?” The dolls were high contrast on the top, black on the bottom, and had a wide band of detail in the middle that was projected as gray when broken in 144 pixels. She reported feeling something but not as much as the faces of the dolls. Her working level of stimulation was around 30% of the maximum 40 V of the system so bright white would provide about 13V. The Gray would be then about 6 or 7 V. This capability was not anticipated so the system was not set up to have exact quantification of the differences she could detect. Subject 3 also started to describe perception of gray scale. Training was conducted in her home facing a corner painted white. All black materials and a board were placed in front of her and training used white stimuli against this black background. She liked to look up at the white ceiling between activities “to get a good tingle” on her tongue. One evening she asked, “What am I looking at now?” She pointed the camera to the intersection of the walls and ceiling. She perceived the slightly darker shade of the wall with less direct light.

When it was realized that subjects could perceive gray scale it was decided to pilot orientation and mobility tasks, as possible, with the relatively non-portable system. The first attempt was with subject 1 trying shorelining down a white hallway with dark doors on either side. The brightness was adjusted and contrast levels to include gray scale and put the system on a cart that could be pushed behind her. She was able to go down the hall, turn a corner and stop before touching a door with a black sign mounted at eye height.

Later in her training orientation skills were tested for walking a street crossing distance without veering. Outdoors in natural light we had a figure in white stand against evergreen trees. Subject 1 had to scan the environment until she found the figure and the walk to the figure. Using an ABAB design she first made three attempts to walk to the figure without the TDU in her mouth. On the first trial she stopped short, second and third she veered approximately 10-15°. With the TDU in she walked directly to the figure. Veering was seen again when the TDU was not used showing that the effect of being able to walk directly to the figure was not due to learning on the first 3 trials. Indeed on one trial she veered right and when she tried to orient again went even further right seeking the figure.

### Example 13

### Surgical Assistance

### Guidance and Control of Surgical Devices

In some embodiments, the systems of the present invention are used to assist in the guidance of surgical probes for surgeries. Current techniques for guiding catheters contain inherent limitations on the level of attainable information about the catheter's environment. The physician at best has only a 2-dimensional view of the catheter's position (a fluoroscopic image that is co-planer with the axis of the catheter). There does exist some force feedback along the axis of the catheter, however this unidirectional information provides only low-level indications regarding impediments to forward catheter motion. These factors greatly limit the surgeon's haptic perception of objects in the immediate vicinity of the catheter tip. For example, when humans touch and manipulate objects, we receive and combine two types of perceptual information. Kinesthetic information describes the relative positions and movements of body parts as well as muscular effort. Tactile information describes spatial pressure patterns on the skin given a fixed body position. Everyday touch perception combines tactile and kinesthetic information and is known as haptic perception. From the surgeon's perspective, little or no tactile or kinesthetic feedback from the catheter can exist because control is generally in the form of thumb and forefinger levers that alter guide-wire tension and therefore control distal probe movements.

The embodiment of the present invention described herein utilizes the tongue as an alternate haptic channel by which both catheter orientation and object contact information can be relayed to the user. In this approach, pressure transducers located on the distal end of the catheter relay sensor-driven information to the tongue via electrotactile stimulation. Thus, based on the perceived stimulator orientation and corresponding tongue stimulation pattern, the physician remotely feels the environment in immediate contact with the catheter tip. In other words, this alternate haptic channel provides sensation that could be perceived as if the surgeon was actually probing with his/her fingertip. If one could “feel” the environment, in conjunction with camera and fluoroscopic images, tissues and organs could be probed for differences in surface qualities and spatial orientation. This Example describes the methods and results of developing and testing two prototype probes in conjunction with a tongue display unit

The overall goal was to demonstrate the feasibility of a novel sensate surgical catheter that could close the control loop in a surgery by providing tactile feedback of catheter orientation and contact information to the user's tongue. To that end, a prototype system was developed that affords a tactile interface between two prototype probes and a human subject.

The first consideration was the need to satisfy a reasonably small size requirement while providing a sensor resolution capable of yielding useful results. Conductive polymer sensors from Interlink Electronics, Inc. (Force Sensing Resistor (FSR), Model #400) and Tekscan, Inc. (Flexiforce, Model A101) were chosen for use because of their small size (diameter and thickness) and variable resistance output to applied forces. Having a resistance output also allowed the design of relatively simple amplification circuitry. A spring-loaded calibrator was designed and built to facilitate repeatable force application over a range of 0 to 500 gm. Testing each sensor for favorable output characteristics aided the decision to proceed with the FSR sensor. The output response, although slightly less linear than the Flexiforce sensor, was determined acceptable given the FSR's smaller physical dimensions. Each sensor was 7.75 mm in diameter, had an interdigitated active sensing area of 5.08 mm, a thickness of 0.38 mm, and 30 mm dual trace leads. This allowed probe size optimization for various sensor patterns and although the final prototypes are much larger than required for surgical application, the idea underlying this project was to prove the utility of the concept. Thus, in surgical devices, these components are used in smaller configurations.

Initial probe design criteria included the probe's ability to detect normally and laterally applied forces. This suggested, at the very least, a cube mounted on a shaft with sensors located on the remaining five sides. This design however, was quickly observed to contain considerable ‘dead space’ for forces not applied within specific angles to each sensor. For example, the probe would not sense a force applied to any of the corners. Many permutations of this preliminary design were considered before reaching two possible solutions: a ball design and a cone design. Each utilizes a piece of High Density Polyethylene (HDPE) machined to form the substrate upon which the FSR sensors were mounted.

The ball probe design uses four FSR sensors located 90° apart, with each attached at 27° taper. Because the active sensing area and trace leads are of similar thickness, a ‘force distributor’ was added to the active area by applying a 3 mm×3 mm×2 mm (W×L×H) square of semi-compliant self-adhesive foam (3M, St. Paul, Minn.). To activate the sensors, a 14.7 mm diameter glass sphere was placed inside the machined taper therefore contacting the foam sensor pads. The lead wires were gathered and inserted into a 12.8 mm×10.6 mm×38 cm aluminum shaft (OD×ID×L), which was then attached to the HDPE tip using an epoxy adhesive. To maintain contact between the sphere and sensors, as well as to protect the probe during testing, a 0.18 mm thick latex sleeve (Cypress, Inc.) was stretched over the distal portion and affixed using conventional adhesive tape (3M, St. Paul, Minn.).

The design of the Ball probe offered a robust and simple solution to the sensing needs of the system. Having the sensors and trace leads mounted internally provides a level of protection from the outside environment. A glass sphere helps forces from a wide range of angles to be detected by one or more sensors. The design, using only the four perimeter sensors, reduces the amount of necessary hardware and utilizes software to calculate the presence of a virtual fifth sensor for detecting and displaying axially normal forces. This software essentially monitors the other sensors to see when similar activation levels exist, then creates an average normal force intensity. The probe does however contain limitations. Even though the ball helps distribute off-axis forces, it cannot distinguish more than one discrete force. For example, if the probe passes through a slit that applies force on two opposing sides, the probe will only detect the varying normal component of the two forces.

The cone probe configuration employs six of the FSR sensors. The substrate is a 17 mm diameter cylinder of HDPE externally machined to a 30° taper. Five sensors are located on the taper in a pentagonal pattern, and the sixth is mounted on the flat tip. The ‘force distributor’ foam pads were also added to each sensor and a 8.5 mm wide ring of polyolefin (FP-301VW, 3M, St. Paul, Minn.) was heat-molded to fit the taper. The purpose of the polyolefin is to help distribute forces that are not normal to one of the five perimeter sensors thereby decreasing the amount of ‘dead space’ between sensors. A common ground wire was used to decrease the amount of necessary wire leads and once bundled, they were ran along the outside of a 6.35 mm×46 cm (OD×L) steel shaft threaded into the HDPE tip. The probe was also protected by a 0.18 mm thick latex sleeve (Cypress, Inc.) attached using 3M electrical tape.

One of the main design features of the Cone probe is the increased sensor resolution. The five perimeter sensors afford detection of forces on more axes than with the Ball probe, and the discrete normal force sensor allows for simple software implementation. The design was pursued because it eliminates the opposing force detection problem found with the Ball probe design. Forces in more than one location can be detected as discrete stimulations regardless of the plane in which they occur. Because each design has merits and limitations, both required testing to determine how subjects react to the stimulations they provide.

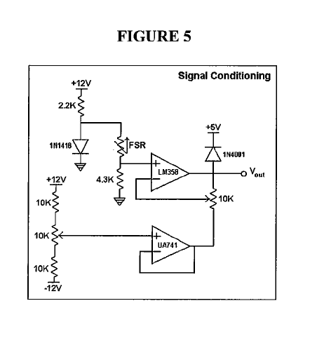

Contact stimulus information is relayed from the sensors and modified by conditioning circuitry to produce 0-5 volt potential changes. These voltages are then connected to the analog input channels of a Tongue Display Unit (TDU 1.1, Wicab, Inc., Madison, Wis.) that converts them into variable intensity electrotactile stimulations on the user's tongue. The TDU is a programmable tactile pattern generator with tunable stimulation parameters accessed via a standard RS-232C serial link to a PC. The circuit in FIG. 5 was replicated for each sensor and serves as an adjustable buffer amplifier with an output voltage limiter. The amplifier and voltage limiter are important for adjusting the sensitivity of each sensor and limiting the output voltage to below the 5-volt maximum input rating on the TDU. To compensate for preloading effects of the force distribution foam on the sensors, the adjustable buffer facilitates ‘no-load’ voltage zeroing. Each sensor is modeled as a variable resistor and labeled as “FSR” in the schematic below.

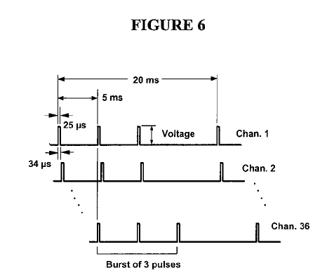

Software was developed for each prototype probe so that sensor information could be monitored and processed. An output voltage (Vout) for each sensor corresponds to the force magnitude applied to each FSR. This voltage is then interfaced to the TDU through an analog input and subsequently converted into a corresponding electrotactile waveform shown in FIG. 6. Using an existing GUI, an image of the probes with discrete areas resembling the actual sensor patterns was created. Data from the analog channels are digitally processed and shown as a varying color dependent upon the voltage magnitude. Therefore, as contact is made with the probe, the graphical regions corresponding to those sensors in contact with the test shape change from black (0 volts) to bright yellow (5 volts), depending on a linear transform of contact force magnitude (vₛ), to voltage amplitude of the stimulation waveform (vᵢ).

This is a graphical representation of what the user should be feeling on their tongue, thus providing a means of self-training and error checking in the sensor-tactile display mapping function. In both cases, the general orientation of the image (i.e. Top, Bottom, Left, Right) corresponds to the probe when viewed from the tail looking forward. Typically the central front portion of the tongue is most sensitive with less sensitivity toward the side and rear. The average intensities for each sensor were adjusted with amplification gains to compensate for this variation.

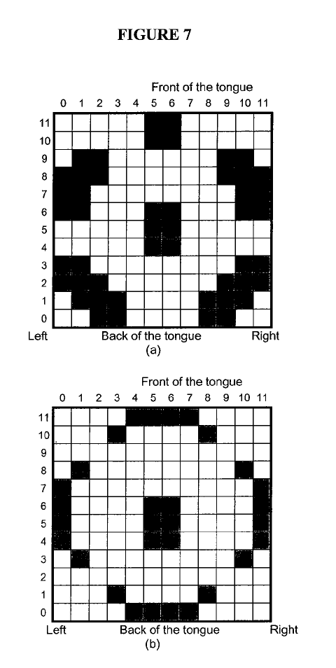

A final software modification provided an electrode stimulation pattern that spatially matched the sensors for each probe. Groups of electrodes were assigned to each sensor and are represented as gray areas in FIG. 7. The stimulation pattern on the user's tongue therefore reflects the spatial information received by the TDU from the sensors and is output to a lithographically-fabricated flexible electrotactile tongue array consisting of 144 electrodes (12×12 matrix). The number of electrodes assigned to each sensor was based on an area weighed average of the local sensitivity of the tongue. Thus, for equal sensor output levels, the intensity of the tactile percept was the same, regardless of location on the tongue. The user can set the overall stimulation intensity with manual dial adjustments, thus allowing individual preference to determine a comfortable suprathreshold operating level.

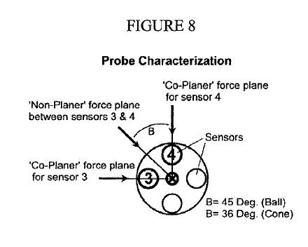

To aid in the understanding of how subjects might perceive object contact information provided by the prototype sensate probes, it was important to first investigate how the probes themselves react to controlled discrete forces. A calibration and characterization experiment was performed on each prototype using a 200 gm force applied at 0° (normal), 30°, 60°, and 90° angles. The test was first employed for angles co-planer to each sensor, and then repeated for non-planer angles between two adjacent sensors (45° for Ball probe, 36° for Cone probe) (see FIG. 8). Tables 3 and 4 show typical sensor output voltages, as a function of applied force angle, for the Ball and Cone probe respectively. The force response data in Tables 3 and 4, presents a quantitative analysis of each probe's technical merits and limitations. The first observation is that, for co-planer forces applied to each sensor, both probes produce output intensities that vary according to each sensor's location.

TABLE 3
Ball probe response for: (a) co-planer forces (performed on all sensors),
(b) forces applied 45° to sensors 3 & 4

Vout (Volts)

| | | | | |
|---|---|---|---|---|
| SENSOR | Co-axial (normal) | 30° | 60° | 90° |

(a)

| | | | | |
|---|---|---|---|---|
| 1 (Top) | 1.03 | 1.7 | 1.9 | 1.3 |
| 2 (R) | 1.4 | 2.7 | 2.9 | 2.4 |
| 3 (Back) | 1.75 | 3.3 | 3.8 | 3.1 |
| 4 (L) | 1.81 | 3 | 3.4 | 2.5 |
| 5* | 1.50 | 0 | 0 | 0 |

(b)

| | | | | |
|---|---|---|---|---|
| 1 (Top) | 1.03 | 0 | 0 | 0 |
| 2 (R) | 1.4 | 0 | 0 | 0 |
| 3 (Back) | 1.75 | 2.6 | 2.5 | 1.6 |
| 4 (L) | 1.81 | 2.7 | 2.5 | 1.7 |
| 5* | 1.50 | 0 | 0 | 0 |

*Phantom center sensor

TABLE 4
Cone probe response for: (a) co-planer forces (performed on all sensors),
(b) forces applied 36° to sensors 3 & 4

Vout (Volts)

| | | | | |
|---|---|---|---|---|
| SENSOR | Co-axial (normal) | 30° | 60° | 90° |

(a)

| | | | | |
|---|---|---|---|---|
| 1 (Top) | 0 | 1 | 1.5 | 1.7 |
| 2 (Upper R) | 0 | 1.6 | 2.1 | 2.5 |
| 3 (Lower R) | 0 | 1.75 | 2.8 | 3.1 |
| 4 (Lower L) | 0 | 1.8 | 3 | 3.1 |
| 5 (Upper L) | 0 | 1.5 | 2.2 | 2.6 |
| 6 (Center) | 0.8 | 0.4 | 0.1 | 0 |

(b)

| | | | | |
|---|---|---|---|---|
| 1 (Top) | 0 | 0 | 0 | 0 |
| 2 (Upper R) | 0 | 0 | 0 | 0 |
| 3 (Lower R) | 0 | 0.4 | 0.9 | 0.5 |
| 4 (Lower L) | 0 | 0.5 | 1.0 | 0.5 |
| 5 (Upper L) | 0 | 0 | 0 | 0 |
| 6 (Center) | 0.8 | 0 | 0 | 0 |

For the Ball probe in Table 3, the results show that peak output occurs when co-planer forces were applied at approximately 63° from the shaft axis. Because of the four sensor Cartesian pattern, forces applied at 45° to the sensor plane activate at most two sensors. Maximum output voltage, at this angle, occurs for forces applied approximately 300 from the shaft axis. By comparison, the Cone probe characterization in Table 4 shows co-planer maximum output for forces at 90° to the shaft axis. This response was somewhat surprising since it was thought that sensitivity would be maximal at about 60°. However, the molded polyolefin ring in contact with the sensors likely distributed the off-axis forces and contributed to this result. Non-planer forces applied at a 36° angle yielded output in two sensors (3 & 4), similar to that of the Ball probe, but with significantly lower magnitudes.

The net result of the tests indicates that the Ball probe provides higher output response to non-planer forces than does the Cone probe. The Cone probe did, however, respond more favorably to transitions from normal to 90° co-planer forces, however, neither probe provided exceptional output for transitions from normal to 90π non-planer forces. Having a limited number of discrete sensors may account for the discontinuous force detection regardless of applied angle. Thus, in other versions of probe design, increased sensor resolution is used to improve the angular transitional response.

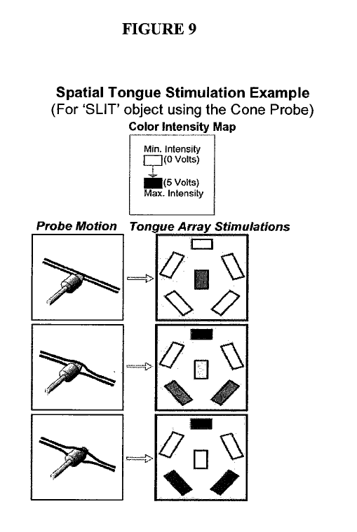

The system was tested on subject. Subjects observed tongue electrotactile stimuli from both probes (i.e. no visual feedback) while contacting one of 4 different test objects. Six adult subjects familiar with electrotactile stimulation participated in this experiment. Each subject was first shown the prototype probe, the 4 possible test shapes, the TDU, and the sensor-to-tongue display interface program. The 4 object stimuli were as follows: A ‘Rigid’ stimulus was created using hard plastic. A ‘Soft’ stimulus was designed from a 3 cm thick piece of compliant foam. A ‘Slit’ force stimulus was achieved using two pieces of foam sandwiched together. A ‘Shear’ force stimulus was realized from a tapering rigid plastic tube. The ‘Rigid’ and ‘Soft’ surfaces were used to test the ability of users to discern normal force intensities as unique characteristics of the test shapes. The ‘Slit’ force stimulus is intended to mimic a catheter passing between two materials (see FIG. 9) and the ‘Shear’ stimulus provided by the tapered tube were used to test if subjects can perceive the orientation of probe contact force.

Subjects were then trained to use the graphical display of sensor activation pattern to aid perception of the electrotactile stimulation on their tongue. The experimenter maintained control over probe movements, and once participants were able to correctly identify each of the four test stimuli without visual feedback, they were blindfolded and the formal experiment began.

During the experiment, subjects were instructed not to adjust the main intensity level. The four test configurations were randomly (without replacement) presented in two blocks of 12 trials (equal representation) with one block given for each probe. Two data values were collected for each trial: (1) first the subjects were asked to identify the stimulus as representing one of the four possible test shapes. If the choice was incorrect, the subject's incorrect choice was recorded and used to check for correlations between test stimuli and/or probes. (2) The participants were then asked to describe what they “visualize” and/or “feel” as the environment in contact with the probe. For example, a subject may comment that the sensations on the left side of their tongue leads them to perceive the probe contacting the left side of the vessel wall and that a lateral shift to the right is necessary. This qualitative information aided in identifying the merits and limitations of the prototype system.

TABLE 5
Confusion matrix for overall subject correct perception using, (a) the Cone
probe and (b) the Ball probe

| | |
|---|---|
| ACTUAL | PERCEIVED STIMULUS |

| | | | | |
|---|---|---|---|---|
| STIMULUS | RIGID | SOFT | SLIT | SHEAR |

(a)

| | | | | |
|---|---|---|---|---|
| RIGID | 77.8 | 5.6 | 0.0 | 16.7 |
| SOFT | 5.6 | 83.3 | 11.1 | 0.0 |
| SLIT | 0.0 | 16.7 | 83.3 | 0.0 |
| SHEAR | 0.0 | 5.6 | 0.0 | 94.4 |

(b)

| | | | | |
|---|---|---|---|---|
| RIGID | 77.8 | 5.6 | 5.6 | 11.1 |
| SOFT | 5.6 | 61.1 | 27.8 | 5.6 |
| SLIT | 5.6 | 22.2 | 66.7 | 5.6 |
| SHEAR | 5.6 | 0.0 | 11.1 | 83.3 |

The results of the study reveal that, overall, subjects were generally able to correctly identify the four test shapes using only electrotactile stimulation on the tongue. Table 5 presents the results of this study as a confusion matrix for the Cone and Ball probe respectively. The results show that subjects attained higher perceptual recognition using the Cone probe (avg. 85% correct) than with the Ball probe (avg. 72% correct). ‘Shear’ force stimuli yielded the highest percentage correct for both probes with one subject scoring perfectly on all trials using the Cone probe. While significantly lower for the Ball probe, the ‘Soft Normal’ and ‘Slit’ force recognition rates are also promising. The results also show evidence of perceptual difficulties in some trials and should be noted. In particular, for the Cone probe trials, confusion between ‘Soft Normal’ and ‘Slit’ stimulus accounted for most errors. It is conceivable that this is because sensor activations can be similar for these two objects. If the central stimulus was not felt during the ‘Soft Normal’ force stimulus (possibly due to lateral masking effects), the percept may be that of the ‘Slit’ condition, which produces a “pinching” stimulus that is felt on the perimeter of the tongue.

During Ball probe trials, misperceptions frequently occurred between the ‘Slit’ and ‘Soft Normal’ force stimuli. The probe lacked the ability to discretely sense two opposing forces, as is the case of the ‘Slit’ shape, and contact information for the ‘Slit’ was therefore presented as a varying normal force. In other trials, it was reported that while scanning the tongue array for stimulation, spatial orientation on the array was sometimes lost, making perception of tip to rear stimulation transitions difficult to distinguish. This problem could be eliminated by incorporating a small nib or bump at the center of the tongue array that would allow users to “feel” their way back to a reference position similar to the home position on a numeric keypad. Another note is that two subjects expressed that having an alternate tongue mapping function may have helped them visualize the probe in contact with the test shapes more accurately. Their main concern was that the top of the probe was mapped to the tip of the tongue whereas mapping it to the back of the tongue may be more spatially intuitive. Thus, with additional training or alternative configurations, accuracy is greatly increased.

With practice, users learn to process substitute sensory information to the point where catheterization tasks are perceived as unconscious extensions of the hands and fingers. Implementation of MEMS-based sensors, partially due to their small size, low power consumption, and mode of sensing flexibility, operational catheters will facilitate spatial perceptions far beyond the results of the results reported above. It was demonstrated that the external sensor design (Cone probe) resulted in better perceptual performance than did the internal sensor design (Ball probe). However, a modified Ball design that provided greater internal sensor resolution through active perimeter sensors located on the ball surface could create an optimal synthesis of the two current designs and their respective performance features.

With the aid of sensor equipped catheters, relaying critical information regarding probe position and tissue/organ surface qualities as patterned electrotactile stimulation is contemplated. The surgeon's new ability to “feel” how the catheter is progressing through the vessel may increase the speed with which probes can be navigated into position. This additional diagnostic tool may therefore decrease the amount of time patients are anesthetized and/or under radiation.

### Retinal Surgery Enhancement

In some operations on the retina, the retinal surgeon must separate the pathalogical tissue in the retina using a pick by vision only, since the forces on the pick are so minimal that they cannot be felt. To enhance such surgeries a surgical pick can be configured with sensors so as to supply information about the surface of the tissue through a tactile device to the operating surgeon. For example, on the pick, several mm behind the tip, a MEMs (tiny) accelerometer or other sensor is placed. The sensor is configured to pick up the tiny vibrations as the pick is used to separate the tissue. The signal from the sensor is sent to an amplifier and to a piezoelectric vibrator or other means of delivering the amplified signal through intensity of signal provided on the pick. A small battery is included in the package. Thus, when the surgeon uses the pick on the retina he/she perceives an amplified version of the forces on the tip of the pick that would be delivered to the brain via the fingers holding the pick. The device may be configured a single-use throw-away instrument, since it is quite inexpensive to make and it might be impractical to sterilize and maintain. However, it could also have other formulations, such as a romovable instrumentation package clipped on the sterile retinal pick

### Robotic Control

In some embodiments, the present invention provides a fingertip tactile stimulator array mounted on the surgical robot controller. The electrode arrays developed for tongue stimulation (12×12 matrix, approx. 3 cm square) are modified to allow mounting (e.g., via pressure-sensitive adhesive) on the hand controller. This is accomplished largely by changing the lithographic artwork used by the commercial flexible-circuits vendor (All-Flex, Inc., St. Paul, Minn.). Software is configured to receive data from the tactile sensors and format it appropriately for controlling the stimulation patterns on the fingertips. The resulting system provides a tactile-feedback-enabled robotic surgery system.

An electrode array is made of a thin (100 μm) strip of flexible polyester material onto which a rectangular matrix of gold-plated circular electrodes have been deposited by a photolithographic process similar to that used to make printed circuit boards. The electrodes are approximately 1.5 mm diameter on 2.3 mm centers. A 2×3 array of 6 electrodes is mounted on the concave surface of the finger-trays. Each array is connected via a 6 mm wide ribbon cable to the Fingertip Display Driver, which generates the highly controlled electrical pulses that are used to produce patterns of tactile sensations.

The electrical stimulus is controlled by a device that generates the spatial patterns of pulses. The sensor displacement data is processed and output by the host PC as serial data via the RS-232 port, to the Fingertip Display Driver (FDD). The FDD electrotactile stimulation pulses are controlled by a 144-channel, microcontroller-based, waveform generator. The waveform signal for each channel is fed to a separate 144-channel current-controlled high voltage amplifier. The driver set-up, according to the particular pattern of stimulation, delivers bursts of positive, functionally-monophasic (zero net dc) current pulses to the electrode array, each electrode having the same waveform. Intensity and pulse timing parameters are controlled individually for each of the electrodes via a simple command scripting language. Operation codes and data are transferred to the TDU via a standard RS-232 serial link at up to 115 kb/s, allowing updating the entire stimulation array every 20 ms (50 Hz).

Sweat-related effects on the fingertip array are addressed by providing means to wick sweat away from the electrode surface via capillary tubes, etc., designed into the electrode array substrate.

Electrotactile stimulation is used to produce controlled texture sensations on the fingertips to allow tactile feedback with much greater realism than existing technology.

In one embodiments a one-to-one, spatially-corresponding mapping of sensor elements to stimulator elements (electrodes) is used. However, given that the robotic end-effector may be very small and irregularly shaped, depending on the particular surgical procedure, other spatial mapping schemes may be employed. For example, the system may employ a level of “zoom” (i.e., ratio of tactile display size to sensor array size), as well as the effects of convergence (multiple sensors feeding each tactile display element) and divergence (use of multiple tactile display elements to represent each sensor).

### Example 14

### Underwater Orientation Experiments

Navy divers, researchers, and recreational divers operating in the littoral and deep-water often must perform activities in murky or black water conditions limiting the effectiveness of visual cues. When performing salvage or rescue/recovery or egress from sunken structures, available visual references may cause individuals to misperceive their orientation and lead to navigational errors. For military personnel, requirements for clandestine operations and the need to maintain dark adaptation for nighttime ops preclude the use of dive lights and make illuminated displays undesirable.

Tasks such as search and rescue, egress, mine countermeasures and salvage are interrupted when using visual aids for navigation and communications. Meanwhile the remaining human sensory systems remain under-utilized, leading to inefficient use of diver cognitive capabilities. The present invention provides a system for military and other divers that enhances navigation and, as desired, provides other desired sensory function (e.g., alarms, chemical sensors, object sensors). This device has been termed BRAINPORT Underwater Sensory Substitution System (BUDS³) and provides additional interface modality for warfighters in the underwater operational environment that increase effectiveness by improving data understanding for navigation, orientation and other underwater sensing needs.

In preferred embodiments, the system is worn in the mouth like a dental bridge or mouth guard and interfaces electrically to the tongue and lips.

DARPA and other research agencies have developed methods of enhancing human and human-system performance by detecting bioelectric signals, both invasively (neural implants) and non-invasively (skin surface or non-contact electrodes) to allow direct control of external systems. Dynamic feedback is a key element for the use of these brain machine interfaces (BMIs). The BUDS³ sensory interface is used to augment both the visual and sensory motor training with current BMIs concepts as well as the accuracy of detection of intent in concert with other bioelectric BMIs. The BUDS³ system exploits the relatively high representation in the cerebral cortex of the tongue and lips.

In some preferred embodiments, in addition to providing navigation information, the BUDS³ is configured to display other underwater data such as sonar or communications (from the surface or from other divers) and has integration of EMG capabilities which would provide a subvocal communication capability and detect operator input commands that could be used to control unmanned underwater (or surface) vehicles. Preferably, the system is fully wireless and self-powered. Non-diving military applications include control of manned and unmanned vehicles, control of multispectral electronic sensing and detection platforms, control and monitoring of automated systems, management of battlespace C4ISR, among others.

Divers using the BUDS³ system operationally will have improved orientation and navigational capabilities and extended sensory capabilities based on sonar and other technologies.

It is widely observed that the mind constructs a virtual space, experiencing the body and the tools attached to it as a single unit filling the space. The nervous system readily extends to experience an external object as if it were a part of the body. Anyone who has ever slowly backed a car into a lamppost, and perceived the collision as direct physical pain has experienced this process. Similarly, a blind person using a long cane perceives objects (a foot, a curb, etc.) in their real spatial location, rather than in the hand, which is the site of the human-device interface. This capacity represents a powerful but untapped resource for process monitoring, with many significant practical applications. Rensink (2004) notes that power is seen in the ability to sense that a situation has changed before being able to identify the change, using “mindsight.” He exposed 40 subjects to a series of images each shown for 0.25 second. Sometimes the image would be repeated throughout the trial; sometimes it would be alternated with a slightly different image. When the image was alternated, about a third of subjects reported feeling that the image had changed before they could identify the change. In control trials, the same subjects were confident that no change had occurred. The systems of the present invention provide a way to exploit this rapid understanding of information.

In some embodiments, the BUDS³ data interface provides an electrotactile tongue interface that is incorporated into a rebreather mouthpiece of the diver. A similar device may be incorporated into emergency air bottles. Molds of current rebreather and scuba system mouthpieces are made and replacement castings are formed with electrotactile arrays embedded into the lingual and buccal surfaces. Additionally, switches are integrated into the bite blocks to allow diver control of the interface. The mouthpiece is connected to drive electronics and power mounted to the dive gear. Two hardware stages are used to control the array. The driver, located close to the mouthpiece, provides the actual waveforms to the individual tactors. An embedded computer/power supply module mounted to the buoyancy control device or dive belt controls the driver via serial link. The control computer connects to sensors such as accelerometers, inertial navigation systems, digital compasses, depth gauges, etc. and runs the software that determines what signal is presented to the diver.

The Institute for Human and Machine Cognition (IHMC) has developed a modular, software agent based integration architecture under the DARPA IPTO Improving Warfighter Information Intake Under Stress Program that may be used to implement the BUDS³ device. This architecture uses Java (or any other programming language that can communicate via Java or TCP/IP). The architecture is cross platform (currently supported on Windows and Linux OSs) and provides a standardized interface protocol for disparate heterogeneous elements. Drivers are provided for each sensor device (digital compass, inertial navigation unit, etc) and for the BUDS³ prototype. This allows for rapid integration and side-by side testing, training, and usage of different sensors. Waterproofing is accomplished through use of waterproof housings, using off the shelf waterproof connectors/cabling and potting of circuits.

Persons with no eyes have learned complex three dimensional perceptual tasks using the systems of the present invention, including hand-“eye” coordination, such as catching a ball rolling across a table, in a single training session. In addition, individuals who have lost vestibular (balance) organ function due to drug toxicity (e.g., gentamycin) have demonstrated rapid improvement in postural sway and gait when using the system to represent tilt sensed by a head worn accelerometer. The key to its operation is the user's nervous system's ability to use the data provided by the system to abstract semantic cues (the meaning of the data stream, or in psychological parlance, analog information, rather than the data values themselves, or digital information) that describe the process being sensed. Sensation can be experienced and unconsciously integrated into the operator's awareness.

Experimental studies of implicit learning show that individuals engaged in a learning task are consciously focused on functional features of the task, rather than the underlying structural characteristics of the material. This is seen in the infant's acquisition of knowledge of the semantic and syntactic structure of its natural language. The infant's attention is directed toward the functional aspects of verbal communication (getting what it needs, understanding the caretakers), not on the structural features of the language. Yet, over time, the child comes to speak in a manner that reflects the complex array of linguistic and paralinguistic rules necessary for successful interaction in social settings—without having acquired conscious knowledge of either the rules that govern its behavior or the ongoing processes of rule acquisition. Remarkably, the process goes beyond learning the rules of a coherent situation; it extends to the ability to identify and engage in interpersonal deception.

Prior research demonstrated that dissimilar but related sensory inputs facilitate the interpretation of data. Rubakhin & Poltorak, (1974), for example, studied visual, auditory and tactile information presented simultaneously under two conditions: identical or duplicated information in all three perceptual systems, or different information in each perceptual system. They found that multi-modally presented information must be processed simultaneously, because sequential processing limits the overall channel capacity of the brain. Deiderich (1995) performed a simple reaction time (RT) experiment in which subjects were asked to react to stimuli from three different modalities (i.e. visual, auditory, and tactile). The stimuli were presented alone, as a pair from two different modalities, or as a triple from all three modalities. Double stimuli conditions showed shorter RTs when compared to single stimulus conditions. Triple modality stimuli showed a further reduction in RT, demonstrating inter-sensory facilitation of RT. Given that the human orientation system is multisensory, it follows that multisensory (e.g., vision augmented with BUDS3) data leads to more rapid and accurate situation awareness and thereby lead to more efficient and effective mission execution.

In preferred embodiments, the system is provided as a wireless communication system. By removing the wired link between the array and the control computer, the system is less obtrusive, dive compatible, and provides intra-oral substrates. For example, orthodontic retainers from a cross-section of orthodontic patients were examined to determine the dimensions of compartments that could be created during the molding process to accommodate the FM receiver, the electrotactile display, the microelectronics package, and the battery. The dimensions and location of compartments that could be built into an orthodontic retainer have been determined. For all the retainers of adolescent and adult persons examined, except for those with the most narrow palates, the following dimensions are applicable: in the anterior part of the retainer, a space of 23×15 mm, by 2 mm deep is available. Two posterior compartments could each be 12×9 mm, and up to 4 mm deep. Knowledge of these dimensions allows the development of a standard components package that could be snapped into individually molded retainers, and the wire dental clips would double as the FM antenna.

These reduced size arrays may be used in conjunction with dive gear, but also open up applications in non-diving environments. For example, divers could use the system underwater and on ground during amphibious operations, switching between display of sonar or orientation to display of night vision, communications and overland navigation data. Similarly, a wireless connection allows incorporation of the system into aviation environments and for civilian use by firefighters rescue workers and the disabled. The transmission of information from the sensor/control computer to the high-density array should be done at high speed using minimal battery power. In some embodiments, near visible infrared (IR) light, which can pass through human is used as a direct IR optical wireless communication method.

In some embodiments, electromyogram/electropalatogram capabilities are added to mouthpiece for efferent control of external systems. The facial muscles, tongue and oropharynx may be exploited as machine interface to external systems. By using a system with an integrated electromyogram (EMG) and electropalatogram (EPG) capability in the orthodontic device, the user gains a precision interface device that finds use to control unmanned aerial/ground/undersea vehicles. In addition, recent research has shown that speech patterns can be detected from EMG/EPG when subjects pretend to speak but make no actual sound. These patterns can be recognized in software and used to generate synthetic speech. This capability, coupled with audio transduction via the system permits clandestine communications between divers on a team or with the surface. With a wireless system, troops on the ground could also communicate without any acoustic emissions.

### Example 15

### MRI Research Applications

Previously developed substitution systems have not been appropriate for MRI studies. However, electrotactile tongue human-machine interface finds use for imaging studies. The tongue is very sensitive and the presence of an electrolytic solution, saliva, assures good electrical contact. The tongue also has a very large cortical representation, similar to that of the fingers, and is capable of mediating complex spatia patterns.

The tongue is an ideal organ for sensory perception. The results obtained with a small electrotactile array developed for a study of form perception with a finger tip demonstrated that perception with electrical stimulation of the tongue is significantly better than with finger-tip electrotactile stimulation, and the tongue requires much less voltage (3-8 V) than the finger-tip (150-500 V), at threshold levels which depend on the individual subject. Electrical stimulation of the fingertips requires currents of approx. 1-3 mA (also subject dependent) to achieve sensation threshold; the tongue requires about half this much current. The electrode-tongue resistance is also more electrically stable than the electrode-fingertip resistance, enabling the use of voltage control circuitry in preference to the more complex current-control circuitry used for the fingertip, abdomen, etc.

To establish initial feasibility of using the tongue tactile display unit in conjunction with MRI, two tests were performed with a 1.5 T G.E. Signa Horizon Magnet equipped with high-speed magnetic field gradients that afford the use of single-shot echo-planar imaging (EPI) pulse sequences. These experiments were designed to determine whether (1) the time-varying magnetic fields in the MRI machine would induce perceptible sensations on the tongue electrode array, and (2) whether the presence of the tongue array and related electrical activity would yield artifacts on the MRI image.

(a)—Calculation of maximal induced emf in tongue electrode array. The maximal emf induced in the tongue electrode array occurs when the RF magnetic field B₁ is perpendicular to the plane of the tongue array. The tongue array is approximately 22 in long, and the largest receiving loop would be created by shorting together the two electrodes at the furthest corners of the array. These two electrodes are approximately 1 inch apart.

Induced emf, E, in a coil placed in a time varying magnetic field, B, is calculated by:

$$E = - N \cdot A \cdot \frac{d B}{d t}$$

where:
N is the number of turns in the coil (1),
A is the area of the coil (0.0142 m²), and

$$\frac{d B}{d t}$$

is the maximal rate of change of the B₁ magnetic field;

(0.012 T)/(150 μs)=80 T/s=80 Wb/s·m²

So, the maximal expected emf, E=1.14 Wb/s=1.14 V.

This prediction was confirmed by direct measurement. The tongue electrode strip was affixed to a calibration phantom, and shorted together the two electrodes on the array corresponding to the flat cable traces encompassing the largest-area loop comprising the electrode-cable assembly. Digital storage oscilloscope measurements on the free ends of the cable during a spin-echo MRI scan (acquisition parameters: 500/8 ms TR/TE, 256×256 matrix, slice thickness=5 mm, 24 cm×24 cm field of view, 1 NEX) showed that the maximal induced emf (for all three perpendicular orientations of the electrode array in the scanner), was no more than 4 V. Both predicted and measured emf for both conditions are near or below the sensation threshold for electrotactile stimulation on the tongue (3-8 V), and hence pose no risk to the subject.

(b) Stimulation waveforms and control method. The electrotactile stimulus consists of 25-μs pulses delivered sequentially to each of the active electrodes in the pattern. Bursts of three pulses each are delivered at a rate of 50 Hz with a 200 Hz pulse rate within a burst to the 36 channels. This structure was shown previously to yield strong, comfortable electrotactile percepts. Positive pulses are used because they yield lower thresholds and a superior stimulus quality on the fingertips and on the tongue. Both current control and voltage control have been tested. It was found that for the tongue, the latter has preferable stimulation qualities and results in simpler circuitry. Output coupling capacitors in series with each electrode guarantee zero dc current to minimize potential skin irritation. The output resistance is approximately 1 kΩ.

(c) Scan with tactile stimulation. The electrode array was placed against the dorsum of the tongue in a healthy volunteer, and the flexible cable passed out of the mouth, stabilized by the lips. A 4-m cable connected the electrode array to the stimulator, located as far as possible from the axis of the main magnet. All 144 electrodes delivered a moderately-strong perceived level of stimulation throughout the experiment. A whole-brain, spin-echo MRI scan (acquisition parameters as in (b) above) was performed and displayed as nine sagittal slices.

None of the images revealed any artifact due to the presence of the electrode array or related stimulation. The subject, who was familiar with the types of sensations normally elicited by the stimulation device, did not feel any unusual sensations during the scan. These results establish proof of concept for using the tongue tactile stimulator in an MRI environment.

However, the equipment (which was not constructed to withstand the MRI environment) was apparently damaged by the induced activity produced by the imaging sequence. Thus, the methods are preferably conducted with electrical isolation via, for example, long lead wires to be able to distance the electronic instruments from the MRI machine.

All of the imaging performed on the GE Signa MR scanner is controlled by software referred to as pulse sequences. Pulse sequences can be provided by General Electric or created by the researcher. Pulse sequences generate digitized gradients, RF waveforms, and data acquisition commands on a common board, the Integrated Pulse Generator (IPG). RF waveforms are then converted to an analog format through an RF modulator on a separate board and then sent to the RF power amplifier housed in another chassis. The pulse sequence is also responsible for generating the necessary control signals to activate the modulator and RF power amplifier during RF excitation. The control signal to activate the RF power amplifier is used to activate the electronic disconnect circuit and thus electrically disconnect the tongue driver from the tongue array,

The pulse sequence software can also generate a control signal at specific points in the imaging sequence. This control signal is used to synchronize and trigger the tongue driver from the imaging sequence. Since the tongue driver sequence has a period of 20 ms, the control signal is generated immediately after the RF excitation and 20 ms later during the imaging sequence. Thus two cycles of the tongue driver sequence are executed for every one repetition period of the imaging sequence. The time during the RF excitation is the only time in the pulse sequence when the MRI procedure can damage the ET device. Allowing for 1 ms of RF excitation where no tongue stimulation is allowed, stimulation can still occur with a duty cycle over 97% if the imaging repetition time is set at 46 ms.

This provides two levels of redundancy. The RF signal to activate the RF amplifier disconnects the tongue driver from the tongue array. The tongue array is also synchronized with the pulse sequence to avoid periods when there is both RF excitation and a connected array. The pulse sequence control signals are flexible and can be coded to synchronize or randomize more elaborate stimulation periods with the imaging sequence.

(a) Scanning Protocol. Scanning is performed on a clinical 1.5 T GE Signa Horizon magnet equipped with gradients for whole-body EPI. The subject's head is positioned within a radio-frequency quadrature birdcage coil with foam padding to provide comfort and to minimize head movements. Aircraft-type earphones with additional foam padding are placed in the external auditory canals to reduce the subject's exposure to ambient scanner noise and to provide auditory communication. Preliminary anatomical scans include a sagittal localizer, followed by a 3D spoiled-GRASS(SPGR) whole-brain volume (21/7 ms TR/TE; 40 degree flip angle; 24 cm FOV; 256×256 matrix; 124 contiguous axial slices including vertex through cerebellum; and 1.2 mm slice thickness). A series of 22 coronal Ti-weighted spin-echo images (500/8 ms TR/TE; 24 cm FOV; 256×192 matrix; 6 mm slice thickness with 1 mm skip) from occipital pole to anterior frontal lobe is acquired. EPI fMRI scanning is acquired at the same slice locations, thickness and gap as the spin-echo coronal anatomical series. EPI parameters: single-shot acquisition, 2000/40 ms TR/TE; 85 degree flip angle; 24 cm FOV; 64×64 matrix (in-plane resolution of 3.75×3.75 mm); +/−62.5 kHz receiver bandwidth. Transmit gain and resonant frequency are also manually tuned prior to the functional scan.

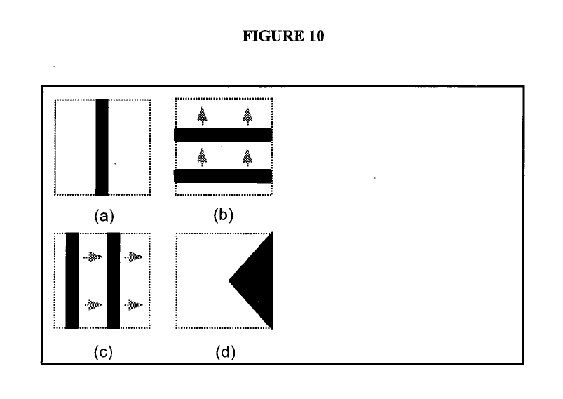

Data has been obtained outside the MRI environment demonstrating how to best present spatial and directional information on the tongue tactile display. However, during this entire process, little information about the cognitive processes are taking place in response to the tactile stimulation is known. This information is useful to improve upon the functionality of the device. Learning how the brain responds to the tactile perception aids in the training process. Knowledge of brain activity allows modifications of the device to speed up the training process and to improve learning. To visualize brain function during navigation using fMRI, a program to create 2- and 3-D virtual environments was developed and a quasi-3-D navigation task was devised through a virtual building. The subjects move through the virtual maze using a joystick. Using the navigation task as a test platform, with the appropriate tactile display interface, users perform a virtual ‘walk-through’ in real time. The users are given tactile directional cues as well as error correction cues. The error correction cues provide navigation information based on the calculated error signal derived from the users' current position and direction vector and the prescribed trajectory between any two nodes along the desire path in the maze. For example, a single line sweeping to the right is very readily perceived, and indicates that the user should “step” to the right. By contrast, an arrow on the right hand side of the tactile display instructs the user to rotate their viewpoint until it is again parallel with the desired trajectory. The error tolerances for the virtual trajectory, and the sensitivity of the controls are programmable, allowing the novice user to get a ‘feel’ for the task and learn the navigation cues, whereas the experienced user would want to train with a tighter set of spatial constraints. A sample of the cues is shown in FIG. 10. If the subject is “on course” and should proceed in their current direction, they sense a single, slowly pulsating line on the ET tongue array as shown in FIG. 10A. If they need to rotate up, they sense 2 distinct lines moving along the array as indicated in FIG. 10B. If a rotation to the right is required, they sense 2 lines moving toward the right (FIG. 10C). A right translation is indicated by a pulsating arrow pointing to the right (FIG. 10D).

During the development of the navigation/orientation icon sets, it was also considered how to integrate “Alert” information to the user to get their attention if they stray from the path in the maze. In the normal Navigation/Orientation Mode, the display intensity level is set at the users preferred or “Comfortable” range. In “Alert” Mode the stimulus intensity is automatically set to the maximum tolerable level (which is above the maximum level of the “Comfortable” range), and pulses at 5-15 Hz. to immediately attract the user's attention and action. Once the subject returns to the correct path, the ET stimulation switchs back to the pattern shown in FIG. 5a. The mode and event sequence as indicated in Table 6 was developed.

TABLE 6
ET mode and corresponding tactile icons.
Comments give information about icon meaning.

| Mode | Tactile Icon | Comments |
|---|---|---|
| Navigation [N] | Moving & Flashing | Tactile display gives specific |
| | Arrows or Bars | directional cues for maintaining |
| | [See FIG. 10] | course on desired trajectory. |
| Orientation [O] | Moving & Flashing | Tactile display gives specific |
| | Arrows or Bars | orientation feedback on present |
| | [See FIG. 10] | body orientation in space. |
| Alert [A!] | Flashing “X” or “Box” | Imminent environmental or |
| | Flashing diagonal line, | physiological hazard. |
| | (or other patterns to | |
| | be defined). | |

Both sighted (blindfolded) and blind subjects (early and late blind) are trained to navigate the maze while outside the MRI environment. Once they are able to navigate the maze successfully within a 10-minute period of time, they are moved on to fMRI analysis.

The fMRI paradigm is patterned after an fMRI study of virtual navigation by Jokeit et al (Jokeit et al. 2001). The paradigm comprises 10, 30s activation blocks and 10, 30s control blocks. Each block is introduced by spoken commands. During the activation block, the subjects is asked to navigate through the maze by moving the joystick in the appropriate direction using the tactile cues learned in the training session. After 30s, their route is interrupted by the control task which consists of covertly counting odd numbers starting from 21. After the rest period, the subjects continue their progress through the maze. EPI scanning is continuous throughout the task with acquisition parameters described above.

fMRI data analysis. Image analysis includes a priori hypothesis testing as well as statistical parametric mapping, on a voxel-by-voxel basis, using a general linear model approach (e.g. Friston, Holmes & Worsley 1995). fMRI analysis using SPM99 and related methods involve: (1) spatial normalization of all data to Talairach atlas space (Talairach & Tournoux 1988), (2) spatial realignment to remove any motion-related artifacts with correction for spin excitation history, (3) temporal smoothing using convolution with a Gaussian kernel to reduce noise, (4) spatial smoothing to a full width half maximum of approximately 5 mm and (5) optimal removal of signals correlated with background respiration and heart rate. Analysis of activation on an individual or group basis is obtained using a variety of linear models including cross-correlation to a reference function and factorial and parametric designs. This method is used to generate statistical images of hypothesis tests. Additionally, a ramp function is partialed out during the cross-correlation to remove any linear drifts during a study. Additional signal processing with high and low pass filters to remove any residual systematic artifacts that can be modeled may be used. The reference function for hypothesis testing in the studies will match the timing pattern of the event stimulation sequences. The output of the fitted functions provides statistical parametric maps (SPM's) for Student's-t, relative amplitude, and signal-to-noise ratio. Pixels with a t-statistic exceeding a threshold value of p<0.001 are mapped onto the anatomic images.

The brain imaging studies allow one to make two very fundamental contributions: (1) gain valuable information about brain plasticity and function in blind vs. sighted individuals or other application of the system of the present invention; and (2) use of fMRI to guide future development of the device to optimize training and learning.

### Example 16

Tongue Mapping

The present invention provides methods for mapping the tongue to assist in optimizing information transfer through the tongue. For any particular application, the location and amount of signal provided by electrodes is optimized. Understanding variations allows normalization of signal to transmit the intended patterns with the intended intensity. In some embodiments, weaker areas of the tongue are utilized for simpler “detection” type applications, while stronger areas are used in application that require “resolution.” Thus, when a multisensory signal is provided, optimal position of the different signals may be selected.

### Tongue Mapping Experiment Procedure

### Materials:

1 Mouth guard
1 Plastic sheet
1 Hole punch
1 Sharpie marker
2 Pull-tabs
Scissors
Warm water

### Procedure

1. a. Fit Mouth Guard
Heat water in microwave (about 4-5 minutes)
Submerge mouth guard and hold until sticky and soft
Insert softened guard into the top of the participant's mouth and have them bite down until a comfortable fit is established
Remove air between guard and teeth by sucking the air out
Close mouth around guard
Mold top teeth and roof of mouth into mouthpiece
Bite down to get an impression of teeth

b. Make Plastic Piece
Place bottom of guard on plastic sheet
Trace around guard with a Sharpie (hold marker perpendicular to the sheet to avoid getting marker on the guard)
Cut this shape out of the plastic sheet
Invert the guard so that the bottom is facing upwards and place the plastic piece on the bottom of the guard
Trim the plastic piece and round the edges as necessary to achieve a smooth shape that will fit the guard and not jut into the participant's mouth

c. Prepare Guard to Attach Plastic Piece
Punch a hole in the front outermost ridge of the last molar on both sides of the guard
Punch a hole in the side adjacent (90°) to each of the existing holes
Align the plastic with the guard and mark the locations of the holes on the sheet with a Sharpie
Punch out the holes in the plastic

d. Attach Plastic Piece to guard
Insert a pull-tab into the left side hole with the notched (rough) side facing the bottom of the guard
Pull the tab through the left molar hole of the guard and then through the plastic
Close the tab by inserting its end into the box portion of the tab
Secure and tighten
Repeat this procedure on the right side so that the plastic is secure and flat on the bottom of the guard
Clip excess parts of the tabs as necessary
Sand the ends to ensure a comfortable fit with no sharp protrusions
Test the device in the participant's mouth and make any further adjustments, if needed
   2. Preparing guard for trials
Superimpose the right strip on the left strip so that the left strip is the upper most part of the array. The upper portion of the array will represent A and B on the display while the lower portion represents areas C and D.
Align array end even with the anterior portion of the last molar imprint
Use double sided tape to attach the array to the plastic
Place guard and array in participant's mouth

### 3. Trials (Minimum Threshold)

Open “TDU Tongue Mapping Experiment” program
Set for remote code
Set for 115 kband communication rate with PC
Always set min. threshold channel to “3”
Always choose “COM 3” in Poll Ports
Begin with 1×1 granularity, sampling a first block of electrodes
Check voltage to verify connection by rotating knob and observing change in voltage value
Set knob so voltage reads 0
Save file
Set file name to include initials, granularity (i.e. 1×1), and block number e.g. ab1×1−1
Hide the display from the participant so they cannot see where the array is activated
Run 1×1 block 1 at minimum threshold only
When block 1 is completed, proceed to block 2—keep all parameters constant and check voltage to verify connection
Save block 2 file as done with block 1, but input new block number in file name
Repeat for 1×1 blocks 2 and 3, doing minimum thresholds only
Collect data for all 3 blocks of 2×2 and 3×3 at minimum thresholds only
There should be a total of 9 files at the end of this testing
Make sure all files are saved in “tests” folder and backup on diskette

### 4. Trials (Maximum Threshold)

Repeat set up procedure as laid out above in “minimum threshold”
Begin with 1×1 block 1
Set file name with initials, granularity, block number, followed by “max” e.g. ab1×1max
Hide the display from the participant
Run the 1×1 blocks at maximum threshold only
Save block 2 as done for block 1, but rename the file to indicate block 2
Repeat for 1×1 blocks 2 and 3, doing maximum thresholds only
Collect data for all 3 blocks of 2×2 and 3×3 at maximum thresholds only
There should be a total of 9 “max” files at the end of this testing
There should be a total of 18 total files for the participant, including minimums and maximums

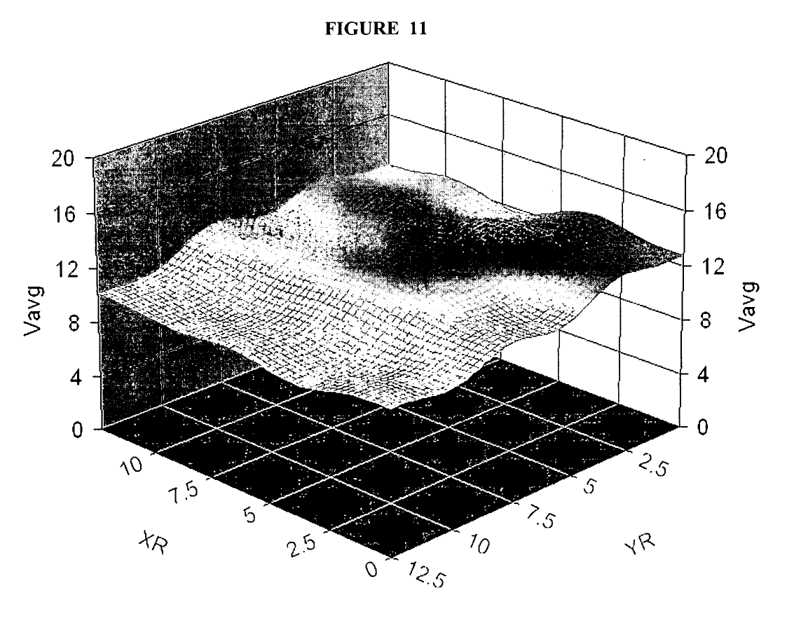

FIGS. 11-14 show data collected using such methods.

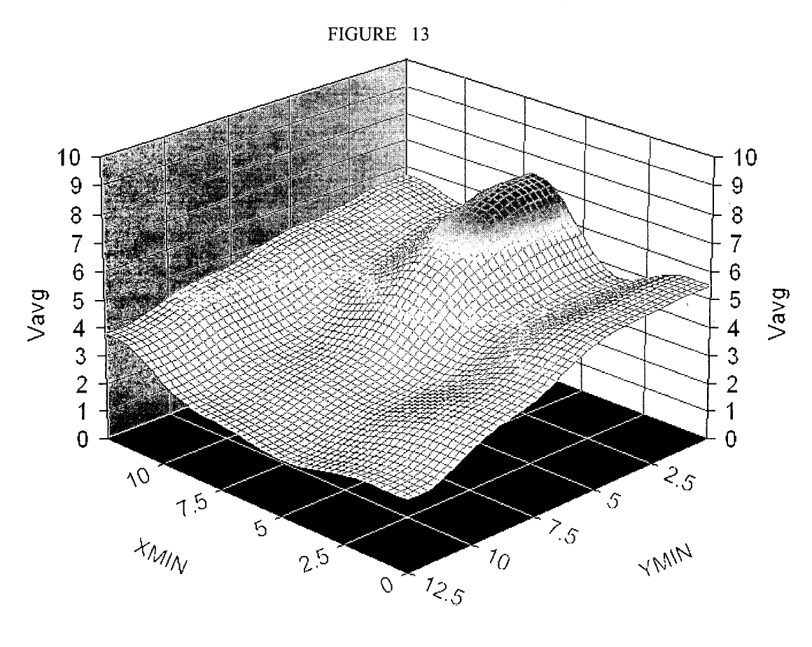

### 1×1 min (FIG. 13)

The figure shows the minimum threshold voltage to detect electrotactile stimulation on randomized parts of the tongue. The stimulus was a 1×1 electrode contiguous pattern on a 12×12 array of electrodes. The function is slightly asymmetric, with a slightly lower average voltage required to stimulate the left side of the tongue towards the front. Thus, this left anterior area of the tongue is most sensitive to electrotactile stimulation. The anterior medial portion of the tongue is generally more sensitive to stimulation than the rest of the tongue. In contrast, the posterior medial section of the tongue had the highest threshold. Therefore, the posterior medial section of the tongue is least sensitive to stimulation.

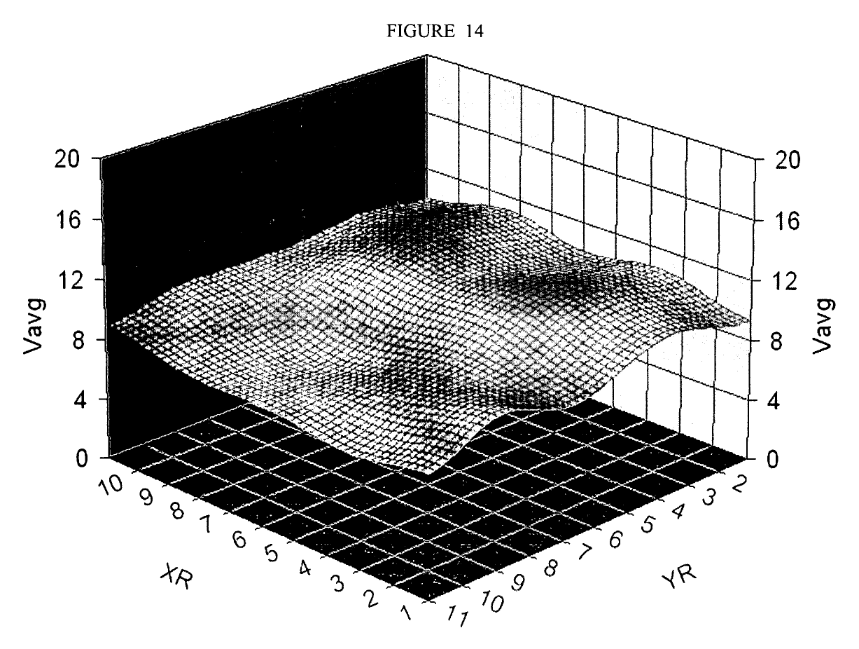

2×2 min (FIG. 14)

The figure shows the minimum threshold voltage necessary to detect electrotactile stimulation on various portions of the tongue. The stimulus was a random pattern of 2×2 square of electrodes on a total array of 12×12 electrodes. Again, the function is slightly skewed to the anterior left side of the tongue. This finding is consistent with the 1×1 minimum figure. The general shape of the curve is also similar to the 1×1 minimum function. The same phenomena are seen in the 2×2 mapping as were observed in the 1×1 map. The anterior medial section of the tongue is most sensitive, requiring the least voltage to sense electrode activation. The medial posterior area of the tongue showed the least sensitivity.

### Comparison of Mins

It is worthwhile to note that the 2×2 minimum curve had a lower overall threshold when compared with the 1×1 minimum curve. The 2×2 minimum function also appears to be flatter and more uniform than the 1×1 minimum. The lower threshold in the 2×2 function could be a result of the larger area activated on the tongue. By increasing the area activated, the stimulus can be felt sooner due to more tongue surface covered and more nerves firing. This is analogous to a pinprick versus the eraser of a pencil on your finger. Covering a larger stimulus area will activate more nerves sooner, causing the voltage to be lower for the 2×2 map.

The uniformity of the 2×2 curve may also be explained by this phenomenon, as the increased stimulus surface area led to less specificity. The 1×1 curve has more contouring because it was more specific to activating certain areas of the tongue and causing certain nerves to fire. On the other hand, the 2×2 square stimulus may have involved multiple nerves that may have been excitatory or inhibitory.

Additionally, there seems to be a diagonal that runs along the tongue from the anterior right side to the posterior left side. It is along this diagonal that the transition from high sensitivity to low sensitivity occurs. Possibly this is caused by the anatomical arrangement of the nerves in the tongue, as the hypoglossal nerve runs in the same direction.

Both the 1×1 and 2×2 curves show decreased sensitivity (represented by higher voltages in the figures) at the sides of the tongue. This can be explained by the spread of nerves in the center of the tongue. Because the nerves are more spread out, there is a higher nerve density at the middle of the tongue when compared with the sides.

### 1×1 Range (FIG. 11)

The 1×1 range was determined by finding the difference between the minimum and maximum voltages for the 1×1 array mapping. The range was slightly higher on the left side of the tongue and also in the posterior region. This may indicate that the anterior and/or right side of the tongue is less variable than the left side and/or the posterior region.

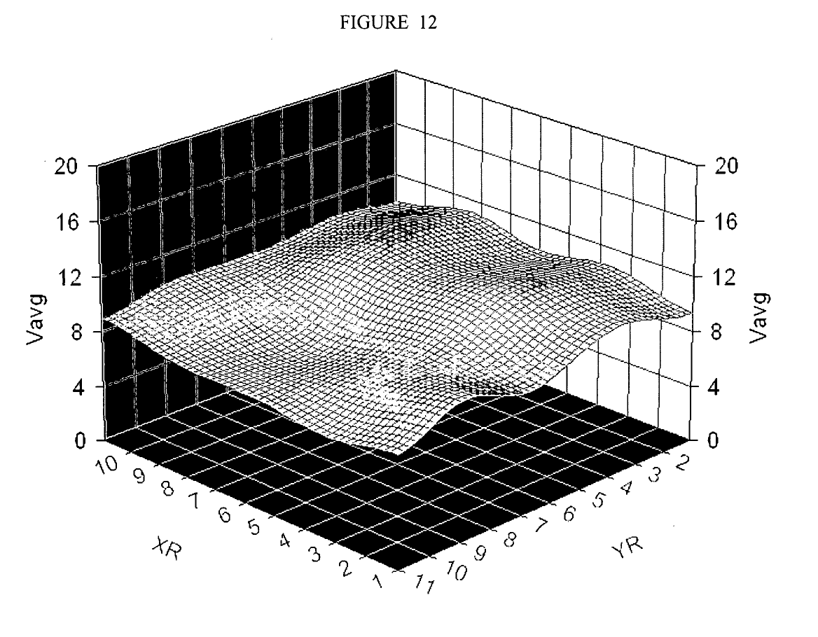

### 2×2 Range (FIG. 12)

The 2×2 range was found as explained above. The 2×2 range figure appears to be flatter than the 1×1 range figure. This can be explained by the loss of specificity when using a larger stimulus area. When the stimulus covers a larger area, less detail can be detected, causing the map to be less particular and more uniform.

### Range Comparison

The ranges were based on the difference between the maximum and the minimum threshold voltages for each array (1×1, 2×2). The ranges were fairly constant among the subjects and both curves (1×1 and 2×2) appear to be similar. The range was slightly higher for the 1×1 stimulus when compared to the 2×2 stimulus for reasons previously explained. More variability is expected for a more specific stimulus that affects a smaller surface area of the tongue.

The shapes of the curves are also similar in their characteristics. Both functions have noticeable “bumps” in the posterior section of the tongue. These bumps indicate that a broader range in threshold levels at the posterior section of the tongue.

The range figures show that there is a small variation in tongue maps across the subjects tested.

Experiments conducted during the development of the present invention identified that the anterior portion of the tongue is an optimal location for providing video information for vision substitution or enhancement.

### Example 17

### Tongue-Based 2-Way Communication for Command & Control

The present invention provides a self-contained intraoral device that permits eyes, ears, and hands-free 2-way communications. Preferably, the device is small, silent, and unobtrusive, yet provides simple command, control and navigation information to the user thereby augmenting their situational awareness while not obstructing or impeding input from the other senses. The device preferably contains a small electrotactile array to present patterned stimulation on the tongue that is automatically or voluntarily switched into a ‘command’ for sending information, a power supply and driver circuitry for these subsystems, and an RF transceiver for wireless transmission.

Human/computer interfaces are most often associated with keyboard/mouse inputs and visual feedback by means of a display. However, in many scenarios this mode may not be optimal. Many scenarios exist where an individual's visual and auditory fields and finger/hand are occupied with other demands. For such scenarios the development of unconventional interfaces is needed.

Tactile displays have been designed for the fingertip and other body locations of relatively larger area. However, few researchers have targeted the oral cavity for housing a tactile interface despite its high sensitivity, principally because the oral cavity is not easily accessible and has an irregular inner surface. Nevertheless, an oral tactile interface provides an innovative approach for information transmission or human-machine interaction by taking advantage of the high sensitivity of the oral structures, with hidden, silent, and hand-free operation. Potential applications may be found in assistance for quadriplegics, navigation guidance for the blind and scuba divers, or personal communication in mobile environments.

In many military relevant situations, it would be advantageous to utilize the tactile sensory channel for communication. While the tactile sensory channel has a limited bandwidth compared to the visual and auditory channels, the tactile channel does offer some potential advantages. The tactile channel is “directly wired” into a spatio-temporal representation on the neocortex of the brain, and as such is less susceptible to disorientation. In addition, the use of the tactile channel reduces the incidence of information overload on the visual and auditory channels and frees those channels to concentrate on more demanding and life-threatening inputs. Finally, the use of the tactile channel allows communication even in conditions where visual and audio silence is required. When combined with intelligent information filters and appropriate personnel training, even a low-bandwidth channel (the tactile channel) is effective in decision making and command & control.

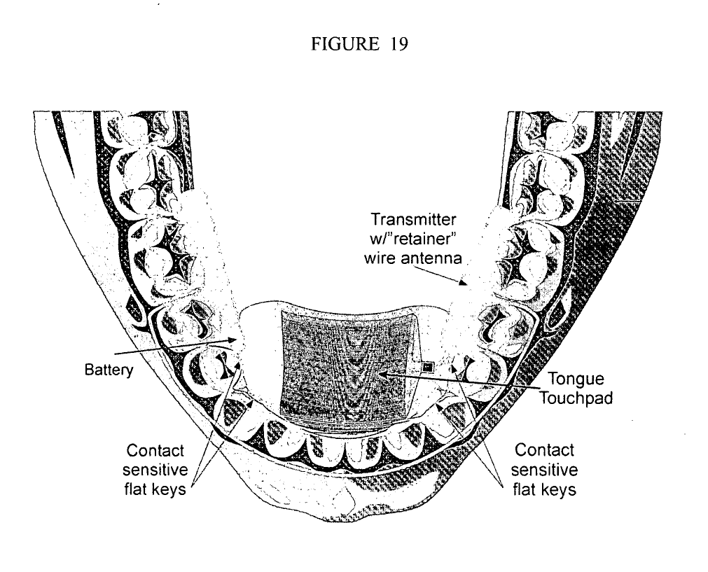

The tongue is capable of very precise, complicated, and elaborate movements. Devices having a switching device can interact with the tongue and provide an alternative method for communication (see e.g., FIG. 19). Tongue operated devices can provide an alternate computer input method for those who are unable to use their hands or need additional input methods besides hands during a specific operation, such as scuba divers and other military personnel. Several companies have recognized the potential merits of tongue-based devices, such as NewAbilities Systems' tongue touch keypad (TTK) (Mountain View, Calif.), and IBM's TonguePoint prototype. Though, innovative, none of these devices are easy to use, and consequently have not achieved commercial success.

Exemplary applications of the system are described briefly below.

Dismounted Soldier Scenario

At the platoon/squad echelon, the dismounted soldier is the primary personnel type. It is imperative for the dismounted soldier to continually scan the immediate surrounding using both visual and auditory sensory channels. Traditional communication visually (hand gestures) or audibly (speaking/shouting) may degrade the soldier's ability to see and hear the enemy. In addition, it is often necessary to maintain auditory silence during maneuvers. Because of the limited bandwidth of the tactile sensory channel the “vocabulary” used via the tactile channel must be limited. Because the dismounted soldier has a fairly narrow relevant area of concern, a few key phrases/commands may be sufficient. The soldier needs to convey to his platoon leader information regarding his physical condition (I'm wounded), location (rally point), target information (enemy sighted), equipment status (need ammunition), etc. Conversely, the platoon/squad leader needs to communicate commands to the soldier (retreat, speed up, rally point, hold position, etc.). Such a limited vocabulary (as well as more complex vocabularies) can be effectively transmitted using the tactile sensory channel.

Command and Control Personnel Scenario

The cocktail party analogy is often used to describe the situation in a command center. It is a crowded, noisy place filled with a range of personnel with different information needs. Often visual and auditory alerts are ineffective and inconvenient. For example, if one person wants to get a subset of the command center personnel to converge their attention to one display area they are currently forced to verbally attempt to redirect each individuals attention to the display of interest or physically go to each person and tap them on the shoulder to get their attention. The confined space in most command posts do not allow for easy movement and the visual means of communication is already overloaded for many personnel. In this environment a silent (auditory and visual) tactile low bandwidth communication system has great use for attention getting, cueing and simple messages. The use of tactile stimulators as “virtual taps” greatly facilitates the coordination within a command center without adding to the auditory and visual noise of a command center. With a single input, a commander can simultaneously “tap” a selected subgroup within the command center. Similar scenarios in video conferencing and virtual sandboxes can be provided where the use of a “virtual tap” is used to redirect an individuals attention or to transmit simple messages.

Navigation Scenario

To facilitate navigation for dismounted soldiers and during underwater scuba operations, geospatial cues are required. With the advent of low cost Global Positioning Systems (GPS), precise absolute position information is available. However, existing methods for communicating navigational information to persons are limited to visual cues (hand signals) and auditory directions. It is important for the auditory and visual channels to remain clear as they provide important situational cues in battlefield scenarios. The tactile channel is ideal for providing geospatial cues. The brain easily adapts to associate semantic content in tactile cues. In some embodiments, the invention provides a tactile interface in the mouth which provides geospatial relevant cues to a subject while underwater. Stimulators in contact with the roof of the mouth provide simple directional cues. An impulse to the back of the mouth might signal stop or slow down depending on its perceived intensity or frequency. Likewise, stimulus to the sides would mean turn and stimulus to the front speed up. Similar cues would be advantageous for extraction operations where silent communication is critical. The incorporation of sensors would also provide an output channel and allow soldiers to relay information silently to one another within a squad for example.

Other Scenarios

Other tasks require continual tactile manipulation (inspection, mixing chemicals, operating equipment). In these situations, it would be advantageous for the subject to be able to adjust weapons parameters, for example, without interrupting the manipulative task. Often relatively high noise levels make speech recognition communication schemes difficult. Similar scenarios, for example, are found in airplane cockpits, where the pilot is overloaded with visual cues/information on a variety of displays and must manipulate a large number of controls. A wide variety of other scenarios exist in which the human operator's interaction with the machine is limited by the other demands on visual and hand/finger manipulations. The use of a mouth-based tactile interface allows the flow of critical communication to continue without interrupting manual manipulation skills thereby increasing task performance.

In addition, an oral interface has many applications in the civilian world (including manufacturing, persons with disabilities, etc.).

An interface with both input and output capability through the oral tactile channel has been developed and tested. A demonstration of two-way tactile communication has been performed to show the application of the tactile interface for navigational guidance. The oral tactile interface is built into a mouthpiece that can be worn in the roof of the mouth. A microfabricated flexible tactor array is mounted on top of the mouthpiece so that it is in contact with the palate, while the tongue operated switch array (TOSA) is located on the bottom side of the mouthpiece. An interfacing system has been developed to control both the tactor array and the tongue touch keypad. The system is programmed to simulate the scenario of navigation guidance with simple geospatial cues. Initial device characterization and system psychophysical studies demonstrated feasibility of an all oral, all-tactile communication device. Subsequent modification and psychophysical analysis of the TOSA configuration yielded superior task performance, improved device reliability, and reduced operator fatigue and errors. Such a signal output system can be combined with a tongue-base tactile information input system to provide two-way communication.

In preferred embodiments, the system operates in one of two modes: command or display. Specifically, when the tongue is making complete (or nearly complete) contact with the electrotactile array, the circuitry detects that there is continuity across the entire array and locks into display mode. When the user removes the tongue from the array, or the sensed average contact area drops below a predetermined threshold (e.g. 25%), the system automatically switches to ‘command’ mode and remains in this state until either all contact is lost or the sensed average contact area is greater than 50%. When in the ‘command’ mode, the sensing circuitry detects all electrodes that are making contact with the tongue by performing a simple, momentary, sub-sensation threshold continuity check. Firmware in the system then calculates the net area that is in contact, and then the centroid of that area. The locus of this point on the display then serves as the command input to be communicated to central command or to other personnel in the area. The commanded signal can then be used by the recipient as either explicit position and orientation information or can be encoded in an iconic form that gives the equivalent and other information.

In between pulses and bursts, the system presently switches all inactive electrodes to ground so that the entire array acts as a distributed ground plane. For the command and control system, there is an addition of a 3^{rd} state, one that allows the injection of a sub-threshold stimulus for the ‘continuity check’ function. These continuity pulses are periodic and synchronous (e.g. every 4^{th} burst) since their only purpose is to poll the array to determine how much of the tongue is making contact with it at any given time. This stimulus, however, should be phase-shifted so that there is no chance that it will occur when the electrodes proximal to an active one need to be switched to the ground state to localize the current and the resultant sensation. Thus the continuity polling takes place continuously in the background so that the system calculates the location of the tongue and instantaneously switches modes when the appropriate state conditions are met. This alleviates the need for manual mode switching unless requested by the user by completely removing the tongue from the array.

In command mode, the device may be configured to send out physiological information for monitoring in-field personnel (or patients, children, etc.). Such information could include salivary glucose levels, hydration, APR's, PCO₂, etc.

### Example 18

### Stimulator Implant

The present invention provides tactile input systems that reduce or eliminate many of the problems encountered in prior systems by providing stimulators that are implanted beneath the epidermis or otherwise positioned under the skin or other tissues. One advantage of such a system is the ability to substantially reduce size of the stimulators because their output is closer to the nerves of the skin (or other tissue) and is no longer “muffled.” Such size reduction allows higher stimulator densities to be achieved. Additionally, interconnectivity problems, and issues inherent in providing input signals from an external camera, microphone, or other input device to an internal/subdermal stimulator (i.e., the need to provide leads extending below the skin), may be avoided by providing one or more transmitters outside the body, and preferably adjacent the area of the skin where the stimulator(s) are embedded, which wirelessly provide the input signals to the embedded stimulator(s).

A description of several exemplary versions of the implanted system follows. In preferred embodiments, the implantable stimulator(s) are implanted in the dermis, the skin layer below the epidermis (the outer layer of skin which is constantly replaced) and above the subcutaneous layer (the layer of cells, primarily fat cells, above the muscles and bones, also sometimes referred to as the hypodermis). Most tactile nerve cells are situated in the dermis, though some are also located in the subcutaneous layer. Therefore, by situating a stimulator in the dermis, the stimulator is not subject to the insulating effect of the epidermis, and more direct input to the tactile nerve cells is possible. Perceptible tactile mechanical (motion) inputs may result from stimulator motion on the order of as little as 1 micrometer, whereas above-the-skin tactile input systems require significantly greater inputs to be perceivable (with sensitivity also depending where on the body the system is located). If the stimulators use electrical stimulation in addition to or instead of mechanical (e.g., motion) stimulation, a problem encountered with prior electrotactile systems—that of maintaining adequate conductivity is also reduced, since the tissue path between the stimulators and the tactile nerve cells is short and generally conductive. Additionally, so long as a stimulators is appropriately encased in a biocompatible material, expulsion of the stimulator from the skin is unlikely. In this respect, it is noted that when tattoos are applied to skin, ink particles (sized on the micrometer scale) are driven about ⅛ inch into the skin (more specifically the dermis), where they remain for many years (and are visible through the translucent, and oven nearly transparent, epidermis). In contrast, implantation in the epidermis would cause eventual expulsion, since the epidermis is constantly replaced. However, expulsion may be desired for certain application.

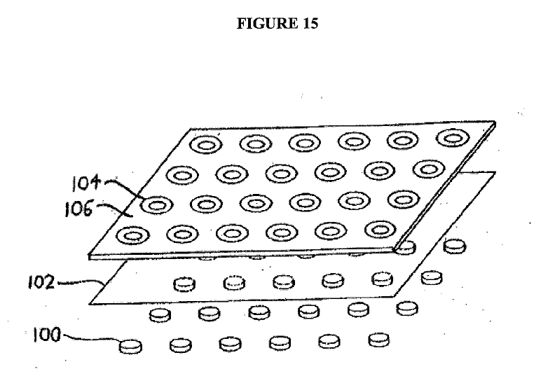

A first exemplary version of the device, as depicted in FIG. 15, involves the implantation of one or more stimulators 100 formed of magnetic material in an array below the skin (with the external surface of the epidermis being depicted by the surface 102), and with the array extending across the area which is to receive the tactile stimulation (e.g., on the abdomen, back, thigh, or other area). Several transmitters 104 are then fixed in an array by connecting web 106 made of fabric or some other flexible material capable of closely fitting above the skin 102 in contour-fitting fashion (with the web 106 being shown above the surface of the skin 102 in FIG. 15 for sake of clarity). The transmitters 104 are each capable of emitting a signal (e.g., a magnetic field) which, when emitted, causes its adjacent embedded stimulator 100 to move. The transmitters 104 may simply take the form of small coils, or may take more complex forms, e.g., forms resembling read/write heads on standard magnetic media data recorders, which are capable of emitting highly focused magnetic beams sufficiently far below the surface 102 to cause the stimulators 100 to move. Thus, when an input signal is applied to a transmitter 104, it is transformed into a signal causing the motion of a corresponding stimulator 100, which is then felt by surrounding nerves and transmitted to the user's brain.

The input signals provided to the transmitters 104 may be generated from camera or microphone data which is subjected to processing (by a computer, ASIC, or other suitable processor) to convert it into desired signals for tranmission by the transmitters 104. (Neither the processor, nor the leads to the transmitters 104, are shown in FIG. 15 for sake of clarity). While the signals transmitted by the transmitters 104 could be simply binary on-off signals or gradually varying signals (in which case the user might feel the signals as a step or slow variation in pressure), it is expected that oscillating signals that cause each of the stimulators 100 to oscillate at a desired frequency and amplitude allows a user to learn to interpret more complex information inputs—for example, inputs reflecting the content of visual data, which has shape, distance, color, and other characteristics.

The stimulators 100 may take a variety of forms and sizes. As examples, in one form, they are magnetic spheres or discs, preferably on the order of 2 mm in diameter or less; in another form, they take the form of magnetic particles having a major dimension preferably sized 0.2 mm or less, and which can be implanted in much the same manner as ink particles in tattooing procedures (including injection by air pressure). The stimulators 100 may themselves be magnetized, and may be implanted so their magnetic poles interact with the fields emitted by the transmitters 104 to provide greater variation in motion amplitudes.

It should be understood that each transmitter 104 might communicate signals to more than one stimulator 100, for example, a very dense array of stimulators 100 might be used with a coarse array of transmitters 104, and with each transmitter 104 in effect communicating with a subarray of several stimulators 100. Arrays of stimulators 100 which are denser than transmitter arrays 104 are also useful for avoiding the need for very precise alignment between stimulators 100 and transmitters 104 (with such alignment being beneficial in arrays where there is one transmitter 104 per stimulator 100), since the web 106 may simply be laid generally over the implanted area and each transmitter 104 may simply send its signal to the closest stimulator(s) 100. If precise alignment is needed, one or more measures may be used to achieve such alignment. For example, a particular tactile signal pattern may be fed to the transmitters 104 as the user fits the web 106 over the stimulators 100, with the user then adjusting the web 106 until it provides a sensation indicating proper alignment; and/or certain stimulators 100 may be colored in certain ways, or the user's skin might be tattooed, to indicate where the boundaries of the web 106 should rest. (Recall that if the stimulators 100 are implanted in the dermis, they will be visible through the translucent epidermis in much the same manner as a tattoo unless they are colored in an appropriate fleshtone).

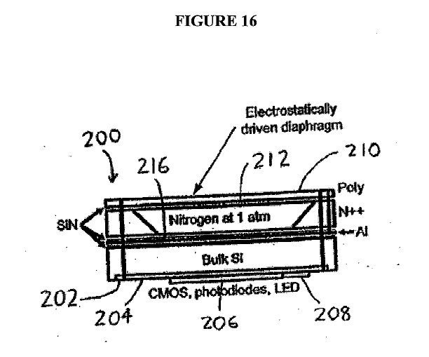

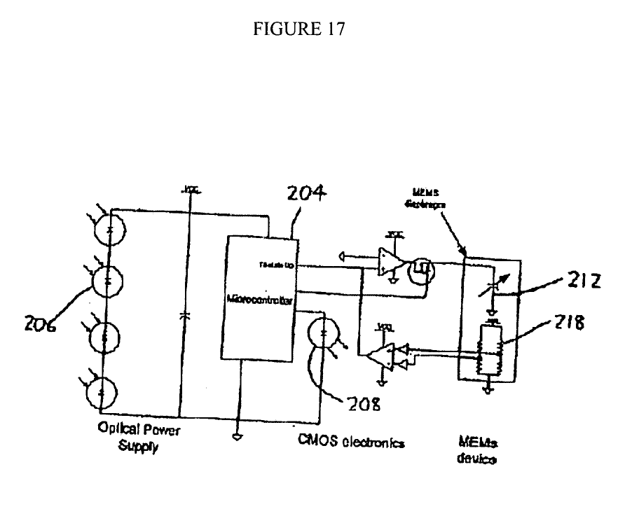

The foregoing version of the invention is “passive” in that the stimulators 100, that are effectively inert structures, are actuated to move by the transmitters 102. However, other versions of the invention wherein the stimulators include more “active” features are may be used, e.g., the stimulators may include features such as mechanical transducers that provide a motion output upon receipt of the appropriate input signal; feedback to the transmitters; onboard processors; and power sources. As in the tactile input system discussed above, these tactile input systems preferably also use wireless communications between implanted stimulators and externally-mounted transmitters. To illustrate, FIGS. 16 and 17 present a second exemplary version of the invention. Here, a stimulator 200 has an external face 202 which includes a processor 204 (e.g., a CMOS for providing logic and control functions), a photocell 206 (e.g., one or more photodiodes) for receiving a wireless (light) signal from a transmitter, and an optional LED 208 or other output device capable of providing an output signal to the transmitter(s) (not shown) in case such feedback is desired. Light send by the transmitter(s) to the photocell 206 both powers the processor 204 and conveys a light-encoded control signal for actuation of the stimulator 200. On the internal face 210 of the stimulator 200, a diaphragm 212 is situated between the dermis or subcutaneous layer and an enclosed gas chamber 214, and an actuating electrode 216 is situated across the gas chamber 214 from the diaphragm 212. Light signals transmitted by the transmitter(s), discussed in greater detail below, are received by the photocell 206, which charges a capacitor included with the processor 204, with this charge then being used to electrostatically deflect the diaphragm 212 toward or away from the actuating electrode 216 when activated by the processor 204. Since the diaphragm 212 only needs to attain peak-to-peak motion amplitude of as little as one micrometer, very little power is consumed in its motion. Piezoelectric resistors (218) (FIG. 17) situated in a Wheatstone bridge configuration on the diaphragm 212 measure the deformation of the diaphragm 212, thereby allowing feedback on its degree of displacement, and such feedback can be transmitted back to the transmitter via output device 208 if desired.

The stimulator 200 is preferably scaled such that it has a major dimension of less than 0.5 mm. With appropriate size and configuration, stimulators 200 may be implanted in the manner of a convention tattoo, with a needle (or array of spaced needles) delivering and depositing each stimulator 200 within the dermis or subcutaneous layer at the desired depth and location. Using state of the MEMS processing procedures, it is contemplated that the stimulator 200 might be constructed with a size as small as a 200 square micrometer face area (e.g., the area across the external face 202 and its internal face 210), with a depth of approximately 70 micrometers. An exemplary MEMS manufacturing process flow for the stimulator 200 is as follows:

| | Side of | |
|---|---|---|
| Step | wafer | Comment |
| 2 um CMOS process | Top | More tolerant to defects |
| Attach handling | Top | |
| wafer | | |
| Planarize (CMP) | Bottom | Thin to approximately 50 um |
| Deposit SiN | Bottom | Insulate lower electrode |
| Sputter Al | Bottom | Lower electrode |
| Lithography | Bottom | Electrode and pads for vias |
| Deposit SiN | Bottom | Insulate lower electrode |
| Deposit poly | Bottom | Approximately 150 um |
| Deposit SiN | Bottom | Mask for cavity |
| Lithography | Bottom | Pattern hole for cavity |
| Etch | — | KOH to form cavity (timed) |
| Deposit poly | Bottom | Seal cavity and strengthen diaphragm |
| Etch (RIE) | Bottom | Vias; 2 through-hole, 1 stops a lower |
| | | electrode metal |
| Fill vias | Bottom | Tungsten |
| Planarize (CMP) | Bottom | Planarize |
| Deposit Ti | Bottom | Titanium (bio-compatible) |
| Lithography | Bottom | Cover only tungsten, or do not do litho |
| | | at all if diaphragm is unaffected |
| Planarize (CMP) | Top | Remove handling wafer |
| Lithography | Top | Pattern for via to pad interconnect |
| Deposit Al | Top | Deposit via a pad interconnect |
| Lithography | Bottom | Pattern for via to pad and via to via |
| | | interconnect |
| Deposit Al | Bottom | Deposit via to pad and via to via |
| | | interconnet |

The transmitter (not shown) may take the form of a flexible electro fluorescent display (in which case it may effectively provide only a single transmitter for all stimulators 200), or it could be formed of an array of LEDs, electro fluorescent displays, or other light sources arrayed across a (preferably flexible) web, as in the transmitter array of FIG. 15. The transmitter(s) supply light to power the photocells 206 of the stimulators 200, with the light bearing encoded information (e.g., frequency and/or amplitude modulated information) which deflects the diaphragms 212 of the stimulators 200 in the desired manner. The light source(s) of the transmitter, as well as the photocells 206 of the stimulator 200, preferably operate in the visible range since photons in the visible range pass through the epidermis for efficient communication with the powering of the stimulators 200 with lower external energy demands.

With appropriate signal tailoring, it is possible to have one transmitter provide distinct communications directed to each of several separate stimulators 200. For example, if the transmitter delivers a frequency modulated signal that is received by all stimulators 200, but each stimulator only responds to a particular frequency or frequency range, each stimulator 200 may provides its own individual response to signals delivered by a single transmitter. An additional benefit of this scheme is that the aforementioned issue of precise alignment between individual transmitters and corresponding stimulators is reduced, since a single transmitter overlaying all stimulators 200 may effectively communicate with all stimulators 200 without being specifically aligned with any one of them.

The description set out above is merely of exemplary versions of the invention. It is contemplated that numerous additions and modifications can be made. As a first example, in active versions of the invention wherein an actuator is used to deliver motion output to the user, actuators other than (or in addition to) a diaphragm 212 may be used, e.g., a piezoelectric bimorph bending motor, an element formed of an electroactive polymer that changes shape when charged, or some other actuator providing the desired degree of output displacement.

As a second example, while the foregoing tactile input systems are particularly suitable for use with their stimulators imbedded below the epidermis, the stimulators could be implemented externally as well, provided the output motion of the stimulators has sufficient amplitude that it can be felt by a user. To illustrate, the stimulators might be provided on a skullcap, and might communicate with one or more transmitters provided on the interior of a helmet.

As an additional example, the foregoing versions of the invention find use with other forms of stimulation, e.g., electrical, thermal, etc., instead of (or in additional to) mechanical stimulation. Greater information is provided in some embodiments by combining multiple types of stimulation. For example, if pressure and temperature sensors are provided in a prosthetic and their output is delivered to a user via mechanical and thermal stimulators, the prosthetic may more accurately mimic the full range of feeling in the missing appendage. As another example, in a vision substitution system, mechanical inputs might deliver information related to the proximity of object (in essence delivering the “contour” of the surrounding environment), and electrical stimulation delivers information regarding color or other characteristics.

These systems may be applied to any of the range of applications described herein.

In some embodiments, the embedded components further serve aesthetic and/or entertainment purposes. Because the embedded components are, or can be designed to be, visible, they may be used to serve tattooing or cosmetic implant functions—i.e., to provide color, texture, and/or shapes under the skin with desired aesthetic features. Additional embedded components without sensory function may be added to enhance or fill out the image provided by the embedded stimulators. LED or other components can provide light to enhance the appearance of the device. For example, stimulators that are in use may be lit. Alternatively lighting patterns are provided randomly or upon cue (e.g., as a timekeeping device, upon receipt of a signal from an external device (e.g., phone)).

In some embodiments, the embedded devices are used as communication methods, much like text messaging of cell phones. Message sent via any desired method (e.g., cell phone) are perceived in the embedded devices. This allows covert communication. In some embodiments, the system is configured to receive a person-specific code in the transmitted message so that only a person with a particular stimulator array receives the code even though the message is transmitted more generally (e.g., via the airwaves). Like Internet community communication systems, groups of users can also be designated to receive the signal.

In some embodiments, the embedded stimulator is used as a covert matchmaking service. A subject has a processor that specifies: 1) criteria of others that they would seek in a relationship (e.g., friendship, romantic relationship, etc.); 2) personal criteria to transmit to others; and/or 3) a set of rules for activating or deactivating the system (e.g., for privacy). When the subject is in the physical vicinity of a match and when the match's system is transmitting a willingness to meet people, the embedded stimulator triggers an alarm and indicates the direction and location of the match. The subject receiving the signal, upon seeing the match can choose to send a reciprocal “are you interested” signal (or perhaps, as a default has been sending such a signal). The match can then choose to initiate actual contact. Because the subject does not know whether the match's system is “on” and therefore whether the match received signal, the subject's ego need not be hurt if the match does not respond.

In some embodiments, a large number of stimulators are provided all over the body. The stimulators may be used much like the tactile body suit described in Example 10.

### Example 19

### Processor Command Set

This Example describes aspects and operation of a Tactile Display Unit, or TDU, device in some embodiments of the present invention. The TDU is a wave generator in its simplest construct. Control of the TDU occurs via a ASCII based communication language. The commands that allow a computer program to communicate with the TDU are described below. Also discussed is the underlying theory behind using the TDU.

### Terminology

Tactor: a single electrode on the array.
Block: a square-shaped group of tactors referenced by the upper left and lower right tactor numbers. Block sizes range from a single tactor to all 144 tactors.
Channel: a single output from the TDU to a tactor.

### TDU Principles

Operating on 144 channels separated into 4 sectors, the TDU uses a scheme of transmitting pulses along an array to the user. An array consists of a 72-pin insulated cable that terminates in a rectangular matrix (12×6) of tactors. Merging two separate arrays provides the square matrix (12×12) formation that is used by the TDU. The 12×12 square matrix is subdivided into four sectors (6×6) denoted as A, B, C, and D. This formation is due to the specific implementation of the hardware and is of little concern to the user or even the developer. Specifically, because of workload and speed requirements, four processors work in parallel to handle the output to the arrays. As one might imagine, each processor corresponds to a sector on the arrays.

Tactor addresses are numbered from left to right, top to bottom. So, the top row of tactors has addresses 1-12 while the bottom row of tactors has addresses 133-144. Due to the numbering construct, it is important to note that the sectors do not contain a single contiguous list of addresses. Although from the standpoint of the user, this is abstracted away and only the addresses are available.

Any imaginable animated display can be presented to the user via the TDU. The TDU runs at a very high frame rate and has the ability to respond very quickly to user feedback. Beyond these properties, the system is mobile which provides an added level of flexibility.

### Analysis of a Waveform

A waveform consists of numerous parts. The most fundamental layer is the outer burst. The waveform is simply a continuous or discrete grouping of outer bursts. Each outer burst consists of a certain number of inner bursts. Within the inner bursts, there are an arbitrary number of pulses.

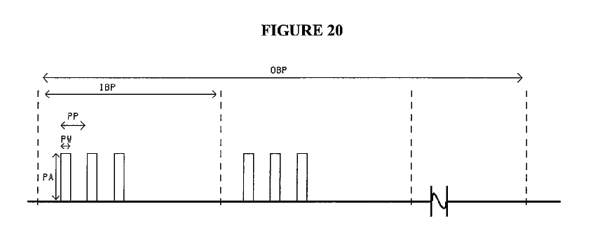

Each pulse has a certain width and height along with a specifiable distance between consecutive pulses. A sample waveform for a single channel is provided in FIG. 20. Properties of this waveform that have been previously alluded to are now discussed. The first property is the outer burst number (OBN), which specifies the number of inner bursts that reside in each outer burst. The outer burst also has a period (OBP), which is its duration. Within the inner burst are the pulses. The inner burst number (IBN) is a parameter, which specifies the number of these pulses. In FIG. 20 the IBN is three. Associated with an inner burst, there is a specifiable period known as the inner burst period (IBP). Beyond the aforementioned parameters, it is possible to specify the pulse width (PW), pulse period (PP) and pulse amplitude (PA).

For each channel the pulse width, pulse amplitude, inner block number and outer block number are specifiable. Hence, each channel is independent and can have its own specific waveform, although the period of each component of the waveform (inner burst, outer burst and inter channel periods) is constant across the entire array. The inner channel period (ICP) is a parameter that ties the channels together. This parameter specifies the time delay between channels corresponding to the beginning of each new outer burst. So, if FIG. 20 specifies channel 1 and it begins at time t=0, and the inner channel period is 100 microseconds, then channel 2 will begin stimulating at time t=100 us. Note that the inner channel period affects each block independently. Hence, for example channels 1 and 7 begin at the same time, since they occupy different blocks (A and B).

Note that valid ranges for each of these parameters are specified in Table 7.

TABLE 7
Valid Ranges for Different Parameters

| Parameter | Range | |
|---|---|---|
| OBN | 0-255 | bursts |
| IBN | 0-255 | pulses |
| OBP | 5-1275 | ms |
| IBP | 100-25500 | μs |
| ICP | 2-510 | μs |
| PP | 2-510 | μs |
| PW | 0-510 | μs |
| PA | 0-40 | Volts |

Since there is an infinite number of possible waveforms that can be generated, some concern should be taken into choosing one that is ‘comfortable’ for the user. Comfort is an important element since electrical current is being passed through a highly conductive and sensitive region.

Communicating with the TDU

One of the most important functions of the TDU is the ability to create dynamic output to the arrays. Hence, there is concern of when and how often a waveform can be updated. Updating a waveform occurs whenever a new command is issued. The change in the TDU's output occurs on the next inner burst or outer burst, whichever comes first (See FIG. 20). When implementing code to run with the TDU, there are specific considerations to be taken into account. The first, and most important is Nyquist's Law or sometimes known as the Sampling Theorem. This law states that in order to accurately reconstruct a time-varying system, samples of the system must be taken at twice the frequency of variation or faster. In the situation presented, the TDU is performing the sampling. It is expected that the most code written to communicate with the TDU will send commands to it at a regular interval. Because the TDU is sampling the incoming signals, it should be running twice as fast as the incoming signals in order to correctly model what the computer code is sending. For example, if one is sending image updates at 25 frames per second to the TDU, then the inner burst period of the TDU should be 20 ms, which corresponds to an update rate of 50 frames per second.

Another consideration when implementing code is the type of communication scheme to use. There are two basic forms of communication in a PC environment. The first can be called “serial communications” while the other form is “parallel communications.” Serial communications occurs in a format where commands are issued one at a time and a command cannot be issued until the previous one is implemented. Parallel communications allows for a multitude of commands to be issued at any given moment. They can align themselves in a queue while waiting to be processed. The TDU works in a communications mode where every command received generates a response. Write commands are followed by a single byte status response while read commands have responses of varying length. While the TDU is processing a command, it cannot receive another command. Thus, the method of communication that is the current version of the TDU utilizes is denoted as serial. In terms of Windows 98/NT/2000 programming, it is called non-overlapped I/O.

### The Command Set

The command set is ASCII in nature and each command is case sensitive. The upper case is a write command, while the lower case is a read. The length of each code varies depending on the type of addressing scheme. Some commands address individual tactors, others address a subset of the array, while other commands operate on the entire array.

After any write command is issued, the TDU issues a single byte response. One must be careful to not send another command until the response has been received. It is possible to eliminate reading the TDU responses, but one must still wait a certain amount of time before sending another command.

Below is an abbreviated list of the commands.

COMMAND: A/a Pulse Amplitude (PA) for a single tactor.
B/b Pulse Width (PW) for a single tactor.
C/c Number of Inner Bursts (Outer Burst Number) for a single tactor.
D/d Number of Pulses per Inner Burst (Inner Burst Number) for a single tactor.
E/d Pulse Amplitude for each tactor in a block F/f Pulse Width for each tactor in a block.
G/g Number of Inner Bursts (Outer Burst Number) for each tactor in a block.
H/h Number of Pulses per Inner Burst (Inner Burst Number) for each tactor in a block.
I/i Pulse Period (PP) for the entire array.
J/j Outer Burst Period (OBP) for the entire array.
K/k Inner Burst Period (IBP) for the entire array.
L/l Inter-channel Period (ICP) for the entire array.
M/m Amplitude Scaling for the entire array.
N/n Update a pre-programmed pattern.
O Start Stimulation of currently loaded pattern.
P Stop Stimulation of currently loaded pattern.
Q Display a pre-programmed pattern.
R Deliver a sequence of outer bursts.
s Current analog value for a channel
T Total comma: Pulse Amplitude, Pulse Width, Outer Burst Number and Inner Burst Number for each tactor in a block.

The command set allows for manipulation of the parameters of a single tactor, a block of tactors or the entire array.

### Using the TDU

The TDU is basically a waveform generator. There is a display panel that provides useful information, a keypad to provide input, a serial communications port, connections for the arrays, and a knob that provides amplitude scaling of the entire array.

### Connection of the Arrays

The arrays connect via the two 72-pin slots on the side of the TDU. The right pin slot is for the lower array, while the left slot is for the upper array. The upper array is defined as the one that stimulates the back of the tongue, while the lower array stimulates the front of the tongue.

### Modes of Operation

The TDU can operate in three distinct modes. These modes are denoted as “standalone,” “remote,” and “programmable.” Standalone mode allows for the TDU to display pre-programmed patterns without the intervention of a computer. Programmable mode allows the TDU to have patterns programmed into its memory. It is possible to program in 64 distinct patterns in the embodiment described in this example. The third mode, remote, allows for the TDU to be controlled from an external source (e.g., a laptop computer). Communication occurs via the serial communications ports on the TDU and the laptop.

### TDU at Startup

On startup, the TDU presents options on its LCD screen to choose the mode of operation. In most cases, remote mode should be chosen. After choosing this mode via the keypad, another set of options is displayed. These options are the for the communications speed of the serial port on the TDU. Unless there is reason in doing so, only choose the third option: the 115,200 baud rate. Note that computer code that implements any communications with the TDU sets the baud rate to the appropriate rate. Hence, no intervention on the configuration of the laptop's communications port is required.

At this point, the TDU is ready to operate remotely and should display the message ‘Status: Remote’. Programs that interact with the TDU generally need to be notified of the status of the TDU. Usually, there is a menu option in a computer program to allow for initialization of the TDU. At the point when the TDU displays the ‘Status: Remote’ message, it is allowable to proceed with remote initialization. After the computer code initializes the TDU, the message on the LCD panel should change to read ‘Stimulation Pattern Active.’ At this point output to the arrays is occurring, although the computer code may have initialized the output to be of zero potential, which causes no apparent stimulation from the arrays.

### Resetting the TDU

It is possible to access the startup menu again by pressing the menu key on the keypad. This is effectively a soft reset of the TDU. A hard reset occurs by turning the TDU off and then on again.

### Selecting Pre-Programmed Patterns

As mentioned previously, the TDU has the ability to display pre-programmed patterns via its standalone mode. Once this mode is selected, all that is required to initiate stimulation is to choose a pattern number via the keypad and press the ‘Enter’ key. If no pattern was programmed into the selected pattern number address, then there will be no stimulation. Also, the TDU will issue a message stating ‘No Pre-programmed Pattern.’ If the selected pattern does exist in memory, the TDU issues the message ‘Pre-programmed pattern #x’, where x is the pattern number chosen.

In preferred embodiments, the TDU is battery powered for portability and can operate for several hours before the internal NiCd batteries need recharging. The TDU can display one of 53 pre-programmed, non-moving patterns in a stand-alone mode; these patterns can be updated using a simple point-and-click pattern editor (Win95/98) which is supplied with the TDU. Alternatively, the TDU can be controlled by an external computer via RS-232 serial link. All of the stimulation waveforms can be controlled in this way; the entire array can be updated up to 55 times per second.

Stand Alone Mode Operation
1. Turn on power and press ‘1’ key to select Stand Alone mode, or wait 10 seconds and this mode will be entered automatically.
2. Turn intensity knob on side panel fully counterclockwise. Operation cannot continue until this is done.
3. Select a pattern (1-53) using the 0-9 numbers or the up/down arrow keys. A brief pattern description will appear on the display. If no pattern is stored for a particular number, ‘NOT INITIALIZED’ will appear on the display and the stimulation cannot be turned on.
4. Press ‘Start’ key to turn on stimulation.
5. Use the intensity knob to control stimulation intensity (voltage). Note that individuals have varying requirements for comfortable stimulation.
6. While stimulation is on, the pattern may be changed by using the number or arrow keys. If an uninitialized pattern is selected, the previous pattern will continue to be displayed.
7. Use the ‘Stop’ key to turn off the stimulation.
8. Use the ‘Menu’ key to exit Stand Alone mode.

Remote Mode Operation
1. Make sure TDU serial port 1 (next to power switch) is connected to the external computer using a “straight-through” serial cable.
2. Turn on power and press ‘2’ key within 10 seconds to select Remote mode.
3. Turn intensity knob on side panel fully counterclockwise. Operation cannot continue until this is done.
4. Press ‘1’, ‘2’, or ‘3’ key to select serial port data rate of 9.6, 19.2, or 115.2 kbps to match the external computer data rate (determined by software used to control the TDU).
5. The TDU can now be controlled by command from the external computer. Note that the pattern number, ‘Start’, and ‘Stop’ keys will not work in Remote Mode. The intensity knob may or may not function according to the commands from the external computer.
6. See the “TDU Command Language/Protocol” document for programming information.
7. Press the ‘Menu’ key to exit Remote Mode.

Update Pattern Mode Operation
1. Make sure TDU serial port 1 (next to power switch) is connected to the external computer using a “straight-through” serial cable.
2. Turn on power and press ‘3’ key within 10 seconds to select Update Pattern mode.
3. Press ‘1’, ‘2’, or ‘3’ key to select serial port data rate of 9.6, 19.2, or 115.2 kbps to match the external computer data rate (determined by software used to control the TDU).
4. Use the TDU Editor program to create and edit TDU patterns.
5. Press the ‘Menu’ key to exit Update Pattern mode.

The waveform parameters in some embodiments of the present invention are as follows:

| | | | |
|---|---|---|---|
| Abbr. | Name | Range (resolution) | Definition |

Parameters controllable tactor-by-tactor

| | | | |
|---|---|---|---|
| PA | Pulse amplitude | 0-40 (0.157) V | Pulse amplitude |
| PW | Pulse Width | 0-510 (2) μs | Width of individual pulse |
| IBN | Inner Burst Number | 0-255 (1) pulses | Number of pulses per inner burst |
| OBN | Outer Burst Number | 0-255 (1) bursts | Number of inner bursts per outer burst |

Array-wide parameters

| | | | |
|---|---|---|---|
| PP | Pulse Period | 2-510 (2) μs | Time between onset of pulses in one channel |
| IBP | Inner Burst Period | 100-25,500 (100) μs | Time between onset of inner bursts |
| OBP | Outer Burst Period | 5-1,275 (5) ms | Time between onset of outer bursts |
| ICP | Inter-Channel Period | 2-510 (2) μs | Time btw onset of adjacent chan inner bursts |
| SQN | Sequence Number | 0-255 (1) bursts | Number of outer bursts in sequence |
| PAS | Pulse amplitude scale | 0-100 (0.392) % | Pulse amplitude scale (Actual pulse output |
| | | | amplitude is PA × PAS.) |

The pulse parameter ranges shown above are intentionally wide so that the TDU may be used for research purposes. Not all parameter combinations are valid or useful for stimulation. The TDU will not attempt to deliver invalid waveforms.

Note also that some parameter values become meaningless under certain conditions. For example, IBP has no meaning when OBN=1, and PP has no meaning when IBN=1. Also, some zero parameter values will result in no stimulation; this is the case for PW, IBN, OBN, PA.

PA, PW, IBN and OBN are individually controllable tactor by tactor and are updated at the beginning of each outer burst sequence. PAS, ICP, PP, IBP, and OBP control the entire array. PAS is optionally assignable to the side panel intensity control.

All burst sequences are completed before changing any parameter values. Outer bursts are normally delivered continuously, but provision is made for delivering a fixed number of outer bursts, after which the stimulation is turned off automatically. The TDU will respond to a stimulation off command during delivery of a fixed number of bursts.

A typical, or baseline, set of stimulation parameters for comfortable stimulation is:
PW 25 μs
PP N/A
IBP 5 ms
OBP 20 ms
ICP 138.9 or 138 μs
IBN 1 pulse
OBN 3 pulses
PA 10V
PAS 100%

### Controls

1. Power switch
2. Number keys 0-9 to select mode and pattern
3. Pattern up (arrow) key
4. Pattern down (arrow) key
5. Start stimulation key
6. Stop stimulation key
7. Intensity knob
8. Reset button (yellow, side panel; same function as power off/on)

### Display

The front-panel LCD display indicates:
1. Operational mode (programmed or stand-alone)
2. Stimulation status (Active/Idle)
3. In Stand Alone mode, indicates pattern number and description
4. Low battery status
5. Value of intensity control (rotation 0-100%)

### Safety Features

1. Hardware power switch: it must turn device off.
2. Internal diagnostic self-check, and watchdog hardware timer power-down.
3. Absence of spurious pulses during mode switching or programming.
4. Electrical isolation: Power and serial connections must be electrically isolated from the rest of the circuitry up to 1000 V.
   Output: Controlled voltage pulses, 0-40 V.
Output resistance is nominally 1 kΩ, but is adjustable by changing internal resistors.
Output is capacitively-coupled by 0.1-μF capacitors.
Output connection is via four 40-pin (20×2) IDC-style male connectors. A separate document “Electrode pinout” provides details.
   Analog in: The TDU has seven 0-5 V analog inputs numbered 0-6; input 0 is reserved for the side panel intensity knob. The others are externally available. All can be read by via command in Remote mode.

The section below provides a more detailed description of command codes. The protocol supports writing commands to the TDU as well as reading the current status and memory contents of the TDU. The opcode for each command is one byte long and is made of a single letter (A\a through P\p). The case of the letter determines whether it is a read (lower case) or write (upper case) command. The opcode byte is the ASCII representation of the letter. In all commands the opcode is followed by a byte [NOF] holding the number of bytes to follow. That is the total number of bytes in any command is equal to 2+NOF. The protocol commands are grouped into three operational categories: I-Electrode-level operations, single electrode, real time (Commands A,B,C,D); II-Electrode-level operations, block udate on array (Commands E,F,G,H,T); and III-Array level operations and system commands (Commands I,J,K,L,M,N,O,P,Q,R,S). In the section below, angle brakets are used to indicate ASCII representation of the information enclosed. For example, [<A>] indicates a byte holding the ASCII representation of A. Data and Parameter ranges are indicated for each parameter. All data are integers. If the data sent to the TDU is below the minimum value, the TDU treats that value as if a zero was sent.

| | Amplitude (PA) for |
|---|---|
| COMMAND: A\a (Write\Read) | one electrode |
| Write Format: (5 bytes) | [A][NOF*][Address][Data][CKSUM] |
| | *[NOF] = Number of bytes to |
| | follow |
| TDU Response: (1 bytes) | [Res*] |
| | *See TDU result codes below |
| Read Format: (3 bytes) | [a][NOF][Address] |
| TDU Response: (1 bytes) | [Data] |

Comment:
Address range 1-144
Data range 0-255 (Parameter range: 0-40 Volts)
Data = 0 No Stimulation
CKSUM is one byte resulting from summing the address and data bytes

| | Pulse width (PW) for |
|---|---|
| COMMAND: B\b (Write\Read) | one electrode |
| Write Format: (5 bytes) | [B][NOF][Address][Data][CKSUM] |
| | *[NOF] = Number of bytes to |
| | follow |
| TDU Response: (1 bytes) | [Res*] |
| | *See TDU result codes below |
| Read Format: (3 bytes) | [b][NOF][Address] |
| TDU Response: (1 bytes) | [Data] |

Comment:
Address range 1-144
Data range 0-255 (Parameter range: 0-510 us)
CKSUM is one byte resulting from summing the address and data bytes
Data = 0 No Stimulation

| | Number of inner bursts |
|---|---|
| | in outer burst (OBN) |
| COMMAND: C\c (Write\Read) | for one electrode |
| Write Format: (5 bytes) | [C][NOF][Address][Data][CKSUM] |
| | *[NOF] = Number of bytes to |
| | follow |
| TDU Response: (1 bytes) | [Res*] |
| | *See TDU result codes below |
| Read Format: (3 bytes) | [b][NOF][Address] |
| TDU Response: (1 bytes) | [Data] |

Comment:
Address range 1-144
Data range 0-255 (Parameter range: 0-255 bursts)
Data = 0 No Stimulation
CKSUM is one byte resulting from summing the address and data bytes

| | Number of pulses per |
|---|---|
| COMMAND: D\d (Write\Read) | inner burst (IBN) for one electrode |
| Write Format: (5 bytes) | [D][NOF][Address][Data][CHSUM] |
| | *[NOF] = Number of bytes to |
| | follow |
| TDU Response: (1 bytes) | [Res*] |
| | *See TDU result codes below |
| Read Format: (3 bytes) | [d][NOF][Address] |
| TDU Response: (1 bytes) | [Data] |

Comment:
Address range 1-144
Data range 0-255 (Parameter range: 0-255 pulses)
Data = 0 No Stimulation
CKSUM is one byte resulting from summing the address and data bytes

| | Pulse Amplitude (PA) |
|---|---|
| COMMAND: E\e (Write\Read) | for each electrode in a block |
| Write Format: (up t0 149 byt.) | [E][NOF*][ul][rl][Data1][Data2] |
| | [Data3] . . . [Datan − |
| | 1][Datan][CHSUM] |
| | *[NOF] = Number of bytes to |
| | follow |
| TDU Response: (1 bytes) | [Res*] |
| | *See TDU result codes below |
| Read Format: (4 bytes) | [e][NOF][ul][lr] |
| TDU Response: (up to 144 by.) | [Data1][Data2][Data3] . . . [Datan − |
| | 1][Datan] |

Comment:
Block Update: block of tactors defined by [ul = upper left tactor] and
[lr = lower right tactor] when ul = 1 and lr = 144 then the entire array
is selected [datan] = [data144]
Data range 0-255 (Parameter range: 0-40 Volts)
Data = 0 No Stimulation
CKSUM is one byte resulting from summing all the bytes following
the [NOF] byte

| | Pulse Width (PW) for |
|---|---|
| COMMAND: F\f (Write\Read) | each electrode in a block |
| Write Format: (up t0 149 byt.) | [F][NOF*][ul][rl][Data1][Data2] |
| | [Data3] . . . [Datan − |
| | 1][Datan][CHSUM] |
| | *[NOF] = Number of bytes to |
| | follow |
| TDU Response: (1 bytes) | [Res*] |
| | *See TDU result codes below |
| Read Format: (4 bytes) | [f][NOF][ul][lr] |
| TDU Response: (up to 144 by.) | [Data1][Data2][Data3] . . . [Datan − |
| | 1][Datan] |

Comment:
Block Update: block of tactors defined by [ul = upper
left tactor] and [lr = lower right tactor]
when ul = 1 and lr = 144 then the entire
array is selected [datan] = [data144]
Data range 0-255 (Parameter range: 0-510 us)
CKSUM is one byte resulting from summing all the bytes following
the [NOF] byte
Data = 0 No Stimulation

| | Number of inner |
|---|---|
| | bursts in outer burst (OBN) for each |
| | electrode in a |
| COMMAND: G\g (Write\Read) | block |
| Write Format: (up t0 149 byt.) | [G][NOF*][ul][rl][Data1] |
| | [Data2][Data3] . . . [Datan − |
| | 1][Datan][CHSUM] |
| | *[NOF] = Number of bytes to |
| | follow |
| TDU Response: (1 bytes) | [Res*] |
| | *See TDU result codes below |
| Read Format: (4 bytes) | [g][NOF][ul][lr] |
| TDU Response: (up to 144 by.) | [Data1][Data2][Data3] . . . [Datan − |
| | 1][Datan] |

Comment:
Block Update: block of tactors defined by [ul = upper
left tactor] and [lr = lower right tactor]
when ul = 1 and lr = 144 then the entire
array is selected [datan] = [data144]
Data range 0-255 (Parameter range: 0-255 bursts)
Data = 0 No Stimulation
CKSUM is one byte resulting from summing all the
bytes following the [NOF] byte

| | Number of pulses per |
|---|---|
| | inner burst (IBN) for each |
| | electrode in a |
| COMMAND: H\h (Write\Read) | block |
| Write Format: (up t0 149 byt.) | [H][NOF*][ul][rl][Data1][Data2] |
| | [Data3] . . . [Datan − |
| | 1][Datan][CHSUM] |
| | *[NOF] = Number of bytes to |
| | follow |
| TDU Response: (1 bytes) | [Res*] |
| | *See TDU result codes below |
| Read Format: (4 bytes) | [h][NOF][ul][lr] |
| TDU Response: (up to 144 by.) | [Data1][Data2][Data3] . . . [Datan − |
| | 1][Datan] |

Comment:
Block Update: block of tactors defined by [ul = upper
left tactor] and [lr = lower right tactor]
when ul = 1 and lr = 144 then the entire
array is selected [datan] = [data144]
Data range 0-255 (Parameter range: 0-255 pulses)
Data = 0 No Stimulation
CKSUM is one byte resulting from summing all the bytes following
the [NOF] byte

| | PA, PW, OBN, IBN for |
|---|---|
| COMMAND: T\t (Write Only) | each electrode in the block |
| Write Format: (up t0 10 byt.) | [H][NOF][ul][rl][field*][Data] . . . |
| | [Datan][CHSUM] |
| | *when field = 0 then [Data] = PA |
| | (n = 1) |
| | when field = 1 then [Data] = |
| | PW (n = 1) |
| | when field = 2 then [Data] = |
| | OBN (n = 1) |
| | when field = 3 then [Data] = |
| | IBN (n = 1) |
| | when field = 4 then [Data] = |
| | [PA][PW][OBN][IBN] (n = 4) |
| TDU Response: (1 bytes) | [Res*] |
| | *See TDU result codes below |

Comment:
Block Update: block of tactors defined by [ul = upper
left tactor] and [lr = lower right tactor]
when ul = 1 and lr = 144 then the entire
array is selected [datan] = [data144]
Data range: as defined for each paramenter
CKSUM is one byte resulting from summing all the bytes following
the [NOF] byte

| | Pulse Period (PP) for |
|---|---|
| COMMAND: I\i (Write\Read) | entire Array |
| Write Format: (4 bytes) | [I][NOF][Data][CKSUM] |
| TDU Response: (1 bytes) | [Res*] |
| | *See TDU result codes below |
| Read Format: (2 bytes) | [i][NOF] |
| TDU Response: (1 bytes) | [Data] |

Comment:
Common to all electrodes
Data range 1-255 (Parameter range: 2-510 us)
CKSUM is a copy of the data byte in this command

| | Outer burst period |
|---|---|
| COMMAND: J\j (Write\Read) | (OBP) for entire Array |
| Write Format: (4 bytes) | [J][NOF][Data][CKSUM] |
| TDU Response: (1 bytes) | [Res*] |
| | *See TDU result codes below |
| Read Format: (2 bytes) | [j][NOF] |
| TDU Response: (1 bytes) | [Data] |

Comment:
Common to all electrodes
Data range 0-255 (Parameter range: 5-1275 ms)
CKSUM is a copy of the data byte in this command

| | Inner burst period |
|---|---|
| COMMAND: K\k (Write\Read) | (IBP) for entire Array |
| Write Format: (4 bytes) | [K][NOF][Data][CKSUM] |
| TDU Response: (1 bytes) | [Res*] |
| | *See TDU result codes below |
| Read Format: (2 bytes) | [k][NOF] |
| TDU Response: (1 bytes) | [Data] |

Comment:
Common to all electrodes
Data range 0-255 (Parameter range: 100-25500 us)
CKSUM is a copy of the data byte in this command

| | Inter-channel period |
|---|---|
| COMMAND: L\l (Write\Read) | (ICP) for entire Array |
| Write Format: (4 bytes) | [L][NOF][Data][CKSUM] |
| TDU Response: (1 bytes) | [Res*] |
| | *See TDU result codes below |
| Read Format: (2 bytes) | [l][NOF] |
| TDU Response: (1 bytes) | [Data] |

Comment:
Common to all electrodes
Data range 1-255 (Parameter range 2-510 us)
CKSUM is a copy of the data byte in this command

| | Amplitude scaling |
|---|---|
| COMMAND: M\m (Write\Read) | (PAS) for entire Array |
| Write Format: (2 or 4 bytes) | [M][NOF][Data][CKSUM]** |
| | **if [data][CKSUM] are omitted |
| | then the TDU uses the local |
| | intensity |
| | control for the PAS value, |
| | otherwise the value in [Data] will |
| | be used |
| | and the local control will be |
| | sampled but not used. The TDU |
| | will continue |
| | to use the last written value |
| | until a new command tells it |
| | otherwise |
| TDU Response: (1 bytes) | [Res*] |
| | *See TDU result codes below |
| Read Format: (2 bytes) | [m][NOF] |
| TDU Response: (1 bytes) | [Data] |

Comment:
Common to all electrodes
Data range 0-255 (Parameter range 0-100%)
CKSUM is a copy of the data byte in this command

| | Update a pre- |
|---|---|
| COMMAND: N\n (Write\Read) | programmed pattern |
| Write For.: (150, 21, 6, or 4 byt.) | [N][NOF][Access][ID][field*] |
| | [Data1] . . . [Data144][CKSUM] |
| | *field = 0: Pulse Amplitude for |
| | each electrode in the array |
| | field = 1: Pulse Width for each |
| | electrode in the array |
| | field = 2: Number of inner |
| | bursts in outer burst for each |
| | electrode |
| | field = 3: Number of pulses per |
| | inner burst for each electrode |
| | [N][NOF][Access][ID][field*] |
| | [Data1] . . . [Data16][CKSUM] |
| | *field = 9: Pattern ID (all bytes |
| | must be included) |
| | [N][NOF][Access][ID][field*][Data] |
| | [CKSUM] |
| | *field = 4: Pulse period for the |
| | entire array |
| | field = 5: Outer burst period for |
| | the entire aray |
| | field = 6: Inner burst period for |
| | the entire array |
| | field = 7: Inner channel period |
| | for the entire array |
| | field = 8: Amplitude scaling for |
| | the entire array |
| | [N][NOF][Access][ID][field*] |
| | [CKSUM] |
| | *field = 10: Load pattern from |
| | memory |
| | field = 11: Store pattern in |
| | memory |
| TDU Response: (1 bytes) | [Res*] |
| | *See TDU result codes below |
| Read Format: (5 bytes) | [n][NOF][Access][ID][field] |
| TDU Response: (1 or 144 bytes) | [Data] |
| | [Data1] . . . [Data144] |

Comment:
ID is the number of pattern being updated
Access is a code used for security. (Access = 199)
Data ranges are the same as indicated in the previuos commands
TDU must be in Pattern Update mode. Otherwise an invalid Opcode
response will be sent
CKSUM is one byte resulting from summing the ID, Access, field,
and data bytes

| | Start stimulation of |
|---|---|
| COMMAND: O (Write ONLY) | the currently loaded pattern |
| Write Format: (2 bytes) | [O][NOF] |
| TDU Response: (1 bytes) | [Res*] |
| | *See TDU result codes below |
| Comment: | |
| COMMAND: P (Write ONLY) | Stop stimulation |
| Write Format: (2 bytes) | [P][NOF] |
| TDU Response: (1 bytes) | [Res*] |
| | *See TDU result codes below |
| Comment: | |
| | Display a pre- |
| COMMAND: Q (Write ONLY) | programmed pattern |
| Write Format: (4 bytes) | [Q][NOF][Data][CKSUM] |
| TDU Response: (1 bytes) | [Res*] |
| | *See TDU result codes below |

Comment:
Data range 0-52 (53 pre-programmed patterns)
CKSUM is a copy of the data byte

| | Deliver a sequence of |
|---|---|
| COMMAND: R (Write ONLY) | outer burst |
| Write Format: (4 bytes) | [R][NOF][Data][CKSUM] |
| TDU Response: (1 bytes) | [Res*] |
| | *See TDU result codes below |

Comment:
Data ramge 0-255 (Parameter range 0-255 bursts)

| | Current analog value |
|---|---|
| COMMAND: s (Read ONLY) | for a channel |
| Read Format: (3 bytes) | [a][NOF][CH] |
| TDU Response: (1 or 7 bytes) | [Data] |
| | [Data1] . . . [Data7] |

Comment:
Data range 0-255 (Parameter range: CH0: Intensity 0-100%)
[CH] = 0 for Intensity
[CH] = 1 for AI1
[CH] = 2 for AI2
[CH] = 3 for AI3
[CH] = 4 for AI4
[CH] = 5 for AI5
[CH] = 6 for AI6
[CH] = 7 for Intensity, AI1, AI2, AI3, AI4, AI5, AI6
Response Byte For Write
Commands:

| | | |
|---|---|---|
| *[Res] = | [1] | Operation Successful |
| | [2] | Parameter(s) not initialized |
| | [3] | Pattern not initialized |
| | [4] | Invalid opcode |
| | [5] | Invalid address |
| | [6] | Invalid |
| | | field |
| | [7] | Wrong check sum |
| | [8] | Invalid |
| | | data |
| | [9] | Parameter combination Invalid |
| | [10] | Stimulation is already ON |
| | [11] | Stimulation is already OFF |
| | [12] | Invalid access code |

### Example 20

### Treatment of Dysphonia

Experiments conducted during the development of the present invention demonstrated that tactile simulation may be used to treat subjects suffering from dysphonia.

### Focal Dystonias (Spasmodic Dysphonia)

Spasmodic dysphonia is one type of a family of disorders called focal dystonias. When a single muscle or small group of muscles contract spontaneously and irregularly without good voluntary control, those muscles are dystonic. While there are dystonias where a large number of muscles or a complete region of the body is involved, focal dystonias are limited to a small area or single muscle. Examples would include torticollis where a spasm of a neck muscle causes the head to rotate. Blepharospasm is when the muscle around the eye spontaneously twitches. Writers cramp is when the muscles of the hand spasm. Spasms of the muscles in the voice box are a laryngeal dystonia.

### Laryngeal Dystonias

There are several types of laryngeal dystonia. The most common type is when the muscles that bring the vocal folds together for speaking intermittantly spasm. Since the voice box serves several functions, including speaking, breathing and preventing food from getting into the lungs when swallowing; laryngeal dystonias can affect more than the voice. When the voice is the primary site affected, then the laryngeal dystonia is called spasmodic dysphonia. It has also been referred to as spastic dysphonia.

Adductor Spasmodic Dysphonia

Adductor spasmodic dysphonia is the most common type of laryngeal dystonia and involves spasms of the muscles that close the vocal folds. It could be appropriately called the strain-strangled voice. The spasms cause a choking off of the voice or interruptions of the voice. Adductor spasmodic dysphonia may also sound just like a tightness or effortfulness without any obvious cutting out type symptoms.

Abductor Spasmodic Dysphonia

Abductor spasmodic dysphonia involves the muscles that open the voice box for breathing. If they spasm while speaking the person develops an involuntary whisper while trying to speak.

Respiratory Dysphonia

Respiratory spasmodic dysphonia is from a spasms of the vocal fold muscles belonging to the adductor group but instead of spasming during speaking, they spasm during breathing. Theses spasms create noisy and difficult breathing even when a subject is not intending to make a noise.

A subject having an inability to speak was treated with the systems and methods of the present invention. Electrotactile tongue training as described in Example 1 was used to cause the subject to concentrate while receiving electrotactile stimulation. The subject was encouraged to try to talk during the training process. After training, the subject regained the ability to speak. The ability to speak was retained after electrotactile stimulation was discontinued.

### Example 21

### Recovery from Traumatic Brain Injury

### Traumatic Brain Injury

Traumatic Brain Injury (TBI) has been defined as “ . . . an acquired injury to the brain caused by an external physical force, resulting in total or partial functional disability or psychosocial impairment, or both.” Therefore, in general, TBI refers to open or closed head injuries, but, generally, does not apply to “injuries that are congenital or degenerative, or to brain injuries induced by birth trauma, although the present invention find use in both categories. (See, e.g., The Individuals with Disabilities Education Act. 34

Code of Federal Regulations §300.7(c)(12)).

TBI can result from, among other things, vehicular accidents, falls, assaults, and sport injuries, in which an external force causes the brain to move, inflicting trauma to the brain. Insufficient oxygen supply to the brain, infection or poisoning may also cause TBI-related dysfunctions.

TBI is generally characterized as a heterogeneous disorder, affecting an individual's physical, cognitive and psychosocial functioning. Due to the extent of trauma inflicted to the brain, the location of injury, and the availability of emergency procedures, TBI can result in serious, and in many cases life-long, impairments.

### Epidemiology of Traumatic Brain Injury in the United States

The report to the United States Congress drafted by the Centers for Disease Control and Prevention indicates the following annual estimates for the years 1995 through 2001:
Annually, at least 1.4 million people sustain a Traumatic Brain Injury. Of these, about 50,000 die, 235,000 are hospitalized, and 1.1 million are treated and released from an emergency department. (Traumatic Brain Injury in the United States: Emergency Department Visits, Hospitalizations, and Deaths.” National Center for Injury Prevention and Control., available at http://www.cdc.gov).

Recently, prevalence of TBI is estimated at 2.5 million to 6.5 million individuals suffering from any kind of impairment resulting from Traumatic Brain Injury in the United States. In 1995, the incidence of hospitalization for TBI was calculated at 100 per 100,000 based on population estimates. When compared with the early 80's estimates of 200 per 100,000 hospitalized cases of head injury, the incidence seems to have decreased. Nevertheless, this assumption has proved misleading due to the fact that many cases of mild Traumatic Brain Injury are not being hospitalized and/or are being undiagnosed and thus underestimated. (See, e.g., Novack “TBI Facts and Stats”. Recovery after TBI Conference. September 1999 http://www.neuroskills.com).

The mortality rate for TBI is 30 per 100,000, resulting in an annual mortality rate of 52,000 individuals. 50% of deaths related to TBI occur within the first 2 hours of injury, which indicates an increased need of immediate medical attention upon an incidence of TBI. As Novack suggests, “the treatment given by paramedics and in the emergency room can make a big difference in terms of an individual's survival.”

Demographic statistics indicate that males are at a greater risk, namely they are twice as likely as females to suffer from TBI. There are also specific age groups that are at a higher risk of inducing TBI than others. The highest incidence is among individuals within the age category of 15-24 years. An increased risk is also associated with people over 75 years of age and children 5 and younger.

Alcohol and drug abuse is closely connected with higher incidence rates. Alcohol abuse is reported in about half of the cases of TBI, in which either the victim or the individual causing the head trauma was under the influence of alcohol or other substances.

The greatest percentage of TBIs are the result of a vehicular accident, involving, among other instruments, vehicles, bicycles, motorbikes, and pedestrians. The second most frequent cause of TBI is falls, mostly affecting the elderly or the very young. About 20 percent of TBIs are a direct cause of violence, both firearm and non-firearm assaults. An alarming statistic regarding TBI victims who are 5 and younger indicates that a leading cause of TBI in children under five is assault. Even though only 25 percent of TBIs in young children are a result of child abuse, the “Shaken baby syndrome” is a significant contributor to high incidence of TBI in infants. Sports-related injuries are only a fraction, namely 3 percent, of all TBI. Nevertheless, approximately 90 percent of these injuries are mild TBIs that are generally unreported, underestimated and thus are not treated properly.

Degrees of severity of Traumatic Brain Injury

Standard clinical assessment distinguishes at least three degrees of Traumatic Brain Injury based on the Glasgow Coma Scale (GCS): severe (GCS range 3-8), moderate (GCS range 9-12) and mild (GCS range 13-15). GCS is a common method of measuring the severity of TBI, generally used in emergency departments, based on the depth of coma (See, e.g., Rappaport et al., Archives of Physical Medicine and Rehabilitation, 63: 118-123 [1982]). Glasgow Coma Scale score of less than 15 during the first 24 hours after the injury is only one of three primary factors that are assessed as they may be crucial indicators of the occurrence of TBI. Besides the Glasgow Coma Scale, a documented loss of consciousness, and/or the occurrence of amnesia for the event of TBI may demonstrate a case of TBI.

A more accurate assessment of a brain injury provides the occurrence of Post-Traumatic Amnesia. The duration of post-traumatic amnesia can determine the severity of brain dysfunction as a result of TBI. Generally, amnesia that lingers up to a week indicates severe injury; if the duration of amnesia is up to a day, TBI can be assessed as moderate; and if amnesia lasts for up to an hour, it may be concluded that the brain suffered mild trauma.

Mild Traumatic Brain Injury, which usually goes undiagnosed, can be characterized by any of the following symptoms or their combinations: “a brief loss of consciousness, loss of memory immediately before or after the injury, any alteration in mental state at the time of the accident, or focal neurological deficits.” Even though the victim of Mild Traumatic Brain Injury may seem “normal” and thus does not seem to need medical attention, in many cases Mild Traumatic Brain Injury results in chronic functional deficit known as Postconcussion Syndrome.

The most severe cases of TBI may result in enduring coma followed by a persistent vegetative state. Persistent Vegetative State is a condition of a complete loss of cognitive neurological functioning and awareness of the environment, but retention of sleep-wake cycle and noncognitive functions. In other words, higher cerebral functions of the brain are diminished, but the functions of the brainstem, such as respiration and circulation, remain intact.

### Focal Cerebral Lesions/Cerebral Contusions

The brain, an extremely delicate tissue composed of about 15 to 20 billion neurons and additional support cells, is extremely sensitive to traumatic injuries. Due to acceleration and deceleration, which generally occur during a traumatic brain injury, the brain strikes the inside of the skull causing bruising. The most vulnerable parts of the brain, located near bony protrusions of the skull, are the brain stem, frontal lobe, and temporal lobes in particular. Consequently, these specific locations are the most frequently damaged parts during an incident of TBI.

Localized damage of the brain stem, located at the base of the brain, may cause disorientation, frustration, and anger. This area of the brain regulates basic arousal and consciousness, but it also plays an important role in normal functioning of short-term memory and attention. Consequently, localized trauma to the brain stem can result in impairment of any of these functions.

The temporal lobes, closely connected to the limbic system regulating human emotions, partake in a variety of cognitive skills, such as memory and language. Left temporal lesions generally cause dysfunction in the area of recognition of words, whereas right temporal damage may cause a loss or inhibition of talking. Similarly, left temporal lesions result in impaired memory for verbal material, while right temporal damage usually causes loss of recollection of non-verbal material. As Blumer and Benson suggest, temporal lobe lesions can result in a number of serious behavioral disorders, such as perseverative speech, paranoia and even aggressive rages. (Blumer and Benson, Frontal Lobe Function, New York: Grune & Stratton, (1975)).

Due to its large dimensions and its location near the front of the skull, the frontal lobe is the most frequently damaged area of the brain in an incidence of TBI. Consequently, the frontal lobe is the most common region of injury, particularly in mild to moderate TBI. Frontal lobe lesions can cause such a wide variety of symptoms that cannot be equaled by injury to any other part of the brain (Kolb and Milner, Neuropsychologia, 19:505-514 (1981)). Damage to the frontal lobe, regulating cognitive functions and controlling an individual's emotions and personality, can result in, among other things, decreased judgment, increased impulsivity, dysfunctional social and sexual behavior, impairment of motor function, problem solving, memory, language, etc. Impairment of motor function can be generally demonstrated by loss of fine movements, loss of strength of the arms, hands and fingers, and an overall dysfunction of complex body movements. Additionally, spatial orientation may be affected.

On the level of social behavior, victims of frontal lobe damage due to TBI may exhibit abnormal “behavioral spontaneity”, such as fewer spontaneous facial movements and excessive or limited speech (Kolb and Milner, Neuropsychologia, 19:505-514 (1981)). Impacts of frontal lesions on an individual's social behavior are massive, causing significant alterations of personality and emotional status. These behavioral changes may vary, according to the area of the frontal lobe that is affected. Damage to the left side generally causes pseudodepression, while right side lesions result primarily in pseudopsychopathic behavior. (Blumer and Benson, Frontal Lobe Function, New York: Grune & Stratton, (1975)).

Even though focal contusions are typically located in the superficial brain structures, they are frequently accompanied by the formation of deep hematomas, affecting deeper layers of the brain tissue.

Hematoma is classified as a localized brain damage caused by a formation of a blood clot in a particular part of the brain. The violent movement of the brain accompanying TBI causes vessels on the brain surface to be pulled, stretched, or torn, often resulting in hematoma. Hematomas are particularly dangerous since they compress the soft brain tissue and if not treated promptly and properly may cause death. There exist several classification of hematomas based primarily on the origin of blood clotting within the brain tissue. A subdural hematoma is a blood clot that forms below one of brain's protective layers. An epidural hematoma occurs when a blot clot forms between the dura and the cranium. An intracerebral hematoma or hemorrhage is caused by bleeding within the brain tissue.

### Diffuse Cerebral Lesions

Diffuse axonal injury occurs when the nerve cells are torn from one another, or rather, when axons pull and tear, disabling the communication between neurons. If axon is damaged, the cell dies, causing neural defects and deficiencies. Consequently, brain damage is no longer localized, but rather diffuse. Diffuse cerebral lesions often coexist with focal lesions, resulting in a wide spectrum of neurological, cognitive, and psychosocial impairment.

Both localized and diffuse injuries are considered primary injuries; they are a direct consequence of traumatic brain injury and, at present, medical treatments are not available to reverse the injury. The so called secondary brain injury are thought to be preventable if immediate medical attention is available.

### Secondary Brain Injuries

Even though the terms anoxia and hypoxia are often used interchangeably, there is a specific difference between these medical conditions. Anoxia refers to a condition in which there is an absence of oxygen supply to an organ's tissue despite adequate blood flow to the tissue. Hypoxia is a condition in which there is a decrease of oxygen to an organ's tissue in spite of adequate blood flow to the particular tissue. The primary cause of an insufficient supply of oxygen to the brain is loss of breathing or rapid decrease of blood pressure. Besides being a potential secondary injury in an incidence of Traumatic Brain Injury, anoxia and hypoxia may also occur due to inhalation of carbon monoxide, exposure to high altitude, anesthetic accidents or poisoning. Anoxia and hypoxia result in additional brain injuries in TBI patients, in severe cases inducing coma ranging from hours to months. In the comatose state, seizures, muscle spasms, and neck stiffness typically occur.

Increased intracranial pressure can cause a severe swelling of the brain, also referred to as edema. Edema may prevent blood flow into the brain, causing a fatal condition. The occurrence of edema simultaneously with hematoma may signify a further deprivation of oxygen supply and thus a higher risk of death.

Secondary injuries to the brain following a case of TBI are reported as more rare due to the advances of current medicine and emergency procedures.

### Effects of Traumatic Brain Injury

Given the heterogeneous character of TBI, there is much difficulty in characterizing it by one specific symptom or impairment. On the contrary, TBI results in sets of dysfunctions, different for each individual. Furthermore, consequences of TBI, even a mild case, often linger all life long, frequently alter their original form and even worsen as an individual meets new challenges, matures and/or ages. Accordingly, in some embodiments, if a subject presents with any of the symptoms discussed herein, the subject may have TBI.

Neurological impairment caused by TBI can affect any region of the neural axis, compromising any motor, sensory and autonomic function. Neurological consequences of TBI can be demonstrated as various movement dysfunctions, paralysis on either one side or both sides of the body, seizures, spasticity (sudden contraction of muscles), vision deficits, headaches and sleep disorders. In many cases, the Post-Trauma Vision Syndrome can be experienced as double vision, movement of stationary objects, visual fatigue, headaches, cognitive impairment, and compromised sense of balance, coordination and spatial orientation. These dysfunctions are not related to any pathology of the eye per se and therefore have often been excluded from the rehabilitation process.

Neurooptometric rehabilitation, in particular, proved to be of significant importance in treatment and management of Post-Trauma Vision Syndrome. Symptoms connected with Post-Trauma Vision Syndrome can be misinterpreted as a learning disability or even as attention deficit disorder. Post Trauma Vision Syndrome is caused by a dysfunction of the ambient visual process, which, if functioning properly, provides information needed for balance, coordination, posture and movement. The ambient visual process coordinates information from the peripheral retina to a specific level of midbrain that provides a sensory-motor feedback. As such, this process can be classified as motoric in function and as correlating the kinesthetic, proprioceptive, vestibular, and tactile systems. In Traumatic Brain Injury, the ambient visual process is unable to organize spatial information with other sensory-motor systems.

Cognitive consequences include, but are not limited to, memory impairment and concentration and attention dysfunctions. Many cognitive problems are closely associated with language use and visual perception. As mentioned previously, frontal lobe functions are frequently compromised, resulting in some cases in difficulties with problem-solving, information processing, organization, abstract reasoning, insight, and judgment.

Consequently, it is problematic for a TBI victim to learn new things and the inability to concentrate and organize one's thoughts often causes frustration, confusion and forgetfulness. Due to dysfunctional abstract thinking, understanding of irony, sarcasm, multiple meanings in jokes and figurative language is difficult to impossible. Regarding language and speech, TBI seldom inflicts a complete impairment of language, but rather causes difficulties with word-finding and sentence formation. The inability to find a term or a word results in lengthy, rather illogical, explanations and frustration when not understood. Since people with TBI are not aware of their language impairment and frequent errors, they tend to blame others for communication difficulties. Dysarthria is a common problem among TBI sufferers, caused by damage of muscles of the speech mechanism. It can be detected as slow, slurred, and indiscernible speech. Dysphagia is also common in individuals with TBI. It generally refers to any problems with swallowing. Apraxia of speech, in which speech muscles are not damaged, results in dysfunctional processing of words and inability to say words correctly and in a consistent way. Additionally, reading and writing are usually more deficient than speech, causing further difficulties in school or at work.

Behavioral deficits following TBI are numerous and difficult to treat. They include verbal and physical aggression, impulsivity, mood disorders, personality changes, depression, anxiety, poor self-awareness, and dysfunctional sexual behavior. These deficits, combined with neurological and cognitive dysfunctions, have broad social consequences. They often result in increased suicidal behavior, divorce, chronic unemployment, economic frustration, and substance abuse. TBI thus impacts heavily not only its immediate victims, but also their family members. Many dysfunctions become obvious when individuals try to return to their normal lives after an extensive medical treatment and rehabilitation. Children with TBI are most susceptible to the complex interrelation of neurological, cognitive, and behavioral impairment, since its full impact can become apparent later on in their lives, as they attempt to learn new things and as they become exposed to new environments and situations.

### Brain Recovery Rehabilitation and Treatments

Evidence suggests that the human brain, even in adult individuals, has the capacity to recover. Brain plasticity is a natural response to loss of neurons through aging. Neurogenesis, it might seem, thus provides a promising alternative for the treatment of many neurological problems, including, among other things, TBI. Nevertheless, “under normal conditions, neurogenesis in the adult brain appears to be restricted to the discrete germinal centers: the subventricular zone and the hippocampal dentate gyrus” Hallbergson et al., The Journal of Clinical Investigation. 112(8): 1128-1133 [2003]).

It has been documented that, due to damage to a particular area of the brain, surrounding tissues are able to assume the functions originally coordinated by the damaged tissue. The so-called sprouting of dendrites can occur following a brain injury; in which case neurons sprout, establishing new connections. The injured brain thus has a capacity to increase the level of chemicals that promote growth of neural connections. Sprouting of dendrites may occur proportionally to the extent that a person remains active. Consequently, brain plasticity can contribute to and positively affect recovery if suitable rehabilitation procedures provide enough stimulation and brain activity.

The process of recuperation from TBI is typically a life-long effort of accommodation to multiple dysfunctions. Effects of a particular therapy depend on numerous factors, such as the extent of brain damage, the choice of a specific rehabilitation, or rather the choice of a set of particular rehabilitation procedures, the frequency and intensity of these treatments, and the level of cooperation from the patient as well as the patient's family members.

The most effective rehabilitation procedure, as reported by NIH Consensus Statement, is a comprehensive interdisciplinary rehabilitation that ensures an individual approach to every TBI patient with a unique set of deficits. This rehabilitation is complex in nature, addressing the heterogeneity of post-Traumatic Brain Injury damage.

### Traumatic Brain Injury and the Systems of the Present Invention

Experiments conducted during the development of the present invention have demonstrated that healthy as well as sick or diseased subjects (e.g., bipolar vestibular dysfunction patients) demonstrate improvement or correction of, among other things, their vestibular function (e.g., balance), proprioception, motor control, vision, posture, cognitive functions, tinnitus, emotional conditions and sleep as a direct consequence of training procedures with the systems of the present invention. Thus, in some embodiments, the present invention provides methods of training with the systems of the present invention in order to treat symptoms (e.g., symptoms mentioned herein) of persons with TBI. Treatment, in some embodiments, permits these persons to incorporate themselves into normal life, to be independent, and to enjoy an increased quality of their lives. In some embodiments of the present invention, dysfunctions are treated and consequently eliminated in patients with TBI. Exemplary benefits are described below.

### General Balance Improvement

In some embodiments, subjects with TBI experience the return of their sense of balance, steadiness, and a sense of being centered after rehabilitation procedures with systems and methods of the present invention (e.g., treatment with the systems of the present invention). In some embodiments, the sense of constant movement is eliminated in the TBI subjects. In some embodiments, subjects who without treatment have difficulty walking unassisted or in crowds or dark environments are capable of doing so after treatments provided by the present invention (e.g., procedures with the systems of the present invention).

TBI patients suffering from Post-Trauma Vision Syndrome have similar deficits of general balance, due to damage to their ambient visual process. The loss of the sense of the midline in TBI patients results in loss of the sense of balance and the sense of being centered. Thus, in some embodiments, the present invention provides systems and methods of using the systems of the present invention to treat (e.g., retrain) the damaged centers of the ambient visual system, thereby resulting in a general improvement of the sense of balance, steadiness, a normal sense of the midline and thus a renewed sense of being centered. It is contemplated that improvement of a TBI patient's general balance would thus have significant consequences on the overall rehabilitation process.

### Posture, Proprioception and Motor Control

In some embodiments, the present invention provides a therapy with the systems of the present invention, whereby a TBI patient's body movements become more fluid, confident, relaxed and quick. In some embodiments, stiffness of movement disappears and fine motor skills return to normal. In some embodiments, posture, gait and body segments alignment return to normal.

Numerous movement dysfunctions, seizures, spasticity, and loss of fine motor movements in Traumatic Brain Injury patients are highly similar in nature with motor deficits resulting from lateral vestibular disorder. Thus, in some embodiments, the present invention provides systems and methods for treating patients with TBI (e.g., subjects displaying symptoms of bipolar vestibular disorder). In preferred embodiments, TBI patients display improvement in functioning of their motor, cognitive, and neurological functions after treatment with the systems and methods of the present invention.

### Vision

In some embodiments, TBI patients display improved vision after receiving treatments according to the present invention. Improved vision includes, but is not limited to, vision becoming clearer, more stable, clearer, and brighter, reduction of oscillopsia, widening of peripheral vision, improvement of depth perception, reduction of or elimination of double vision, and reduction of or elimination of movement of stationary objects and visual fatigue.

### Cognitive Functions

In some embodiments, treatments (e.g., treatments with the systems of the present invention) provided by the present invention to a subject (e.g., a TBI patient) increases, among other things, mental awareness, creativity, clarity of thinking, multitasking skills, memory retention, concentration, the ability to track conversations, and the ability to focus. In some embodiments, subjects experience less “noise” in the head, much improvement in intensity of thinking, problem solving, and decision making. Furthermore, there is improvement of major executive skills thereby resulting in increased confidence and improved self-assessment.

### Sleep

Sleep disorders have been reported in most cases of TBI, resulting in complications of rehabilitation. Accordingly, in some embodiments, treatments (e.g., treatments with the systems of the present invention) provided by the present invention to a subject (e.g., a TBI patient) improve sleep. Sleep improvement occurs and is perceived as being fuller, longer, and more restful, often with no awakenings during the night. As an additional impact, in some embodiments, treatment with the systems of the present invention results in improved sleep patterns.

### Exemplary Treatment

Systems and methods of the present invention were utilized for balance training in two subjects with traumatic brain injury (TBI) presenting cerebellar type ataxia.

Ataxia is frequently observed following severe TBI. It very often accompanies other motor deficiencies and thought to clinically resemble other cerebellar symptoms. CT and MRI investigations rarely show direct lesions in this part of the brain. It forms part of a mixed clinical picture; general diffused axonal lesions and extra dural haematoma being the main identifiable cerebral lesions.

Unlike other neurological symptoms, ataxia remains typically unresponsive to traditional treatment techniques.

Patients presenting with early signs of tremor, severe dysmetria and other motor based coordination problems at the onset of treatment often find they are forced to live the rest of their lives trying to come to terms with it as therapists, neurologists and neurosurgeons have yet to find a solution. Voice control and excessive salivation are also frequent. Fine manual motor skills are severely impaired and simple activities of daily life and basic social skills are permanently perturbed. Therapists can only offer over-training and compensatory strategies for this debilitating condition.

Severe psychological suffering, despair and depression often accompany the physical aspects as the frustration of possessing full limb and trunk movement but not being able to control it is a permanent and omnipresent challenge.

Two fully informed adults willingly gave their consent to participate in a study to evaluate the use of the systems and methods of the present invention and physical exercise to try and improve balance and thus regain function and mobility in traumatic ataxia following TBI.

Both subjects received emergency acute care then received regular, intensive physical therapy throughout their rehabilitation, largely provided by the same therapists.

Subject 1 was a male, 26 years old who left the treatment facility 7 years previous to experiments conducted during the development of the present invention and after two and a half years in treatment. His clinical picture remained the same since leaving the treatment facility. Initial CT scan showed with a Glasgow coma scale of 3.

He suffered from severe coordination disturbance, dysmetria, a very poor force/task correlation (inappropriately high muscle recruitment, resulting in disastrous motor responses, fatigue and a general musculature largely exceeding his actual activity level).

Motor asymmetry was also present following initial right-sided paralysis, which had recovered well (e.g., full range movements against resistance in all muscle groups). The shoulder and pelvic girdles and other segmental levels rarely moved independently. Falling was frequent with inappropriate parachute reactions and frequent minor injury.

Subject 2 is a female, 25 years old who left the treatment facility 2 years prior to treatment with the methods of the present invention, after 12 months in treatment. Her clinical picture had remained the same since leaving. She displayed an initial Glasgow coma scale of 5. Medium frequency permanent tremor accompanied movement and was present throughout the muscular system. Voice, articulation and the muscles of facial expression were also affected.

At day 1 of the trial.

Subject 1 (male). Severe in coordination forces him to use a wheel chair for all outdoor mobility and much indoor use. Some use of a 4-wheeled walker or walking between 2 people is used indoors. Independent transfers are possible though falls occur. All limb and vertebral movements are achieved in the presence of low frequency tremor and dysmetria (over or undershooting) by fixing levers with excessive muscular control and rigidity. Standing with one handhold is possible. Independent standing is possible but precarious (10 to 20 sec. before intervention of a helper is necessary).

Subject 2 (female). Outdoor walking with a stick is possible. Short distance indoor walking is independent but gait is interrupted for balance at each pace. Standing with eyes closed and feet spaced at shoulder width was impossible.

Training. Patients were trained for 7 days (5 consecutive, weekend pause then 2 consecutive).

The subjects used the systems and methods of the present invention during two sessions a day for a maximum of 40 minutes per session including one 20 minute uninterrupted stabilization exercise in standing or on an 80 cm diameter Klein (Swiss) type ball with eyes closed. Each session included exercises for shoulder and pelvic girdle and other segmental level disassociation; for general and segmental relaxation and for gait analysis and retraining.

Results of training were documented by the physical therapist's observations, patients own remarks, and external observers' spontaneous remarks (e.g., family, other health professionals etc.).

Physical therapist's (PT) observations. PT found that patients tolerated the systems and methods of the present invention well with no adverse effects. Patients reported no discomfort or problems using the device. PT was pleasantly surprised that patients with this pathology were able to follow the usual general training program. PT noted that fatigue and cognitive problems did not force modification of the training regime and the patients remained motivated throughout the trial.

PT noted that the two subjects have no language problems. PT noted that memory and organizational handicaps did not affect learning as the subjects acquired personal strategies (increased question asking and checking, note pads, etc.) and were provided repeat instructions (e.g., “key word” reminders).

At the end of training, PT noticed a significant improvement in static posture, both in terms of stability, endurance and in the quality of vertical segmental alignment in both subjects. Muscular tension in postural groups was more appropriate—accessory movements and inappropriate muscle group recruitment diminished in both subjects resulting in a more energy effective work rate and lower general and muscular fatigue.

PT noted that Subject 1 was able to stand for several minutes with closed eyes or sit on the ball for 20 minutes un-assisted with eyes closed and feet at 40 cm (e.g., compared to day 1, when Subject 1 sat for 5 minutes feet were wide spread eyes open and the ball partially deflated with severe muscular tremor from fatigued over-active quadriceps femoris.)

PT noted that Subject 2 was able to stand for 20 min un-assisted with feet together and eyes closed after training (e.g., versus feet apart, eyes open and rapid onset of severe tremor before treatment with systems and method of the present invention).

PT noted that the two subjects saw transfers from sit/stand and from stand/sit improve both in quality of movement an in security. Gait improved in both subjects. PT noted that Subject 1 was able to take up to 8 steps un-assisted under close surveillance; whereas he had not been able to take any independent steps since his accident. Use of a 4 wheeled walker un-assisted was improved on flat ground with a smoother movement flow and the integration of several gait components previously absent such as weight transfer, knee flexion in stepping, foot positioning, more equal and appropriate step length, shoulder girdle coordination and more efficient upper limb work (elbows flexed rather than in hyperextension). PT also noted that endurance increased progressively during training, as did walking on un-even surfaces.

Subject 2 was able to step cleanly over an obstacle of 40 cm un-aided (whereas, clearing a 14 cm obstacle was impossible on day 1). Walking on uneven and sloping grass surfaces without the stick became possible and endurance and gait quality improved.

The patients own remarks. Subject 1 reported feeling generally more supple with general muscle tone more “relaxed”. He reported his gait is smoother with steps less “jerky”. He feels he uses less muscle work to achieve the same actions and with less tiredness. He noticed that knee bending during walking became possible whereas previously he reported always walking with lower limbs “stiff” (knees remained in extension or hyperextension). He finds general balance much improved especially regarding stability in standing which is possible for longer periods. He reported a better tactile awareness of the ground with more equal weight distribution throughout the soles of the feet where as he only perceived contact at the heels before. He thinks this is due to a transfer of learning from the concentration on lingual tactile sensation in a signal of the system of the present invention to adjust balance, to an application of a similar procedure for an increase in awareness of tactile sensation and adjustment of posture in foot sensitivity.

He also reported that transfers are performed more easily and smoothly. He felt that the systems and methods of the present invention aided postural stability during use and allowed muscular relaxation of non-involved groups. He found using the device simple after initial training and stimulation was comfortable. He also reported an improved length and quality of sleep.

Subject 2 reported feeling more supple in the whole vertebral region and in muscle groups controlling the knees. She finds all movement smoother. Shoulder girdle relaxation is much improved and she is able to stand still for longer periods without the onset of tremor. Loss of balance is markedly reduced. She finds her speech is more easily understood by others and postulates that this is due to better respiratory control and/or better articulation of words.

She reports that heel strike and push off phases in gait are better perceived. She is more able to maintain a “head-up, looking straight ahead” posture in walking (she had previously complained that she looked at feet while walking).

She found the physical exercises accompanying training to be well adapted and important. She found the systems and methods of the present invention were easy to use and she found it quite straightforward to learn to maintain balance with a device of the present invention and found it especially useful to rely on it towards the end of the 20 minute training sessions when balance became difficult through fatigue. She reported really trusting the systems and methods of the present invention during fatigue to maintain upright posture. She also reported that physical endurance improved and that the training period was a positive experience. No adverse sensations were reported.

Other external observers' spontaneous remarks (e.g., family, other health professionals etc.).

Friends of Subject 1 found Subject 1's speech more easy to understand. Walking with the support of two people was easier, they reported “carrying” less and noticed the improved quality of gait especially in stepping with knee flexion, reduced foot drag, narrower gait base and appropriate step length (reduction in exaggerated paces).

Subject 2's family noted improved speech, and general smoothness of movement. During a longer walk on grass with no assistance (2×500 m) accompanied by a family member, both observed a better quality of stepping, (suppleness and smoother leg movements), and an improved head position. The family found improved respiratory coordination in speech and longer sentence length.

### Example 22

### Pervasive Developmental Disorders

### Pervasive Developmental Disorders

Autism is a complex developmental disability that typically manifests itself within the first three years of life. The result of a neurological disorder that affects the functioning of the brain, autism impacts normal development of the brain in areas of social interaction and communication skills. Children and adults with autism typically have difficulties with verbal and non-verbal communication, social interactions, and leisure or play activities.

Autism is one of five disorders covered under the umbrella term Pervasive Developmental Disorders (PDD), a category of neurological disorders characterized by severe and pervasive impairment in several areas of development, including social interaction and communication skills.

PDD can be classified as follows: Autistic Disorder, Asperger's Disorder, Childhood Disintegrative Disorder (CDD), Rett's Disorder, and PDD-Not Otherwise Specified (PDD-NOS). Each of these five disorders has specific diagnostic criteria as outlined by the American Psychiatric Association (APA) in its Diagnostic & Statistical Manual of Mental Disorders.

In spite of meaningful successes in diagnosis, classification and understanding of Autism Spectrum Disorders (ASDs), many uncertainties and challenges for research still remain. For example, the causes of the various autistic disorders remain, to a large extent, unidentified. There has not been a “cure” for autism, although some management strategies exist that seem to be effective for some individuals. Individuals with autism also suffer from a number of physiological problems the significance of which—in terms of cause and development of ASDs—is unclear and sometimes controversial.

### Prevalence of Autism

Autism is the most common Pervasive Developmental Disorder, affecting an estimated 1 in 250 births (Centers for Disease Control and Prevention, 2003). This means that as many as 1.5 million Americans today are believed to have some form of autism. Based on statistics from the U.S. Department of Education and other governmental agencies, autism is growing at a rate of 10-17 percent per year. At these rates, the Autism Society of America estimates that autism could affect 4 million Americans in the next decade. The overall incidence of autism is consistent around the globe, though it appears to be four times more prevalent in boys than girls. Autism is a national health crisis that some estimate costs our economy $90 billion a year in programs and services, according to the Autism Society of America.

### Sensory Integration

The phenomenon of sensory integration provides a theoretical means of explaining and understanding brain dysfunction in many PDD cases. Simultaneously, it has become a popular practical method of helping many individuals with autism. It is believed that children and adults with autism, as well as those with other developmental disabilities, often have a dysfunctional sensory system. Sometimes one or more senses are either over- or under-reactive to stimulation. Such sensory problems may be the underlying reason for such behaviors as rocking, spinning, and hand-flapping. Although receptors for the senses are located in the peripheral nervous system (which includes everything but the brain and spinal cord), it is believed that the problem stems from neurological dysfunction in the central nervous system—the brain. As observed in individuals with autism, sensory integration techniques, such as pressure-touch, can facilitate attention and awareness, and they can reduce overall arousal.

Sensory integration is an innate neurobiological process that refers to the integration and interpretation of sensory stimulation from the environment by the brain. In contrast, sensory integrative dysfunction is a disorder in which sensory input is not integrated or organized appropriately in the brain, which may produce varying degrees of problems in cognitive development, information processing, and behavior.

Sensory integration focuses primarily on three basic senses—tactile, vestibular, and proprioceptive. Their interconnections start forming before birth and continue to develop as a person matures and interacts with his/her environment. The three senses are not only interconnected, but they are also connected with other systems in the brain. Although these three sensory systems are less familiar to our awareness than our visual and auditory systems, they are critical to our basic survival. The inter-relationship among these three senses is complex. Basically, they allow us to experience, interpret, and respond to different stimuli in our environment.

According toLoma Jean King, OTR, FAOTA (the Founder and Director of the Center for Neurodevelopmental Studies, Inc. in Phoenix, Ariz.) 85 to 90 percent of children with autism have sensory integration problems, some of which are much more obvious than others. A therapist's trained eye may recognize subtle signs that may prove quite significant, whereas a parent may not realize their significance. Often small changes in helping the child to be less sensitive to sensory input produced significant changes in behavior. For instance, sitting on a beach ball or a T-stool can help the child to improve his/her attention. It is believed that increased vestibular and proprioceptive input might help the nervous system to organize and process information better.

### Tactile System

The tactile system includes nerves under the skin's surface that send information to the brain. This information encompasses light touch, pain, temperature, and pressure. These play an important role in perceiving the environment as well as in protective reactions for survival.

Dysfunction in the tactile system can be observed as withdrawing when being touched, refusing to eat certain ‘textured’ foods and/or to wear certain types of clothing, complaining about having one's hair or face washed, avoiding getting one's hands dirty (e.g., glue, sand, mud, finger-paint), and using one's finger tips rather than whole hands to manipulate objects. A dysfunctional tactile system may lead to a misperception of touch and/or pain (hyper- or hyposensitive) and may lead to self-imposed isolation, general irritability, distractibility, and hyperactivity.

Tactile defensiveness is a condition in which an individual is extremely sensitive to a light touch. Theoretically, when the tactile system is immature and working improperly, abnormal neural signals are sent to the cortex in the brain, which can interfere with other brain processes. This, in turn, causes the brain to be overly stimulated resulting in excessive brain activity, which can neither be turned off nor organized. This type of over-stimulation in the brain can make it difficult for an individual to organize one's behavior and concentration, and may lead to a negative emotional response to touch sensations.

### Vestibular System

The vestibular system refers to structures within the inner ear (the semi-circular canals) that detect movement and changes in the position of the head. For example, the vestibular system tells you when your head is upright or tilted (even with your eyes closed). Dysfunction within this system may manifest itself in two different ways. Some children with autism may be hypersensitive to vestibular stimulation and have fearful reactions to ordinary movement activities (e.g., swings, slides, ramps, inclines). They may also have trouble learning to climb or descend stairs or hills; and they may be apprehensive walking or crawling on uneven or unstable surfaces. As a result, they seem fearful in space. In general, these children appear clumsy. On the other extreme, some children may actively seek very intense sensory experiences such as excessive body whirling, jumping, and/or spinning. These children demonstrate signs of a hypo-reactive vestibular system; that is, they are trying continuously to stimulate their vestibular systems.

### Proprioceptive System

The proprioceptive system refers to components of muscles, joints, and tendons that provide a person with a subconscious awareness of body position. When proprioception is functioning efficiently, an individual's body position is automatically adjusted to different situations; for example, the proprioceptive system is responsible for providing the body with the necessary signals to allow us to sit properly in a chair and to step off a curb smoothly. It also allows us to manipulate objects using fine motor movements, such as writing with a pencil, using a spoon to drink soup, and buttoning one's shirt.

Some common signs of proprioceptive dysfunction are clumsiness, a tendency to fall, a lack of awareness of body position in space, odd body posturing, minimal crawling when young, difficulty manipulating small objects (buttons, snaps), eating in a sloppy manner, and resistance to new motor movement activities.

Another dimension of proprioception is praxis or motor planning. This is the ability to plan and execute different motor tasks. In order for this system to work properly, it must rely on obtaining accurate information from the sensory systems and then to organize and interpret this information efficiently and effectively.

### Implications

In general, dysfunction within these three systems manifests itself in many ways. Autistic children may be over- or under-responsive to sensory input; their activity level may be either unusually high or unusually low; they may be in constant motion or may get fatigued easily. In addition, some children with autism may fluctuate between these extremes. Gross and/or fine motor coordination problems are also common when these three systems are dysfunctional. Consequently, speech/language delays and academic under-achievement may occur. Behaviorally, the child may become impulsive, easily distractible, and show a general lack of planning. Some children may also have difficulty adjusting to new situations and may react with frustration, aggression, or withdrawal. Usually, evaluation and treatment of basic sensory integrative processes is performed by occupational therapists and/or physical therapists. The therapist's general goals are: (1) to provide the child with sensory information, which helps to organize the central nervous system, (2) to assist the child in inhibiting and/or modulating sensory information, and (3) to assist the child in processing a more organized response to sensory stimuli.

### Application of the Systems of the Present Invention for Autism and Related Conditions

The systems of the present invention have been developed in order to enhance sensory integration and address sensory dysfunction. Experiments conducted during the development of the present invention have demonstrated that healthy as well as sick or diseased subjects (e.g., bipolar vestibular dysfunction patients) demonstrate improvement or correction of, among other things, their vestibular function (e.g., balance), proprioception, motor control, vision, posture, cognitive functions, tinnitus, emotional conditions and sleep as a direct consequence of training procedures with the systems of the present invention.

In some embodiments, the present invention provides systems and treatments for treating or improving misperception of touch and/or pain (hyper- or hyposensitive), self-imposed isolation, general irritability, distractibility, tactile defensiveness, vestibular dysfunction, and activity level (e.g., hyper- or hypo-activity) in a subject with a Pervasive Developmental Disorder (PDD), including, but not limited to an Autistic Disorder, Asperger's Disorder, Childhood Disintegrative Disorder (CDD), Rett's Disorder, and PDD-Not Otherwise Specified (PDD-NOS). In some embodiments the present invention provides systems and methods of treatment to intensify and extend vestibular performance, posture control, sensory-motor coordination and sensory integration; provide stress relief and relaxation; improve sleep patterns and cognitive function; and to extend the range of everyday physical and mental activity in subjects with autism.

It is contemplated that, in some embodiments of the present invention, the systems of the present invention are used in combination with other treatments (e.g., drugs currently used to treat PDDs in general or Autism in particular) for treating a subject with a PDD (e.g., autism). Thus, the present invention provides complimentary or supplementary treatments that can be used in combination with other known treatments. It is contemplated that systems and methods of the present invention (e.g., systems of the present invention with training) intensify the positive effects of current treatments for Autism, and decrease or prevent adverse side effects. In some embodiments, use of systems and methods of the present invention permits a decrease in the dosage of a drug prescribed to treat Autism or a related PDD.

### General Balance.

In some embodiments, autistic subjects experience the return of their sense of balance, increased body control, steadiness, and a sense of being centered after treatment with the systems and methods of the present invention. In some embodiments, a constant sense of moving is eliminated. In some embodiments, subjects are able to walk unassisted, and experience an increase in the ability to walk in dark environments, to walk briskly, to walk in crowds, and to walk on patterned surfaces after treatment with the systems and methods of the present invention. In some embodiments, subjects gain the ability to stand with their eyes closed, with or without a soft base, to walk a straight line, to walk while looking side to side and to walk while looking up and down. In some embodiments, subjects gain the ability to carry items, walk on uneven surfaces, walk up and down embankments, and to ride a bike. In some embodiments, a subject with a Pervasive Developmental Disorder (PDD), (e.g., including, but not limited to an Autistic Disorder, Asperger's Disorder, Childhood Disintegrative Disorder (CDD), Rett's Disorder, and PDD-Not Otherwise Specified (PDD-NOS)) becomes more physically active after treatment with the systems and methods of the present invention.

### Posture, Proprioception and Motor Control.

In some embodiments, a subject with a Pervasive Developmental Disorder (PDD), enjoys more fluid body movements, and movements that are more confident, light, relaxed and quick after treatment with the systems and methods of the present invention. In some embodiments, fine motor skills are refined and gait improves. In some embodiments, subjects enjoy improved posture, body segment alignment, stamina, and general energy levels.

### Vision

In some embodiments, PDD patients display improved vision after receiving treatments according to the present invention. Improved vision includes, but is not limited to, vision becoming clearer, more stable, clearer, and brighter, reduction of oscillopsia, widening of peripheral vision, improvement of depth perception, reduction of or elimination of double vision, and reduction of or elimination of movement of stationary objects and visual fatigue.

In some embodiments, PDD subjects experience improvements of all components of sensory integration when exposed to BrainPort balance therapy.

### Stress Relief and Relaxation

Since individuals with autism typically have communication problems, they are more likely to experience stress in their daily life than individuals with good communication skills. June Groden, PhD (Director of the Groden Center in Providence, R.I.), suggests that a relaxation program constituted of teaching subjects, including individuals with autism, how to discriminate between tense muscles and relaxed muscles can be highly effective.

Children and adults are taught the relaxation procedure, usually in a one-on-one teaching session lasting for as long as the participant can maintain attention. This usually ranges from a few minutes to twenty minutes. The person learns to tighten and relax the arms, hands, and legs, and to practice deep breathing in a sitting position.

The patient is then taught relaxing without tensing. Finally, the person is taught to tighten and relax all remaining muscle groups of the body.

Such relaxation program can be used to develop self-control by the individual learning to achieve a relaxation response in place of the typical maladaptive behavior he or she exhibits during stressful situations.

Accordingly, in some embodiments, PDD subjects experience an improvement in relaxation ability after treatment with the systems and methods of the present invention.

In some embodiments, use of systems of the present invention with training results in physical and emotional relaxation in PDD patients. In some embodiments, deep muscular and emotional relaxation is achieved. In further embodiments, the state of relaxation is reproducible or increases through subsequent sessions. Importantly, because the systems and methods of the present invention do not possess negative side effects, such systems and methods avoid the unwanted side effects of antidepressants, which often cause significant difficulties in individuals with autism.

### Sleep Adjustment

Sleep abnormalities are common in individuals with autism.

Accordingly, in some embodiments, treatments (e.g., treatments with the systems of the present invention) provided by the present invention to a subject (e.g., a PDD subject) improves sleep. It is contemplated that sleep improvement occurs and is perceived as being fuller, longer, and more restful, often with no awakenings during the night. As an additional impact, in some embodiments, treatment with the systems of the present invention results in improved sleep patterns.

It is further contemplated that the systems and methods of the present invention provide both direct (e.g., balance, etc.) and indirect (e.g., sense of well being) benefits that provide a general therapeutic value. For at least some subjects, it is contemplated that use of the systems of the present invention provides temporary or permanent reduction or removal of symptoms associated with PDD. For example, through use of the systems and methods of the present invention, a subject may be trained or treated to perceive and/or filter out (e.g., ignore) sensory information so as to effect an improvement in function. The associated indirect effects further improve the subject's capabilities. In one exemplary embodiment, a subject that has difficulty filtering sound is provided with audio information (e.g., a parent's voice) via electrotactile stimulation of the tongue so as to provide second source of the information. Likewise, in other embodiments, sensory information that is perceived as unpleasant is masked by the addition of electrotactile stimulation of the tongue that provides an alternative or counteracting sensory response. In some embodiments, the general improvements to cognitive function and overall well-being provided by the systems of the present invention reduce or eliminate symptoms of the diseases and conditions. Thus, it is contemplated that such treatments, at least for some subjects, may be curative or substantially curative of the disease or condition.

### Example 23

### Parkinson's Disease

### Parkinson's Disease

Parkinson's disease is a slowly progressive neurodegenerative disorder caused by damaged or dead dopamine-neurons in the substantia nigra, a region of the brain that controls balance and coordinates muscle movement. Dopamine is a neurotransmitter that carries information from neuron to neuron and eventually out to the muscles. When these dopamine neurons start to die, the lines of communication between the brain and the body become progressively weaker. Eventually, the brain is no longer able to direct or control muscle movement in a normal manner.

Parkinson's disease causes substantial morbidity and results in a shortened life span. Mortality rates in 1967 for patients with Parkinson's disease were three times those of control subjects; 30 years later, mortality rates were found to be largely unchanged. Thus, despite breakthroughs in medical treatment and the availability of exciting new surgical procedures, chronic progression to severe disability is still the rule. Nevertheless, current therapy can slow symptom progression and improve quality of life.

Parkinson's disease severely compromises quality of life. Patients with this illness can find it difficult to read, write and drive. With advanced disease, they often cannot manage basic activities of daily living. Thus, Parkinson's disease can result in loss of employment and, ultimately, loss of personal autonomy.

### Prevalence and Cost

Parkinson's disease is the most common neurodegenerative disease after Alzheimer's disease, with an estimated incidence of 20 per 100,000 and a prevalence of 150 per 100,000. The disease has a roughly equal sex distribution, with a slight male predominance, and no ethnic group is spared.

The mean age at onset of Parkinson's disease is 55 to 60 years. An estimated 1% of the US population over 50 years of age, or about 1 million people, have the disease. However, some physicians have reportedly noticed more cases of “early-onset” Parkinson's disease in the past several years.

Pesticides and other toxins have been suspected, but none has been proved to be a definite causative factor. On the other hand, the search for genetic causes has yielded at least four independent gene loci in various forms of familial Parkinson's disease. The autosomal dominant adult-onset type is linked to a site on chromosome 4q6 and the gene for autosomal recessive juvenile parkinsonism maps to chromosome 6q. Because most patients do not have a clear history of either familial or environmental risk factors, the disorder may be due to a combination of genetic and environmental “influences” or “causes.”

In 1990, more than half of all patients with a diagnosis of Parkinson's disease were being treated in the primary care setting. Although in its later stages the condition can be very difficult to treat, initial diagnosis and early management can usually be accomplished by primary care physicians. These physicians are also in an ideal position to help address the impact that the illness has on the patient's lifestyle and on his or her spouse and family.

According to the National Parkinson Foundation, each patient spends an average of $2,500 a year for medications. After factoring in office visits, Social Security payments, nursing home expenditures, and lost income, the total cost to the Nation is estimated to exceed $5.6 billion annually.

### Primary Symptoms

People with Parkinson's disease may have trouble walking, talking, or completing simple tasks that depend on coordinated muscle movements. The four primary symptoms of Parkinson's disease often appear gradually but increase in severity with time. They are:

Tremor or trembling in hands, arms, legs, jaw, and face; Rigidity or stiffness of the limbs and trunk; Bradykinesia, Slowness of motor movements; and Postural instability or impaired balance and coordination

### Tremor

The tremor of Parkinson's disease is one of the most common presenting signs, being the initial complaint in 70% to 75% of cases. Typically, it is a 4- to 6-Hz resting tremor that may be intermittent in early stages. The tremor associated with Parkinson's disease has a characteristic appearance. Typically, the tremor takes the form of a rhythmic back-and-forth motion of the thumb and forefinger at three beats per second. This is sometimes called “pill rolling.” Tremor usually begins in a hand, although sometimes a foot or the jaw is affected first. It is most obvious when the hand is at rest or when a person is under stress. In three out of four patients, the tremor may affect only one part or side of the body, especially during the early stages of the disease. Later it may become more general. Tremor is rarely disabling and it usually disappears during sleep or improves with intentional movement.

Stress or anxiety may precipitate the tremor. It usually begins unilaterally, affecting one or both limbs, but it can also involve the jaw, lips, and lower facial muscles. It is possible to distinguish the tremor of Parkinson's disease from essential tremor. One study of patients diagnosed with Parkinson's disease by a normeurologist showed that about 25% actually had essential tremor only.

Essential tremor is typically postural and is not usually seen at rest. It may become more prominent at the termination of a movement. It is faster (6 to 9 Hz) than a parkinsonian tremor and is usually bilateral. A pill-rolling quality is usually not present, but a head tremor (titubation) often occurs. The voice of a patient with essential tremor may be tremulous. The patient often has a family history of tremor, which usually resolves temporarily with ingestion of small amounts of alcohol, whereas a parkinsonian tremor is not usually relieved by alcohol. A parkinsonian tremor generally responds to antiparkinsonian medication, whereas essential tremor generally does not.

### Rigidity

Rigidity, or a resistance to movement, affects most parkinsonian patients. A major principle of body movement is that all muscles have an opposing muscle. Rigidity is an increase in muscle tone that is noted as an increase in resistance to passive maneuvers. Movement is possible not just because one muscle becomes more active, but because the opposing muscle relaxes. In Parkinson's disease, rigidity comes about when, in response to signals from the brain, the delicate balance of opposing muscles is disturbed. The muscles remain constantly tensed and contracted so that the person aches or feels stiff or weak. The rigidity becomes obvious when another person tries to move the patient's arm, which will move only in ratchet-like or short, jerky movements known as “cogwheel” rigidity. It can be elicited by having the patient perform similar movements in the opposite limb (activated rigidity). Parkinsonian rigidity is usually more prominent in the extremities than axially. A cogwheeling phenomenon may also be superimposed on the rigidity. As illness progresses, rigidity becomes more severe and the patient may acquire a characteristic stooped posture with the head tilted forward and the arms flexed at the elbows and wrists.

### Akinesia (or Bradykinesia):

Patients with Parkinson's disease often have evidence of akinesia, which is a lack or poverty of movement. They are also likely to display bradykinesia, that is, a slowness and fatiguing of voluntary movement. Bradykinesia, or the slowing down and loss of spontaneous and automatic movement, is particularly frustrating because it is unpredictable. One moment the patient can move easily. The next moment he or she may need help. This may well be the most disabling and distressing symptom of the disease because the patient cannot rapidly perform routine movements. Activities once performed quickly and easily—such as washing or dressing—may take several hours. As noted, these abnormalities may be manifested as decreased facial expression, slowness of movement, or clumsiness in an extremity. A patient may also be slow in such activities as getting dressed or writing. The fatiguing of voluntary movement can be seen in the phenomenon of micrographia, in which a patient's handwriting decreases in fullness and legibility from the beginning of a sentence to the end. Fatiguing can also be elicited by having a patient repeatedly tap a finger or perform another repetitive motion. Amplitude and continuance of motion are gradually lost.

All of these symptoms can progress in severity. Later in the course of the illness, akinesia and bradykinesia contribute to disabling postural difficulties.

Deficits in Gait and Postural instability

Initially, the only change in a patient's gait may be decreased arm swing or, possibly, easy fatigability. Later, the stride becomes shortened, and eventually it becomes a shuffle. A patient may drag the foot on the predominantly affected side. As the disease progresses, patients may have “freezing episodes,” particularly when turning. They may also have difficulty initiating a gait.

In later stages of the disease, deficits in postural reflexes develop. Postural instability, or impaired balance and coordination, causes patients to develop a forward or backward lean and to fall easily. When bumped from the front or when starting to walk, patients with a backward lean have a tendency to step backwards, which is known as retropulsion. Postural instability can cause patients to have a stooped posture in which the head is bowed and the shoulders are drooped. As the disease progresses, walking may be affected. Patients may halt in mid-stride and “freeze” in place, possibly even toppling over. Or patients may walk with a series of quick, small steps as if hurrying forward to keep balance. This is known asfestination. Ultimately, this leads to falls, which greatly increase morbidity and mortality rates.

When postural reflexes are inadequate, patients may fall if they are pushed even slightly forward or backward, or if they are standing in a moving vehicle such as a bus or train. Clinical scales rating the presence and severity of these signs are useful.

### Additional Symptoms

Various other symptoms accompany Parkinson's disease; some are minor, others are more bothersome. Many can be treated with appropriate medication or physical therapy. No one can predict which symptoms will affect an individual patient, and the intensity of the symptoms also varies from person to person. None of these symptoms is fatal, although swallowing problems can cause choking.

Depression. Depression is a common problem and may appear early in the course of the disease, even before other symptoms are noticed. Depression may not be severe, but it may be intensified by the drugs used to treat other symptoms of Parkinson's disease.

Emotional changes. Some people with Parkinson's disease become fearful and insecure. Perhaps they fear they cannot cope with new situations. They may not want to travel, go to parties, or socialize with friends. Some lose their motivation and become dependent on family members. Others may become irritable or uncharacteristically pessimistic. Memory loss and slow thinking may occur, although the ability to reason remains intact. Whether people actually suffer intellectual loss (also known as dementia) from Parkinson's disease is a controversial area still being studied.

Difficulty in swallowing and chewing. Muscles used in swallowing may work less efficiently in later stages of the disease. In these cases, food and saliva may collect in the mouth and back of the throat, which can result in choking or drooling. Medications can often alleviate these problems.

Speech changes. About half of all parkinsonian patients have problems with speech. They may speak too softly or in a monotone, hesitate before speaking, slur or repeat their words, or speak too fast. A speech therapist may be able to help patients reduce some of these problems.

Urinary problems or constipation. In some patients bladder and bowel problems can occur due to the improper functioning of the autonomic nervous system, which is responsible for regulating smooth muscle activity. Some people may become incontinent while others have trouble urinating. In others, constipation may occur because the intestinal tract operates more slowly. Constipation can also be caused by inactivity, eating a poor diet, or drinking too little fluid. It can be a persistent problem and, in rare cases, can be serious enough to require hospitalization.

Skin problems. In Parkinson's disease, it is common for the skin on the face to become very oily, particularly on the forehead and at the sides of the nose. The scalp may become oily too, resulting in dandruff. In other cases, the skin can become very dry. These problems are also the result of an improperly functioning autonomic nervous system. Standard treatments for skin problems help. Excessive sweating, another common symptom, is usually controllable with medications used for Parkinson's disease.

Sleep problems. These include difficulty staying asleep at night, restless sleep, nightmares and emotional dreams, and drowsiness during the day. It is unclear if these symptoms are related to the disease or to the medications used to treat Parkinson's disease. Patients should never take over-the-counter sleep aids without consulting their physicians.

It is estimated that dementia occurs in 20% to 25% of patients with Parkinson's disease, making the illness difficult to distinguish from Alzheimer's disease. However, the dementia of Parkinson's disease is usually a late feature. Prominent early dementia may indicate coexisting Alzheimer's disease or another illness.

### Current Treatments

Presently, there is no cure for Parkinson's disease. Since most of the symptoms are due to the lack of dopamine in the brain, effective medications aim at temporarily replenishing or mimicking dopamine's actions. These drugs—levodopa and the dopamine agonists ropinirole, pramipexole, and pergolide—reduce muscle rigidity, improve speed and coordination of movement, and relieve tremor.

Without doubt, the gold standard of present therapy is the drug levodopa (also called L-dopa). L-Dopa (from the full name L-3,4-dihydroxyphenylalanine) is a simple chemical found naturally in plants and animals. Levodopa is the generic name used for this chemical when it is formulated for drug use in patients. Nerve cells can use levodopa to make dopamine and replenish the brain's dwindling supply. Dopamine itself cannot be given because it doesn't cross the blood-brain barrier, the elaborate meshwork of fine blood vessels and cells that filters blood reaching the brain. Usually, patients are given levodopa combined with carbidopa. When added to levodopa, carbidopa delays the conversion of levodopa into dopamine until it reaches the brain, preventing or diminishing some of the side effects that often accompany levodopa therapy. Carbidopa also reduces the amount of levodopa needed.

Levodopa's success in treating the major symptoms of Parkinson's disease is a triumph of modern medicine. First introduced in the 1960s, it delays the onset of debilitating symptoms and allows the majority of parkinsonian patients—who would otherwise be very disabled to extend the period of time in which they can lead relatively normal, productive lives.

Levodopa is not a cure. Although it can diminish the symptoms, it does not replace lost nerve cells and it does not stop the progression of the disease. Although levodopa helps at least three-quarters of parkinsonian cases, not all symptoms respond equally to the drug. Bradykinesia and rigidity respond best, while tremor may be only marginally reduced. Problems with balance and other symptoms may not be alleviated at all.

### Side Effects of Levodopa

The most common side effects are nausea, vomiting, low blood pressure, involuntary movements, and restlessness. In rare cases patients may become confused. Dyskinesias, or involuntary movements such as twitching, nodding, and jerking, most commonly develop in people who are taking large doses of levodopa over an extended period. These movements may be either mild or severe and either very rapid or very slow. The only effective way to control these drug-induced movements is to lower the dose of levodopa or to use drugs that block dopamine, but these remedies usually cause the disease symptoms to reappear. Doctors and patients must work together closely to find a tolerable balance between the drug's benefits and side effects.

In addition, many doctors recommend physical therapy or muscle-strengthening exercises to help people handle their daily activities. Because movements are affected in Parkinson's disease, exercising may help people improve their mobility. Some doctors prescribe physical therapy or muscle-strengthening exercises to tone muscles and to put underused and rigid muscles through a full range of motion. Exercises will not stop disease progression, but they may improve body strength so that the person is less disabled. Exercises improve balance, helping people overcome gait problems, and they can also strengthen certain muscles so that people can speak and swallow better. Exercises can also improve the emotional well-being of parkinsonian patients by giving them a feeling of accomplishment. Although structured exercise programs help many patients, more general physical activities, such as walking, gardening, swimming, calisthenics, and using exercise machines, also appear to provide some benefit.

In some cases, surgery may be appropriate if the disease doesn't respond to drugs. A therapy called deep brain stimulation has been approved by the U.S. Food and Drug Administration, as well, as Globus pallidus internal-segment pallidotomy and Fetal nigral transplantation.

In deep brain stimulation, electrodes are implanted into the brain and connected to a small electrical device called a pulse generator that can be externally programmed. Deep brain stimulation can reduce the need for levodopa and related drugs, which in turn decreases the involuntary movements called dyskinesias. It also helps to alleviate fluctuations of symptoms and to reduce tremors, slowness of movements, and gait problems. Deep brain stimulation requires careful programming of the stimulator device in order to work correctly.

### Prognosis

Although medications can relieve symptoms for a period of time, they do not slow or stop the natural progression of the disease. The course of the disease varies widely. Some people have mild symptoms for many years, while others have severe symptoms and a quicker progression. Despite new medical and surgical therapy, mortality rates for Parkinson's disease remain unchanged.

Although Levodopa is the most effective drug for Parkinson's disease, its long-term use is associated with significant motor complications. Dopamine agonists hold promise because of more sustained stimulation of dopamine receptors and possibly an antioxidant effect. Selegiline, amantadine, and anticholinergics are still used but must be employed with caution in the elderly. COMT inhibitors may be useful adjuncts to levodopa therapy but are plagued with serious adverse effects.

### Parkinson's and the Systems of the Present Invention

Experiments conducted during the development of the present invention have demonstrated that healthy as well as sick or diseased subjects (e.g., bipolar vestibular dysfunction patients) demonstrate improvement or correction of, among other things, their vestibular function (e.g., balance), proprioception, motor control, vision, posture, cognitive functions, tinnitus, emotional conditions and sleep as a direct consequence of training procedures with the systems of the present invention.

Accordingly, in some embodiments, the present invention provides systems and methods for correcting or improving motor control (e.g., walking, talking, or completing simple tasks that depend on coordinated muscle movements) in a subject with Parkinson's disease.

In some embodiments, the present invention provides systems and methods for correcting or improving tremor or trembling in hands, arms, legs, jaw, and face; correcting or improving rigidity or stiffness of the limbs and trunk; correcting or improving bradykinesia, correcting or improving slowness of motor movements; and correcting or improving postural instability or impaired balance and coordination in a subject with Parkinson's disease.

In some embodiments, the present invention provides systems and treatments for correcting or improving depression, emotional changes, difficulty in swallowing and chewing, speech changes, urinary problems or constipation, and sleep problems in a subject with Parkinson's disease.

In some embodiments, the present invention provides systems and methods for low cost, highly sensitive diagnostic tremor tool. In some embodiments, the device provides spectral analysis of head stability can be especially useful for diagnosis of the Parkinson's tremor, no matter which body part is affected. Even though the head is the most sensitive part of the body, in some embodiments, the present invention uses an external accelerometer instead of an internal one (e.g. hand-based, instead of head-based).

In some embodiments, the systems of the present invention differentiates peaks within a frequency range of 2-10 Hz, which is important for separation of Parkinson's and essential tremors. In other embodiments, the device differentiates between peaks in a range of 5-10 Hz, 10-20 Hz, 15-25 Hz, 1-10 Hz, or 10-100 Hz. It is contemplated that diagnostic procedures with quantitatively measurable and scaleable data are used for early diagnosis of tremor and balance problems. The present invention provides a portable system designed to be comparable with desktop and laptop computers. It is contemplated that data recording and analytical routines will quantify postural stability, thereby enabling description of postural stability.

The systems of the present invention have been shown to improve and recover postural control and gait stability in both BVD patients and normal subjects. Thus, in some embodiments, the present invention provides systems and methods that provide and facilitate the muscular relaxation in all muscular groups in subjects who typically suffer from rigidity in neck and upper back muscles (e.g., Parkinson's subjects). Festination and Parkinson's jerk movement are similar to the sharp, spike- and step-like movement in BVD patients. These abnormal movements were completely eliminated after training. Consequently, BVD patients achieved a “superstability” stage. Accordingly, the present invention provides systems and methods to eliminate or correct jerk like movements associated with Parkinson's disease.

In addition, it is contemplated that, in some embodiments of the present invention, the systems of the present invention are used in combination with other treatments (e.g., Levadopa or similar drugs) for treating a subject with Parkinson's disease. Thus, the present invention provides complimentary or supplementary treatments that can be used in combination with other known treatments. It is contemplated that systems and methods of the present invention intensify the positive effects of current treatments for Parkinson's (e.g., Levadopa), and decrease or prevent adverse side effects (e.g., prevent abnormal motor pattern associated with Levadopa). In some embodiments, use of systems and methods of the present invention will permit a decrease in the dosage of a drug prescribed to treat Parkinson's.

In some embodiments, the systems and methods of the present invention are used in combination with a training regiment based on advanced physical therapy. In some embodiments, such combination results in an overall improvement of motor control, posture and balance, among other things.

In some embodiments, the systems and methods of the present invention are used in place of, or in combination with, surgically invasive procedures (e.g., deep brain stimulation) for treating Parkinson's patients. Long term potentiation, the systems and methods of the present invention, and deep brain stimulation share a few common features, including: long therapy times (more than few minutes); electrical stimulation (rectangular impulses); similar pulse rates (100-200 Hz) of the neural (or sensory) tissue; and long lasting (from hours to days) effects. Accordingly, it is contemplated that, in some embodiments, subjects undergoing treatment with the systems of the present invention experience long term potentiation (e.g., long lasting changes lasting from hours to days to weeks or longer) in brain and body functions.

In some embodiments, the present invention provides systems and methods for reducing or correcting speech problems resulting from tongue mobility loss associated with Parkinson's disease or other diseases. For example, in some embodiments, the systems of the present invention are used to keep muscular tonus within normal range as a consequence of antidromic stimulation (e.g., stimulation from the tongue to the nerve center) of the hypoglossal nerve (major motor nerve of the tongue).

The present invention also provides systems and methods for improving or correcting cognitive decline observed in a Parkinson's subject.

In some embodiments, the present invention provides systems and methods for preventing or diminishing involuntary movements. For example, in some embodiments, it is contemplated that the systems and methods of the present invention are capable of changing the signal-to-noise ratio in vestibular and motor-control circuitries in the human brain, and of suppressing the “noise” and “error” signals in posture control groups of muscles.

In some embodiments, the present invention provides systems and methods for improving or correcting motor control (e.g., improvement of fine finger movement control); relieving stress; eliminating depression; and improving the emotional status of Parkinson's patients.

### Systems and Methods of the Present Invention Treat Parkinson's Disease Symptoms

Balance-affected Parkinson's patients with peripheral, central, and vestibulo-cerebellar disorders that used (e.g., trained with) the systems and methods of the present invention regained functional posture, gait, and motor control, resulting in improved balance for extended periods of time after use. Symptoms common to Parkinson's such as muscle rigidity, involuntary movements, and posture and gait dysfunction were improved or alleviated in balance-affected patients.

Data generated in these studies indicated clear improvement in balance and posture control as measured by computerized dynamic posturography after just one week of training, with an increase in the average composite equilibrium SOT score of 22.3% (n=3). Significant improvement in walking speed and distance was also demonstrated after one week, as evidenced with 6-minute walk tests showing an average speed increase of 50.8% and an average distance increase of 50.0% (n=2). In addition, a measure of upper limb akinesia (index finger tapping) demonstrated 43% improvement in coordination between the two index fingers in bimanual tapping (n=1). Thus, in some embodiments, systems and methods of the present invention can be used for treatment of Parkinson's symptoms (e.g., delaying and/or reducing the need for neuropharmacologic treatments and/or surgical interventions).

### Example 24

### Stroke

### Stroke in General

More than 2,400 years ago the father of medicine, Hippocrates, recognized and described stroke, the sudden onset of paralysis. Until recently, modern medicine has had very little control over this disease, but the world of stroke medicine is changing and new and better therapies are being developed. Today, some people who suffer from stroke can recover from the attack with no or few disabilities if they are treated promptly. Doctors can finally offer stroke patients and their families the one thing that until now has been so hard to give—hope.

In ancient times, stroke was called apoplexy, a general term that physicians applied to any condition in which a patient was suddenly struck with paralysis. Because many conditions can cause sudden paralysis, the term apoplexy did not indicate a specific diagnosis or cause.

Scientists now know that there is a very short window of opportunity for treatment of the most common form of stroke. Nevertheless, systems and methods of the present invention, used alone or in combination with other advances in the field of cerebrovascular disease, provide stroke patients a chance for survival and recovery.

A stroke is a sudden interruption of the blood supply in the brain. Most strokes are caused by an abrupt blockage of arteries leading to the brain (ischemic stroke). Other strokes are caused by bleeding into brain tissue when a blood vessel bursts (hemorrhagic stroke). A stroke, also called a brain attack, happens when brain cells die because of inadequate blood flow. A stroke is considered to be a cardiovascular disease and a neurological disorder. When the symptoms of a stroke last only a short time (less than an hour), this is called a transient ischemic attack (TIA) or mini-stroke.

Stroke has many consequences. The effects of a stroke depend on which part of the brain is injured, and how severely it is injured. Stroke may cause sudden weakness, loss of sensation, or difficulty with speaking, seeing, or walking. Since different parts of the brain control different areas and functions, it is usually the area immediately surrounding the stroke that is affected. Stroke can be accompanied by a headache, but it can also be completely painless. It is very important to recognize the warning signs of stroke and to get immediate medical attention if they occur.

There are several other types of injury that can affect the brain, including aneurysms, subdural hematomas (bleeding adjacent to the brain), trauma, infection, among others, that are also contemplated to be treatable via systems and methods of the present invention.

Stroke appears to run in some families who may either have a genetic mutation that predisposes them to stroke, or share a lifestyle that contributes to stroke risk factors. Other than genetic predisposition, additional risk factors for stroke are high blood pressure, heart disease, smoking, diabetes, and high cholesterol. Controlling these risk factors can decrease the likelihood of getting a stroke.

### Health Statistics

Each year, more than 700,000 strokes occur in the United States, making stroke the third leading cause of death (behind heart disease and cancer) and the leading cause of long-term disability in the U.S. About 500,000 of these are first attacks, and 200,000 are recurrent attacks. Stroke killed 275,000 people in 2002 and accounted for about 1 in almost 15 deaths in the United States.

On average, someone in the United States suffers from a stroke every 45 seconds; every 3.1 minutes someone dies of a stroke. 22% of men and 25% of women who have an initial stroke die within a year. At all ages, 40,000 more women than men have a stroke. 28% of people who suffer a stroke in a given year are under age 65.

According to the National Stroke Association: 10% of stroke survivors recover almost completely; 25% recover with minor impairments; 40% experience moderate to severe impairments that require special care; 10% require care in a nursing home or other long-term facility; 15% die shortly after the stroke; and approximately 14% of stroke survivors experience a second stroke in the first year following the initial stroke.

About 4.7 million stroke survivors (2.3 million men, 2.4 million women) are alive today. In addition, there are millions of husbands, wives, children and friends who care for stroke survivors and whose own lives are personally affected. Approximately 10 percent of stroke survivors resume prior activity levels. Mild to moderate disability results in about 50 percent of strokes, while severe disability affects the remaining 40 percent of individuals who survive a stroke.

Cost of Stroke to the United States (Data from 1997)

The total cost of stroke to the United States: estimated at about $43 billion/year. The direct costs for medical care and therapy: estimated at about $28 billion/year while indirect costs from lost productivity and other factors: estimated at about $15 million/year. The average cost of care for a patient up to 90 days after a stroke: $15,000 (The Stroke/Brain Attack Reporter's Handbook, National Stroke Association, Englewood, Colo., 1997).

### Symptoms

The most common sign of a stroke is sudden weakness of the face, arm or leg, most often on one side of the body. Other warning signs can include sudden changes, such as: numbness of the face, arm, or leg, especially on one side of the body; confusion, trouble speaking or understanding speech; vision disturbances, trouble seeing in one or both eyes; trouble walking, dizziness, loss of balance or coordination; severe headache with no known cause; slurred speech, inability to speak or understand speech; difficulty reading or writing; swallowing difficulties or drooling; loss of memory; vertigo (spinning sensation); personality changes; mood changes (depression, apathy); drowsiness, lethargy, or loss of consciousness; and uncontrollable eye movements or eyelid drooping

The warning signs of a stroke depend on such factors as which side and what part of the brain are affected, and how severely the brain is injured. Therefore, each person may have different stroke warning signs. Stroke may be associated with a headache, or may be completely painless. If one or more of these symptoms are present for less than 24 hours, it may be a transient ischemic attack (TIA). A TIA is a temporary loss of brain function and a warning sign for a possible future stroke.

### Stroke Effects

Stroke can affect people in different ways. It depends on the type of stroke, the area of the brain affected and the extent of the brain injury. Brain injury from a stroke can affect the senses, motor activity, speech and the ability to understand speech. It can also affect behavioral and thought patterns, memory and emotions.

Paralysis or weakness on one side of the body is common. Most of these problems can improve over time. In some patients they will disappear completely. Motor deficits can result from damage to the motor cortex in the frontal lobes of the brain or from damage to the lower parts of the brain, such as the cerebellum, which controls balance and coordination.

Loss of awareness: Stroke often causes people to lose mobility and/or feeling in an arm and/or leg. If this affects the left side of the body (caused by a stroke on the right side of the brain), stroke survivors may also forget or ignore their weaker side. This problem is called neglect. As a result, they may ignore items on their affected side and not think that their left arm or leg belongs to them. They also may dress only one side of their bodies and think they're fully dressed. Bumping into furniture or door jambs is also common.

Perception: A stroke can also affect seeing, touching, moving and thinking, so a person's perception of everyday objects may be changed. Stroke survivors may not be able to recognize and understand familiar objects the way they did before.

When vision is affected, objects may look closer or farther away than they really are. This causes survivors to have spills at the table and collisions or falls when they walk.

Hearing and speech: Stroke usually doesn't cause hearing loss, but people may have problems understanding speech. They also may have trouble saying what they're thinking. This is called aphasia. Aphasia affects the ability to talk, listen, read and write. It's most common with a stroke affecting the left side of the brain, which may also weaken the body's right side.

A related problem is that a stroke can affect muscles used in talking (those in the tongue, palate and lips). Speech can be slowed, slurred or distorted, so stroke survivors can be hard to understand. This is called dysarthria. It may require the help of a speech expert.

Chewing and swallowing food: The problem with chewing and swallowing food is called dysphagia. It can occur when muscles on one side of the mouth are weak. One or both sides of the mouth can also lack feeling, increasing the risk of choking.

Ability to think clearly: Specific parts of the brain allow us to form long-term and short-term memories. (Short-term memories help us remember why we got up and walked into the next room, for example.) With injury to these areas, it may be hard to plan and carry out even simple activities. Stroke survivors may not know how to start a task, they confuse the sequence of logical steps in tasks, or forget how to do tasks they've done many times before.

Emotions: Some areas of the brain produce emotions, just as other parts produce movement or allow us to see, hear, smell or taste. If these areas are injured by a stroke, a survivor may cry easily or have sudden mood swings, often for no apparent reason. This is called emotional lability. Laughing uncontrollably may also occur, though it isn't as common as crying.

Depression is common as stroke survivors recover and as they come to terms with any permanent impairment. It is a clinical behavioral problem that can hamper recovery and rehabilitation and may even lead to suicide. Post-stroke depression is treated as any other depression, namely, with antidepressant medications and therapy.

Stroke patients may experience pain, uncomfortable numbness, or strange sensations after a stroke. These sensations may be due to many factors, including damage to the sensory regions of the brain, stiff joints, or a disabled limb. An uncommon type of pain resulting from stroke is called central stroke pain or central pain syndrome (CPS). CPS results from damage to an area in the mid-brain called the thalamus.

The pain is a mixture of sensations, including heat and cold, burning, tingling, numbness, sharp stabbing and underlying aching pain. The pain is often worse in the extremities—the hands and feet—and is increased by movement and temperature changes, cold temperatures in particular. Unfortunately, since most pain medications provide little relief from these sensations, very few treatments or therapies exist to combat CPS. It's important for stroke survivors to receive appropriate rehabilitation to help alleviate these deficits.

### Stroke Treatment

Physicians have a range of therapies to choose from when determining a stroke patient's individual therapeutic plan. The type of stroke therapy a patient should receive depends upon the stage of disease. Generally, there are three treatment stages for stroke: prevention, therapy immediately after stroke, and post-stroke rehabilitation.

### Prevention

Therapies to prevent a first or recurrent stroke are based on treating an individual's underlying risk factors for stroke, such as hypertension, atrial fibrillation, and diabetes, or preventing the widespread formation of blood clots that can cause ischemic stroke in everyone, whether or not risk factors are present.

Prevention is the best possible stroke treatment. Many stroke risk factors can be modified with lifestyle changes, so taking an active role in reducing risk factors can help prevent strokes. Practicing stroke prevention has other health benefits—many aspects of stroke prevention also reduce the risk of heart attack, hypertension, and diabetes. To prevent bleeding strokes, it is recommended to take steps to avoid falls and injuries.

Therapies for stroke include immediate (or acute) treatment: medications, surgery and long-term rehabilitation.

### Acute Stroke Therapies

Acute stroke therapies try to stop a stroke while it is happening by quickly dissolving a blood clot causing the stroke or by stopping the bleeding of a hemorrhagic stroke.

Medication or drug therapy is the most common treatment for stroke. The most popular classes of drugs used to prevent or treat stroke are antithrombotics (antiplatelet agents and anticoagulants), thrombolytics, and neuroprotective agents. Other medications may be needed to control associated symptoms. Analgesics (pain killers) may be needed to control severe headache. Anti-hypertensive medication may be needed to control high blood pressure.

Surgery can be used to prevent stroke, to treat acute stroke, or to repair vascular damage or malformations in and around the brain. There are two prominent types of surgery for stroke prevention and treatment: carotid endarterectomy and extracranial/intracranial (EC/IC) bypass.

For hemorrhagic stroke, surgery is often required to remove pooled blood from the brain and to repair damaged blood vessels. Life support and coma treatment are performed as needed.

### Long Term Stroke Treatment

The purpose of post-stroke rehabilitation is to overcome disabilities that result from stroke damage. The goal of long-term treatment is to recover as much function as possible and prevent future strokes. Depending on the symptoms, rehabilitation includes physical therapy, occupational therapy, speech therapy and psychological therapy. The recovery time differs from person to person.

Physical Therapy (PT): Helps stroke victims to relearn walking, sitting, lying down, switching from one type of movement to another. For most stroke patients, physical therapy (PT) is the cornerstone of the rehabilitation process. A physical therapist uses training, exercises, and physical manipulation of the stroke patient's body with the intent of restoring movement, balance, and coordination. The aim of PT is to have the stroke patient relearn simple motor activities such as walking, sitting, standing, lying down, and the process of switching from one type of movement to another.

Occupational Therapy (OT): Helps stroke patients to relearn eating, drinking, swallowing, dressing, bathing, cooking, reading, writing, toileting. The goal of OT is to help the patient become independent or semi-independent

Speech Therapy The focus of speech therapy is on relearning language and communication skills. Speech and language problems arise when brain damage occurs in the language centers of the brain. Due to the brain's great ability to learn and change (called brain plasticity), other areas can adapt to take over some of the lost functions (See, e.g., Ptito et al., Brain, 128(Pt 3):606-14 [2005]). Speech therapy helps stroke patients relearn language and speaking skills, or learn other forms of communication. Speech therapy is appropriate for patients who have no deficits in cognition or thinking, but have problems understanding speech or written words, or problems forming speech. A speech therapist helps and instructs stroke patients on how to improve their language skills, to develop alternative ways of communicating, and to expand coping skills enabling them to deal with the frustration of not being able to communicate fully. With time and patience, a stroke survivor should be able to regain some, and sometimes all, language and speaking abilities.

Psychological/Psychiatric Therapy: These methods alleviate some mental and emotional problems. Many stroke patients require psychological or psychiatric help after a stroke. Psychological problems, such as depression, anxiety, frustration, and anger, are common post-stroke disabilities. Talk therapy, along with appropriate medication, can help alleviate some of the mental and emotional problems that result from stroke. Sometimes it is beneficial for family members of the stroke patient to seek psychological help as well.

### Stroke and the Systems of the Present Invention

Experiments conducted during the development of the present invention have demonstrated that healthy as well as sick or diseased (e.g., bipolar vestibular dysfunction patients) subjects demonstrated improvement or correction of, among other things, their vestibular function (e.g., balance), proprioception, motor control, vision, posture, cognitive functions, tinnitus, emotional conditions and sleep as a direct consequence of training procedures with the systems of the present invention. Thus, the systems of the present invention benefits stroke patients in numerous ways.

In some embodiments, the present invention provides systems and treatments for correcting or improving loss of awareness, pain or numbness, the senses (e.g., seeing, touching, and balancing), motor activity, speech, perception and thinking (e.g., the ability to understand/comprehend speech), behavioral and thought patterns, chewing and swallowing food, memory (e.g., long and short term memory), and emotions in a subject displaying stroke-like symptoms.

In some embodiments, systems and methods of the present invention are used in combination with other treatments (e.g., antithrombotics including antiplatelet agents and anticoagulants, thrombolytics, and neuroprotective agents) or therapies (e.g., physical therapy, occupational therapy, speech therapy and psychological therapy) for treating a stroke subject. Thus, the present invention provides complimentary or supplementary treatments that can be used in combination with other known treatments. It is contemplated that systems and methods of the present invention intensify the positive effects of current treatments for stroke, and decrease or prevent adverse side effects. In some embodiments, use of systems and methods of the present invention permits a decrease in the dosage of a drug prescribed to treat stroke or a subject exhibiting stroke-like symptoms.

It is contemplated that as a part of stroke prevention therapy, focusing on the prevention of falls and injuries, a training regimen based on advanced physical therapy reinforced with the systems of the present invention improves posture, balance, and motor control.

Additionally, it is contemplated that as a part of long term stroke treatment, the systems of the present invention combined with a training regimen are effective in post-stroke rehabilitation, enabling stroke victims to overcome disabilities (e.g., slurred speech and other disabilities mentioned herein) that result from stroke damage.

The systems of the present invention have been shown to improve and recover postural control and gait stability in both BVD patients and normal subjects. Data recording and analytical routines are capable of quantifying postural stability, enabling the quantitative description of postural stability and the ability to control the recovery process. As such, the systems of the present invention fully correspond to the general intent of recovery of stroke patients' movement, balance, and coordination. Accordingly, in some embodiments, the present invention provides systems and treatments for correcting or improving movement, balance, and coordination in a stroke patient. In further embodiments, walking, talking, and completing simple tasks that depend on coordinated muscle movements are improved or corrected in a stroke patient.

In some embodiments, training with the systems of the present invention overcomes patient paralysis and weakness and provides and facilitates muscular relaxation in all muscular groups, (e.g., as observed in BVD patients suffering from typical rigidity in neck and upper back muscles).

In some embodiments, recovery of perceptual and sensory deficits (including loss of awareness) is reinforced with systems of the present invention (e.g., BVD patients with such deficits improved not only their balance and coordination, but also their vision, hearing and proprioception).

In some embodiments, systems of the present invention assist the amelioration of mental and emotional problems associated with stroke. For example, in some embodiments, systems and methods of the present invention improve sleep, reduce stress and depression and improve emotional status in a stroke patient. In some embodiments, training improves cognitive functions (e.g., the ability to think clearly, to remember and to act in multitasking environments). These functions are typically affected in BVD patients.

In some embodiments, the present invention provides systems and methods for reducing or correcting speech problems resulting from tongue mobility loss associated with stroke. For example, in some embodiments, the systems of the present invention are used to keep muscular tonus within normal range as a consequence of antidromic stimulation (e.g., stimulation from the tongue to the nerve center) of the hypoglossal nerve (major motor nerve of the tongue).

In some embodiments, the systems of the present invention are used to regain brain function by activating, utilizing, and/or training a portion of the brain to learn a task that was previously facilitated by a region of the brain now damaged.

A subject with a central cerebellar lesion due to stroke was treated for one week with the systems and methods of the present invention. The subject's response to treatment is documented in Table 8 below.

TABLE 8

| Test | Pre-treatments Score | Post-treatment Score |
|---|---|---|
| Neurocom SOT composite | 48 | 61 |
| Total # of falls on SOT | 3 | 0 |
| # of falls on SOT 5 and 6 | 3 | 0 |
| Dynamic Gait Index | 18/24 | 18/24 (24 best) |
| Activities-Specific Balance | 46/100 | 55/100 (100 best) |
| Confidence Scale (ABC) | | |
| Dizziness Handicap | 52/100 | 38/100 (0 best) |
| Inventory (DHI) | | |

As described in Table 8 above, the subject demonstrated improvements with the quality of life indicators (ABC, DHI), and on the SOT. Additionally, walking in crowds became significantly easier for the subject.

### Example 25

### Meniere's Disease

A subject with Meniere's disease was treated with the systems and methods of the present invention. The subject responded well to treatment. For example, post-treatment, the subject enjoyed stable, smooth and rhythmic motion in his gait, with the ability to turn with his eyes closed. The subject further enjoyed the ability to look at walls and the ceiling while he walked (e.g., down a hallway). His visual acuity improved providing the subject with the ability to change his visual focus more smoothly and without impairment or disorientation (e.g., the subject was able to change his focus from the instrument panel of a car to outside traffic and surrounding environments in a smooth, focused manner).

No adverse events were observed or reported by the subject

### Example 26

### Migraine

A subject with migraines as well as bilateral vestibular loss was treated twice a day over a period of 4½ days with the systems and methods of the present invention. The subject displayed positive results from treatment.

Prior to treatment, the subject exhibited a wide base of support in normal gait and was unable to stand in a tandem Romberg position with eyes closed or open. She was further unable to stand on one leg without falling to one side. She suffered from functional defects including daily headaches, balance difficulty, inability to walk on uneven surfaces, difficulty walking up stairs without a railing and walking in the dark. She had difficulty sleeping and driving at night. The subject suffered from an impaired ability to carry out multitasking functions. Slightly more than a year prior to treatment, the subject had a NEUROCOM test with a composite score of 55, below normal for her age group.

Post treatment with the systems and methods of the present invention, the subject enjoyed a normal base of support in gait and was able to stand with eyes open and closed in a tandem Romberg position. The subject was also able to stand on one leg without falling. She noted functional improvement including experiencing no difficulty walking up stairs, no headaches, improved sleeping, decreased difficulty with driving, improved clarity of vision, and the ability to walk on a treadmill without dizziness thereafter. She noted that her overall confidence increased. Additionally, the subject gained the ability to perform physical/mental multitask routines (e.g., walking, tossing a ball, and counting). Her composite score on the NEUROCOM test was 65, with the NEUROCOM test taking place two days after her final treatment.

### Example 27

### Mal de Debarquement

Mal de debarquement (MDD), literally “sickness of disembarkment,” refers generally to inappropriate sensations of movement after exposure to motion. For example, the syndrome (e.g., recurrence of symptoms associated with the syndrome) typically follows a sea voyage (e.g., a sea cruise), but similar sensations have been described following extended train travel, space flight (See, e.g., Stott, In: Crampton, ed. Motion and Space Sickness. Boca Raton, Fla.: CRC Press; 1990), and experience within a slowly rotating room (See, e.g., Graybiel, Aerospace Med. 1969; 40:351-367). Symptoms usually include vague unsteadiness (e.g., imbalance) and disequilibrium or sensations of rocking and swaying, and may also include tilting sensations, ear symptoms, nausea and headache. Mal de debarquement can be distinguished from motion sickness, airsickness, simulator sickness, or seasickness (e.g., mal de mer) because subjects are predominantly symptom free during the period of motion (e.g., as opposed to experiencing symptoms during the period of motion). Mal de debarquement can also be distinguished from “landsickness” or postmotion vertigo by the duration of the syndrome (e.g., the duration of the symptoms associated with the syndrome—e.g., unsteadiness or sensations of rocking and swaying). Landsickness typically lasts less than 48 hours (See, e.g., Cohen, J Vestib Res. 1996; 6:31-35; Gordon et al., J Vestib Res. 1995; 5:363-369). Most researchers reporting on MDD define it as a syndrome presenting symptoms that generally persists for at least 1 month (See, e.g., Brown et al., Am J Otolaryngol 1987; 8:219-222; Murphy, Otolaryngol Head Neck Surg. 1993; 109:10-13; Mair, J Audiol Med. 1996; 5:21-25). Others refer to the common short-lived postmotion vertigo as MDD, and the longer duration form as “persistent MDD” (See, e.g., Gordon et al., J Vestib Res. 1995; 5:363-369).

Two patients with MDD were treated over the period of one week with the systems and methods of the present invention. Prior to treatment, both patients exhaustively sought and received treatment for their symptoms, but received no benefit (e.g., no reduction of symptoms) from such treatments. The results of treatment with the systems and methods of the present invention are shown in Tables 9 and 10 below. Both patients experienced significant improvement of their symptoms after treatment (e.g., training) with the systems and methods of the present invention.

TABLE 9
Patient 1 data.

| Test | Pre-treatments Score | Post-treatment Score |
|---|---|---|
| Dynamic Gait Index | 22/24 | 24/24 |
| ABC Scale (higher = better) | 75/100 | 96/100 |
| Dizziness Handicap | 60/100 | 24/100 |
| Inventory (lower = better) | | |
| Neurocom SOT Composite | 64 | 80 |
| Total # of falls on SOT | 0 | 0 |
| # of falls on SOT 5 & 6 | 0 | 0 |

TABLE 10
Patient 2 data.

| Test | Pre-treatments Score | Post-treatment Score |
|---|---|---|
| Dynamic Gait Index | 24/24 | 24/24 |
| ABC Scale (higher = better) | 94/100 | 100/100 |
| Dizziness Handicap | 56/100 | 8/100 |
| Inventory (lower = better) | | |
| Neurocom SOT Composite | 58 | 81 |
| Total # of falls on SOT | 0 | 0 |
| # of falls on SOT 5 & 6 | 0 | 0 |

### Example 28

### Acute Vestibular Labyrinthitis

Vestibular system problems affect a person's overall balance. The vestibular system, commonly referred to as the inner ear (and described in detail above), is a series of canals filled with fluid. The fluid inside the canals detects head movement and this information is passed along to the brain via the vestibular nerve, which lies close to the ear. If the balance organs of one ear are inflamed, the information sent to the brain conflicts with the information sent from the unaffected ear. This conflict of information results in vertigo, ultimately affecting balance.

Vestibular neuritis and labyrinthitis are infections of the inner ear that cause symptoms such as dizziness, nausea atop imbalance. In some cases, these conditions resolve by themselves (e.g. within 2 to 3 weeks), and in other cases. the symptoms linger. Treatment typically includes drugs and balance exercises. any patients never fully recover. Compositions and methods of the present invention were utilized for treating a patient with an acute onset of vestibular labyrinthitis.

### Case History

The patient was a male diagnosed with vestibular labyrinthitis. The patient reported he woke up one night and the room was spinning. He was unable to stand or sit on the edge of the bed, and had to crawl on the floor to get across the room. These symptoms continued at this level of intensity for approximately 2 days. He was unable to work. For the following 2 weeks, he needed to hold on to things to walk across the room, and lean against something when standing to prevent him from falling. He also had difficulty when scanning a computer screen from left to right (due to segmented eye movement). The intensity of his symptoms decreased somewhat, then did not change for 4 more weeks.

This patient is a former high performance athlete who continues to train. His symptoms interfered not just with his daily activities, but also with his training. He experienced nausea and visual disturbances after running or biking for 30 minutes. It was difficult for him to maintain his balance and re-focus his visual attention when he turned his head (e.g., when running or biking). When biking, he was unable to train in a group for fear of falling. When swimming, he became disoriented. He felt like he was not able to walk or run in a straight line. Quick head movements caused a temporary feeling of being off balance.

### Intervention

The patient began using the systems and methods of the present invention 7 weeks after the onset of his condition. At this point, he was still having difficulty with balance after exercise (e.g., running, biking and swimming for periods of 30 or more minutes). He was instructed to use the systems and methods of the present invention twice daily for a minimum of 20 minutes per training session. He used the systems and methods either 1) standing on a couch cushion, 2) standing in tandem Romberg position, 3) standing on an Airex foam pad, or 4) when walking. He used the device for 10 weeks. There were some days he was unable to perform both training sessions due to scheduling problems.

### Results

The patient reported the following results after using systems and methods of the present invention:
Day 2: Less visual distortion when walking and able to run straighter.
Day 6: Able to bike for 30 miles and get through an entire das with no symptoms. Feels almost 100% normal. Able to move any direction with no return of symptoms. He felt that this was a “breakthrough” day. He described the results as “amazing.”
After 1 week: Able to bike, run and swim with no visual disruptions with head movement or symptoms of imbalance. Bike 45 miles followed by a 4 mile run/walk.
After 2 weeks: Able to return to group riding and train long distances without any disorientation, even when turning his head quickly, and without having symptoms return. Able to swim his full workout in an open lake without getting disoriented.
After 3 weeks: Symptom-free. Able to return to his prior level of training without having symptoms return. His training ride felt “normal” and he was even able to help push others. Able to scan a computer screen smoothly in both directions.
After 7 weeks: Partricipatedin a triathlon without any balance-related problems for the entire race.

The patient continued to use the device for 3 more weeks. The patient reported that this helped “fine tune” his system to the elite levee that he had previously attained. The patient is no longer using the present invention provides that patients with an acute onset of vestibular neuritis or labyrinthitis will benefit from using (e.g., training with and using (e.g., one or more times a day) systems and methods of the present invention. In some preferred embodiments, improvements in symptoms will occur after 1 week. In further preferred embodiments, the longer systems and methods of the present invention are used (e.g., once or more times daily) the greater the improvement.

### Example 29

Exemplary Intraoral Device System

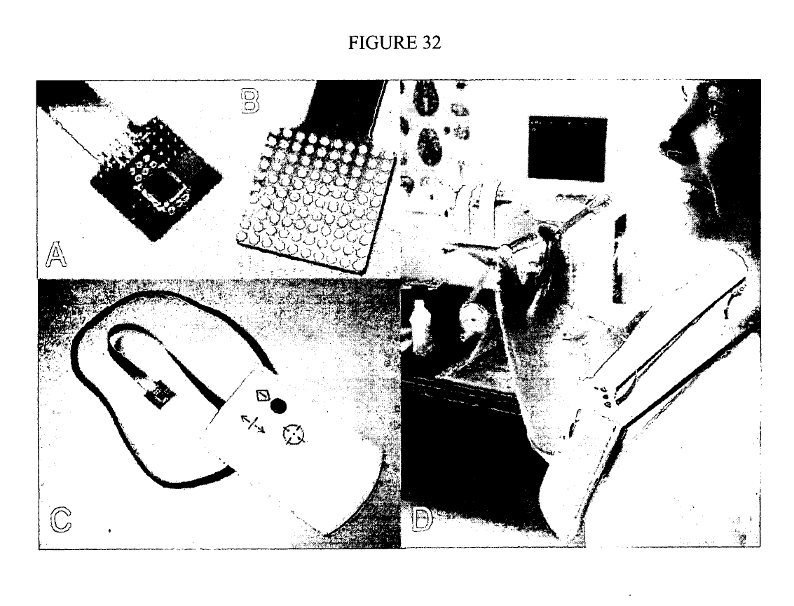

In some embodiments, the intraoral device (IOD) comprises several elements: an electrotactile array and a tether (See, e.g., FIG. 32B), and a MEMS accelerometer (See, e.g., FIG. 32A). In some embodiments, electrotactile stimuli are delivered to the dorsum of the tongue by a tactile array. The array can be fabricated using industry standard photolithographic techniques for flexible circuit technology and may employ a polyimide substrate. In some embodiments, all 100 electrodes (1.5 mm diameter, on 2.32 mm centers) on the 24 mm×24 mm array can be electroplated with a 1.5 μm thick layer of gold (See, e.g., FIG. 32B). In some embodiments, the design employs a “distributed ground” wherein the switching circuitry allows all electrodes that are not “active” (e.g., being stimulated any instant) to serve as the electrical ground for the array. This eliminates the need for discrete ground electrodes while affording a return path for the stimulation current through a 1 k-ohm resistor. Elimination of the ground plane simplifies electrode design, and allows the use of larger electrodes for increased percept quality without sacrifice of spatial resolution or dynamic range of sensation intensity. In some embodiments, the accelerometer is mounted on the superior surface of the array (away from the tongue) for sensing head position in both the anterior/posterior and medial/lateral directions. In some embodiments, this component and associated flex circuit is encapsulated in a silicone material to fix the accelerometer to the superior surface of the array and to ensure electrical isolation for the subject. The tether (e.g., 12 mm wide×2 mm thick) connects the electrotactile array and accelerometer to the Controller (See, e.g., FIGS. 32C and 32D). In some embodiments, the tether is easily detachable so that one or more subjects with their own tethers and/or mouth pieces can take turns using the same base unit. In some embodiments, most of the 109 conductors in the tether activate the tongue array electrodes, while the remaining conductors provide power and accelerometer communication data. In some embodiments, a subject (e.g., a patient being treated with systems and methods of the present invention) is able to wear the device (e.g., around the neck or waist).

### Example 30

### Systems and Methods for Vision Orientation and Mobility Aid

Experiments were conducted to demonstrate the systems and methods of the present invention with adult, legally blind subjects. Ten legally blind subjects participated in experiments comprising training and performance exercises. The group of ten consisted of four congenitally blind subjects and six subjects that had been legally blind for greater than ten years. There were three women and six men with an age range of 36-67 and a median age of 50.5 years. Each subject underwent four 2.5 hour long training sessions using the systems and methods described in Example 12.

A number of training parameters were established. These included the identification of 2D lines with varying orientations (e.g., in 4 directions), identification of orientation of the letter “C” (e.g., in 8 directions), selection of one of three balls that differs from the other two (e.g., softball, baseball, and golf ball), selection of one of three balls that is closer to the subject than the other two balls (from a softball, baseball and a golf ball), identification of and location of occupied and unoccupied seats among three chairs, and navigation and obstacle detection (e.g., hallways, lines on floor and carpeted pathways).

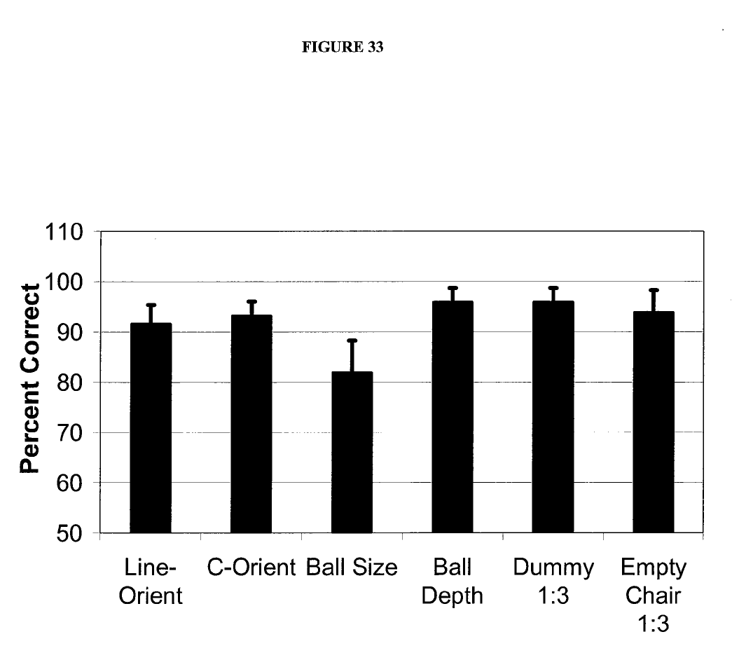

Results from performance tests are depicted in FIG. 33. Line orientation, C-orientation, ball depth, location of a dummy in a chair and location of an empty chair were all performed successfully greater than 90% of the time by the legally blind adult subjects using a sensory substitution device described in Example 12. Selection of one of the three balls that differs from the other two was also performed with a success rate of greater than 80%. All subjects were able to find and follow lines and paths. The speed at which subjects were able to find and follow the paths increased with practice using the sensory substitution device and methods of the present invention. Subjects could identify objects (e.g., towers 5 feet tall and 1.5 feet in diameter) from a distance of 10-12 feet away using the widest field of view setting on the camera. Additionally, subjects were able to orientate themselves with regard to a bicycle tire placed 8-10 feet away and walk towards the tire, and even place one foot inside the tire. Subjects described sensitivity toward variable contrast situations, could identify distinguishing features of objects, could discern extrinsic object motion and object parallax, and became familiar with depth from relative size, intensity, and shadows.

Accordingly, in some embodiments, systems and methods of the present invention (e.g., a sensory substitution device comprising a single sensor (e.g., a video camera)) can be used to provide visual information (e.g., comprising orientation, depth, object identification, motion and parallax, etc.) to adult individuals. The present invention is not limited by the type of adult individual provided visual information. In some embodiments, the adult is legally blind. In some embodiments, the adult is not legally blind. In some embodiments, the adult suffers from some visual impairment. In some embodiments, the adult suffers from vision loss associated with aging. In some embodiments, additional training with the systems and methods of the present invention provides the subject a greater ability to perceive the visual information. In some embodiments, the visual information provided by a sensory substitution system of the present invention provides a subject with improved vision or treats a vision-associated condition.

### Example 31

### Vision Substitution Utilizing Multiple Sensors

During development of the present invention, it was determined that the presentation of visual information (e.g., via coded pulse trains (e.g., via electrotactile stimulation of the tongue (e.g., of visual information translated by a processor present within a computer that detects information (e.g., energy) transduced from a sensor (e.g., video camera)))) described in the previous examples encountered some limitations. For example, subjects utilizing a device of the present invention reported a limited field of view (e.g., based upon limitations of the field of view of a single camera) and felt constrained by the lack of controllable zoom, focus, contrast inversion and contrast adjustment features. Furthermore, the previously utilized systems (e.g., in Examples 12 and 30) lacked progressive image processing/enhancement features. Thus, there existed a need to provide the ability to control some if not all of these features, as well as a need to be able to present this information to the user in a useful and constructive manner.

Accordingly, a device was generated during development of the present invention to address these needs. In general, the device was generated with user-controllable options, multiple sensors (e.g., cameras), increased information processing and presentation capabilities (e.g., via use of a mobile computer and increased array density of tongue display) and new and optimized training programs. For example, in some embodiments, the present invention provides a device comprising integrated user controls (e.g., including digital zoom and contrast adjustable functions). In additional embodiments, the present invention provides a device comprising a mobile platform for receiving, processing, storing and distributing visual information (e.g., obtained from a sensor). These and other embodiments are described in detail below.

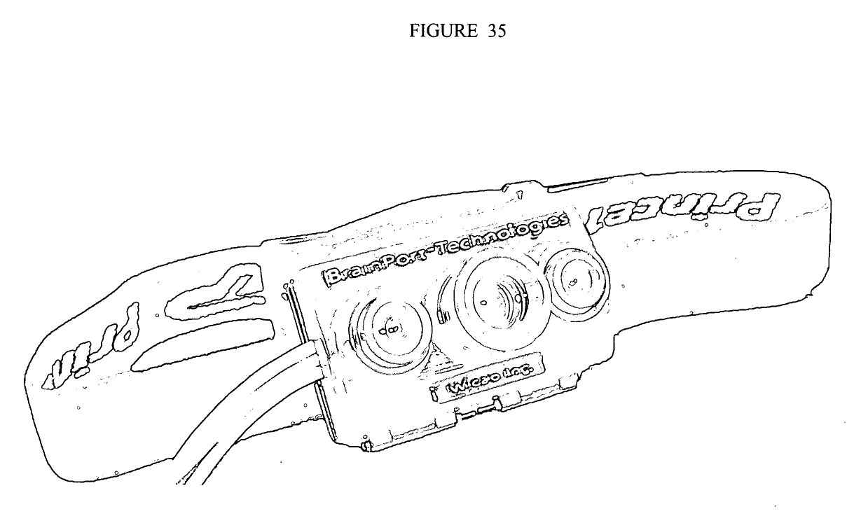

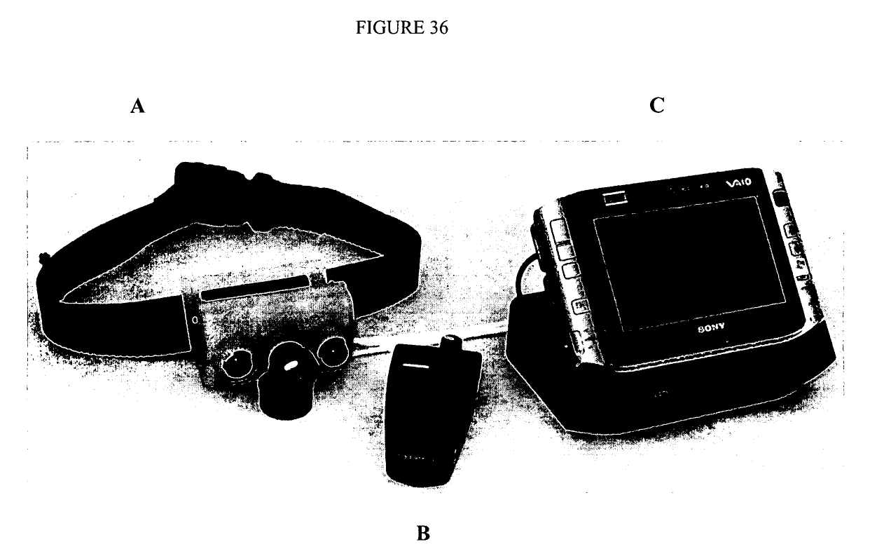

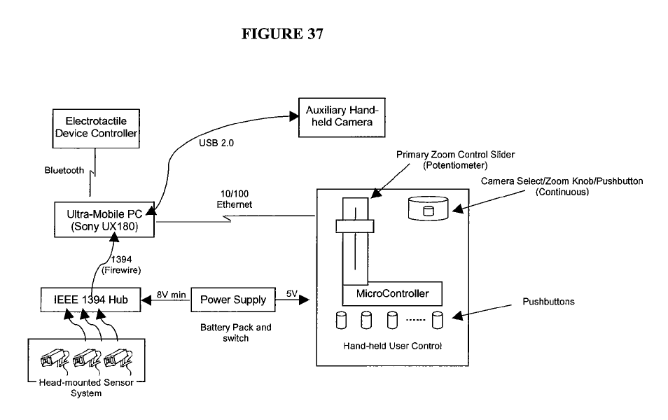

A sensory substitution device was generated to provide visual information to blind and low-vision individuals (e.g. to assist in daily living tasks (e.g., navigation, obstacle avoidance, and reading) (See, e.g., FIG. 37). However, the device may also be used by sighted individuals for any number of uses (e.g., entertainment, vision enhancement, night vision, etc.). As further described below, the present invention is not limited to the configuration shown in FIG. 37. In some embodiments, the sensory substitution device comprises a chain of high resolution sensors (e.g., a chain of three cameras (e.g., in parallel axis configuration mounted side by side (See, e.g., FIGS. 35 and 36)), integrated user controls (e.g., to control sensor (e.g., camera) zoom, focus, and/or contrast) and a portable (e.g., ultra-portable) platform (e.g., ultra-compact personal computer). In some embodiments, the sensors and user controls communicate with the platform and the platform communicates with the sensors and user controls (e.g., thereby providing an adjustable, two-way communication system). The present invention is not limited by the configuration of the sensory substitution device. In some embodiments, the cameras are directly connected to the portable platform (e.g., via a hardwire connection (e.g., IEEE 1394 FIREWIRE, USB (e.g., 1.0 or 2.0) connection, etc.). In some embodiments, the cameras communicate with the platform via wireless communication technology (e.g., via WIFI (e.g., wireless LAN and/or wireless WAN), infrared signals, BLUETOOTH, etc.). In some embodiments, the platform also communicates with a tactile device for providing visual information to the subject (e.g., an electrotactile device (e.g., comprising an array of electrodes (e.g., an intraoral device (IOD) describe herein (e.g., in Examples 1 or 29)))). In some embodiments, the communication between the electrotactile device and the platform is via a hardwire connection (e.g., IEEE 1394 FIREWIRE, USB (e.g., 1.0 or 2.0) connection, etc.). In some embodiments, the communication between the electrotactile device and the platform is via wireless communication technology (e.g., via WIFI (e.g., wireless LAN and/or wireless WAN), infrared signals, BLUETOOTH, etc.). In some embodiments, the platform also communicates with a microcontroller (e.g., for controlling sensor (e.g., camera) contrast, zoom, focus, recording, etc.). In some embodiments, the microcontroller comprises a means for controlling the zoom and/or focus and/or contrast of the camera (e.g., a slide, dimmer, potentiometer, or other type of adjustable control). In some embodiments, the microcontroller comprises a means (e.g., a switch, knob, dimmer, pushbutton, potentiometer, etc.) for selecting one or more sensors (e.g., cameras) from the chain of sensors. In some embodiments, the sensory substitution device also comprises a IEEE 1394 Hub.

In some embodiments, the sensory substitution device further comprises a power supply. The present invention is not limited by the type of power supply utilized. In some embodiments, the power supply is a rechargeable battery pack.

In some embodiments, the sensory substitution device is wearable by a user (e.g., the cameras are placed on the head (e.g., on a headband) and the other components may be placed in a belt or fanny pack). Thus, in some embodiments, the present invention provides a wearable aid for visual enhancement (WAVE). In some embodiments, the microcontroller is a hand-held user control, although any type of controller may be used (e.g., voice control, tongue control, pressure control, etc.). In some embodiments, the hand-held user control comprises a slider (e.g., for continuous zoom) and a button (e.g., invert button) separate from the controller. In some embodiments, the controller and batteries for operating the same are in the same case (e.g., within a fanny pack). In some embodiments, the microcontroller case comprises a clip (e.g., for attachment to a belt or fanny pack strap). In some embodiments, the slider is easily discernable (e.g., by touch) from the knob/button (e.g., due to shape or material texture). In some embodiments, the sensory substitution device comprises one or more buttons/knobs. In some embodiments, a button/knob can be configured to invert a perceived image. In some embodiments, a button/knob can be configured to adjust contrast. In some embodiments, a button/knob can be configured to enable and/or disable auto-gain. In some embodiments, a button/knob can be configured to select and/or de-select one or more of the sensors (e.g., cameras present in the array of three cameras).

In some embodiments, two or more sensors (e.g., video cameras, or other imaging device) can be integrated to provide one continuous image stream from a given sensor (e.g., a camera, a camera selected by the user or a camera selected automatically) or can be interlaced to provide images from different sensors (e.g., cameras) in a predefined interleaving schema.

In some embodiments, sensors may be passive or active. For example, a passive sensor may simply acquire data from the ambient environment (e.g., a video image (e.g., of topography, physical surroundings, etc.), whereas an active sensor may inject energy into the environment and acquire resulting data (e.g., an active infrared sensor may illuminate a scene with infrared light and acquire image data at the appropriate wavelength). There may be any combination of passive and active sensors. In some embodiments, a sensor (e.g., passive or active) may comprise the ability to detect a signal (e.g., type of visual information (e.g., including, but not limited to, information perceived by the eyes of a healthy individual (e.g., objects, conditions (e.g., wind, rain, fire, etc.), depth, dimension, light, movement, orientation, object identification, object parallax, etc. as well as information that can be detected using mechanical devices (e.g., cameras (e.g., heat, distance, global positioning, movement, etc.)))) within any given environment, and, together with other components of the sensory substitution device (e.g., a processor and software present within portable platform and the eletrotactile stimulation component) function to warn a subject of the presence of the signal. The present invention is not limited to any particular signal. Indeed, a variety of signals are contemplated to be detectable by a sensor of the present invention including, but not limited to, a moving object (e.g., a motor vehicle), a hazardous condition (e.g., a hole, ice, broken surface, etc.) and electronic wavelengths of information (e.g., transmitted by another user of a sensory substitution device (e.g., worn by a friendly soldier), infrared signals transmitted by the device, or by a cross-walk indicator at a traffic signal).

The present invention is not limited by the type of sensor used. In some embodiments, the sensor is a video camera. However, any active or passive data acquisition device is contemplated to be useful as a sensor. In some embodiments, a sensor comprises a device for detecting range data acquired via laser or ultrasound, or a global positioning device. For example, in some embodiments, standard camera luminance data can be couple with range data acquired via ultrasound or laser ranging. In some embodiments, GPS and/or radar can be integrated into data flow. In some embodiments, video feed (e.g., not from a sensor of the device) can be integrated into the data flow (e.g., from a television, cable, IPOD, computer or the internet (e.g., via any type of communication (e.g., USB, serial port, wireless connection, etc.).

In some embodiments, a camera used as a sensor is a digital camera. In some embodiments, the camera is a monochrome camera. In some embodiments, the camera records images in color. In some embodiments, the camera has an auto-brightness function. In some embodiments, the camera has a fixed focus lens. In some embodiments, the camera has an adjustable lens. The present invention is not limited by the type of lens utilized. Indeed, a variety of lenses may be used including, but not limited to, pinhole lenses, multi-element lenses, plastic lenses, glass lenses, removable lenses, telephoto lenses, and other types of lenses. In some embodiments, the field of view (FOV) of a camera has a maximum horizontal angle of 90 degrees. In some embodiments, the FOV maximum horizontal angle between 75-85 degrees. In some embodiments, the FOV minimum horizontal angle is 8 degrees. In some embodiments, the maximum horizontal angle is greater than 90 degrees and the minimum horizontal angle is less than 8 degrees.

In some embodiments, multiple cameras are utilized in the string of two or more sensors. In some embodiments, four cameras are utilized (e.g., permitting a user to select 1 of 4 cameras in an up/down sequence (e.g., 90 degrees, 45 degrees, 20 degrees, 8 degrees)). In some embodiments, three cameras are utilized (e.g., permitting a user to selected 1 of 3 cameras in a up/down sequence (e.g., 90 degrees, 30 degrees, 8 degrees)). In some embodiments, two cameras are utilized (e.g., permitting a user to select 1 of two cameras in an up/down sequence). In some embodiments, digital zoom is utilized to make a smooth transition from camera to camera.

In some embodiments, a three camera coaxial configuration is utilized. For example, two beam splitters may be used to create small rectangular packages of visual information. In some embodiments, the cameras are mounted side-by-side with parallel optical axes. In some embodiments, the cameras are mounted side-by-side with converging optical axes. In some embodiments, the camera nodal points are co-located. For example, with one beam splitter and two cameras, it is possible to co-locate nodal points with two cameras exactly (i.e., the path lengths from far field object point to either image plane are identical). In some embodiments, a common lens system located forward of a beam splitter is utilized.

In some embodiments, an analogue output camera is used. In some embodiments, a digital output camera is used. A camera may have a rolling shutter or a global shutter (e.g., to prevent motion induced distortion). In some embodiments, a device will be configured to have two or more cameras each with a different lens. For example, a device with three cameras each with a different lens permits a subject to perceive objects far away, close up and somewhere in between the two (e.g., by toggling between using each camera, or, by software configured to process data acquired from each camera and to present the processed information to a user). If digital cameras are utilized, one embodiment utilizes a FIREWIRE connection between the camera and the mobile platform. In some embodiments, 4 or more, 8 or more, 10 or more, 12 or more, 14 or more, or 16 or more cameras can be connected in a single wire daisy-chain connection to a FIREWIRE mobile platform interface. Thus, in some embodiments, each camera has its own lens (e.g., its own M12-0.5 lens). In some embodiments, the digital cameras are synchronized. In some embodiments, the FIREWIRE powers the digital cameras. In some embodiments, two or more analogue cameras are used (e.g., with a common digital interface to the mobile platform).

In some embodiments, the sensory substitution device further comprises a hand-held camera system with an active sensor (e.g., that can be used by a subject like a flashlight (e.g., with captured data displayed on the tongue)). In some embodiments, changing the sensor characteristics permits one to alter and/or plug into different auxiliary modules (e.g., the hand-held camera can be used for purposes separate and distinct from head-mounted cameras (e.g., for reading without need to change overall system design)).

In some embodiments, information from one or more data sources (e.g., captured by one or more sensors) can be fused (e.g., sensor fusion (e.g., using software run by the mobile platform)) to provide meta-data to the tongue. In some embodiments, raw data from multiple sources (e.g., multiple sensors) is presented to the tongue and the subject's brain provides the integration of data.

In some embodiments, sensors (e.g., cameras or other vision systems) are arranged in a configuration to provide data ranging from monocular vision to binocular vision (e.g., stereo for depth perception) or other multidimensional viewing arrangements (e.g., viewing in 2-dimensions, viewing in 3-dimensions and/or viewing in 4 dimensions). In some embodiments, data from acquisition sources (e.g., sensors) can be enhanced or otherwise modified by hardware or software algorithms in order to provide enhanced and/or additional information (e.g., in addition to data acquired by the sensor) to the subject.

In some embodiments, data presented to a subject is not limited to externally acquired data, but may also comprise pre-stored data. In some embodiments, the data is selected automatically (e.g., by a computer program (e.g., a video game or other form of programmed electronic entertainment)). In some embodiments, the data is selected by the user. The present invention is not limited by the type of pre-stored data. In some embodiments, the pre-stored data comprises informational symbol sets that provide aggregate data to the subject (e.g., that can be automatically selected based on external events (e.g., an object moving at the subject)), as well as, stored images and/or sensations (e.g., the sequence flow of which can be modified under user control (animation sequences, pre-stored pictures frames, etc)).

In some embodiments, the sensory substitution device also comprises a means for recording data acquired from one or more sensors (e.g., cameras). For example, in some embodiments, the sensory substitution device comprises a button on the handheld component that, when pressed, activates a program in the mobile platform for recording sensor acquired data (e.g., that is stored (e.g., on a hard drive or type of removable media (e.g., a DVD)) by the mobile platform). In some embodiments, the sensory substitution device is configured for a user to be able to replay recorded data at will (e.g., via a button present on the handheld component and software permitting the same). For example, a user unfamiliar with a certain setting can, after arriving in the setting and scanning the area for a period of time while recording the same, can replay the captured data stored by the mobile platform whenever he or she desires. Similarly, a user can replay any recorded data at any time for any purpose (e.g., to learn a route, to enjoy a previously experienced event, etc.).

The present invention is not limited by the type of mobile platform. Indeed, any mobile platform that can receive, process, store and distribute information associated with a substitute sensory device can be used. In some embodiments, the mobile platform is an ultra-compact personal computer (e.g., Sony Model #UX180 or similar device).

In some embodiments, the portable platform of the sensory substitution device (e.g., a wearable aid for visual enhancement (WAVE)) comprises software that monitors the operation (e.g., data input, processing and data output) of the device (See, e.g., FIG. 37). In some embodiments, the software application comprises several threads that monitor various system components and triggers events utilizing a “subscription provider” architecture (e.g., similar to that of an event listener). In some embodiments, these threads comprise a Main Application Thread, a DataStream Thread, an Electrotactile Device (e.g., Intra-Oral Device) Thread, a Hand-held Controller (HHC) Thread, a GUI Thread, and a Remote Host Thread.

Main Application Thread. In some embodiments, the main application thread configures the other threads and serves as the entry point to the application. A primary focus is initialization of the other threads and setup of subscriptions between subscribers/providers based on the aims of the specific application. In some embodiments, this functionality is configurable using .xml files.

DataStream Thread. In some embodiments, the data stream thread initializes a DataStream object comprising a set of Filters to be iterated over in a fixed order continuously passing FilterData generated by the DataSource through the filters and sending the resulting FilterData to the appropriate DataSink.

Electrotactile Device Thread. In some embodiments, the Electrotactile Device thread handles all communications with the Electrotactile Device. In some embodiments, the Thread manages a buffer containing data that is to be sent to the Electrotactile Device and subscribes to subscription providers that generate data that needs to be sent to the Electrotactile Device.

Hand-held Controller (HHC) Thread. In some embodiments, the HHC thread handles communications with the hand-held controller device. In some embodiments, this device triggers zoom adjustments and other parameter changes (e.g., contrast, focus, sensor (e.g., camera) selection, record, playback, etc.) that will be listened to by various filters.

GUI Thread. In some embodiments, the GUI thread handles updates to the GUI, responding to events from the GUI and maintenance of GUI state. In some embodiments, this thread listens to various filters and other subscription providers to display a “window” into the system.

Remote Host Thread. In some embodiments, the remote host thread handles communications with a remote host connected to the WAVE application. In some embodiments, the remote host sends messages that affect the behavior of the WAVE application.

The present invention is not limited to these particular threads. Similarly, the present invention is not limited by the functionality of any particular thread. In some embodiments, the Main Application Thread executes one or more of the following, 1-5:

1.) Instantiate a DataStream Thread and start it
a. Load a DataStream from a FilterConfig.xml file
b. When “Start( )” is called, change state to “Running” and continuously call the following on each filter:
   i. Filter->SetData(in Data);
   ii. Filter->Apply( );
   iii. outData=Filter->GetData( );
c. Feed the output of each filter to the input of the next until the end of the filter chain is reached. Once the end has been reached, start the process over again using the first filter and applying filters through to the last one in the chain.

2.) Instantiate a HHC Thread
a. Open a TCPListener to listen to requests on the HHC socket
b. Send messages to subscribers when packets are received and recognized from the HHC

3.) Instantiate a Remote Host Thread
a. Open a TCPListener to listen to requests on the Remote Host socket
b. Send/Receive messages to/from the remote host and send events to subscribers when packets are received and recognized from the remote host.

4.) Instantiate a GUI thread
a. Instantiate a DirectX surface
b. Continuously loop updating the display surface from the data in the DataStream thread as needed

5.) Instantiate a Electrotactile Device Thread
a. Open the serial port associated with the Bluetooth port
b. Monitor the serial port and wait for the ping from the Electrotactile Device
c. Maintain the status of the connection to the Electrotactile Device (connected/disconnected)
d. Subscribe to the appropriate portions of the DataStream to be notified when new images are available
e. Continuously send packets to the Electrotactile Device containing the appropriate data from the DataStream.

### Example 32

### Vision Assistance and/or Augmentation Device

Macular degeneration (MD), a progressive disease that gradually destroys the central vision, affects more than 1.75 million people in the U.S. The deteriorating retina can create one or more blind spots (e.g., a scotoma) that may eventually obscure a person's vision (e.g., in spots, centrally, peripherally, etc.). While age-related MD (AMD) is the leading cause of vision loss in people older than 60, diseases (e.g., hereditary disease (e.g., Stargardt's Disease) and other vision related disease described herein) and toxic side effects of some medications (e.g., mellaril, chloroquine) also cause vision loss (e.g., MD in younger people). Although, in some situations, peripheral vision remains unaffected, its acuity cannot fully compensate for the loss of central vision, even with assistive low-vision devices to enhance the least impaired portion of the field of vision. As described above, these assistive low-vision devices suffer from multiple limitations. For many legally blind (20/200 vision) individuals with vision loss (e.g., MD), the activities of daily living (ADL) (e.g., reading, watching TV, walking, driving, etc.) become more and more difficult if not impossible. Indeed, for most people, the quality of life plummets as vision deteriorates.

Accordingly, the present invention provides a vision assistance and/or augmentation device. A vision assistance and/or augmentation device is configured to provide a resolved (e.g., high resolution) image of a user's environment (e.g., field of view). As described below, in some embodiments, the device is configured to track a user's gaze point (e.g., thereby being configured to provide information from a certain portion of the field of view regardless of where or how a subject's eyes move). Thus, a device of the present invention is able to augment a user's existing (e.g., peripheral) vision (e.g., with a high-resolution image of the environment), rather than obscure it. As described below, a vision assistance and/or augmentation device is easily customizable and upgradeable as technology improves.

As described in Examples 12, 30 and 31, the present invention provides a device, and methods of using the same, that can be used for providing visual information to a subject (e.g., a vision device comprising two or more sensors; a handheld component comprising a microcontroller; an electrotactile device; and a mobile information gathering, processing, storing and distributing platform). The present invention also provides a vision assistance and/or augmentation device, and methods of using the same, for providing visual stimulation to a subject.

In some embodiments, a vision assistance and/or augmentation device of the present invention can transmit external sensory information to the brain by electrical stimulation of the tongue, so that it acts as a substitute sensory channel (e.g., thereby providing information (e.g., that generates and/or stimulates neuronal activation potentials) to one or more visual cortical areas). The brain can correctly interpret information from a sensory substitution device, even when the information is not presented in the same pathway as the natural sensory system. For example, the optical image actually received by the eye travels no farther than the retina, which converts the image into spatio-temporal patterns of action potentials along the optic nerve fibers. By analyzing these impulse patterns, the brain recreates the image. These impulses are not unique for vision. In fact, most sensory systems code information using the same ‘language’: neuronal action potentials. Thus, although an understanding of the mechanism is not necessary to practice the present invention, and the present invention is not limited to any particular mechanism, in some embodiments, sensory substitution requires only that action potentials be accurately entrained in the alternate sensory information channel. Accordingly, with training, the brain can learn to appropriately interpret information from the alternate channel (e.g., tactile stimulation (e.g., electrotactile, thermotactile, propriotactile, etc.)) and then process that information much as it would data from the intact natural sense.

Although not limited to any particular sensory substitution target (e.g., indeed, as described herein, multiple targets of sensory substitution are contemplated), and as described herein (e.g., in Examples 12, 30 and 31), the tongue is uniquely qualified as a sensory substitution target for electrical impulses (e.g., because of the density and sensitivity of nerve fibers in the surface of the tongue and the chemical environment of the tongue). This chemical environment of the tongue enables the tongue to readily receive and maintain electrical contacts, so that the electrical energy required and potential skin irritation at the point of contact are minimized. Van Boven et al. (See, e.g., Proc. Natl. Acad. Sci. U.S. A 102, 12601-12605 (2005)) have described spatial acuity of the fingertip of 0.8-1.0 mm. Experiments (e.g., internal two-point discrimination studies) conducted during development of embodiments of the invention have provided that a human subject can resolve two small points (each 167 μm in diameter) of electrical stimulation (500-750 μm apart) on the tongue. Accordingly, in some embodiments, the present invention provides that the resolution afforded by the tongue is around 10,000 individual points of resolution (e.g., each point is presented to the tongue by a single electrode, also referred to herein as a ‘pixel’, analogous to digital videography). In some embodiments, the present invention provides an electrode array (e.g., a 100×100, postage-stamp-sized electrode array (e.g., tongue display)) that fits on a user's tongue. In addition, the present invention also provides that the tongue can detect both time gaps of a little as 50 ms (flicker fusion) and variable intensity ‘contrast’ levels, extending the analogy to video.

A vision assistance and/or augmentation device of the present invention can be utilized to benefit a user by stimulating the tongue with information about the environment. Success does not require integrating tongue stimulation with a perception of vision. Nonetheless, in some embodiments, a user of a vision device of the present invention can perceive tactile information as eye-based vision. For example, Ramos-Estebanez et al. (J. Neurosci. 27, 4178-4181 (2007)) has reported that sighted participants who received a sub-threshold peripheral electrical stimulation to the right hand 60 ms before they received a subthreshold transcranial magnetic stimulation (providing localized neuronal stimulation) to the left primary visual cortex experienced the paired stimuli as a visual phospheme (a flash of light) in the left visual field. When the participants received either sub-threshold stimulus alone, they did not perceive touch or light. This provides that eliciting visual perception required spatial and temporal congruency between the stimuli (e.g., vision and touch), and that specific and direct pathways exist between different senses (e.g., perhaps involving a separate multi-modal area, such as the parietal cortex (See, e.g., Ramos-Estebanez et al., J. Neurosci. 27, 4178-4181 (2007))). Others have used functional magnetic resonance imaging (fMRI) to follow brain activity in blindfolded, sighted participants who received various tactile stimuli. They have found that touch stimulated the visual cortex, indicating that the visual cortex can be involved in processing tactile signals (See, e.g., Merabet et al., Neuron 42, 173-179 (2004)). Still others have found that sound could also change visual perception (See, e.g., Violentyev et al., NeuroReport 16, 1107-1110 (2005)). Thus, although a mechanism is not necessary to practice the present invention, and the invention is not limited to any particular mechanism of action, in some embodiments, the present invention provides that a user of a vision assistance and/or augmentation device receives tactile signals that can be processed by the user as visual perception.

In some embodiments, the present invention provides a device and methods for vision substitution in completely blind participants (See, e.g., Examples 12, 30 and 31). This device comprises a postage-stamp-size electrode array for the tongue (e.g., a tongue display), a control box, and a digital video camera. Visual information is collected from one or more head-mounted sensors (e.g., cameras) and sent to a controller. The controller translates the visual information into an electrical pattern that is displayed on the tongue. This device has permitted users to recognize high-contrast objects, their location, movement, and some aspects of perspective and depth. Trained blind participants have utilized the tongue display to develop a frame of reference for their environment. Moreover, users have described the experience as resembling a low-resolution version of the vision they once had. Because the camera image frame has many more pixels than the tongue display, software subsamples the digital image to create a data set that matches the electrode array. Simulation of the tongue via the electrode array is not described by users as being at all painful. For example, in some embodiments, a device emits only 11.25 μJ per pulse (e.g., the regulatory limit is 300 mJ). In fact, users often report the sensation as being like champagne bubbles effervescing on the tongue. Users report no discomfort nor a change in the feel or taste of food.

In some embodiments, the present invention provides a device for assisting and/or augmenting vision (e.g., for subjects with slight to near complete vision loss (e.g., due to disease or disorder (e.g., macular degeneration))). Such a vision device will augment, rather than replace, visual capabilities. A vision assistance and/or augmentation device can be based on a platform that meets numerous biomedical safety standards. Although the present invention provides devices and methods for vision replacement and/or substitution (e.g., See Examples 12, 30 and 31), these devices have limitations with regard to subjects that have partial to more complete vision loss (e.g., due to disease or disorder (e.g., that lead to one or more scotomas or other regions of vision loss within a users field of view (e.g., macular degeneration))). For example, a device described in Example 31 comprises a tongue display created from one or more camera images that reproduce an entire scene, not a specific area of vision loss (e.g., a scotoma or blind spot). Second, a device of Example 31 is not specifically aligned with a user's gaze point (e.g., such that the tongue display does not accurately present the image a user might wish to view (e.g., a defined by the user's gaze point (e.g., the camera moves with the user's head, not with the user's eye))). Although this feature works very well for blind individuals; individuals with residual sight rely more often on eye, rather than head, movements to explore (e.g., perceive and/or see) their surroundings.

Accordingly, the present invention provides a vision assistance and/or augmentation device (e.g., that augments a user's existing visual field (e.g., without blocking a portion of a user's existing field of view)). The device is compatible with many non-invasive vision assisting devices (e.g., eyeglasses), and will not interfere with an individual's preferred retinal location strategy. In some embodiments, a vision assistance and/or augmentation device provides an image (e.g., that is displayed on a user's tongue) that represents an image hidden by a user's scotoma, blind spot or other void in a user's field of view. In some embodiments, the vision assistance and/or augmentation device displays images on a user's tongue with electrodes (e.g., in some embodiments, each electrode is perceived as a pixel). Although pixels may not translate directly into phosphemes, blind participants using vision substitution devices of the present invention have described perceiving images on their tongues as points of light (See, e.g., Example 31). Accordingly, in some embodiments, a user of a vision assistance and/or augmentation device can, with training, learn to rely upon tactile stimulation (e.g., electrotactile stimulation (e.g., provided by a tongue display)) to provide additional visual cues (e.g., that assist and/or supplement a user's field of view). As described herein, although a mechanism is not necessary to practice the present invention, and the present invention is not limited to any particular mechanism of action, in some embodiments, a user's brain performs perceptual filling in (e.g., to merge two data sets (e.g., one or more visual and/or tactile data sets) into one recognizable visual scene (See, e.g., Komatsu, Neuroscience 7, 220-231 (2006); Violentyev et al., NeuroReport 16, 1107-1110 (2005); and Zur and Ullman, Vision Res. 43, 971-982 (2003)).

In some embodiments, a vision assistance and/or augmentation device (e.g., that augments a user's existing visual field (e.g., without blocking a portion of a user's existing field of view)) comprises a computer (e.g., portable computer, desktop computer, handheld computer, etc.) that integrates a tongue display with a commercially available eye-tracking device (e.g., a VIEW POINT PC-60 EYEFRAME SCENE CAMERA package (ARRINGTON Research, Scottsdale, Ariz.), or other type of eye tracking device (e.g., a described herein). In some embodiments, a vision assistance and/or augmentation device can acquire, store, and load a portion of a user's field of view (FOV) that corresponds to a user's FOV lost (e.g., due to disease (e.g. MD (herein termed “the region of interest” or “ROI”))) and can display the ROI to an electrode array (e.g., a 611-pixel electrode array (2.5 cm×2.5 cm) held on the tongue (the tongue display) (See, e.g., FIG. 41). The array of electrodes and the small size of the electrode array create an information rich image. To describe the display in terms of a printed image, in some embodiments, the electrode array presents an image with 25 dots per inch (DPI) resolution, although higher or lower resolution can also be achieved. The integrated eye tracking system ensures that the partial image displayed on the tongue always correlates with the lost FOV as the participant's eyes scan across the image.

The present invention is not limited by the type of eye tracking system utilized. Nonetheless, in some embodiments, the eye tracking system is, or is similar to, the VIEW POINT PC-60 EYEFRAME SCENE CAMERA package (ARRINGTON Research, Scottsdale, Ariz.). The head-mounted eye tracking system uses a micro-camera and an infra-red illumination system to follow the eye. The camera system, fixated on the user's pupil, generates eye position estimates that are used to determine the participant's gaze point. In some embodiments, the eye tracker connects to a PCI slot in a computer using a manufacturer-supplied cable and PCI card. In some embodiments, the present invention utilizes a customized eye tracker system that is configured to be a portable, wearable unit (e.g., that communicates with a computer via wireless technology). In some embodiments, software is configured to interpolate the gaze position between the updated eye-position provided by the eye-tracking system (e.g., in order to accelerate the speed at which an eye tracking system is able to determine a user's gaze point).

A vision assistance and/or augmentation device may, in some embodiments, utilize camera inputs acquired by a control computer to generate tongue stimulation patterns. For example, a vision assistance and/or augmentation system may utilize one or a plurality of cameras, with camera input acquired by a controller that is coupled with a 25 DPI electrode array (e.g., comprising an array of electrodes or pixels (e.g., 611 electrodes) that is capable of updating images presented to the tongue display (e.g., at 20-30 frames/sec). Research conducted during embodiments of the invention provide that a rate of 20-30 frames/sec provides a relatively smooth image. However, instead of presenting the entire image (e.g., as is done with a device described in Example 31), camera input is replaced by inputs (e.g., utilizing software configured to receive camera input, sample the data, and provide as input to the display), comprising only that portion of the visual field that relates to a user's vision loss (e.g., scotoma, blind spot or other type of vision loss). Software can be configured to display patterns on a LCD monitor, and to transmit only the regions identified by the gaze point directly to the vision assistance and/or augmentation device as a tongue stimulation pattern. In some embodiments, components of a vision assistance and/or augmentation device are manufactured under a quality management system that is certified to ISO 9001: 2000 and ISO 13485: 2003.

The eye tracking system reports the (x, y) coordinates of a user's gaze point to the computer. Software can then combine this information and a user's scotoma map to display an ‘image’ of the participant's ROI scaled to fit on the electrode array on the tongue (tongue display). In some embodiments, the tongue display displays black and white luminents by translating the hue into varying levels of stimulation intensity (from 0 to 25 V) on the tongue. As described herein, these type of stimulation parameters do not cause pain or injury to users. Software can be configured to update images presented to the tongue display at 20-30 frames/sec, so that any changes to the tongue-displayed image will match the eye's movements (e.g., even as the eyes track across a scene (e.g., a LCD monitor). As a comparison, movies generally run at 24 frames/sec; at that speed, the eye detects smooth movement. Thus, core integration software can be designed with functionality to: 1) load and store a user's scotoma maps used to create a custom ROI mask for each participant; 2) interface and interact with eye-tracking software for calibration and run-time eye-tracking; 3) create and display images on a LCD screen for participant training and testing; 4) determine participant gaze point based on eye-tracking data; 5) extract ROI data from the displayed image; 6) generate tongue stimulation patterns based on the ROI data and deliver those patterns to the tongue display; and/or 7) provide a user interface to enable a third party (e.g., a researcher) to direct the software, configure and conduct experiments, and collect and store data. A vision assistance and/or augmentation device can be configured to use standard electrical and communication interfaces. Hardware integration can include mechanical and electrical designs to ensure safe, ergonomic connections between the equipment and the participant and between individual components. A vision assistance and/or augmentation device can be configured to calibrate in only a few minutes (e.g., an eye tracker can be calibrated with a user's central gaze using the manufacturer's calibration software).

Integration software can be configured to appropriately scale each user's scotoma map to create a personalized ROI that a controller can display on the tongue. In some embodiments, the ROI can be configured to encompass a slightly larger portion of the FOV than the scotoma map (e.g., to account for any imprecision). Redundant (e.g., overlapping) information may help users explicitly and/or implicitly connect multiple sensory modalities (e.g., sight and touch). In some embodiments, a scotoma map is generated for each eye. In some embodiments, a user may require a customized tongue display beyond clinical scotoma map integration (e.g., a tongue display may need to be personalized (e.g., meaning that components of a vision assistance and/or augmentation device may require a prescription)).

In some embodiments, a user of a vision assistance and/or augmentation device will have a scotoma map performed prior to using the device. For example, a SITA-24 visual field test (See, e.g., FIG. 42) can be used. This diagnostic test maps areas of preserved and compromised visual sensitivity (e.g., central vision) by presenting flashes of light around the central 24 degrees of vision (e.g., the macula) to a subject. The subject is instructed to respond upon seeing a light flash. In this way, a unique scotoma map can be created for each user, with the map being incorporated into the integration software to create a personalized ROI.

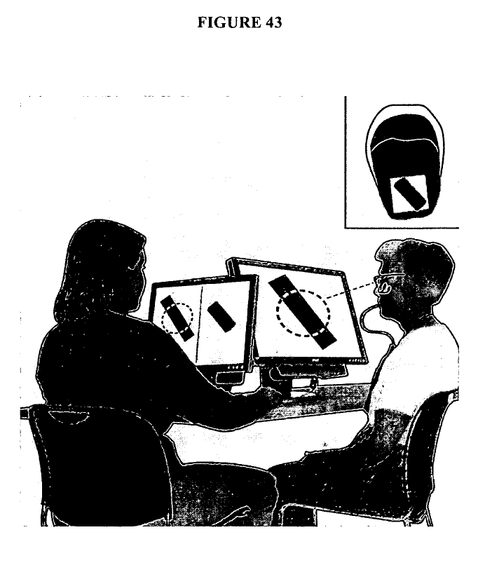

In some embodiments, a vision assistance and/or augmentation device can be used in training and/or testing methods. For example, a user can be seated in a fixed chair placed two feet from the center of a fixed monitor (See, e.g., FIG. 43). User will be instructed to sit back in the chair and to move their eyes, not their heads, to ensure that each user's central gaze continues to match the ROI, and remains constant within and between a training and/or testing session. Head movement (e.g., forwards or backwards) can change the aspect ratio (the relative sizes of the ROI displayed on the LCD monitor and the participant's central gaze), because the user will not have a camera attached to the head or eyeglasses (e.g., versus other embodiments described herein where a camera can be mounted to a user's eyeglasses (e.g., so head movement does not affect the aspect ratio)). Calibrating can be performed using the ROI with the user's central gaze before each test and monitoring the user's movements reduces aspect ratio errors. FIG. 43 shows a schematic of a training and/or testing setup. The inset of FIG. 43 provides a schematic of an image that can be presented to the tongue display.

Successful results obtained in Example 31 stimulated the interest to explore whether this technology could also benefit individuals with low or reduced vision (e.g., individuals with MD). Studies and data generated during development of the present invention training older adults with MD to use a vision-assisting device have yielded promising results. However, as described above, data indicated that the device of Example 31 required modifications to meet the specific needs of user's harboring residual, but less than total, vision capabilities (e.g., the need to track a user's gaze point (e.g., need to track a user's field of view and provide a subset of the field of view to a user (e.g., via a tongue display))). No adverse effects have been observed to date during or after use and training with devices. All participants, young or old, could localize and feel sensation on their tongue and could track the stimulation across space; age was not a barrier.

Individuals with MD have used a vision system developed for those who have no usable vision (e.g., described in Example 31). In general, participants with MD have encountered somewhat more difficulty and have required somewhat more training and time to become accustomed to using a vision device than have blind users. For example, the information displayed on the tongue was often confusing. Although an understanding of the mechanism is not necessary to practice the invention, and the invention is not limited to any particular mechanism of action, in some embodiments, a user's confusion is attributable to the mismatch between the FOV from the head-mounted camera (e.g., displayed on the tongue) and the FOV from the user's remaining vision (e.g., the eye moving in its orbit).

In order to test this hypothesis, participants were asked to close their eyes during training and testing. Although performance improved, this modification eliminated the important skill of residual vision. Thus, as described above, the device of Example 31 can be reconfigured in order to create a functionally viable device for individuals with low to almost complete vision loss (e.g., subjects with MD).

One subject was clinically tested pre- and post-training/testing. Interestingly, that participant reported an improvement in his quality of life (QOL) on nine questions in the NEI visual functioning questionnaire-25 (NEI VFQ-25). Despite the QOL improvement, the participant did not show measurable improvements in his vision with the device. This finding provides that a vision device of the present invention can improve the well-being and quality of life of individuals with MD.

Training included line orientation and the FrACT test. Line orientation provides early training, and the FrACT test involves a simplified letter recognition task, a highly useful and relevant application for people with MD. The participants were trained and tested for varying lengths of time. A participant with Stargardt's Disease (his bilateral acuity measured 20/400) was trained on a vision system described in Example 31 and qualitatively tested. Initially, the participant could not visually perceive the face of the monitor. Once the vision system was in place, however, he kept his eyes fixed on the target (so he could not perceive the monitor), and swept his head back and forth to have the monitor image displayed on his tongue. Within seconds, he correctly aimed the camera at the target and could identify the orientation of a line displayed on the tongue. The subject then identified moving bars presented in all directions. Although the subject correctly identified the direction of movement, he had some initial difficulty correlating the direction of movement displayed on the tongue with that displayed on the screen. In a rotating ‘C’ test, the subject correctly identified the location of the opening in 6 of 7 trials.

Another subject with wet AMD in one eye and dry AMD in the other with an overall acuity of 20/200 was also tested. H is central scotoma prevents him from seeing people's faces clearly, even when using his preferred retinal location. He uses multiple vision-assisting devices (each for different purposes), but has found that some do not help at all. When blindfolded, the participant could identify line orientations and report on the direction of a rotated C. Interestingly, he tended to place the target object only inches from the camera on the vision system (e.g., it appeared as if he was transferring the skills and expectations from his other assistive devices to the new technology).

Another subject tested has wet AMD with bilateral 20/200 vision. She spent 2 hours exploring the vision system and could describe line orientations and discriminate between shapes, such as a square and a circle. The subject found the rotating ‘C’ test difficult at first, but after an additional 2 hours of training, she could successfully perform the task. The subject considered the exploratory training session a game or puzzle and worked to master the system. She kept her eyes closed, because she could see the lines presented with her peripheral vision.

Yet another subject tested had wet AMD in both eyes and his uncorrected vision measured 20/400 in both eyes. Nonetheless, the subject functions well with his remaining vision. He travels by bus and can read some materials with the aid of a hand-held magnifier. The subject explored the vision system for 3 hours. The subject could immediately localize and accurately describe the stimulation patterns on his tongue. Further, the subject could identify line orientations (horizontal, vertical or diagonal), although he occasionally erred in identifying spatial orientation (left/right and up/down), although previous experiments have shown that such errors are generally reduced with further practice. The subject considered the tongue display very understandable, “as if someone was drawing the shape on my tongue.”

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields, are intended to be within the scope of the following claims.

We claim:

1. A sensory substitution device for providing visual information to a subject comprising:
a) a sensor;
b) a portable microcontroller;
c) a device configured for electrical stimulation; and
d) a mobile information gathering, processing, storing and distributing platform.

2. The sensory substitution device of claim 1, wherein said device is configured to provide video information captured by said sensor to a subject.

3. The sensory substitution device of claim 1, wherein said sensor comprises a video camera.

4. The sensory substitution device of claim 1, wherein said portable microcontroller comprises means for controlling said sensor.

5. The sensory substitution device of claim 4, wherein said means for controlling said sensor controls a sensor function selected from the group consisting of zoom, contrast, focus and inversion.

6. The sensory substitution device of claim 1, wherein said sensor communicates with said mobile platform.

7. The sensory substitution device of claim 1, wherein said device configured for electrical stimulation comprises an array of electrodes.

8. The sensory substitution device of claim 7, wherein said array of electrodes are present on an intra-oral tongue display unit.

9. The sensory substitution device of claim 7, wherein said array of electrodes is configured to provide visual information to a subject via said subject's tongue.

10. The sensory substitution device of claim 1, further comprising a power supply.

11. The sensory substitution device of claim 10, wherein said power supply comprises a battery pack.

12. The sensory substitution device of claim 1, wherein said sensor is utilized for infrared vision.

13. The sensory substitution device of claim 1, wherein said sensor is utilized for ultraviolet vision.

14. The sensory substitution device of claim 1, wherein said device creates a multidimensional electrical image on a subject's tongue.

15. A method of providing visual information to a subject comprising:
a) providing:
1) a subject; and
2) a sensory substitution device, wherein said sensory substitution device comprises:
a) a sensor;
b) a portable microcontroller;
c) a device configured for electrical stimulation; and
d) a mobile information gathering, processing, storing and distributing platform; and
b) exposing said subject to said sensory substitution device under conditions such that said subject receives visual information from said sensory substitution device.

16. The method of claim 15, wherein said visual information is real-time information regarding said subject's immediate surroundings.

17. The method of claim 15, wherein said visual information is recorded information.

18. The method of claim 15, wherein said subject is legally blind.

19. The method of claim 15, wherein said subject is visually impaired.

20. The method of claim 15, wherein said visual information is received from said device configured for electrical stimulation.

21. The method of claim 20, wherein said device configured for electrical stimulation is an array of electrodes.

22. The method of claim 21, wherein said array of electrodes provide visual information to said subject via said subject's tongue.

23. The method of claim 22, wherein said visual information comprises information captured by said sensor that is processed by said mobile platform.

24. A method of providing visual information to a visually healthy subject desiring visual stimulation, comprising:
a) providing:
1) a subject; and
2) a sensory substitution device, wherein said sensory substitution device comprises:
a) a device configured for electrical stimulation; and
b) a mobile information gathering, processing, storing and distributing platform; and
b) exposing said subject to said sensory substitution device under conditions such that said subject receives visual information from said sensory substitution device.

25. The method of claim 24, wherein said subject desires visual stimulation associated with a video game.

26. The method of claim 24, wherein said visual information is received from said device configured for electrical stimulation.

27. The method of claim 26, wherein said device configured for electrical stimulation is an array of electrodes.

28. The method of claim 27, wherein said array of electrodes provide visual information to said subject via said subject's tongue.

29. The method of claim 28, wherein said visual information comprises recorded information present within video game software.

30. The method of claim 28, wherein said subject perceives visual information that is not viewed by said subject's eyes.

31. A method of providing visual information to a subject, wherein said subject is legally blind, comprising:
a) providing:
1) a subject; and
2) a sensory substitution device, wherein said sensory substitution device comprises:
a) a sensor;
b) a portable microcontroller;
c) a device configured for electrical stimulation; and
d) a mobile information gathering, processing, storing and distributing platform; and
b) exposing said subject to said sensory substitution device under conditions such that said subject receives visual information from said sensory substitution device.

32. The method of claim 31, wherein said subject is able to visualize, using said system, images in space.

33. The method of claim 31, wherein said subject is able to perform a coordination task selected from the group consisting of facial recognition, determine speed of a moving object, determine direction of movement of an object, and hitting a moving object.

34. The method of claim 31, wherein said visual information is received from said device configured for electrical stimulation.

35. The method of claim 34, wherein said device configured for electrical stimulation is an array of electrodes.

36. The method of claim 35, wherein said array of electrodes provide visual information to said subject via said subject's tongue.

37. The method of claim 36, wherein said visual information comprises information captured by said sensor that is processed by said mobile platform.

38. The method of claim 37, wherein said sensor is a video camera.
